(19) United States
(12) Patent Application Publication
(10) Pub. No.: US 2025/0146030 A1
(43) Pub. Date: May 8, 2025

(54) METHOD FOR PRODUCING DRIMANYL ACETATE COMPOUNDS

(71) Applicant: Firmenich SA, Satigny (CH)

(72) Inventors: Daniel SOLIS ESCALANTE, Satigny (CH); Christian GÖRNER, Satigny (CH); Laurent DAVIET, Satigny (CH); Qi WANG, Shanghai (CN)

(21) Appl. No.: 19/006,763

(22) Filed: Dec. 31, 2024

Related U.S. Application Data
(62) Division of application No. 17/271,474, filed on Feb. 25, 2021, now Pat. No. 12,252,727.

(30) Foreign Application Priority Data
Oct. 15, 2018 (EP) 18200539.7

(51) Int. Cl. C12P 7/62; C12P 7/02

(52) U.S. Cl. CPC C12P 7/62; C12P 7/02

(57) ABSTRACT

Described herein are methods for the acetyl transferase-catalyzed production of drimanyl-acetate compounds by the acetylation of the respective drimanyl alcohol sources performed in vitro or in vivo. Also described herein is the identification of enzymes having corresponding acetyl transferase activity from different microbial and plant sources. Also described herein is the provision of enzyme mutants derived from the newly identified enzymes. Further described herein is the provision of corresponding coding sequences of such enzymes and mutants, recombinant vectors, and recombinant host cells suitable for the production of such acetyl transferases and mutants and for performing the novel production methods of drimanyl acetate compounds. Still further described herein is a method of using such drimanyl acetates as intermediates for the production of odorant, flavor or fragrance or insect/pest control ingredients.

### CROSS-REFERENCE TO RELATED APPLICATIONS

This application is a divisional application of U.S. patent application Ser. No. 17/271,474, filed Feb. 25, 2021, which is a U.S. National Phase Application of International Patent Application No. PCT/EP19/77716, filed Oct. 14, 2019, which claims the benefit of priority to European Patent Application No. 18200539.7, filed Oct. 15, 2018, the entire contents of which are hereby incorporated by reference herein.

### REFERENCE TO AN ELECTRONIC SEQUENCE LISTING

The contents of the electronic sequence listing (sequence_listing_36803-448 DIV.xml; Size: 345,960 bytes; and Date of Creation: Dec. 20, 2024) is herein incorporated by reference in its entirety.

### TECHNICAL FIELD

The present invention provides novel methods for the acetyl transferase-catalyzed production of drimanyl-acetate compounds by the acetylation of the respective drimanyl alcohol sources performed in vitro or in vivo. The present invention also relates to the identification of enzymes having corresponding acetyl transferase activity from different microbial and plant sources. The present invention also relates to the provision of enzyme mutants derived from said newly identified enzymes. A further aspect of the present invention relates to the provision of corresponding coding sequences of such enzymes and mutants, recombinant vectors, and recombinant host cells suitable for the production of such acetyl transferases and mutants and for performing the novel production methods of drimanyl acetate compounds. Another aspect of the invention relates to the use of such drimanyl acetates, as obtained according to the present invention, as intermediates for the production of odorant, flavor or fragrance or insect/pest control ingredients.

### BACKGROUND

Terpenes are found in most organisms (microorganisms, animals and plants). These compounds are made up of five carbon units called isoprene units and are classified by the number of these units present in their structure, which may comprise cyclic structural elements. Thus monoterpenes, sesquiterpenes and diterpenes are terpenes containing 10, 15 and 20 carbon atoms, respectively. Sesquiterpenes, for example, are widely found in the plant kingdom. Many sesquiterpene molecules are known for their flavor and fragrance properties and their cosmetic, medicinal and antimicrobial effects. Numerous sesquiterpene hydrocarbons and sesquiterpenoids have been identified. Chemical synthesis approaches have been developed but are still complex and not always cost-effective.

Biosynthetic production of terpenes involves enzymes called terpene synthases. There are numerous sesquiterpene synthases present in the plant kingdom, all using the same substrate (farnesyl diphosphate, FPP), but having different product profiles. Genes and cDNAs encoding sesquiterpene synthases have been cloned and the corresponding recombinant enzymes characterized.

Many of the main sources for sesquiterpenes, for example compounds with a drimane structure, like drimanyl alcohols, in particular albicanol or drimenol, are plants or microorganisms naturally containing the sesquiterpene; however, the content of sesquiterpenes in these natural sources can be low. Even if available, such drimanyl alcohols are difficult to handle during their further processing, mainly in view of the fact that they are solid at ambient temperature. Providing easier to handle derivatives would be an improved approach and simplify the further processing of drimanyl alcohols.

Akita, H. et al describe in Tetrahedron: Asymmetry 11 (2000). 1375-1388 the lipase catalyzed asymmetric synthesis of (+) albicanyl acetate. This prior art approach requires at least eight steps of chemical synthesis and two sequential reactions catalyzed by lipases to obtain an enantiomerically pure albicanyl acetate. In addition, the acetylation reactions occurred in the presence of diisopropyl ether and isopropenyl acetate at 33° C., conditions not compatible with the physiological conditions required for in a biosynthetic route. Moreover, under physiological conditions, lipases, to the best of our knowledge, are incapable to catalyze the required transesterification reaction. Although ester formation by lipases is possible, this activity is strongly dependent on the content of water present. Thus, in aqueous environments such as the in vivo setting of the present invention, lipases catalyse the hydrolysis of ester bonds rather than acetylation (Jaeger K. et al; FEMS Microbiology Reviews, 1994, 15:1 pp 29-63).

There still remains a need for the provision of novel methods of producing drimanyl acetate compounds, in particular methods which may be implemented into the fully biochemical synthesis of drimanyl acetates in an aqueous environment, as for example in a host cell-based process, which provides the drimanyl alcohol precursor by metabolization of sugar substrate.

### SUMMARY

The above-mentioned problem could be solved by providing a new class of enzymes which show acetyl transferase activity and produce a drimanyl acetate, like albicanyl acetate or drimenyl acetate, from the respective drimane alcohol precursor, like albicanol or drimenol via acetylation and using acetyl-CoA as acetyl group donor. Due to their physicochemical properties, in particular as they are liquid at ambient temperature, acetylated derivatives of drimanyl alcohols could serve as more appropriate materials.

### DESCRIPTION OF THE DRAWINGS

FIG. 1A: Structure of (+)-albicanol, (−)-drimenol and bicyclofarnesol, as well as of a drimane moiety more specific than the drimane structure of FIG. 1C.

FIG. 1B: Reaction scheme illustrating the cellular biological production of albicanyl acetate via the acyclic sesquiterpene precursor FPP and albicanol.

FIG. 1C: More generic “drimane structure”, the potential positions of a C═C-double are indicated.

FIG. 2A: GC-FID analysis of albicanyl acetate produced using the modified S. cerevisiae strain YST069 co-expressing the albicanol synthase XP_007369631.1 with the acetyltransferase CrDAT.

FIG. 2B: GC-FID analysis of albicanyl acetate produced using the modified S. cerevisiae strain YST069 co-expressing the albicanol synthase XP_007369631.1 with the acetyltransferase FgaAT.

FIG. 2C: The MS spectra of the produced albicanyl acetate by the acetyltransferase CrDAT from FIG. 2A. It is identical to the MS spectra from an albicanyl acetate standard.

FIG. 2D: The MS spectra of an albicanyl acetate standard.

FIG. 3: Relative quantity of albicanyl acetate produced by the nine acetyltransferases (CrDAT, FgaAT, OAH94415.1, TcTAT, CrMAT, LiAAT-4, GAO81666.1, CfACT1-6 and CfACT1-8), which were found to be active on albicanol (as described in example 2).

FIG. 4: Relative quantity of drimenyl acetate produced by the nine acetyltransferases (CrDAT, FgaAT, OAH94415.1, TcTAT, CrMAT, LiAAT-4, GAO81666.1, CfACT1-6 and CfACT1-8), which were found to be also active on albicanol (as described in example 2).

FIG. 5: Relative quantities of albicanyl acetate produced by S. cerevisiae cells expressing each of the acetyltransferases: CrDAT, FgaAT, OAH94415.1, TcTAT, CrMAT, LiAAT-4, GAO81666.1, CfACT1-6, CfACT1-8, ERR364415-1_contig_8546 and DfATC13.

FIG. 6: Relative quantities of drimenyl acetate produced by S. cerevisiae cells expressing each of the acetyltransferases: CrDAT, FgaAT, OAH94415.1, TcTAT, GAO81666.1, CfACT1-6, CfACT1-8, XP_001258079.1, ERR364415-1_contig_8546 and DfATC13.

FIG. 7A: The MS spectra of the produced drimenyl acetate by the acetyltransferase CrDAT. It is identical to the MS spectra from a drimenyl acetate standard as shown in FIG. 7B.

FIG. 7B: The MS spectra of a drimenyl acetate standard.

FIG. 8A: The MS spectra of the produced bicyclofarnesyl acetate by the acetyltransferase CrDAT. It is identical to the MS spectra from a bicyclofarnesyl acetate standard as shown in FIG. 8B.

FIG. 8B: The MS spectra of a bicyclofarnesyl acetate standard.

FIG. 9: Relative quantities of bicyclofarnesyl acetate produced by S. cerevisiae cells expressing each of the acetyltransferases: CrDAT, FgaAT, TcTAT, CrMAT, GAO81666.1, CfACT1-6, CfACT1-8, BAU61551.1, PsSalAT, XP_001217250.1, ERR364415-1_contig_8546, PYI04555.1 and DfACT13.

### ABBREVIATIONS USED

bp base pair
kb kilo base
CoA Coenzyme A
DNA deoxyribonucleic acid
cDNA complementary DNA
DTT dithiothreitol
FPP farnesyl diphosphate
GC gas chromatograph
MS mass spectrometer/mass spectrometry
MVA mevalonic acid
PCR polymerase chain reaction
RNA ribonucleic acid
mRNA messenger ribonucleic acid
miRNA micro RNA
siRNA small interfering RNA
rRNA ribosomal RNA
tRNA transfer RNA

### Definitions

Unless otherwise stated the following definitions of technical terms shall apply:

An “acetyl transferase” or “polypeptide having acetyl transferase activity” or “polypeptide capable of transferring an acetyl group” for the purpose of this invention refers more generally to an enzyme of the class of acyl transferases E.C. 2.3.1, and in particular to an acetyl-CoA: alcohol O-acetyl transferase E.C. 2.3.1.84. It shows the capability of acetylating at least one drimanyl alcohol, selected from albicanol, drimenol and bicyclofarnesol with acetyl-CoA as the acetyl group donor. The drimanyl acetate(s) may be produced in the form of any of its stereoisomers or as a mixture thereof. Albicanyl acetate, drimenyl acetate or bicyclofarnesyl acetate may be the only product if the corresponding alcohol precursor is present as the single acetyl-group acceptor or may be part of a mixture of two or more drimanyl acetates if a mixture of two or more drimanyl alcohols is provided and the acetyl transferase is not substrate specific. In case of increased selectivity the acetyl transferase may predominantly form one single drimanyl acetate. Acetyl transferases as described herein may show identical or different preference or specificity to different drimanyl alcohols as substrate. For example, a first type of acetyl transferases may predominantly acetylate albicanol, a second type of acetyl transferases may predominantly acetylate drimenol and a third type of acetyl transferases May predominantly acetylate bicyclofarnesol. In such cases albicanol acetate, drimenol acetate or bicyclofarnesyl acetate would be formed as the respective main product, if a mixture of such drimanyl alcohols is used as substrate. In case of substrate specificity the acetyl transferase may selectively form one single drimanyl acetate, even if a mixture of such drimanyl alcohols would be used as substrate. In particular, the acetylation is performed under retention of the respective stereochemichal configuration of the drimanyl alcohol substrate.

An “acetyl group donor” refers to a chemical entity or molecule which acts as a source for an acetyl group to be enzymatically transferred from said donor to an acceptor molecule, like a molecule having a functional hydroxyl group, which in turn may react with said acetyl group to form the corresponding acetic acid ester. A particular acetyl group donor is acetyl-Coenzyme A (acetyl-CoA).

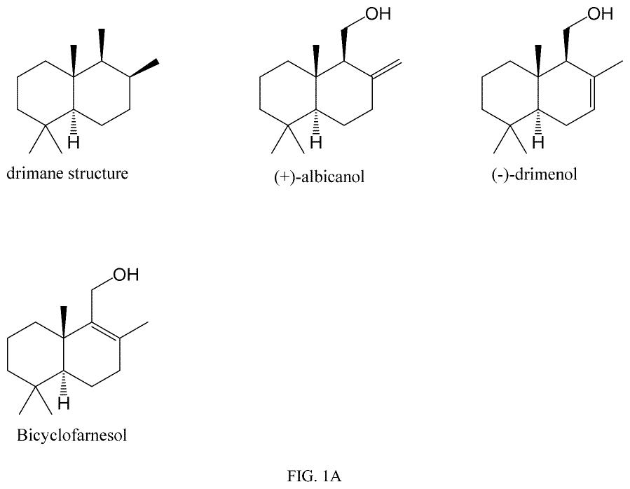

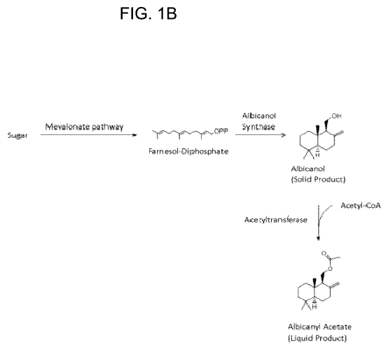

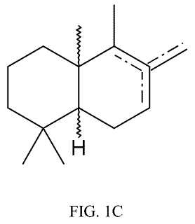

The term “drimane sesquiterpene” or “drimane” relates to a cyclic terpene having a drimane-like carbon skeleton structure as depicted in FIG. 1A, or more particularly to the more generic structure of FIG. 1C, wherein the potential positions of an optionally present C═C-double bond are marked with dotted lines.

The term “drimanyl alcohol” relates to hydroxylated derivative of a “drimane sesquiterpene” or “drimane”. Examples thereof are albicanol, drimenol and bicyclofarnesol in any stereoisomeric form.

The term “drimanyl acetate” relates to the acetyl ester derivative of such drimanyl alcohol, like albicanyl acetate, drimenyl acetate, and bicyclofarnesyl acetate.

“Albicanol” for the purpose of this application particularly relates to (+)-albicanol (CAS: 54632 April 1).

“Drimenol” for purposes of this application particularly relates to (−)-drimenol (CAS: 468-68-8).

“Bicyclofarnesol” for purposes of this application particularly relates to (+)-bicyclofarnesol or [(4aS,8aS)-2,5,5,8a-Tetramethyl-3,4,4a,5,6,7,8,8a-octahydro-1-naphthalenyl]methanol (IUPAC Name)

“Farnesyl diphosphate” refers to (2E,6E)-3,7,11-trimethyldodeca-2,6,10-triene-1-pyrophosphate (FPP).

“Ambrox” for purposes of this application relates to IUPAC Name: (−)-(3aR,5aS,9aS,9bR)-3a,6,6,9a-tetramethyldodecahydronaphto [2,1-b]furan (CAS: 6790-58-5).

The terms “terpene synthase” or “sesquiterpene synthase” or “drimane sesquiterpene synthase” are used herein interchangeably.

The terms “bifunctional terpene synthase” or “polypeptide having bifunctional terpene synthase activity” relate to a polypeptide as further defined in PCT/EP2018/064344, filed May 31, 2018.

The terms “albicanyl diphosphate synthase” or “polypeptide having albicanyl diphosphate synthase activity” or “albicanyl diphosphate synthase protein” or “having the ability to produce albicanyl diphosphate” relate to a polypeptide capable of catalyzing the synthesis of albicanyl diphosphate, in the form of any of its stereoisomers or a mixture thereof, starting from an acyclic terpene pyrophosphate, particularly farnesyl diphosphate (FPP). Albicanyl diphosphate may be the only product or may be part of a mixture of sesquiterpenes. Said mixture may comprise albicanyl monophosphate and/or albicanol. Such polypeptides are described for example in PCT/CN2018/088902, filed May 29, 2018.

“Albicanyl diphosphate synthase activity” is determined under “standard conditions” as described in PCT/CN2018/088902.

The terms “albicanol synthase” or “polypeptide having albicanol synthase activity” or “albicanol synthase protein” relate to a polypeptide capable of catalyzing the synthesis of albicanol, in the form of any of its stereoisomers or a mixture thereof, starting from an acyclic terpene pyrophosphate, particularly farnesyl diphosphate (FPP). Albicanol may be the only product or may be part of a mixture of two or more sesquiterpenes.

The terms “drimenol synthase” or “polypeptide having a drimenol synthase activity” or “drimenol synthase protein” relate to a polypeptide capable of catalyzing the synthesis of drimenol, in the form of any of its stereoisomers or a mixture thereof, starting from an acyclic terpene pyrophosphate, particularly farnesyl diphosphate (FPP). Drimenol may be the only product or may be part of a mixture of two or more sesquiterpenes.

The “albicanol synthase activity” and the drimenol synthase activity are determined as described for example in PCT/EP2018/064344, WO2015/169871 or WO 2015/176959.

A “phosphatase” enzyme as used in the present invention has the ability to convert an orthophosphoric ester under consumption of water into the respective alcohol and orthophosphate. Herein encompassed are acidic phosphatases (EC.3.1.3.2 with acidic reaction optimum) and alkaline phosphatases (EC.3.1.3.1 with alkaline reaction optimum).

The terms “biological function,” “function,” “biological activity” or “activity” refer to the ability of the terpene synthase as described herein a) to catalyze the formation of albicanyl diphosphate and/or albicanol or a mixture of compounds comprising albicanyl diphosphate, and/or albicanyl monophosphate and/or albicanol and/or and one or more other terpenes, in particular albicanyl diphosphate; or b) to catalyze the formation of a drimanyl alcohol or a mixture of two or more drimanyl alcohols and optionally one or more other terpenes.

The terms “biological function”, “function”, “biological activity” or “activity” refer to the ability of an acetyl transferase as described herein, to catalyze the formation of a drimanyl acetate or a mixture of two or more drimanyl acetates and optionally one or more other acetylated compounds.

The terms “mixture of terpenes” or “mixture of sesquiterpenes” refer to a mixture of terpenes or of sesquiterpenes that comprises at least one of albicanol, drimenol and bicyclofarnesol, and may also comprise one or more additional terpenes and/or one or more additional sesquiterpenes.

The “mevalonate pathway” also known as the “isoprenoid pathway” or “HMG-COA reductase pathway” is an essential metabolic pathway present in eukaryotes, archaea, and some bacteria. The mevalonate pathway begins with acetyl-CoA and produces two five-carbon building blocks called isopentenyl pyrophosphate (IPP) and dimethylallyl pyrophosphate (DMAPP). Key enzymes are acetoacetyl-CoA thiolase, HMG-COA synthase, HMG-COA reductase, mevalonate kinase, phosphomevalonate kinase, a mevalonate diphosphate decarboxylase, and an isopentenyl diphosphate isomerase. Combining the mevalonate pathway with enzyme activity to generate the terpene precursors GPP, FPP or GGPP, like in particular FPP synthase, allows the recombinant cellular production of terpenes.

As used herein, the term “host cell” or “transformed cell” refers to a cell (or organism) altered to harbor at least one nucleic acid molecule, for instance, a recombinant gene encoding a desired protein or nucleic acid sequence which upon transcription yields at least one functional polypeptide of the present invention required for performing a biocatalytic method or other recombinant method as described herein. In particular such host cells or transformed cells provide an acetyl transferase useful to prepare at least one drimanyl acetate from the corresponding drimanyl alcohol. They also may provide other enzymes like albicanyl diphosphate synthase protein useful to produce albicanyl diphosphate and/or albicanyl monophosphate and/or albicanol or corresponding mixtures of terpenes containing albicanyl diphosphate and/or albicanyl monophosphate and/or albicanol. They may also provide terpene synthases useful to prepare at least one drimanyl alcohol. The host cell is particularly a bacterial cell, a fungal cell or a plant cell or plants. The host cell may contain a recombinant gene which has been integrated into the nuclear or organelle genomes of the host cell. Alternatively, the host may contain the recombinant gene extra-chromosomally.

The term “organism” refers to any non-human multicellular or unicellular organisms such as a plant, or a microorganism. Particularly, a micro-organism is a bacterium, a yeast, an algae or a fungus.

The term “plant” is used interchangeably to include plant cells including plant protoplasts, plant tissues, plant cell tissue cultures giving rise to regenerated plants, or parts of plants, or plant organs such as roots, stems, leaves, flowers, pollen, ovules, embryos, fruits and the like. Any plant can be used to carry out the methods of an embodiment herein.

A particular organism or cell is meant to be “capable of producing FPP” when it produces FPP naturally or when it does not produce FPP naturally but is transformed to produce FPP with a nucleic acid as described herein. Organisms or cells transformed to produce a higher amount of FPP than the naturally occurring organism or cell are also encompassed by the “organisms or cells capable of producing FPP”.

A particular organism or cell is meant to be “capable of producing drimanyl acetate” when it produces a drimanyl acetate naturally or when it does not produce a drimanyl acetate naturally but is transformed to produce a drimanyl acetate with a nucleic acid as described herein. Organisms or cells transformed to produce a higher amount of a drimanyl acetate than the naturally occurring organism or cell are also encompassed by the “organisms or cells capable of producing a drimanyl acetate”.

A particular organism or cell is meant to be “capable of producing a drimanyl alcohol” when it produces a drimanyl alcohol naturally or when it does not produce a drimanyl alcohol naturally but is transformed to produce drimanyl diphosphate, and optionally further transformed with a nucleic acid to produce enzyme activity converting drimanyl diphosphate to drimanyl alcohol. Organisms or cells transformed to produce a higher amount of a drimanyl alcohol than the naturally occurring organism or cell are also encompassed by the “organisms or cells capable of producing a drimanyl alcohol”.

For the descriptions herein and the appended claims, the use of “or” means “and/or” unless stated otherwise. Similarly, “comprise”, “comprises”, “comprising”, “include”, “includes”, and “including” are interchangeable and not intended to be limiting.

It is to be further understood that where descriptions of various embodiments use the term “comprising”, those skilled in the art would understand that in some specific instances, an embodiment can be alternatively described using language “consisting essentially of” or “consisting of”.

The terms “purified,” “substantially purified”, and “isolated” as used herein refer to the state of being free of other, dissimilar compounds with which a compound of the invention is normally associated in its natural state, so that the “purified”, “substantially purified”, and “isolated” subject comprises at least 0.5%, 1%, 5%, 10% or 20%, or at least 50% or 75% of the mass, by weight, of a given sample. In one embodiment, these terms refer to the compound of the invention comprising at least 95, 96, 97, 98, 99 or 100% of the mass, by weight, of a given sample. As used herein, the terms “purified”, “substantially purified”, and “isolated” when referring to a nucleic acid or protein, of nucleic acids or proteins, also refers to a state of purification or concentration different than that which occurs naturally, for example in an prokaryotic or eukaryotic environment, like, for example in a bacterial or fungal cell, or in the mammalian organism, especially human body. Any degree of purification or concentration greater than that which occurs naturally, including (1) the purification from other associated structures or compounds or (2) the association with structures or compounds to which it is not normally associated in said prokaryotic or eukaryotic environment, are within the meaning of “isolated”. The nucleic acid or protein or classes of nucleic acids or proteins, described herein, may be isolated, or otherwise associated with structures or compounds to which they are not normally associated in nature, according to a variety of methods and processes known to those of skill in the art.

The term “about” indicates a potential variation of ±25% of the stated value, in particular ±15%, ±10%, more particularly ±5%, ±2% or +1%.

The term “substantially” describes a range of values of from about 80 to 100%, such as, for example, 85-99.9%, in particular 90 to 99.9%, more particularly 95 to 99.9%, or 98 to 99.9% and especially 99 to 99.9%.

“Predominantly” refers to a proportion in the range of above 50%, as for example in the range of 51 to 100%, particularly in the range of 75 to 99.9%; more particularly 85 to 99.5%, like 95 to 99%.

A “main product” in the context of the present invention designates a single compound or a group of at least 2 compounds, like 2, 3, 4, 5 or more, particularly 2 or 3 compounds, which single compound or group of compounds is “predominantly” prepared by a reaction as described herein, and is contained in said reaction in a predominant proportion based on the total amount of the constituents of the product formed by said reaction. Said proportion may be a molar proportion, a weight proportion or, preferably based on chromatographic analytics, an area proportion calculated from the corresponding chromatogram of the reaction products.

A “side product” in the context of the present invention designates a single compound or a group of at least 2 compounds, like 2, 3, 4, 5 or more, particularly 2 or 3 compounds, which single compound or group of compounds is not “predominantly” prepared by a reaction as described herein.

Because of the reversibility of enzymatic reactions, the present invention relates, unless otherwise stated, to the enzymatic or biocatalytic reactions described herein in both directions of reaction.

“Functional mutants” of herein described polypeptides include the “functional equivalents” of such polypeptides as defined below.

The term “stereoisomers” includes in particular conformational isomers.

Included in general are, according to the invention, all “stereoisomeric forms” of the compounds described herein, such as constitutional isomers and, in particular, stereoisomers and mixtures thereof, e.g. optical isomers, or geometric isomers, such as E- and Z-isomers, and combinations thereof. If several asymmetric centers are present in one molecule, the invention encompasses all combinations of different conformations of these asymmetry centers, e.g. enantiomeric pairs

“Stereoselectivity” describes the ability to produce a particular stereoisomer of a compound in a stereoisomerically pure form or to specifically convert a particular stereoisomer in an enzyme catalyzed method as described herein out of a plurality of stereoisomers. More specifically, this means that a product of the invention is enriched with respect to a specific stereoisomer, or an educt may be depleted with respect to a particular stereoisomer. This may be quantified via the purity % ee-parameter calculated according to the formula:

% ee=[X_{A}−X_{B}]/[X_{A}+X_{B}]*100,
wherein X_{A} and X_{B} represent the molar ratio (Molenbruch) of the stereoisomers A and B.

The terms “selectively converting” or “increasing the selectivity” in general means that a particular stereoisomeric form, as for example the E-form, of an unsaturated hydrocarbon, is converted in a higher proportion or amount (compared on a molar basis) than the corresponding other stereoisomeric form, as for example Z-form, either during the entire course of said reaction (i.e. between initiation and termination of the reaction), at a certain point of time of said reaction, or during an “interval” of said reaction. In particular, said selectivity may be observed during an “interval” corresponding 1 to 99%, 2 to 95%, 3 to 90%, 5 to 85%, 10 to 80%, 15 to 75%, 20 to 70%, 25 to 65%, 30 to 60%, or 40 to 50% conversion of the initial amount of the substrate. Said higher proportion or amount may, for example, be expressed in terms of:
a higher maximum yield of an isomer observed during the entire course of the reaction or said interval thereof;
a higher relative amount of an isomer at a defined % degree of conversion value of the substrate; and/or
an identical relative amount of an isomer at a higher % degree of conversion value;
each of which preferably being observed relative to a reference method, said reference method being performed under otherwise identical condition with known chemical or biochemical means.

Generally also comprised in accordance with the invention are all “isomeric forms” of the compounds described herein, such as constitutional isomers and in particular stereoisomers and mixtures of these, such as, for example, optical isomers or geometric isomers, such as E- and Z-isomers, and combinations of these. If several centers of asymmetry are present in a molecule, then the invention comprises all combinations of different conformations of these centers of asymmetry, such as, for example, pairs of enantiomers, or any mixtures of stereoisomeric forms.

“Yield” and/or the “conversion rate” of a reaction according to the invention is determined over a defined period of, for example, 4, 6, 8, 10, 12, 16, 20, 24, 36 or 48 hours, in which the reaction takes place. In particular, the reaction is carried out under precisely defined conditions, for example at “standard conditions” as herein defined.

The different yield parameters (“Yield” or Y_{P/S}; “Specific Productivity Yield”; or Space-Time-Yield (STY)) are well known in the art and are determined as described in the literature.

“Yield” and “Y_{P/S}” (each expressed in mass of product produced/mass of material consumed) are herein used as synonyms.

The specific productivity-yield describes the amount of a product that is produced per h and L fermentation broth per g of biomass. The amount of wet cell weight stated as WCW describes the quantity of biologically active microorganism in a biochemical reaction. The value is given as g product per g WCW per h (i.e. g/gWCW⁻¹ h⁻¹). Alternatively, the quantity of biomass can also be expressed as the amount of dry cell weight stated as DCW. Furthermore, the biomass concentration can be more easily determined by measuring the optical density at 600 nm (OD₆₀₀) and by using an experimentally determined correlation factor for estimating the corresponding wet cell or dry cell weight, respectively.

The term “fermentative production” or “fermentation” refers to the ability of a microorganism (assisted by enzyme activity contained in or generated by said microorganism) to produce a chemical compound in cell culture utilizing at least one carbon source added to the incubation.

The term “fermentation broth” is understood to mean a liquid, particularly aqueous or aqueous/organic solution which is based on a fermentative process and has not been worked up or has been worked up, for example, as described herein.

An “enzymatically catalyzed” or “biocatalytic” method means that said method is performed under the catalytic action of an enzyme, including enzyme mutants, as herein defined. Thus the method can either be performed in the presence of said enzyme in isolated (purified, enriched) or crude form or in the presence of a cellular system, in particular, natural or recombinant microbial cells containing said enzyme in active form, and having the ability to catalyze the conversion reaction as disclosed herein.

If the present disclosure refers to features, parameters and ranges thereof of different degree of preference (including general, not explicitly preferred features, parameters and ranges thereof) then, unless otherwise stated, any combination of two or more of such features, parameters and ranges thereof, irrespective of their respective degree of preference, is encompassed by the disclosure of the present description.

### DETAILED DESCRIPTION

a. Particular Embodiments of the Invention
1. A biocatalytic method of producing at least one, in particular one, two or three, particularly one or two drimanyl acetate compounds comprising the steps of
   (1) contacting in the presence of an acetyl group donor at least one, in particular one drimanyl alcohol, in stereoisomerically pure form or in the form of a mixture of stereoisomers, with at least one, in particular one polypeptide having acetyl transferase activity capable of, in particular transferring an acetyl group from said acetyl group donor to said at least one, in particular one drimanyl alcohol to obtain at least one drimanyl acetate as main product, in particular one drimanyl acetate as main product; and
   (2) optionally isolating said at least one, in particular one drimanyl acetate compound from the reaction product of step (1).
   If more than one drimanyl acetate is formed, the mixture may be further separated and individual acetates may be purified.
2. The method of embodiment 1, wherein said drimanyl acetate compound is selected from the group consisting of albicanyl acetate, drimenyl acetate, and bicyclofarnesyl acetate, each in stereoisomerically pure form or as a mixture of at least two stereoisomers thereof, or combinations thereof comprising at least two members of said group of acetates. In a particular embodiment merely one drimanyl alcohol is used as substrate and merely one drimanyl acetate is obtained as product in stereoisomerically pure form or as a mixture of at least two stereoisomers thereof, in particular in stereoisomerically pure form.
3. The method of embodiment 1 or 2, wherein said drimanyl alcohol is selected from the group consisting of albicanol, in particular (+)-albicanol, drimenol, in particular (−)-drimenol, and bicyclofarnesol, in particular (+)-bicyclofarnesol, each in stereoisomerically pure form or as a mixture of at least two stereoisomers thereof, or combinations thereof comprising at least two members of said group of alcohols. In a particular embodiment merely one drimanyl alcohol, in particular in stereoisomerically pure form is used as substrate.
4. The method of anyone of the preceding embodiments, wherein said acetyl group donor is acetyl-Coenzyme A (acetyl-CoA). Said donor may be exogenously added to the reaction mixture, for example in an in vitro process applying isolated, enriched or purified enzyme, or more particularly be endogenously present, for example in an in vivo process applying a host cell system producing acetyl-CoA as metabolite and expressing the required polypeptide or polypeptides for performing the intended acetylation or a more complex, multistep process encompassing said acetylation as one step.
5. The method of anyone of the preceding embodiments, wherein said acetyl transferase is selected from
   a) polypeptides comprising an amino acid sequence selected from SEQ ID NO: 9, 11, 13, 15, 17, 19, 21, 23, 25, 118, 121, 124, 127, 130, 133,136, 143 and 144 and
   b) polypeptides having acetyl transferase activity and comprising an amino acid sequence showing an degree of sequence identity of at least 40%, 45%, 50%, 55%, 60%, 65%, 70%, 75%, 80%, 85%, 90%, 95%, 96%, 97%, 98%, or 99% to at least one of said amino acid sequences of SEQ ID NO: 9, 11, 13, 15, 17, 19, 21, 23, 25, 118, 121, 124, 127, 130, 133, 136, 143 and 144.
   The ability of particular acetyl transferases of the invention to produce one or more drimane acetate compounds selected from the group consisting of albicanyl acetate, drimenyl acetate, and bicyclofarnesyl acetate is illustrated by the following listing:

| Acetyl | SEQ | Albicanyl | Drimenyl | Bicyclofarnesyl |
|---|---|---|---|---|
| tansferase | ID NO | acetate | acetate | acetate |
| CrDAT | 9 | + | + | + |
| TcTAT | 11 | + | + | + |
| CrMAT | 13 | + | | + |
| LiAAT-4 | 15 | + | | |
| FgaAT | 17 | + | + | + |
| GAO81666.1 | 19 | + | + | + |
| CfACT1-6 | 21 | + | + | + |
| CfACT1-8 | 23 | + | + | + |
| OAH94415.1 | 25 | + | + | |
| DfACT13 | 118 | + | + | + |
| PYI04555.1 | 121 or 143 | | | + |
| ERR364415- | 124 or 144 | + | + | + |
| 1_contig_8546 | | | | |
| XP_001258079.1 | 127 | | + | |
| XP_001217250.1 | 130 | | | + |
| BAU61551.1 | 133 | | | + |
| PsSalAT | 136 | | | + |

6. The method of anyone of the preceding embodiments, further comprising, prior to step 1) the biocatalytic formation of said at least one, in particular one drimanyl alcohol compound.
7. The method of embodiment 6, wherein said drimanyl alcohol compound may endogenously be present in the reaction mixture, for example in an in vivo process applying a host cell system producing said drimanyl alcohol compound as metabolite and expressing the required polypeptide or polypeptides for performing the intended drimanyl alcohol synthesis or a more complex, multistep process encompassing said drimanyl alcohol synthesis as one step whereby said drimanyl alcohol is enzymatically synthesized from a non-cyclic sesquiterpene precursor.
   Alternatively, said drimanyl alcohol compound is either chemically or enzymatically produced, and is exogenously added to the reaction mixture, for example in an in vitro process applying an isolated, enriched or purified synthase enzyme required for its formation as defined below.
8. The method of embodiment 7, wherein said non-cyclic sesquiterpene precursor is farnesyl pyrophosphate (FPP).
9. The method of anyone of the embodiments 7 to 8, wherein said enzymatic synthesis of the drimanyl alcohol is catalysed by one or more polypeptides having the ability to convert said non-cyclic sesquiterpene precursor to at least one drimanyl alcohol, in particular one drimanyl alcohol, in one or more enzymatic steps, in particular as main product.
10. The method of anyone of the embodiments 6 to 9, wherein said at least one drimanyl alcohol is produced in a single or more, in particular two enzymatic steps from FPP.
11. The method of embodiment 10, wherein said at least one drimanyl alcohol is produced by the enzymatic conversion of FPP, catalysed by
   a) a polypeptide having drimane sesquiterpene synthase activity forming said drimanyl alcohol (single step biosynthesis); or
   b) a combination of a polypeptide having drimanyl phosphate synthase activity forming at least one drimanyl phosphate intermediate, and a polypeptide having phosphatase activity converting said at least one drimanyl phosphate (monophosphate and/or diphosphate) intermediate to at least one drimanyl alcohol (two step biosynthesis).
12. The method of embodiment 11, wherein
   a) said polypeptide having drimane sesquiterpene synthase activity is selected from a polypeptide having albicanol synthase activity, drimenol synthase activity, bicyclofarnesol synthase activity or any combination of such activities, in particular showing one of said activities preferentially, and more particular, showing one of said activities specifically; and
   b) said combination of polypeptides comprises a drimanyl diphosphate synthase activity, in particular an albicanyl diphosphate synthase activity and a phosphatase enzyme, as for example a bacterial alkaline phosphatase.
13. The method of embodiment 12, wherein
   a) said polypeptide having drimane sesquiterpene synthase activity is selected from drimane synthases as described in PCT/EP2018/064344 (filed May 31, 2018) and drimenol synthases as described in published WO2015/169871 and WO 2015/176959;
   b) said polypeptide having drimanyl phosphate synthase activity is an albicanyl diphosphate synthase as described in PCT/CN2018/088902, filed on May 29, 2018, comprising the ability to produce an albicanyl phosphate derivative like a monophosphate and more particularly an albicanyl diphosphate from farnesyl diphosphate (FPP) as substrate.
   Albicanyl diphosphate synthases as described in PCT/CN2018/088902 (filed May 29, 2018) are:
   DfHAD, DfHAD-9 (V274A), DfHAD-His_GST, and DfHAD-8 (K532R) of Dryopteris fragrans
   and polypeptides derived therefrom having at least 40%, 45%, 50%, 55%, 60%, 65%, 70%, 75%, 80%, 85%, 90%, 95%, 96%, 97%, 98%, or 99% sequence identity thereto.
   Drimane synthases (i.e. albicanol synthases or drimenol synthases) as described in PCT/EP2018/064344 (filed May 31, 2018) are

| Name or NCBI | | |
|---|---|---|
| accession number | Source | Product |
| CvTps1 | Cryptoporus volvatus | Albicanol |
| LoTps1 | Laricifomes officinalis | Albicanol |
| OCH93767.1 | Obba rivulosa | Albicanol |
| EMD37666.1 | Gelatoporia subvermispora | Albicanol |
| EMD37666-B | Gelatoporia subvermispora | Albicanol |
| XP_001217376.1 | Aspergillus terreus | Albicanol |
| OJJ98394.1 | Aspergillus aculeatus | Albicanol |
| GAO87501.1 | Aspergillus udagawae | Albicanol |
| XP_008034151.1 | Trametes versicolor | Albicanol |
| XP_007369631.1 | Dichomitus squalens | Albicanol |
| ACg006372 | Antrodia cinnamomea | Albicanol |
| KIA75676.1 | Aspergillus ustus | Drimenol |
| XP_001820867.2 | Aspergillus oryzae | Drimenol |
| CEN60542.1 | Aspergillus calidoustus | Drimenol |
| XP_009547469.1 | Heterobasidion irregulare | Drimenol |
| KLO09124.1 | Schizopora paradoxa | Drimenol |
| OJI95797.1 | Aspergillus versicolor | Drimenol |
| XP_006461126 | Agaricus bisporus | Drimenol |

and polypeptides derived therefrom having at least 40%, 45%, 50%, 55%, 60%, 65%, 70%, 75%, 80%, 85%, 90%, 95%, 96%, 97%, 98%, or 99% sequence identity thereto.
Drimenol synthases as described in WO2015/169871 are

| Name | Source |
|---|---|
| DITps589 | Drimys lanceolota |
| SCH51_3228_11 | Drimys winteri |
| SCH51_3228_9 | Drimys winteri |
| SCH51_998_28 | Drimys winteri |
| SCH52_13163_6 | Drimys lanceolota |

and polypeptides derived therefrom having at least 40%, 45%, 50%, 55%, 60%, 65%, 70%, 75%, 80%, 85%, 90%, 95%, 96%, 97%, 98%, or 99% sequence identity thereto.
Drimenol synthases as described in WO 2015/176959 are
VaTPS3 of Valeriana amurensis
and polypeptides derived therefrom having at least 40%, 45%, 50%, 55%, 60%, 65%, 70%, 75%, 80%, 85%, 90%, 95%, 96%, 97%, 98%, or 99% sequence identity thereto.
The SEQ ID NOs of respective amino acid and nucleotide sequences of the above-mentioned synthase enzymes are listed at the end of the current description. These polypeptides and nucleic acids as well as polypeptides and nucleic acids derived therefrom having at least 40%, 45%, 50%, 55%, 60%, 65%, 70%, 75%, 80%, 85%, 90%, 95%, 96%, 97%, 98%, or 99% sequence identity to at least one of these sequences are also part of the current disclosure.
14. The method of embodiment 13, wherein said drimane sesquiterpene synthase is selected from
   a) a polypeptide having (bifunctional) albicanol synthase activity and comprising an amino acid sequence of SEQ ID NO: 5 or a mutant or variant polypeptide having albicanol synthase activity and comprising an amino acid sequence having at least 40%, 45%, 50%, 55%, 60%, 65%, 70%, 75%, 80%, 85%, 90%, 95%, 96%, 97%, 98%, or 99% sequence identity to SEQ ID NO: 5 as described in PCT/EP2018/064344;
   b) a polypeptide having (bifunctional) drimenol synthase activity and comprising an amino acid sequence of SEQ ID NO: 7 or a mutant or variant polypeptide having drimenol synthase activity and comprising an amino acid sequence having at least 40%, 45%, 50%, 55%, 60%, 65%, 70%, 75%, 80%, 85%, 90%, 95%, 96%, 97%, 98%, or 99% sequence identity to SEQ ID NO: 7 as described in PCT/EP2018/064344.
15. The method of anyone of the preceding embodiments, performed in vivo in host cell culture or in vitro in a liquid reaction medium comprising a host cell lysate or enriched or isolated polypeptides required for producing at least one drimanyl acetate, each under conditions conducive to the production of at least one drimanyl acetate.
   In particular, the reaction is performed in the presence of endogenously formed or exogenously added acetyl-CoA as acetyl group donor. In particular, the reaction is performed in the presence of endogenously formed or exogenously added FPP. Endogenously formed FPP in particular is the result of the metabolization of at least one carbon source, as for example a sugar substrate, bioconvertible to FPP. A cellular in vivo method is of particular interest, wherein FPP and acetyl-CoA are endogenously formed.
   Some of these host cells or organisms do not produce FPP naturally. To be suitable to carry out the method of an embodiment as described herein, organisms or cells that do not produce an acyclic terpene pyrophosphate precursor, e.g. FPP, naturally are genetically modified to produce said precursor. They can be, for example, so transformed either before the modification with the nucleic acid described according to any of the above embodiments or simultaneously. Methods to transform organisms so that they produce an acyclic terpene pyrophosphate precursor, e.g. FPP, are already known in the art. For example, introducing enzyme activities of the mevalonate pathway, is a suitable strategy to make the organism produce FPP.
16. The method of embodiment 15 performed in a recombinant non-human host cell or a recombinant non-human host organism capable of functionally expressing,
   a) at least one acetyl transferase as defined in embodiment 5; optionally
   b) at least one polypeptide having the ability to convert the non-cyclic sesquiterpene precursor FPP to at least one drimanyl alcohol as defined in anyone of the embodiments 9 to 14; and optionally
   c) at least one enzyme selected from enzymes involved in the mevalonate pathway as defined above.
      In a particular embodiment enzymes a) and b) or enzymes a), b) and c) are functionally expressed by such cellular system as applied in an in vivo method of the invention.
17. The method of embodiment 16, wherein said non-human host cell or host organism is selected from a prokaryotic or eukaryotic microorganism, or a cell derived therefrom.
18. The method of embodiment 17, wherein said non-human host cell or host organism is selected from bacterial, fungal and plant cells or plants.
19. The method of embodiment 18, wherein said fungal cells are yeast cells, in particular selected from the genus Saccharomyces, Pichia or Yarrowia, in particular from the species Saccharomyces cerevisiae, Pichia pastori or Yarrowia lipolytica.
20. The method of embodiment 18 wherein said bacterial cells are selected from the genus Rhodococcus, Pseudomonas, Bacillus or Escherichia, in particular from the species E. coli.
21. The method of anyone of the preceding embodiments, further comprising as step (3) the processing of the at least one drimanyl acetate of step (1) or of step (2) to obtain a derivative using chemical or biocatalytic synthesis or a combination of both.
22. The method of embodiment 21, wherein the derivative is a hydrocarbon, alcohol, diol, triol, acetal, ketal, aldehyde, acid, ether, amide, ketone, lactone, epoxide, acetate, glycoside, ester and/or polycyclic compound.
23. The method of anyone of the preceding embodiments, wherein, the drimanyl acetate comprises albicanyl acetate, drimenyl acetate or bicyclofarnesyl acetate, in particular albicanyl acetate or drimenyl acetate, as main drimanyl alcohol product or, in particular as the single drimanyl alcohol product.
24. The method of any one of the preceding embodiments which comprises providing, in particular by transforming, a non-human host organism or host cell with
   a) at least one nucleic acid, expression construct or vector comprising a nucleic acid sequence encoding at least one polypeptide having acetyl transferase activity capable of transferring an acetyl group from an acetyl group donor to drimanyl alcohol, optionally stably integrated into the genome; optionally
   b) at least one nucleic acid, expression construct or vector comprising a nucleic acid sequence encoding at least one polypeptide having drimanyl alcohol synthase activity capable of producing a drimanyl alcohol from a non-cyclic sesquiterpene precursor, optionally stably integrated into the genome; and optionally
   c) at least one nucleic acid, expression construct or vector comprising a nucleic acid sequence encoding at least one polypeptide involved in the biosynthetic pathway for producing said non-cyclic sesquiterpene precursor, optionally stably integrated into the genome.
      In a particular embodiment the non-human host organism or host cell is transformed with a) and b) or a), b) and c), and more particularly contains said nucleic acids stably integrated into the genome. The nucleic acids a), b) and/or c) may be located on the same or on two or more different vectors.
25. A polypeptide having acetyl transferase activity, capable of transferring an acetyl group from an acetyl group donor to drimanyl alcohol in order to produce a drimanyl acetate, which comprises an amino acid sequence having a sequence identity of 40%, 45%, 50%, 55%, 60%, 65%, 70%, 75%, 80%, 85%, 90%, 95%, 96%, 97%, 98%, or 99% to less than 100% to a least one amino acid sequence selected from SEQ ID NO: 9, 11, 13, 15, 17, 19, 21, 23, 25, 118, 121, 124, 127, 130, 133, 136, 143 and 144.
26. An isolated nucleic acid molecule
   a) comprising a nucleotide sequence encoding the polypeptide of embodiment 5; or
   b) comprising a nucleotide sequence having least 40%, 45%, 50%, 55%, 60%, 65%, 70%, 75%, 80%, 85%, 90%, 95%, 96%, 97%, 98%, or 99% and less than 100% sequence identity to a nucleotide sequence selected from SEQ ID NO:8, 10, 12, 14, 16, 18, 20, 22, 24, 116, 117, 119, 120, 122, 123, 125, 126, 128, 129, 131, 132, 134 and 135; or
   c) comprising a nucleotide sequence comprising a sequence complementary to one of the sequences of a) or b); or
   d) comprising a nucleotide sequence hybridizing under stringent conditions to a nucleotide sequence of a), b) or c).
27. An expression construct comprising at least one nucleic acid molecule of embodiment 26.
28. A vector comprising at least one nucleic acid molecule of embodiment 25 or at least one expression construct of embodiment 26.
29. The vector of embodiment 28, wherein the vector is a prokaryotic, viral or eukaryotic vector.
30. The vector of embodiment 28 or 29, wherein the vector is an expression vector.
31. The vector of anyone of the embodiments 28 to 30, which is a plasmid vector.
32. A recombinant host cell or a recombinant non-human host organism comprising
   a) at least one isolated nucleic acid molecule of embodiment 26, optionally stably integrated into the genome; or
   b) at least one expression construct of embodiment 27, optionally stably integrated into the genome; or
   c) at least one vector of any one of embodiments 28 to 31.
   In a particular embodiment the non-human host organism or host cell is transformed with a) and b) or a), b) and c), and more particularly contains said nucleic acids stably integrated into the genome.
33. The host cell or host organism of embodiment 32, selected from a prokaryotic or eukaryotic microorganism, or a cell derived therefrom.
34. The host cell or host organism of embodiment 33, selected from bacterial, fungal and plant cells or plants.
35. The host cell or host organism of embodiment 34, wherein said fungal cells are yeast cells.
36. The host cell or host organism of embodiment 35, wherein said bacterial cells are selected from the genus Escherichia, in particular from the species E. coli and said yeast cells are selected from the genus Saccharomyces, Pichia or Yarrowia, in particular from the species Saccharomyces cerevisiae or Pichia pastoris, or Yarrowia lipolytica.
37. A method for producing at least one catalytically active polypeptide according to embodiment 25 comprising:
   a) culturing a non-human host organism or host cell of one of the embodiments 32 to 34 to express or over-express at least one polypeptide according to embodiment 25; and
   b) optionally isolating the polypeptide from the non-human host cell or organism cultured in step a).
38. The method of embodiment 37, further comprising, prior to step a), providing, in particular transforming a non-human host organism or cell with at least one nucleic acid according to claim 26, or at least one construct of claim 27, or at least one vector of anyone of the claims 28 to 31 so that it expresses or over-expresses the polypeptide according to claim 25.
39. A method for preparing a mutant polypeptide comprising acetyl transferase activity, capable of transferring an acetyl group from an acetyl group donor to at least one, in particular one drimanyl alcohol in order to produce at least one, in particular one drimanyl acetate, which method comprises the steps of:
   a) selecting a nucleic acid molecule encoding a polypeptide selected from SEQ ID NO: 9, 11, 13, 15, 17, 19, 21, 23, 25, 118, 121, 124, 127, 130, 133, 136, 143 and 144,
   b) modifying the selected nucleic acid molecule to obtain at least one mutant nucleic acid molecule;
   c) transforming host cells or unicellular host organisms with the mutant nucleic acid sequence to express a polypeptide encoded by the mutant nucleic acid sequence;
   d) screening the expression product for at least one mutant comprising acetyl transferase activity; and,
   e) optionally, if the polypeptide has no desired mutant activity, repeat the process steps a) to d) until a polypeptide with a desired mutant activity is obtained; and
   f) optionally, if a polypeptide having a desired mutant activity was identified in step d), isolating the corresponding mutant nucleic acid obtained in step c).
40. The use of an acetyltransferase as defined in anyone of the preceding embodiments, for preparing odorants, flavours or fragrance ingredients or insect/pest control as for example for use in the preparation of a composition selected from body care, home care, or fragrance compositions.
   b. Polypeptides Applicable According to the Invention

In this context the following definitions apply:

The generic terms “polypeptide” or “peptide”, which may be used interchangeably, refer to a natural or synthetic linear chain or sequence of consecutive, peptidically linked amino acid residues, comprising about 10 up to more than 1.000 residues. Short chain polypeptides with up to 30 residues are also designated as “oligopeptides”.

The term “protein” refers to a macromolecular structure consisting of one or more polypeptides. The amino acid sequence of its polypeptide(s) represents the “primary structure” of the protein. The amino acid sequence also predetermines the “secondary structure” of the protein by the formation of special structural elements, such as alpha-helical and beta-sheet structures formed within a polypeptide chain. The arrangement of a plurality of such secondary structural elements defines the “tertiary structure” or spatial arrangement of the protein. If a protein comprises more than one polypeptide chains said chains are spatially arranged forming the “quaternary structure” of the protein. A correct spacial arrangement or “folding” of the protein is prerequisite of protein function. Denaturation or unfolding destroys protein function. If such destruction is reversible, protein function may be restored by refolding.

A typical protein function referred to herein is an “enzyme function”, i.e. the protein acts as biocatalyst on a substrate, for example a chemical compound, and catalyzes the conversion of said substrate to a product. An enzyme may show a high or low degree of substrate and/or product specificity.

A “polypeptide” referred to herein as having a particular “activity” thus implicitly refers to a correctly folded protein showing the indicated activity, as for example a specific enzyme activity.

Thus, unless otherwise indicated the term “polypeptide” also encompasses the terms “protein” and “enzyme”.

Similarly, the term “polypeptide fragment” encompasses the terms “protein fragment” and “enzyme fragment”.

The term “isolated polypeptide” refers to an amino acid sequence that is removed from its natural environment by any method or combination of methods known in the art and includes recombinant, biochemical and synthetic methods.

“Target peptide” refers to an amino acid sequence which targets a protein, or polypeptide to intracellular organelles, i.e., mitochondria, or plastids, or to the extracellular space (secretion signal peptide). A nucleic acid sequence encoding a target peptide may be fused to the nucleic acid sequence encoding the amino terminal end, e.g., N-terminal end, of the protein or polypeptide, or may be used to replace a native targeting polypeptide.

The present invention also relates to “functional equivalents” (also designated as “analogs” or “functional mutations”) of the polypeptides specifically described herein.

For example, “functional equivalents” refer to polypeptides which, in a test used for determining enzymatic activity, display at least a 1 to 10%, or at least 20%, or at least 50%, or at least 75%, or at least 90% higher or lower enzymatic activity, as that of the polypeptide specifically described herein and serving as basis for said comparison.

“Functional equivalents”, according to the invention, also cover particular mutants, which, in at least one sequence position of an amino acid sequences stated herein, have an amino acid that is different from that concretely stated one, but nevertheless possess one of the aforementioned biological activities, as for example enzyme activity. “Functional equivalents” thus comprise mutants obtainable by one or more, like 1 to 20, in particular 1 to 15 or 5 to 10 amino acid additions, substitutions, in particular conservative substitutions (i.e. as a result thereof the amino acid in question is replaced by an amino acid of the same charge, size, polarity and/or solubility), deletions and/or inversions, where the stated changes can occur in any sequence position, provided they lead to a mutant with the profile of properties according to the invention. Functional equivalence is in particular also provided if the activity patterns coincide qualitatively between the mutant and the unchanged polypeptide, i.e. if, for example, interaction with the same agonist or antagonist or substrate, however at a different rate, (i.e. expressed by a EC₅₀ or IC₅₀ value or any other parameter suitable in the present technical field) is observed. Examples of suitable (conservative) amino acid substitutions are shown in the following table:

| Original residue | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

“Functional equivalents” in the above sense are also “precursors” of the polypeptides described herein, as well as “functional derivatives” and “salts” of the polypeptides.

“Precursors” are in that case natural or synthetic precursors of the polypeptides with or without the desired biological activity.

The expression “salts” means salts of carboxyl groups as well as salts of acid addition of amino groups of the protein molecules according to the invention. Salts of carboxyl groups can be produced in a known way and comprise inorganic salts, for example sodium, calcium, ammonium, iron and zinc salts, and salts with organic bases, for example amines, such as triethanolamine, arginine, lysine, piperidine and the like. Salts of acid addition, for example salts with inorganic acids, such as hydrochloric acid or sulfuric acid and salts with organic acids, such as acetic acid and oxalic acid, are also covered by the invention.

“Functional derivatives” of polypeptides according to the invention can also be produced on functional amino acid side groups or at their N-terminal or C-terminal end using known techniques. Such derivatives comprise for example aliphatic esters of carboxylic acid groups, amides of carboxylic acid groups, obtainable by reaction with ammonia or with a primary or secondary amine; N-acyl derivatives of free amino groups, produced by reaction with acyl groups; or O-acyl derivatives of free hydroxyl groups, produced by reaction with acyl groups.

“Functional equivalents” naturally also comprise polypeptides that can be obtained from other organisms, as well as naturally occurring variants. For example, areas of homologous sequence regions can be established by sequence comparison, and equivalent polypeptides can be determined on the basis of the concrete parameters of the invention.

“Functional equivalents” also comprise “fragments”, like individual domains or sequence motifs, of the polypeptides according to the invention, or N- and/or C-terminally truncated forms, which may or may not display the desired biological function. Preferably such “fragments” retain the desired biological function at least qualitatively.

“Functional equivalents” are, moreover, fusion proteins, which have one of the polypeptide sequences stated herein or functional equivalents derived therefrom and at least one further, functionally different, heterologous sequence in functional N-terminal or C-terminal association (i.e. without substantial mutual functional impairment of the fusion protein parts). Non-limiting examples of these heterologous sequences are e.g. signal peptides, histidine anchors or enzymes.

“Functional equivalents” which are also comprised in accordance with the invention are homologs to the specifically disclosed polypeptides. These have at least 60%, preferably at least 75%, in particular at least 80 or 85%, such as, for example, 90, 91, 92, 93, 94, 95, 96, 97, 98 or 99%, homology (or identity) to one of the specifically disclosed amino acid sequences, calculated by the algorithm of Pearson and Lipman, Proc. Natl. Acad, Sci. (USA) 85 (8), 1988, 2444-2448. A homology or identity, expressed as a percentage, of a homologous polypeptide according to the invention means in particular an identity, expressed as a percentage, of the amino acid residues based on the total length of one of the amino acid sequences described specifically herein.

The identity data, expressed as a percentage, may also be determined with the aid of BLAST alignments, algorithm blastp (protein-protein BLAST), or by applying the Clustal settings specified herein below.

In the case of a possible protein glycosylation, “functional equivalents” according to the invention comprise polypeptides as described herein in deglycosylated or glycosylated form as well as modified forms that can be obtained by altering the glycosylation pattern.

Functional equivalents or homologues of the polypeptides according to the invention can be produced by mutagenesis, e.g. by point mutation, lengthening or shortening of the protein or as described in more detail below.

Functional equivalents or homologs of the polypeptides according to the invention can be identified by screening combinatorial databases of mutants, for example shortening mutants. For example, a variegated database of protein variants can be produced by combinatorial mutagenesis at the nucleic acid level, e.g. by enzymatic ligation of a mixture of synthetic oligonucleotides. There are a great many methods that can be used for the production of databases of potential homologues from a degenerated oligonucleotide sequence. Chemical synthesis of a degenerated gene sequence can be carried out in an automatic DNA synthesizer, and the synthetic gene can then be ligated in a suitable expression vector. The use of a degenerated genome makes it possible to supply all sequences in a mixture, which code for the desired set of potential protein sequences. Methods of synthesis of degenerated oligonucleotides are known to a person skilled in the art.

In the prior art, several techniques are known for the screening of gene products of combinatorial databases, which were produced by point mutations or shortening, and for the screening of cDNA libraries for gene products with a selected property. These techniques can be adapted for the rapid screening of the gene banks that were produced by combinatorial mutagenesis of homologues according to the invention. The techniques most frequently used for the screening of large gene banks, which are based on a high-throughput analysis, comprise cloning of the gene bank in expression vectors that can be replicated, transformation of the suitable cells with the resultant vector database and expression of the combinatorial genes in conditions in which detection of the desired activity facilitates isolation of the vector that codes for the gene whose product was detected. Recursive Ensemble Mutagenesis (REM), a technique that increases the frequency of functional mutants in the databases, can be used in combination with the screening tests, in order to identify homologues.

An embodiment provided herein provides orthologs and paralogs of polypeptides disclosed herein as well as methods for identifying and isolating such orthologs and paralogs. A definition of the terms “ortholog” and “paralog” is given below and applies to amino acid and nucleic acid sequences.

c. Coding Nucleic Acid Sequences Applicable According to the Invention

In this context the following definitions apply:

The terms “nucleic acid sequence,” “nucleic acid,” “nucleic acid molecule” and “polynucleotide” are used interchangeably meaning a sequence of nucleotides. A nucleic acid sequence may be a single-stranded or double-stranded deoxyribonucleotide, or ribonucleotide of any length, and include coding and non-coding sequences of a gene, exons, introns, sense and anti-sense complimentary sequences, genomic DNA, cDNA, miRNA, siRNA, mRNA, rRNA, tRNA, recombinant nucleic acid sequences, isolated and purified naturally occurring DNA and/or RNA sequences, synthetic DNA and RNA sequences, fragments, primers and nucleic acid probes. The skilled artisan is aware that the nucleic acid sequences of RNA are identical to the DNA sequences with the difference of thymine (T) being replaced by uracil (U). The term “nucleotide sequence” should also be understood as comprising a polynucleotide molecule or an oligonucleotide molecule in the form of a separate fragment or as a component of a larger nucleic acid.

An “isolated nucleic acid” or “isolated nucleic acid sequence” relates to a nucleic acid or nucleic acid sequence that is in an environment different from that in which the nucleic acid or nucleic acid sequence naturally occurs and can include those that are substantially free from contaminating endogenous material.

The term “naturally-occurring” as used herein as applied to a nucleic acid refers to a nucleic acid that is found in a cell of an organism in nature and which has not been intentionally modified by a human in the laboratory.

A “fragment” of a polynucleotide or nucleic acid sequence refers to contiguous nucleotides that is particularly at least 15 bp, at least 30 bp, at least 40 bp, at least 50 bp and/or at least 60 bp in length of the polynucleotide of an embodiment herein. Particularly the fragment of a polynucleotide comprises at least 25, more particularly at least 50, more particularly at least 75, more particularly at least 100, more particularly at least 150, more particularly at least 200, more particularly at least 300, more particularly at least 400, more particularly at least 500, more particularly at least 600, more particularly at least 700, more particularly at least 800, more particularly at least 900, more particularly at least 1000 contiguous nucleotides of the polynucleotide of an embodiment herein. Without being limited, the fragment of the polynucleotides herein may be used as a PCR primer, and/or as a probe, or for anti-sense gene silencing or RNAi.

As used herein, the term “hybridization” or hybridizes under certain conditions is intended to describe conditions for hybridization and washes under which nucleotide sequences that are significantly identical or homologous to each other remain bound to each other. The conditions may be such that sequences, which are at least about 70%, such as at least about 80%, and such as at least about 85%, 90%, or 95% identical, remain bound to each other. Definitions of low stringency, moderate, and high stringency hybridization conditions are provided herein below. Appropriate hybridization conditions can also be selected by those skilled in the art with minimal experimentation as exemplified in Ausubel et al. (1995, Current Protocols in Molecular Biology, John Wiley & Sons, sections 2, 4, and 6). Additionally, stringency conditions are described in Sambrook et al. (1989, Molecular Cloning: A Laboratory Manual, 2nd ed., Cold Spring Harbor Press, chapters 7, 9, and 11).

“Recombinant nucleic acid sequences” are nucleic acid sequences that result from the use of laboratory methods (for example, molecular cloning) to bring together genetic material from more than on source, creating or modifying a nucleic acid sequence that does not occur naturally and would not be otherwise found in biological organisms.

“Recombinant DNA technology” refers to molecular biology procedures to prepare a recombinant nucleic acid sequence as described, for instance, in Laboratory Manuals edited by Weigel and Glazebrook, 2002, Cold Spring Harbor Lab Press; and Sambrook et al., 1989, Cold Spring Harbor, NY, Cold Spring Harbor Laboratory Press.

The term “gene” means a DNA sequence comprising a region, which is transcribed into a RNA molecule, e.g., an mRNA in a cell, operably linked to suitable regulatory regions, e.g., a promoter. A gene may thus comprise several operably linked sequences, such as a promoter, a 5′ leader sequence comprising, e.g., sequences involved in translation initiation, a coding region of cDNA or genomic DNA, introns, exons, and/or a 3′non-translated sequence comprising, e.g., transcription termination sites.

“Polycistronic” refers to nucleic acid molecules, in particular mRNAs, that can encode more than one polypeptide separately within the same nucleic acid molecule

A “chimeric gene” refers to any gene which is not normally found in nature in a species, in particular, a gene in which one or more parts of the nucleic acid sequence are present that are not associated with each other in nature. For example the promoter is not associated in nature with part or all of the transcribed region or with another regulatory region. The term “chimeric gene” is understood to include expression constructs in which a promoter or transcription regulatory sequence is operably linked to one or more coding sequences or to an antisense, i.e., reverse complement of the sense strand, or inverted repeat sequence (sense and antisense, whereby the RNA transcript forms double stranded RNA upon transcription). The term “chimeric gene” also includes genes obtained through the combination of portions of one or more coding sequences to produce a new gene.

A “3′ UTR” or “3′ non-translated sequence” (also referred to as “3′ untranslated region,” or “3′end”) refers to the nucleic acid sequence found downstream of the coding sequence of a gene, which comprises, for example, a transcription termination site and (in most, but not all eukaryotic mRNAs) a polyadenylation signal such as AAUAAA or variants thereof. After termination of transcription, the mRNA transcript may be cleaved downstream of the polyadenylation signal and a poly (A) tail may be added, which is involved in the transport of the mRNA to the site of translation, e.g., cytoplasm.

The term “primer” refers to a short nucleic acid sequence that is hybridized to a template nucleic acid sequence and is used for polymerization of a nucleic acid sequence complementary to the template.

The term “selectable marker” refers to any gene which upon expression may be used to select a cell or cells that include the selectable marker. Examples of selectable markers are described below. The skilled artisan will know that different antibiotic, fungicide, auxotrophic or herbicide selectable markers are applicable to different target species.

The invention also relates to nucleic acid sequences that code for polypeptides as defined herein.

In particular, the invention also relates to nucleic acid sequences (single-stranded and double-stranded DNA and RNA sequences, e.g. cDNA, genomic DNA and mRNA), coding for one of the above polypeptides and their functional equivalents, which can be obtained for example using artificial nucleotide analogs.

The invention relates both to isolated nucleic acid molecules, which code for polypeptides according to the invention or biologically active segments thereof, and to nucleic acid fragments, which can be used for example as hybridization probes or primers for identifying or amplifying coding nucleic acids according to the invention.

The present invention also relates to nucleic acids with a certain degree of “identity” to the sequences specifically disclosed herein. “Identity” between two nucleic acids means identity of the nucleotides, in each case over the entire length of the nucleic acid.

The “identity” between two nucleotide sequences (the same applies to peptide or amino acid sequences) is a function of the number of nucleotide residues (or amino acid residues) or that are identical in the two sequences when an alignment of these two sequences has been generated. Identical residues are defined as residues that are the same in the two sequences in a given position of the alignment. The percentage of sequence identity, as used herein, is calculated from the optimal alignment by taking the number of residues identical between two sequences dividing it by the total number of residues in the shortest sequence and multiplying by 100. The optimal alignment is the alignment in which the percentage of identity is the highest possible. Gaps may be introduced into one or both sequences in one or more positions of the alignment to obtain the optimal alignment. These gaps are then taken into account as non-identical residues for the calculation of the percentage of sequence identity. Alignment for the purpose of determining the percentage of amino acid or nucleic acid sequence identity can be achieved in various ways using computer programs and for instance publicly available computer programs available on the world wide web.

Particularly, the BLAST program (Tatiana et al, FEMS Microbiol Lett., 1999, 174:247-250, 1999) set to the default parameters, available from the National Center for Biotechnology Information (NCBI) website at ncbi.nlm.nih.gov/BLAST/b12seq/wblast2.cgi, can be used to obtain an optimal alignment of protein or nucleic acid sequences and to calculate the percentage of sequence identity.

In another example the identity may be calculated by means of the Vector NTI Suite 7.1 program of the company Informax (USA) employing the Clustal Method (Higgins D G, Sharp P M. (1989)) with the following settings:

### Multiple Alignment Parameters:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |

### Pairwise Alignment Parameter:

| FAST algorithm | on |
|---|---|
| K-tuple size | 1 |
| Gap penalty | 3 |
| Window size | 5 |
| Number of best diagonals | 5 |

Alternatively the identity may be determined according to Chenna, et al. (2003), the web page: http://www.ebi.ac.uk/Tools/clustalw/index.html# and the following settings

| | |
|---|---|
| DNA Gap Open Penalty | 15.0 |
| DNA Gap Extension Penalty | 6.66 |
| DNA Matrix | Identity |
| Protein Gap Open Penalty | 10.0 |
| Protein Gap Extension Penalty | 0.2 |
| Protein matrix | Gonnet |
| Protein/DNA ENDGAP | -1 |
| Protein/DNA GAPDIST | 4 |

All the nucleic acid sequences mentioned herein (single-stranded and double-stranded DNA and RNA sequences, for example cDNA and mRNA) can be produced in a known way by chemical synthesis from the nucleotide building blocks, e.g. by fragment condensation of individual overlapping, complementary nucleic acid building blocks of the double helix. Chemical synthesis of oligonucleotides can, for example, be performed in a known way, by the phosphoamidite method (Voet, Voet, 2nd edition, Wiley Press, New York, pages 896-897). The accumulation of synthetic oligonucleotides and filling of gaps by means of the Klenow fragment of DNA polymerase and ligation reactions as well as general cloning techniques are described in Sambrook et al. (1989), see below.

The nucleic acid molecules according to the invention can in addition contain non-translated sequences from the 3′ and/or 5′ end of the coding genetic region.

The invention further relates to the nucleic acid molecules that are complementary to the concretely described nucleotide sequences or a segment thereof.

The nucleotide sequences according to the invention make possible the production of probes and primers that can be used for the identification and/or cloning of homologous sequences in other cellular types and organisms. Such probes or primers generally comprise a nucleotide sequence region which hybridizes under “stringent” conditions (as defined herein elsewhere) on at least about 12, preferably at least about 25, for example about 40, 50 or 75 successive nucleotides of a sense strand of a nucleic acid sequence according to the invention or of a corresponding antisense strand.

“Homologous” sequences include orthologous or paralogous sequences. Methods of identifying orthologs or paralogs including phylogenetic methods, sequence similarity and hybridization methods are known in the art and are described herein.

“Paralogs” result from gene duplication that gives rise to two or more genes with similar sequences and similar functions. Paralogs typically cluster together and are formed by duplications of genes within related plant species. Paralogs are found in groups of similar genes using pair-wise Blast analysis or during phylogenetic analysis of gene families using programs such as CLUSTAL. In paralogs, consensus sequences can be identified characteristic to sequences within related genes and having similar functions of the genes.

“Orthologs”, or orthologous sequences, are sequences similar to each other because they are found in species that descended from a common ancestor. For instance, plant species that have common ancestors are known to contain many enzymes that have similar sequences and functions. The skilled artisan can identify orthologous sequences and predict the functions of the orthologs, for example, by constructing a polygenic tree for a gene family of one species using CLUSTAL or BLAST programs. A method for identifying or confirming similar functions among homologous sequences is by comparing of the transcript profiles in host cells or organisms, such as plants or microorganisms, overexpressing or lacking (in knockouts/knockdowns) related polypeptides. The skilled person will understand that genes having similar transcript profiles, with greater than 50% regulated transcripts in common, or with greater than 70% regulated transcripts in common, or greater than 90% regulated transcripts in common will have similar functions. Homologs, paralogs, orthologs and any other variants of the sequences herein are expected to function in a similar manner by making the host cells, organism such as plants or microorganisms producing terpene synthase proteins.

The term “selectable marker” refers to any gene which upon expression may be used to select a cell or cells that include the selectable marker. Examples of selectable markers are described below. The skilled artisan will know that different antibiotic, fungicide, auxotrophic or herbicide selectable markers are applicable to different target species.

An “isolated” nucleic acid molecule is separated from other nucleic acid molecules that are present in the natural source of the nucleic acid and can moreover be substantially free from other cellular material or culture medium, if it is being produced by recombinant techniques, or can be free from chemical precursors or other chemicals, if it is being synthesized chemically.

A nucleic acid molecule according to the invention can be isolated by means of standard techniques of molecular biology and the sequence information supplied according to the invention. For example, cDNA can be isolated from a suitable cDNA library, using one of the concretely disclosed complete sequences or a segment thereof as hybridization probe and standard hybridization techniques (as described for example in Sambrook, (1989)).

In addition, a nucleic acid molecule comprising one of the disclosed sequences or a segment thereof, can be isolated by the polymerase chain reaction, using the oligonucleotide primers that were constructed on the basis of this sequence. The nucleic acid amplified in this way can be cloned in a suitable vector and can be characterized by DNA sequencing. The oligonucleotides according to the invention can also be produced by standard methods of synthesis, e.g. using an automatic DNA synthesizer.

Nucleic acid sequences according to the invention or derivatives thereof, homologues or parts of these sequences, can for example be isolated by usual hybridization techniques or the PCR technique from other bacteria, e.g. via genomic or cDNA libraries. These DNA sequences hybridize in standard conditions with the sequences according to the invention.

“Hybridize” means the ability of a polynucleotide or oligonucleotide to bind to an almost complementary sequence in standard conditions, whereas nonspecific binding does not occur between non-complementary partners in these conditions. For this, the sequences can be 90-100% complementary. The property of complementary sequences of being able to bind specifically to one another is utilized for example in Northern Blotting or Southern Blotting or in primer binding in PCR or RT-PCR.

Short oligonucleotides of the conserved regions are used advantageously for hybridization. However, it is also possible to use longer fragments of the nucleic acids according to the invention or the complete sequences for the hybridization. These “standard conditions” vary depending on the nucleic acid used (oligonucleotide, longer fragment or complete sequence) or depending on which type of nucleic acid—DNA or RNA—is used for hybridization. For example, the melting temperatures for DNA:DNA hybrids are approx. 10° C. lower than those of DNA: RNA hybrids of the same length.

For example, depending on the particular nucleic acid, standard conditions mean temperatures between 42 and 58° C. in an aqueous buffer solution with a concentration between 0.1 to 5×SSC (1×SSC=0.15 M NaCl, 15 mM sodium citrate, pH 7.2) or additionally in the presence of 50% formamide, for example 42° C. in 5×SSC, 50% formamide. Advantageously, the hybridization conditions for DNA:DNA hybrids are 0.1×SSC and temperatures between about 20° C. to 45° C., preferably between about 30° C. to 45° C. For DNA: RNA hybrids the hybridization conditions are advantageously 0.1×SSC and temperatures between about 30° C. to 55° C., preferably between about 45° C. to 55° C. These stated temperatures for hybridization are examples of calculated melting temperature values for a nucleic acid with a length of approx. 100 nucleotides and a G+C content of 50% in the absence of formamide. The experimental conditions for DNA hybridization are described in relevant genetics textbooks, for example Sambrook et al., 1989, and can be calculated using formulae that are known by a person skilled in the art, for example depending on the length of the nucleic acids, the type of hybrids or the G+C content. A person skilled in the art can obtain further information on hybridization from the following textbooks: Ausubel et al. (eds), (1985), Brown (ed) (1991).

“Hybridization” can in particular be carried out under stringent conditions. Such hybridization conditions are for example described in Sambrook (1989), or in Current Protocols in Molecular Biology, John Wiley & Sons, N.Y. (1989), 6.3.1-6.3.6.

As used herein, the term hybridization or hybridizes under certain conditions is intended to describe conditions for hybridization and washes under which nucleotide sequences that are significantly identical or homologous to each other remain bound to each other. The conditions may be such that sequences, which are at least about 70%, such as at least about 80%, and such as at least about 85%, 90%, or 95% identical, remain bound to each other. Definitions of low stringency, moderate, and high stringency hybridization conditions are provided herein.

Appropriate hybridization conditions can be selected by those skilled in the art with minimal experimentation as exemplified in Ausubel et al. (1995, Current Protocols in Molecular Biology, John Wiley & Sons, sections 2, 4, and 6). Additionally, stringency conditions are described in Sambrook et al. (1989, Molecular Cloning: A Laboratory Manual, 2nd ed., Cold Spring Harbor Press, chapters 7, 9, and 11).

As used herein, defined conditions of low stringency are as follows. Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×106 32P-labeled probe is used. Filters are incubated in hybridization mixture for 18-20 h at 40° C., and then washed for 1.5 h at 55° C. In a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography.

As used herein, defined conditions of moderate stringency are as follows. Filters containing DNA are pretreated for 7 h at 50° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×106 32P-labeled probe is used. Filters are incubated in hybridization mixture for 30 h at 50° C., and then washed for 1.5 h at 55° C. In a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography.

As used herein, defined conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in the prehybridization mixture containing 100 μg/ml denatured salmon sperm DNA and 5-20×106 cpm of 32P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 minutes.

Other conditions of low, moderate, and high stringency well known in the art (e.g., as employed for cross-species hybridizations) may be used if the above conditions are inappropriate (e.g., as employed for cross-species hybridizations).

A detection kit for nucleic acid sequences encoding a polypeptide of the invention may include primers and/or probes specific for nucleic acid sequences encoding the polypeptide, and an associated protocol to use the primers and/or probes to detect nucleic acid sequences encoding the polypeptide in a sample. Such detection kits may be used to determine whether a plant, organism, microorganism or cell has been modified, i.e., transformed with a sequence encoding the polypeptide.

To test a function of variant DNA sequences according to an embodiment herein, the sequence of interest is operably linked to a selectable or screenable marker gene and expression of said reporter gene is tested in transient expression assays, for example, with microorganisms or with protoplasts or in stably transformed plants.

The invention also relates to derivatives of the concretely disclosed or derivable nucleic acid sequences.

Thus, further nucleic acid sequences according to the invention can be derived from the sequences specifically disclosed herein and can differ from it by one or more, like 1 to 20, in particular 1 to 15 or 5 to 10 additions, substitutions, insertions or deletions of one or several (like for example 1 to 10) nucleotides, and furthermore code for polypeptides with the desired profile of properties.

The invention also encompasses nucleic acid sequences that comprise so-called silent mutations or have been altered, in comparison with a concretely stated sequence, according to the codon usage of a special original or host organism.

According to a particular embodiment of the invention variant nucleic acids may be prepared in order to adapt its nucleotide sequence to a specific expression system. For example, bacterial expression systems are known to more efficiently express polypeptides if amino acids are encoded by particular codons. Due to the degeneracy of the genetic code, more than one codon may encode the same amino acid sequence, multiple nucleic acid sequences can code for the same protein or polypeptide, all these DNA sequences being encompassed by an embodiment herein. Where appropriate, the nucleic acid sequences encoding the polypeptides described herein may be optimized for increased expression in the host cell. For example, nucleic acids of an embodiment herein may be synthesized using codons particular to a host for improved expression.

The invention also encompasses naturally occurring variants, e.g. splicing variants or allelic variants, of the sequences described therein.

Allelic variants may have at least 60% homology at the level of the derived amino acid, preferably at least 80% homology, quite especially preferably at least 90% homology over the entire sequence range (regarding homology at the amino acid level, reference should be made to the details given above for the polypeptides). Advantageously, the homologies can be higher over partial regions of the sequences.

The invention also relates to sequences that can be obtained by conservative nucleotide substitutions (i.e. as a result thereof the amino acid in question is replaced by an amino acid of the same charge, size, polarity and/or solubility).

The invention also relates to the molecules derived from the concretely disclosed nucleic acids by sequence polymorphisms. Such genetic polymorphisms may exist in cells from different populations or within a population due to natural allelic variation. Allelic variants may also include functional equivalents. These natural variations usually produce a variance of 1 to 5% in the nucleotide sequence of a gene. Said polymorphisms may lead to changes in the amino acid sequence of the polypeptides disclosed herein. Allelic variants may also include functional equivalents.

Furthermore, derivatives are also to be understood to be homologs of the nucleic acid sequences according to the invention, for example animal, plant, fungal or bacterial homologs, shortened sequences, single-stranded DNA or RNA of the coding and noncoding DNA sequence. For example, homologs have, at the DNA level, a homology of at least 40%, preferably of at least 60%, especially preferably of at least 70%, quite especially preferably of at least 80% over the entire DNA region given in a sequence specifically disclosed herein.

Moreover, derivatives are to be understood to be, for example, fusions with promoters. The promoters that are added to the stated nucleotide sequences can be modified by at least one nucleotide exchange, at least one insertion, inversion and/or deletion, though without impairing the functionality or efficacy of the promoters. Moreover, the efficacy of the promoters can be increased by altering their sequence or can be exchanged completely with more effective promoters even of organisms of a different genus.

d. Generation of Functional Polypeptide Mutants

Moreover, a person skilled in the art is familiar with methods for generating functional mutants, that is to say nucleotide sequences which code for a polypeptide with at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity to anyone of amino acid related SEQ ID NOs as disclosed herein and/or encoded by a nucleic acid molecule comprising a nucleotide sequence having at least 70% sequence identity to anyone of the nucleotide related SEQ ID NOs as disclosed herein.

Depending on the technique used, a person skilled in the art can introduce entirely random or else more directed mutations into genes or else noncoding nucleic acid regions (which are for example important for regulating expression) and subsequently generate genetic libraries. The methods of molecular biology required for this purpose are known to the skilled worker and for example described in Sambrook and Russell, Molecular Cloning. 3rd Edition, Cold Spring Harbor Laboratory Press 2001.

Methods for modifying genes and thus for modifying the polypeptide encoded by them have been known to the skilled worker for a long time, such as, for example
site-specific mutagenesis, where individual or several nucleotides of a gene are replaced in a directed fashion (Trower M K (Ed.) 1996; In vitro mutagenesis protocols. Humana Press, New Jersey),
saturation mutagenesis, in which a codon for any amino acid can be exchanged or added at any point of a gene (Kegler-Ebo D M, Docktor C M, DiMaio D (1994) Nucleic Acids Res 22:1593; Barettino D, Feigenbutz M, Valcárel R, Stunnenberg H G (1994) Nucleic Acids Res 22:541; Barik S (1995) Mol Biotechnol 3:1),
error-prone polymerase chain reaction, where nucleotide sequences are mutated by error-prone DNA polymerases (Eckert K A, Kunkel T A (1990) Nucleic Acids Res 18:3739);
the SeSaM method (sequence saturation method), in which preferred exchanges are prevented by the polymerase. Schenk et al., Biospektrum, Vol. 3, 2006, 277-279
the passaging of genes in mutator strains, in which, for example owing to defective DNA repair mechanisms, there is an increased mutation rate of nucleotide sequences (Greener A, Callahan M, Jerpseth B (1996) An efficient random mutagenesis technique using an E. coli mutator strain. In: Trower M K (Ed.) In vitro mutagenesis protocols. Humana Press, New Jersey), or
DNA shuffling, in which a pool of closely related genes is formed and digested and the fragments are used as templates for a polymerase chain reaction in which, by repeated strand separation and reassociation, full-length mosaic genes are ultimately generated (Stemmer W P C (1994) Nature 370:389; Stemmer W P C (1994) Proc Natl Acad Sci USA 91:10747).

Using so-called directed evolution (described, inter alia, in Reetz M T and Jaeger K-E (1999), Topics Curr Chem 200:31; Zhao H, Moore J C, Volkov A A, Arnold F H (1999), Methods for optimizing industrial polypeptides by directed evolution, In: Demain A L, Davies J E (Ed.) Manual of industrial microbiology and biotechnology. American Society for Microbiology), a skilled worker can produce functional mutants in a directed manner and on a large scale. To this end, in a first step, gene libraries of the respective polypeptides are first produced, for example using the methods given above. The gene libraries are expressed in a suitable way, for example by bacteria or by phage display systems.

The relevant genes of host organisms which express functional mutants with properties that largely correspond to the desired properties can be submitted to another mutation cycle. The steps of the mutation and selection or screening can be repeated iteratively until the present functional mutants have the desired properties to a sufficient extent. Using this iterative procedure, a limited number of mutations, for example 1, 2, 3, 4 or 5 mutations, can be performed in stages and assessed and selected for their influence on the activity in question. The selected mutant can then be submitted to a further mutation step in the same way. In this way, the number of individual mutants to be investigated can be reduced significantly.

The results according to the invention also provide important information relating to structure and sequence of the relevant polypeptides, which is required for generating, in a targeted fashion, further polypeptides with desired modified properties. In particular, it is possible to define so-called “hot spots”, i.e. sequence segments that are potentially suitable for modifying a property by introducing targeted mutations.

Information can also be deduced regarding amino acid sequence positions, in the region of which mutations can be effected that should probably have little effect on the activity, and can be designated as potential “silent mutations”.

e. Constructs for Expressing Polypeptides of the Invention

In this context the following definitions apply:

“Expression of a gene” encompasses “heterologous expression” and “over-expression” and involves transcription of the gene and translation of the mRNA into a protein. Overexpression refers to the production of the gene product as measured by levels of mRNA, polypeptide and/or enzyme activity in transgenic cells or organisms that exceeds levels of production in non-transformed cells or organisms of a similar genetic background.

“Expression vector” as used herein means a nucleic acid molecule engineered using molecular biology methods and recombinant DNA technology for delivery of foreign or exogenous DNA into a host cell. The expression vector typically includes sequences required for proper transcription of the nucleotide sequence. The coding region usually codes for a protein of interest but may also code for an RNA, e.g., an antisense RNA, siRNA and the like.

An “expression vector” as used herein includes any linear or circular recombinant vector including but not limited to viral vectors, bacteriophages and plasmids. The skilled person is capable of selecting a suitable vector according to the expression system. In one embodiment, the expression vector includes the nucleic acid of an embodiment herein operably linked to at least one “regulatory sequence”, which controls transcription, translation, initiation and termination, such as a transcriptional promoter, operator or enhancer, or an mRNA ribosomal binding site and, optionally, including at least one selection marker. Nucleotide sequences are “operably linked” when the regulatory sequence functionally relates to the nucleic acid of an embodiment herein.

An “expression system” as used herein encompasses any combination of nucleic acid molecules required for the expression of one, or the co-expression of two or more polypeptides either in vivo of a given expression host, or in vitro. The respective coding sequences may either be located on a single nucleic acid molecule or vector, as for example a vector containing multiple cloning sites, or on a polycistronic nucleic acid, or may be distributed over two or more physically distinct vectors.

As used herein, the terms “amplifying” and “amplification” refer to the use of any suitable amplification methodology for generating or detecting recombinant of naturally expressed nucleic acid, as described in detail, below. For example, the invention provides methods and reagents (e.g., specific degenerate oligonucleotide primer pairs, oligo dT primer) for amplifying (e.g., by polymerase chain reaction, PCR) naturally expressed (e.g., genomic DNA or mRNA) or recombinant (e.g., cDNA) nucleic acids of the invention in vivo, ex vivo or in vitro.

“Regulatory sequence” refers to a nucleic acid sequence that determines expression level of the nucleic acid sequences of an embodiment herein and is capable of regulating the rate of transcription of the nucleic acid sequence operably linked to the regulatory sequence. Regulatory sequences comprise promoters, enhancers, transcription factors, promoter elements and the like.

A “promoter”, a “nucleic acid with promoter activity” or a “promoter sequence” is understood as meaning, in accordance with the invention, a nucleic acid which, when functionally linked to a nucleic acid to be transcribed, regulates the transcription of said nucleic acid. “Promoter” in particular refers to a nucleic acid sequence that controls the expression of a coding sequence by providing a binding site for RNA polymerase and other factors required for proper transcription including without limitation transcription factor binding sites, repressor and activator protein binding sites. The meaning of the term promoter also includes the term “promoter regulatory sequence”. Promoter regulatory sequences may include upstream and downstream elements that may influences transcription, RNA processing or stability of the associated coding nucleic acid sequence. Promoters include naturally-derived and synthetic sequences. The coding nucleic acid sequences is usually located downstream of the promoter with respect to the direction of the transcription starting at the transcription initiation site.

In this context, a “functional” or “operative” linkage is understood as meaning for example the sequential arrangement of one of the nucleic acids with a regulatory sequence. For example the sequence with promoter activity and of a nucleic acid sequence to be transcribed and optionally further regulatory elements, for example nucleic acid sequences which ensure the transcription of nucleic acids, and for example a terminator, are linked in such a way that each of the regulatory elements can perform its function upon transcription of the nucleic acid sequence. This does not necessarily require a direct linkage in the chemical sense. Genetic control sequences, for example enhancer sequences, can even exert their function on the target sequence from more remote positions or even from other DNA molecules. Preferred arrangements are those in which the nucleic acid sequence to be transcribed is positioned behind (i.e. at the 3′-end of) the promoter sequence so that the two sequences are joined together covalently. The distance between the promoter sequence and the nucleic acid sequence to be expressed recombinantly can be smaller than 200 base pairs, or smaller than 100 base pairs or smaller than 50 base pairs.

In addition to promoters and terminator, the following may be mentioned as examples of other regulatory elements: targeting sequences, enhancers, polyadenylation signals, selectable markers, amplification signals, replication origins and the like. Suitable regulatory sequences are described, for example, in Goeddel, Gene Expression Technology: Methods in Enzymology 185, Academic Press, San Diego, CA (1990).

The term “constitutive promoter” refers to an unregulated promoter that allows for continual transcription of the nucleic acid sequence it is operably linked to.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous. The nucleotide sequence associated with the promoter sequence may be of homologous or heterologous origin with respect to the plant to be transformed. The sequence also may be entirely or partially synthetic. Regardless of the origin, the nucleic acid sequence associated with the promoter sequence will be expressed or silenced in accordance with promoter properties to which it is linked after binding to the polypeptide of an embodiment herein. The associated nucleic acid may code for a protein that is desired to be expressed or suppressed throughout the organism at all times or, alternatively, at a specific time or in specific tissues, cells, or cell compartment. Such nucleotide sequences particularly encode proteins conferring desirable phenotypic traits to the host cells or organism altered or transformed therewith. More particularly, the associated nucleotide sequence leads to the production of the product or products of interest as herein defined in the cell or organism. Particularly, the nucleotide sequence encodes a polypeptide having an enzyme activity as herein defined.

The nucleotide sequence as described herein above may be part of an “expression cassette”. The terms “expression cassette” and “expression construct” are used synonymously. The (preferably recombinant) expression construct contains a nucleotide sequence which encodes a polypeptide according to the invention and which is under genetic control of regulatory nucleic acid sequences.

In a process applied according to the invention, the expression cassette may be part of an “expression vector”, in particular of a recombinant expression vector.

An “expression unit” is understood as meaning, in accordance with the invention, a nucleic acid with expression activity which comprises a promoter as defined herein and, after functional linkage with a nucleic acid to be expressed or a gene, regulates the expression, i.e. the transcription and the translation of said nucleic acid or said gene. It is therefore in this connection also referred to as a “regulatory nucleic acid sequence”. In addition to the promoter, other regulatory elements, for example enhancers, can also be present.

An “expression cassette” or “expression construct” is understood as meaning, in accordance with the invention, an expression unit which is functionally linked to the nucleic acid to be expressed or the gene to be expressed. In contrast to an expression unit, an expression cassette therefore comprises not only nucleic acid sequences which regulate transcription and translation, but also the nucleic acid sequences that are to be expressed as protein as a result of transcription and translation.

The terms “expression” or “overexpression” describe, in the context of the invention, the production or increase in intracellular activity of one or more polypeptides in a microorganism, which are encoded by the corresponding DNA. To this end, it is possible for example to introduce a gene into an organism, replace an existing gene with another gene, increase the copy number of the gene(s), use a strong promoter or use a gene which encodes for a corresponding polypeptide with a high activity; optionally, these measures can be combined.

Preferably such constructs according to the invention comprise a promoter 5′-upstream of the respective coding sequence and a terminator sequence 3′-downstream and optionally other usual regulatory elements, in each case in operative linkage with the coding sequence.

Nucleic acid constructs according to the invention comprise in particular a sequence coding for a polypeptide for example derived from the amino acid related SEQ ID NOs as described therein or the reverse complement thereof, or derivatives and homologs thereof and which have been linked operatively or functionally with one or more regulatory signals, advantageously for controlling, for example increasing, gene expression.

In addition to these regulatory sequences, the natural regulation of these sequences may still be present before the actual structural genes and optionally may have been genetically modified so that the natural regulation has been switched off and expression of the genes has been enhanced. The nucleic acid construct may, however, also be of simpler construction, i.e. no additional regulatory signals have been inserted before the coding sequence and the natural promoter, with its regulation, has not been removed. Instead, the natural regulatory sequence is mutated such that regulation no longer takes place and the gene expression is increased.

A preferred nucleic acid construct advantageously also comprises one or more of the already mentioned “enhancer” sequences in functional linkage with the promoter, which sequences make possible an enhanced expression of the nucleic acid sequence. Additional advantageous sequences may also be inserted at the 3′-end of the DNA sequences, such as further regulatory elements or terminators. One or more copies of the nucleic acids according to the invention may be present in a construct. In the construct, other markers, such as genes which complement auxotrophisms or antibiotic resistances, may also optionally be present so as to select for the construct.

Examples of suitable regulatory sequences are present in promoters such as cos, tac, trp, tet, trp-tet, lpp, lac, lpp-lac, lacl4, T7, T5, T3, gal, trc, ara, rhaP (rhaPBAD) SP6, lambda-PR or in the lambda-PL promoter, and these are advantageously employed in Gram-negative bacteria. Further advantageous regulatory sequences are present for example in the Gram-positive promoters amy and SPO2, in the yeast or fungal promoters ADC1, MFalpha, AC, P-60, CYC1, GAPDH, TEF, rp28, ADH. Artificial promoters may also be used for regulation.

For expression in a host organism, the nucleic acid construct is inserted advantageously into a vector such as, for example, a plasmid or a phage, which makes possible optimal expression of the genes in the host. Vectors are also understood as meaning, in addition to plasmids and phages, all the other vectors which are known to the skilled worker, that is to say for example viruses such as SV40, CMV, baculovirus and adenovirus, transposons, IS elements, phasmids, cosmids and linear or circular DNA or artificial chromosomes. These vectors are capable of replicating autonomously in the host organism or else chromosomally. These vectors are a further development of the invention. Binary or cpo-integration vectors are also applicable.

Suitable plasmids are, for example, in E. coli pLG338, pACYC184, pBR322, pUC18, pUC19, pKC30, pRep4, pHS1, pKK223-3, pDHE19.2, pHS2, pPLc236, pMBL24, pLG200, pUR290, pIN-III113-B1, 2gt11 or pBdCI, in Streptomyces pIJ101, pIJ364, pIJ702 or pIJ361, in Bacillus pUB110, pC194 or pBD214, in Corynebacterium pSA77 or pAJ667, in fungi pALS1, pIL2 or pBB116, in yeasts 2alphaM, pAG-1, YEp6, YEp13 or pEMBLYe23 or in plants pLGV23, pGHlac⁺, pBIN19, pAK2004 or pDH51. The abovementioned plasmids are a small selection of the plasmids which are possible. Further plasmids are well known to the skilled worker and can be found for example in the book Cloning Vectors (Eds. Pouwels P. H. et al. Elsevier, Amsterdam-New York-Oxford, 1985, ISBN 0 444 904018).

In a further development of the vector, the vector which comprises the nucleic acid construct according to the invention or the nucleic acid according to the invention can advantageously also be introduced into the microorganisms in the form of a linear DNA and integrated into the host organism's genome via heterologous or homologous recombination. This linear DNA can consist of a linearized vector such as a plasmid or only of the nucleic acid construct or the nucleic acid according to the invention.

For optimal expression of heterologous genes in organisms, it is advantageous to modify the nucleic acid sequences to match the specific “codon usage” used in the organism. The “codon usage” can be determined readily by computer evaluations of other, known genes of the organism in question.

An expression cassette according to the invention is generated by fusing a suitable promoter to a suitable coding nucleotide sequence and a terminator or polyadenylation signal. Customary recombination and cloning techniques are used for this purpose, as are described, for example, in T. Maniatis, E. F. Fritsch and J. Sambrook, Molecular Cloning: A Laboratory Manual, Cold Spring Harbor Laboratory, Cold Spring Harbor, NY (1989) and in T. J. Silhavy, M. L. Berman and L. W. Enquist, Experiments with Gene Fusions, Cold Spring Harbor Laboratory, Cold Spring Harbor, NY (1984) and in Ausubel, F. M. et al., Current Protocols in Molecular Biology, Greene Publishing Assoc. and Wiley Interscience (1987).

For expression in a suitable host organism, the recombinant nucleic acid construct or gene construct is advantageously inserted into a host-specific vector which makes possible optimal expression of the genes in the host. Vectors are well known to the skilled worker and can be found for example in “cloning vectors” (Pouwels P. H. et al., Ed., Elsevier, Amsterdam-New York-Oxford, 1985).

An alternative embodiment of an embodiment herein provides a method to “alter gene expression” in a host cell. For instance, the polynucleotide of an embodiment herein may be enhanced or overexpressed or induced in certain contexts (e.g. upon exposure to certain temperatures or culture conditions) in a host cell or host organism.

Alteration of expression of a polynucleotide provided herein may also result in ectopic expression which is a different expression pattern in an altered and in a control or wild-type organism. Alteration of expression occurs from interactions of polypeptide of an embodiment herein with exogenous or endogenous modulators, or as a result of chemical modification of the polypeptide. The term also refers to an altered expression pattern of the polynucleotide of an embodiment herein which is altered below the detection level or completely suppressed activity.

One embodiment, provided herein is also an isolated, recombinant or synthetic polynucleotide encoding a polypeptide or variant polypeptide provided herein.

In one embodiment, several polypeptide encoding nucleic acid sequences are co-expressed in a single host, particularly under control of different promoters. In another embodiment, several polypeptide encoding nucleic acid sequences can be present on a single transformation vector or be co-transformed at the same time using separate vectors and selecting transformants comprising both chimeric genes. Similarly, one or polypeptide encoding genes may be expressed in a single plant, cell, microorganism or organism together with other chimeric genes.

f. Hosts to be Applied for the Present Invention

Depending on the context, the term “host” can mean the wild-type host or a genetically altered, recombinant host or both.

In principle, all prokaryotic or eukaryotic organisms may be considered as host or recombinant host organisms for the nucleic acids or the nucleic acid constructs according to the invention.

Using the vectors according to the invention, recombinant hosts can be produced, which are for example transformed with at least one vector according to the invention and can be used for producing the polypeptides according to the invention. Advantageously, the recombinant constructs according to the invention, described above, are introduced into a suitable host system and expressed. Preferably common cloning and transfection methods, known by a person skilled in the art, are used, for example co-precipitation, protoplast fusion, electroporation, retroviral transfection and the like, for expressing the stated nucleic acids in the respective expression system. Suitable systems are described for example in Current Protocols in Molecular Biology, F. Ausubel et al., Ed., Wiley Interscience, New York 1997, or Sambrook et al. Molecular Cloning: A Laboratory Manual. 2nd edition, Cold Spring Harbor Laboratory, Cold Spring Harbor Laboratory Press, Cold Spring Harbor, NY, 1989.

Advantageously, microorganisms such as bacteria, fungi or yeasts are used as host organisms. Advantageously, gram-positive or gram-negative bacteria are used, preferably bacteria of the families Enterobacteriaceae, Pseudomonadaceae, Rhizobiaceae, Streptomycetaceae, Streptococcaceae or Nocardiaceae, especially preferably bacteria of the genera Escherichia, Pseudomonas, Streptomyces, Lactococcus, Nocardia, Burkholderia, Salmonella, Agrobacterium, Clostridium or Rhodococcus. The genus and species Escherichia coli is quite especially preferred. Furthermore, other advantageous bacteria are to be found in the group of alpha-Proteobacteria, beta-Proteobacteria or gamma-Proteobacteria. Advantageously also yeasts of families like Saccharomyces or Pichia are suitable hosts.

Alternatively, entire plants or plant cells may serve as natural or recombinant host. As non-limiting examples the following plants or cells derived therefrom may be mentioned the genera Nicotiana, in particular Nicotiana benthamiana and Nicotiana tabacum (tobacco); as well as Arabidopsis, in particular Arabidopsis thaliana.

Depending on the host organism, the organisms used in the method according to the invention are grown or cultured in a manner known by a person skilled in the art. Culture can be batchwise, semi-batchwise or continuous. Nutrients can be present at the beginning of fermentation or can be supplied later, semicontinuously or continuously. This is also described in more detail below.

g. Recombinant Production of Polypeptides According to the Invention

The invention further relates to methods for recombinant production of polypeptides according to the invention or functional, biologically active fragments thereof, wherein a polypeptide-producing microorganism is cultured, optionally the expression of the polypeptides is induced by applying at least one inducer inducing gene expression and the expressed polypeptides are isolated from the culture. The polypeptides can also be produced in this way on an industrial scale, if desired.

The microorganisms produced according to the invention can be cultured continuously or discontinuously in the batch method or in the fed-batch method or repeated fed-batch method. A summary of known cultivation methods can be found in the textbook by Chmiel (Bioprozesstechnik 1. Einführung in die Bioverfahrenstechnik [Bioprocess technology 1. Introduction to bioprocess technology] (Gustav Fischer Verlag, Stuttgart, 1991)) or in the textbook by Storhas (Bioreaktoren und periphere Einrichtungen [Bioreactors and peripheral equipment] (Vieweg Verlag, Braunschweig/Wiesbaden, 1994)).

The culture medium to be used must suitably meet the requirements of the respective strains. Descriptions of culture media for various microorganisms are given in the manual “Manual of Methods for General Bacteriology” of the American Society for Bacteriology (Washington D. C., USA, 1981).

These media usable according to the invention usually comprise one or more carbon sources, nitrogen sources, inorganic salts, vitamins and/or trace elements.

Preferred carbon sources are sugars, such as mono-, di- or polysaccharides. Very good carbon sources are for example glucose, fructose, mannose, galactose, ribose, sorbose, ribulose, lactose, maltose, sucrose, raffinose, starch or cellulose. Sugars can also be added to the media via complex compounds, such as molasses, or other by-products of sugar refining. It can also be advantageous to add mixtures of different carbon sources. Other possible carbon sources are oils and fats, for example soybean oil, sunflower oil, peanut oil and coconut oil, fatty acids, for example palmitic acid, stearic acid or linoleic acid, alcohols, for example glycerol, methanol or ethanol and organic acids, for example acetic acid or lactic acid.

Nitrogen sources are usually organic or inorganic nitrogen compounds or materials that contain these compounds. Examples of nitrogen sources comprise ammonia gas or ammonium salts, such as ammonium sulfate, ammonium chloride, ammonium phosphate, ammonium carbonate or ammonium nitrate, nitrates, urea, amino acids or complex nitrogen sources, such as corn-steep liquor, soya flour, soya protein, yeast extract, meat extract and others. The nitrogen sources can be used alone or as a mixture.

Inorganic salt compounds that can be present in the media comprise the chloride, phosphorus or sulfate salts of calcium, magnesium, sodium, cobalt, molybdenum, potassium, manganese, zinc, copper and iron.

Inorganic sulfur-containing compounds, for example sulfates, sulfites, dithionites, tetrathionates, thiosulfates, sulfides, as well as organic sulfur compounds, such as mercaptans and thiols, can be used as the sulfur source.

Phosphoric acid, potassium dihydrogen phosphate or dipotassium hydrogen phosphate or the corresponding sodium-containing salts can be used as the phosphorus source.

Chelating agents can be added to the medium, in order to keep the metal ions in solution. Especially suitable chelating agents comprise dihydroxyphenols, such as catechol or protocatechuate, or organic acids, such as citric acid.

The fermentation media used according to the invention usually also contain other growth factors, such as vitamins or growth promoters, which include for example biotin, riboflavin, thiamine, folic acid, nicotinic acid, pantothenate and pyridoxine. Growth factors and salts often originate from the components of complex media, such as yeast extract, molasses, corn-steep liquor and the like. Moreover, suitable precursors can be added to the culture medium. The exact composition of the compounds in the medium is strongly dependent on the respective experiment and is decided for each specific case individually. Information on media optimization can be found in the textbook “Applied Microbiol. Physiology, A Practical Approach” (Ed. P. M. Rhodes, P. F. Stanbury, IRL Press (1997) p. 53-73, ISBN 0 19 963577 3). Growth media can also be obtained from commercial suppliers, such as Standard 1 (Merck) or BHI (brain heart infusion, DIFCO) and the like.

All components of the medium are sterilized, either by heat (20 min at 1.5 bar and 121° C.) or by sterile filtration. The components can either be sterilized together, or separately if necessary. All components of the medium can be present at the start of culture or can be added either continuously or batchwise.

The culture temperature is normally between 15° C. and 45° C., preferably 25° C. to 40° C. and can be varied or kept constant during the experiment. The pH of the medium should be in the range from 5 to 8.5, preferably around 7.0. The pH for growing can be controlled during growing by adding basic compounds such as sodium hydroxide, potassium hydroxide, ammonia or ammonia water or acid compounds such as phosphoric acid or sulfuric acid. Antifoaming agents, for example fatty acid polyglycol esters, can be used for controlling foaming. To maintain the stability of plasmids, suitable selective substances, for example antibiotics, can be added to the medium. To maintain aerobic conditions, oxygen or oxygen-containing gas mixtures, for example ambient air, are fed into the culture. The temperature of the culture is normally in the range from 20° C. to 45° C. The culture is continued until a maximum of the desired product has formed. This target is normally reached within 10 hours to 160 hours.

The fermentation broth is then processed further. Depending on requirements, the biomass can be removed from the fermentation broth completely or partially by separation techniques, for example centrifugation, filtration, decanting or a combination of these methods or can be left in it completely.

If the polypeptides are not secreted in the culture medium, the cells can also be lysed and the product can be obtained from the lysate by known methods for isolation of proteins. The cells can optionally be disrupted with high-frequency ultrasound, high pressure, for example in a French press, by osmolysis, by the action of detergents, lytic enzymes or organic solvents, by means of homogenizers or by a combination of several of the aforementioned methods.

The polypeptides can be purified by known chromatographic techniques, such as molecular sieve chromatography (gel filtration), such as Q-sepharose chromatography, ion exchange chromatography and hydrophobic chromatography, and with other usual techniques such as ultrafiltration, crystallization, salting-out, dialysis and native gel electrophoresis. Suitable methods are described for example in Cooper, T. G., Biochemische Arbeitsmethoden [Biochemical processes], Verlag Walter de Gruyter, Berlin, New York or in Scopes, R., Protein Purification, Springer Verlag, New York, Heidelberg, Berlin.

For isolating the recombinant protein, it can be advantageous to use vector systems or oligonucleotides, which lengthen the cDNA by defined nucleotide sequences and therefore code for altered polypeptides or fusion proteins, which for example serve for easier purification. Suitable modifications of this type are for example so-called “tags” functioning as anchors, for example the modification known as hexa-histidine anchor or epitopes that can be recognized as antigens of antibodies (described for example in Harlow, E. and Lane, D., 1988, Antibodies: A Laboratory Manual. Cold Spring Harbor (N.Y.) Press). These anchors can serve for attaching the proteins to a solid carrier, for example a polymer matrix, which can for example be used as packing in a chromatography column, or can be used on a microtiter plate or on some other carrier.

At the same time these anchors can also be used for recognition of the proteins. For recognition of the proteins, it is moreover also possible to use usual markers, such as fluorescent dyes, enzyme markers, which form a detectable reaction product after reaction with a substrate, or radioactive markers, alone or in combination with the anchors for derivatization of the proteins.

h. Polypeptide Immobilization

The enzymes or polypeptides according to the invention can be used free or immobilized in the method described herein. An immobilized enzyme is an enzyme that is fixed to an inert carrier. Suitable carrier materials and the enzymes immobilized thereon are known from EP-A-1149849, EP-A-1 069 183 and DE-OS 100193773 and from the references cited therein. Reference is made in this respect to the disclosure of these documents in their entirety. Suitable carrier materials include for example clays, clay minerals, such as kaolinite, diatomaceous earth, perlite, silica, aluminum oxide, sodium carbonate, calcium carbonate, cellulose powder, anion exchanger materials, synthetic polymers, such as polystyrene, acrylic resins, phenol formaldehyde resins, polyurethanes and polyolefins, such as polyethylene and polypropylene. For making the supported enzymes, the carrier materials are usually employed in a finely-divided, particulate form, porous forms being preferred. The particle size of the carrier material is usually not more than 5 mm, in particular not more than 2 mm (particle-size distribution curve). Similarly, when using dehydrogenase as whole-cell catalyst, a free or immobilized form can be selected. Carrier materials are e.g. Ca-alginate, and carrageenan. Enzymes as well as cells can also be crosslinked directly with glutaraldehyde (cross-linking to CLEAs). Corresponding and other immobilization techniques are described for example in J. Lalonde and A. Margolin “Immobilization of Enzymes” in K. Drauz and H. Waldmann, Enzyme Catalysis in Organic Synthesis 2002, Vol. III, 991-1032, Wiley-VCH, Weinheim. Further information on biotransformations and bioreactors for carrying out methods according to the invention are also given for example in Rehm et al. (Ed.) Biotechnology, 2nd Edn, Vol 3, Chapter 17, VCH, Weinheim.

i. Reaction Conditions for Biocatalytic Production Methods of the Invention

The reaction of the present invention may be performed under in vivo or in vitro conditions.

The at least one polypeptide/enzyme which is present during a method of the invention or an individual step of a multistep-method as defined herein above, can be present in living cells naturally or recombinantly producing the enzyme or enzymes, in harvested cells. i.e. under in vivo conditions, or, in dead cells, in permeabilized cells, in crude cell extracts, in purified extracts, or in essentially pure or completely pure form, i.e. under in vitro conditions. The at least one enzyme may be present in solution or as an enzyme immobilized on a carrier. One or several enzymes may simultaneously be present in soluble and/or immobilised form.

The methods according to the invention can be performed in common reactors, which are known to those skilled in the art, and in different ranges of scale, e.g. from a laboratory scale (few millilitres to dozens of litres of reaction volume) to an industrial scale (several litres to thousands of cubic meters of reaction volume). If the polypeptide is used in a form encapsulated by non-living, optionally permeabilized cells, in the form of a more or less purified cell extract or in purified form, a chemical reactor can be used. The chemical reactor usually allows controlling the amount of the at least one enzyme, the amount of the at least one substrate, the pH, the temperature and the circulation of the reaction medium. When the at least one polypeptide/enzyme is present in living cells, the process will be a fermentation. In this case the biocatalytic production will take place in a bioreactor (fermenter), where parameters necessary for suitable living conditions for the living cells (e.g. culture medium with nutrients, temperature, aeration, presence or absence of oxygen or other gases, antibiotics, and the like) can be controlled. Those skilled in the art are familiar with chemical reactors or bioreactors, e.g. with procedures for up-scaling chemical or biotechnological methods from laboratory scale to industrial scale, or for optimizing process parameters, which are also extensively described in the literature (for biotechnological methods see e.g. Crueger und Crueger, Biotechnologie-Lehrbuch der angewandten Mikrobiologie, 2. Ed., R. Oldenbourg Verlag, München, Wien, 1984).

Cells containing the at least one enzyme can be permeabilized by physical or mechanical means, such as ultrasound or radiofrequency pulses, French presses, or chemical means, such as hypotonic media, lytic enzymes and detergents present in the medium, or combination of such methods. Examples for detergents are digitonin, n-dodecylmaltoside, octylglycoside, Triton® X-100, Tween® 20, deoxycholate, CHAPS (3-[(3-Cholamidopropyl) dimethylammonio]-1-propansulfonate), Nonidet R P40 (Ethylphenolpoly (ethyleneglycolether), and the like.

Instead of living cells biomass of non-living cells containing the required biocatalyst(s) may be applied of the biotransformation reactions of the invention as well.

If the at least one enzyme is immobilised, it is attached to an inert carrier as described above.

The conversion reaction can be carried out batch wise, semi-batch wise or continuously. Reactants (and optionally nutrients) can be supplied at the start of reaction or can be supplied subsequently, either semi-continuously or continuously.

The reaction of the invention, depending on the particular reaction type, may be performed in an aqueous, aqueous-organic or non-aqueous reaction medium.

An aqueous or aqueous-organic medium may contain a suitable buffer in order to adjust the pH to a value in the range of 5 to 11, like 6 to 10.

In an aqueous-organic medium an organic solvent miscible, partly miscible or immiscible with water may be applied. Non-limiting examples of suitable organic solvents are listed below. Further examples are mono- or polyhydric, aromatic or aliphatic alcohols, in particular polyhydric aliphatic alcohols like glycerol.

The non-aqueous medium may contain is substantially free of water, i.e. will contain less that about 1 wt.-% or 0.5 wt.-% of water.

Biocatalytic methods may also be performed in an organic non-aqueous medium. As suitable organic solvents there may be mentioned aliphatic hydrocarbons having for example 5 to 8 carbon atoms, like pentane, cyclopentane, hexane, cyclohexane, heptane, octane or cyclooctane; aromatic carbohydrates, like benzene, toluene, xylenes, chlorobenzene or dichlorobenzene, aliphatic acyclic and ethers, like diethylether, methyl-tert.-butylether, ethyl-tert.-butylether, dipropylether, diisopropylether, dibutylether; or mixtures thereof.

The concentration of the reactants/substrates may be adapted to the optimum reaction conditions, which may depend on the specific enzyme applied. For example, the initial substrate concentration may be in the 0.1 to 0.5 M, as for example 10 to 100 mM.

The reaction temperature may be adapted to the optimum reaction conditions, which may depend on the specific enzyme applied. For example, the reaction may be performed at a temperature in a range of from 0 to 70° C., as for example 20 to 50 or 25 to 40° C. Examples for reaction temperatures are about 30° C., about 35° C., about 37° C., about 40° C., about 45° C., about 50° C., about 55° C. and about 60° C.

The process may proceed until equilibrium between the substrate and then product(s) is achieved, but may be stopped earlier. Usual process times are in the range from 1 minute to 25 hours, in particular 10 min to 6 hours, as for example in the range from 1 hour to 4 hours, in particular 1.5 hours to 3.5 hours. These parameters are non-limiting examples of suitable process conditions.

If the host is a transgenic plant, optimal growth conditions can be provided, such as optimal light, water and nutrient conditions, for example.

Particular reaction conditions for performing the preparation of drimanyl acetate compound are as follows. In an aqueous environment, incubated at 20 to 35° C. and a pH of 4 to 7, the acetyltransferase enzyme can be present as a purified polypeptide or in a whole-cell system. Substrate concentration might vary between 10 and 100 mM.

k. Product Isolation

The methodology of the present invention can further include a step of recovering an end or intermediate product, optionally in stereoisomerically or enantiomerically substantially pure form. The term “recovering” includes extracting, harvesting, isolating or purifying the compound from culture or reaction media. Recovering the compound can be performed according to any conventional isolation or purification methodology known in the art including, but not limited to, treatment with a conventional resin (e.g., anion or cation exchange resin, non-ionic adsorption resin, etc.), treatment with a conventional adsorbent (e.g., activated charcoal, silicic acid, silica gel, cellulose, alumina, etc.), alteration of pH, solvent extraction (e.g., with a conventional solvent such as an alcohol, ethyl acetate, hexane and the like), distillation, dialysis, filtration, concentration, crystallization, recrystallization, pH adjustment, lyophilization and the like.

Identity and purity of the isolated product may be determined by known techniques, like High Performance Liquid Chromatography (HPLC), gas chromatography (GC), Spektroskopy (like IR, UV, NMR), Colouring methods, TLC, NIRS, enzymatic or microbial assays. (see for example: Patek et al. (1994) Appl. Environ. Microbiol. 60:133-140; Malakhova et al. (1996) Biotekhnologiya 11 27-32; und Schmidt et al. (1998) Bioprocess Engineer. 19:67-70. Ullmann's Encyclopedia of Industrial Chemistry (1996) Bd. A27, VCH: Weinheim, S. 89-90, S. 521-540, S. 540-547, S. 559-566, 575-581 und S. 581-587; Michal, G (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, Bd. 17.)

The cyclic terpene compound produced in any of the method described herein can be converted to derivatives such as, but not limited to hydrocarbons, esters, amides, glycosides, ethers, epoxides, aldehydes, ketons, alcohols, diols, acetals or ketals. The terpene compound derivatives can be obtained by a chemical method such as, but not limited to oxidation, reduction, alkylation, acylation and/or rearrangement. Alternatively, the terpene compound derivatives can be obtained using a biochemical method by contacting the terpene compound with an enzyme such as, but not limited to an oxidoreductase, a monooxygenase, a dioxygenase, a transferase. The biochemical conversion can be performed in-vitro using isolated enzymes, enzymes from lysed cells or in-vivo using whole cells.

l. Fermentative Production of a Drimenyl Acetate

The invention also relates to methods for the fermentative production of drimanyl acetate.

A fermentation as used according to the present invention can, for example, be performed in stirred fermenters, bubble columns and loop reactors. A comprehensive overview of the possible method types including stirrer types and geometric designs can be found in “Chmiel: Bioprozesstechnik: Einfuhrung in die Bioverfahrenstechnik, Band 1”. In the process of the invention, typical variants available are the following variants known to those skilled in the art or explained, for example, in “Chmiel, Hammes and Bailey: Biochemical Engineering”, such as batch, fed-batch, repeated fed-batch or else continuous fermentation with and without recycling of the biomass. Depending on the production strain, sparging with air, oxygen, carbon dioxide, hydrogen, nitrogen or appropriate gas mixtures may be effected in order to achieve good yield (YP/S).

The culture medium that is to be used must satisfy the requirements of the particular strains in an appropriate manner. Descriptions of culture media for various microorganisms are given in the handbook “Manual of Methods for General Bacteriology” of the American Society for Bacteriology (Washington D. C., USA, 1981).

These media that can be used according to the invention may comprise one or more sources of carbon, sources of nitrogen, inorganic salts, vitamins and/or trace elements.

Preferred sources of carbon are sugars, such as mono-, di- or polysaccharides. Very good sources of carbon are for example glucose, fructose, mannose, galactose, ribose, sorbose, ribulose, lactose, maltose, sucrose, raffinose, starch or cellulose. Sugars can also be added to the media via complex compounds, such as molasses, or other by-products from sugar refining. It may also be advantageous to add mixtures of various sources of carbon. Other possible sources of carbon are oils and fats such as soybean oil, sunflower oil, peanut oil and coconut oil, fatty acids such as palmitic acid, stearic acid or linoleic acid, alcohols such as glycerol, methanol or ethanol and organic acids such as acetic acid or lactic acid.

Sources of nitrogen are usually organic or inorganic nitrogen compounds or materials containing these compounds. Examples of sources of nitrogen include ammonia gas or ammonium salts, such as ammonium sulfate, ammonium chloride, ammonium phosphate, ammonium carbonate or ammonium nitrate, nitrates, urea, amino acids or complex sources of nitrogen, such as corn-steep liquor, soybean flour, soy-bean protein, yeast extract, meat extract and others. The sources of nitrogen can be used separately or as a mixture.

Inorganic salt compounds that may be present in the media comprise the chloride, phosphate or sulfate salts of calcium, magnesium, sodium, cobalt, molybdenum, potassium, manganese, zinc, copper and iron.

Inorganic sulfur-containing compounds, for example sulfates, sulfites, di-thionites, tetrathionates, thiosulfates, sulfides, but also organic sulfur compounds, such as mercaptans and thiols, can be used as sources of sulfur.

Phosphoric acid, potassium dihydrogenphosphate or dipotassium hydrogenphosphate or the corresponding sodium-containing salts can be used as sources of phosphorus.

Chelating agents can be added to the medium, in order to keep the metal ions in solution. Especially suitable chelating agents comprise dihydroxyphenols, such as catechol or protocatechuate, or organic acids, such as citric acid.

The fermentation media used according to the invention may also contain other growth factors, such as vitamins or growth promoters, which include for example biotin, riboflavin, thiamine, folic acid, nicotinic acid, pantothenate and pyridoxine. Growth factors and salts often come from complex components of the media, such as yeast extract, molasses, corn-steep liquor and the like. In addition, suitable precursors can be added to the culture medium. The precise composition of the compounds in the medium is strongly dependent on the particular experiment and must be decided individually for each specific case. Information on media optimization can be found in the textbook “Applied Microbiol. Physiology, A Practical Approach” (1997) Growing media can also be obtained from commercial suppliers, such as Standard 1 (Merck) or BHI (Brain heart infusion, DIFCO) etc.

All components of the medium are sterilized, either by heating (20 min at 1.5 bar and 121° C.) or by sterile filtration. The components can be sterilized either together, or if necessary separately. All the components of the medium can be present at the start of growing, or optionally can be added continuously or by batch feed.

The temperature of the culture is normally between 15° C. and 45° C., preferably 25° C. to 40° C. and can be kept constant or can be varied during the experiment. The pH value of the medium should be in the range from 5 to 8.5, preferably around 7.0. The pH value for growing can be controlled during growing by adding basic compounds such as sodium hydroxide, potassium hydroxide, ammonia or ammonia water or acid compounds such as phosphoric acid or sulfuric acid. Antifoaming agents, e.g. fatty acid polyglycol esters, can be used for controlling foaming. To maintain the stability of plasmids, suitable substances with selective action, e.g. antibiotics, can be added to the medium. Oxygen or oxygen-containing gas mixtures, e.g. the ambient air, are fed into the culture in order to maintain aerobic conditions. The temperature of the culture is normally from 20° C. to 45° C. Culture is continued until a maximum of the desired product has formed. This is normally achieved within 1 hour to 160 hours.

The methodology of the present invention can further include a step of recovering said drimanyl acetate.

The term “recovering” includes extracting, harvesting, isolating or purifying the compound from culture media. Recovering the compound can be performed according to any conventional isolation or purification methodology known in the art including, but not limited to, treatment with a conventional resin (e.g., anion or cation exchange resin, non-ionic adsorption resin, etc.), treatment with a conventional adsorbent (e.g., activated charcoal, silicic acid, silica gel, cellulose, alumina, etc.), alteration of pH, solvent extraction (e.g., with a conventional solvent such as an alcohol, ethyl acetate, hexane and the like), distillation, dialysis, filtration, concentration, crystallization, recrystallization, pH adjustment, lyophilization and the like.

Before the intended isolation the biomass of the broth can be removed. Processes for removing the biomass are known to those skilled in the art, for example filtration, sedimentation and flotation. Consequently, the biomass can be removed, for example, with centrifuges, separators, decanters, filters or in flotation apparatus. For maximum recovery of the product of value, washing of the biomass is often advisable, for example in the form of a diafiltration. The selection of the method is dependent upon the biomass content in the fermenter broth and the properties of the biomass, and also the interaction of the biomass with the product of value.

In one embodiment, the fermentation broth can be sterilized or pasteurized. In a further embodiment, the fermentation broth is concentrated. Depending on the requirement, this concentration can be done batch wise or continuously. The pressure and temperature range should be selected such that firstly no product damage occurs, and secondly minimal use of apparatus and energy is necessary. The skillful selection of pressure and temperature levels for a multistage evaporation in particular enables saving of energy.

The following examples are illustrative only and are not intended to limit the scope of the claims an embodiments described herein.

The numerous possible variations that will become immediately evident to a person skilled in the art after heaving considered the disclosure provided herein also fall within the scope of the invention.

### Experimental Part

### Materials:

Unless otherwise stated, all chemical and biochemical materials and microorganisms or cells employed herein are commercially available products.

Unless otherwise specified, recombinant proteins are cloned and expressed by standard methods, such as, for example, as described by Sambrook, J., Fritsch, E. F. and Maniatis, T., Molecular cloning: A Laboratory Manual, 2nd Edition, Cold Spring Harbor Laboratory, Cold Spring Harbor Laboratory Press, Cold Spring Harbor, NY, 1989.

### Gas Chromatography Mass Spectrometry (GC-MS)

Agilent Intuvo 9000 series GC system equipped with a DB-5 MS UI column (10 m×0.25 mm×0.25 μm film thickness (custom column made by Agilent Technologies Inc, Santa Clara, CA). The GC was coupled to two detectors by a 1:1 Detector Splitter Chip (G4588-60502, Agilent Technologies Inc, Santa Clara, CA). The first detector was a Agilent 5977B series mass spectrometer, while the second detector was a standard Intuvo 9000 Flame ionization detector (FID). The carrier gas was helium at a constant flow of 2.5 ml/min. Injection was in split (1:100) mode with the injector temperature set at 240° C. The oven temperature was programmed from 150° C. (0.1 min hold) to 240° C. at 40° C./min, then to 325° C. at 180° C./min and held 0.5 min)

### Example 1

Selection of Acetyltransferase Candidates for the Conversion of Drimane Sesquiterpenes into Drimanyl Acetylated Sesquiterpenes.

Acetyltransferases constitute a genetically diverse class of enzymes with more than 8,000 known representatives (PFAM database: PF02458 transferase family). Although the repertoire of molecules accepted as substrates by acetyltransferases is vast, none of them have been reported to accept sesquiterpene alcohols as substrates. In order to identify acetyltransferases capable to acetylate drimane-type sesquiterpene alcohols, fifty four (Table 1) out of the thousands known acetyltransferases originating from plants, fungi and bacteria were selected based on the following rationale:

Two out of five clades known from the plant acetyltransferase BAHD family, clades 3 and 5, have members identified for acetylation of alkaloids and terpenoids with acetyl-CoA as acyl donor (Curr Opin Plant Biol. 2006, 9 (3): 331-40). In addition, some substrates used by acetyltransferases from clades 3 and 5 are bulky, polycyclic and bear sterically hindered alcohol groups as acyl acceptors (BMC Genomics 2011, 12:236; Curr Opin Plant Biol. 2006, 9:331-40; Elife. 2017 Mar. 14; 6: e23001; Planta. 2015, 242:709-19). Based on this, twenty one candidates were selected from the plant BAHD family clades 3 and 5, these candidates include the identified acetyltransferases involved in the biosynthesis of the polycyclic diterpene paclitaxel (taxol) (Proc Natl Acad Sci USA. 2000, 18; 97 (2): 583-587) and two acetyltransferases from Plectranthus barbatus involved in the biosynthesis of the bicyclic, labdane diterpene Forskolin (Elife 2017, 14; 6: e23001).

Similarly to the plant candidates, seven fungal acetyltransferases were selected based on their putative or experimentally confirmed ability to accept acetyl-CoA as acyl donor and the structural characteristics of their substrates: bulky, polycyclic compounds, bearing sterically hindered alcohol groups (FEMS Microbiol Lett. 2005, 251:193-201; Chembiochem. 2009, 10:2325-8; Biotechnol Biotechnol Equip. 2014, 28 (5): 818-826; Nat Chem. 2010, 2:858-64). Among these, the protein AstG (from Aspergillus oryzae, NCBI accession No XP_023091083.1) involved in the biosynthesis of the aryl acid esterified drimane-type sesquiterpene lactone, astellolide (Sci Rep. 2016, 6:32865) was selected.

In addition, twenty one putative fungal acetyltransferases from the NCBI protein database were retrieved based on their sequence similarity with AstG using a PSI Blast search (Trends Biochem Sci. 2002, 27:161-4.) (standard parameters & 2 iterations). The results were visualized on a phylogenetic tree using the NCBI Blast Tree View function to show homology between AstG and 500 PSI-Blast derived sequences. Candidates were selected by homology to the query sequence, which were derived from a single branch of the phylogenetic tree.

Finally, chloramphenicol acetyltransferases are known to be promiscuous enzymes and able to accommodate bulky, larger than chloramphenicol, substrates (Protein Sci. 2012, 21 (4): 520-530). Thus, five bacterial chloramphenicol acetyltransferases from classes 1 to 3 were selected from the NCBI protein database as putative chloramphenicol acetyltransferase or were selected from literature (Biochem J. 1990, 272:505-10).

TABLE 1
Acetyltransferases selected for the conversion of drimane sesquiterpenes
into drimanyl acetylate sesquiterpenes

| Name | Accession Number | Organism |
|---|---|---|
| CrDAT | NCBI: AAC99311.1 | Catharanthus roseus |
| TcDBAT | NCBI: AAF27621.1 | Taxus cuspidate |
| TcTAT | NCBI: AAF34254.1 | Taxus cuspidate |
| FaSAAT | NCBI: AAG13130.1 | Fragaria x ananassa |
| PsSalAT | NCBI: AAK73661.1 | Papaver somniferum |
| AtCHAT | NCBI: AAN09797.1 | Arabidopsis thaliana |
| CrMAT | NCBI: AAO13736.1 | Catharanthus roseus |
| MdAAT2 | NCBI: AAS79797.1 | Malus domestica |
| RhAAT1 | NCBI: AAW31948.1 | Rosa x hybrid |
| AGZ20197.1 | NCBI: AGZ20197.1 | Penicillium crustosum |
| LiAAT-3 | NCBI: AIW81431.1 | Lavandula x intermedia |
| LiAAT-4 | NCBI: AIW81432.1 | Lavandula x intermedia |
| BAU61551.1 | NCBI: BAU61551.1 | Penicillium simplicissimum |
| tri3 | NCBI: BAX01959.1 | Fusarium asiaticum |
| FvVAAT | NCBI: CAC09062.1 | Fragaria vesca |
| RsVISY | NCBI: CAD89104.2 | Rauvolfia serpentine |
| CEL05746.1 | NCBI: CEL05746.1 | Aspergillus calidoustus |
| FgaAT | NCBI: EAL94101.1 | Aspergillus fumigatus |
| GAO81666.1 | NCBI: GAO81666.1 | Aspergillus udagawae |
| GAQ06029.1 | NCBI: GAQ06029.1 | Aspergillus lentulus |
| KIA75847.1 | NCBI: KIA75847.1 | Aspergillus ustus |
| CfACT1-6 | NCBI: KT382361.1 | C. forskohlii |
| CfACT1-8 | NCBI: KT382363.1 | C. forskohlii |
| CfACT2 | NCBI: KT382364.1 | C. forskohlii |
| CfACT3 | NCBI: KT382365.1 | C. forskohlii |
| CfACT4 | NCBI: KT382366.1 | C. forskohlii |
| CfACT5 | NCBI: KT382367.1 | C. forskohlii |
| CfACT6 | NCBI: KT382368.1 | C. forskohlii |
| CfACT8 | NCBI: KT382370.1 | C. forskohlii |
| KUM59602.1 | NCBI: KUM59602.1 | Penicillium freii |
| OAH94415.1 | NCBI: OAH94415.1 | Proteus mirabilis |
| OJJ08848.1 | NCBI: OJJ08848.1 | Aspergillus versicolor |
| OQD79314.1 | NCBI: OQD79314.1 | Penicillium antarcticum |
| OXN37635.1 | NCBI: OXN37635.1 | Aspergillus turcosus |
| P00484.1 | NCBI: P00484.1 | Escherichia coli |
| P22615.1 | NCBI: P22615.1 | Escherichia coli |
| PIG69200.1 | NCBI: PIG69200.1 | Aspergillus arachidicola |
| ppb8 | NCBI: FW308713.1; | Penicillium coprobium |
| | Nucleotides 23205-24773 | |
| ppb9 | NCBI: FW308713.1; | Penicillium coprobium |
| | Nucleotides 25824 27178 | |
| pyr8 | NCBI: Q4WLC8.1 | Aspergillus fumigatus |
| pyr7 | NCBI: Q4WLC9.1 | Aspergillus fumigatus |
| RAQ51407.1 | NCBI: RAQ51407.1 | Aspergillus flavus |
| WP_061323745.1 | NCBI: WP_061323745.1 | Clostridium botulinum |
| XP_001214750.1 | NCBI: XP_001214750.1 | Aspergillus terreus |
| XP_001217250.1 | NCBI: XP_001217250.1 | Aspergillus terreus |
| XP_001218652.1 | NCBI: XP_001218652.1 | Aspergillus terreus |
| XP_001258079.1 | NCBI: XP_001258079.1 | Aspergillus fischeri |
| XP_020058309.1 | NCBI: XP_020058309.1 | Aspergillus aculeatus |
| AstG | NCBI: XP_023091083.1 | Aspergillus oryzae |
| XP_024675087.1 | NCBI: XP_024675087.1 | Aspergillus candidus |
| XP_024675090.1 | NCBI: XP_024675090.1 | Aspergillus candidus |
| XP_024682561.1 | NCBI: XP_024682561.1 | Aspergillus novofumigatus |
| XP_024683987.1 | NCBI: XP_024683987.1 | Aspergillus novofumigatus |
| YP_006162226.1 | NCBI: YP_006162226.1 | Escherichia coli |

### Example 2

In Vivo Albicanyl Acetate Production in Saccharomyces cerevisiae Co-Expressing an Albicanol Synthase and Different Acetyltransferase Candidates.

The enzyme candidates were each screened for the in vivo bioconversion of albicanol to albicanyl acetate. For the screening, acetyltransferase candidates were each co-expressed with the gene encoding for the albicanol synthase XP_007369631.1, NCBI accession No XP_007369631.1, from Dichomitus squalens in an engineered Saccharomyces cerevisiae strain with increased level of endogenous farnesyl-diphosphate (FPP).

To increase the level of endogenous FPP pool in S. cerevisiae, an extra copy of all the yeast endogenous genes involved in the mevalonate pathway, from ERG10 coding for acetyl-CoA C-acetyltransferase to ERG20 coding for FPP synthase, were integrated in the genome of the S. cerevisiae strain CEN.PK2-1C (Euroscarf, Frankfurt, Germany) under the control of galactose-inducible promoters, similarly as described in Paddon et al., Nature, 2013, 496:528-532. Briefly, three cassettes were integrated in the LEU2, TRP1 and URA3 loci respectively. A first cassette containing the genes ERG20 and a truncated HMG1 (tHMG1 as described in Proc Natl Acad Sci USA, 1997, 109: E111-8) under the control of the bidirectional promoter of GAL10/GAL1 and the genes ERG19 and ERG13 also under the control of GAL10/GAL1 promoter, the cassette was flanked by two 100 nucleotides regions corresponding to the up- and down-stream sections of LEU2. A second cassette where the genes IDI1 and tHMG1 were under the control of the GAL10/GAL1 promoter and the gene ERG13 under the control of the promoter region of GAL7, the cassette was flanked by two 100 nucleotides regions corresponding to the up- and down-stream sections of TRP1. A third cassette with the genes ERG10, ERG12, tHMG1 and ERG8, all under the control of GAL10/GAL1 promoters, the cassette was flanked by two 100 nucleotides regions corresponding to the up- and down-stream sections of URA3. All genes in the three cassettes included 200 nucleotides of their own terminator regions. Also, an extra copy of GAL4 under the control of a mutated version of its own promoter, as described in Proc Natl Acad Sci USA, 1991, 88:8597-8601, was integrated upstream the ERG9 promoter region. In addition, the expression of ERG9 was modified by promoter exchange. The GAL7, GAL10 and GAL1 genes were deleted using a cassette containing the HIS3 gene with its own promoter and terminator. The resulting strain was mated with the strain CEN.PK2-1D (Euroscarf, Frankfurt, Germany) obtaining a diploid strain termed YST045 which was induced for sporulation according to Solis-Escalante et al, FEMS Yeast Res, 2015, 15:2. Spore separation was achieved by resuspension of asci in 200 μL 0.5M sorbitol with 2 μL zymolyase (1000 U mL-1, Zymo research, Irvine, CA) and incubated at 37° C. for 20 minutes. The mix then was plated on media containing 20 g/L peptone, 10 g/L yeast extract, 20 g/L glucose and 20 g/L agar, one germinated spore was isolated and termed YST069.

For expression of XP_007369631.1 and the evaluated acetyltransferases in YST069, plasmids were constructed in vivo using yeast endogenous homologous recombination as previously described in Kuijpers et al., Microb Cell Fact., 2013, 12:47. The plasmids are composed of four DNA fragments which were used for S. cerevisiae co-transformation. The fragments were:
a) The plasmid pF167 (SEQ ID NO: 1) linearized by enzymatic restriction with BsmBI. pF167 was previously constructed by in vivo assembly in yeast, it contains the yeast marker LEU2 with its own promoter and terminator, the Escherichia coli marker AmpR, the 2μ yeast origin of replication, the E. coli pUC replication origin and the sequences 5′-GCACTTGCTACACTGTCAGGATAGCTTCCGTCACATGGTGGCGATCACC GTACATCTGAG-3′ (SEQ ID NO: 2) and 5′-AGGTGCAGTTCGCGTGCAATTATAACGTCGTGGCAACTGTTATCAGTCG TACCGCGCCAT-3′ (SEQ ID NO: 3) for homologous recombination;
b) a fragment composed by the sequence 5′-GCACTTGCTACACTGTCAGGATAGCTTCCGTCACATGGTGGCGATCACC GTACATCTGAG-3′ (SEQ ID NO: 2), the terminator region of the yeast gene PGK1 and the sesquiterpene synthase XP_007369631.1 DNA sequence codon optimized for its expression in S. cerevisiae (SEQ ID NO: 4), this fragment was obtained by DNA synthesis (ATUM, Menlo Park, CA 94025). The bidirectional GAL1/GAL10 promoter region from yeast was added to this fragment by PCR overlap extension (Yolov and Shabarova., Nucleic Acids Res. 1990, 18 (13): 3983-6);
c) a fragment composed by 60 bp corresponding to the first nucleotides of the yeast GAL10 promoter region, one of the to be evaluated acetyltransferase DNA coding sequences (codon optimized for its expression in S. cerevisiae) and 60 bp of the yeast CYC1 terminator region, this fragment was obtained by DNA synthesis (ATUM, Menlo Park, CA 94025); and
d) a fragment composed by the terminator region of the yeast gene CYC1 and the sequence 5′-AGGTGCAGTTCGCGTGCAATTATAACGTCGTGGCAACTGTTATCAGTCG TACCGCGCCAT-3′ (SEQ ID NO: 3), this fragment was obtained by DNA synthesis (ATUM, Menlo Park, CA 94025).

YST069 was transformed with the fragments required for in vivo plasmid assembly. Yeast transformations were performed with the lithium acetate protocol as described in Gietz and Woods, Methods Enzymol., 2002, 350:87-96. The transformation mixtures were plated on SmLeu-media containing 6.7 g/L of Yeast Nitrogen Base without amino acids (BD Difco, New Jersey, USA), 1.6 g/L Dropout supplement without leucine (Sigma Aldrich, Missouri, USA), 20 g/L glucose and 20 g/L agar. Plates were incubated for 3-4 days at 30° C. Individual colonies were used to produce albicanol and albicanyl acetate in deep well plates containing 250 μL media as described in Westfall et al., Proc Natl Acad Sci USA, 2012, 109: E111-118 and 50 μL adipic acid diisodecyl ester (aber GmbH, Germany) as organic overlay. The deep well plates were incubated for 3 days at 30° C. in a plate incubator. To extract albicanol and albicanyl acetate produced by the yeast cells, each well of the deep well plate was extracted with 700 μL of ethyl acetate containing an internal standard. The production of albicanol and albicanyl acetate was identified using GC-MS analysis and quantified by GC-FID using the above mentioned internal standard.

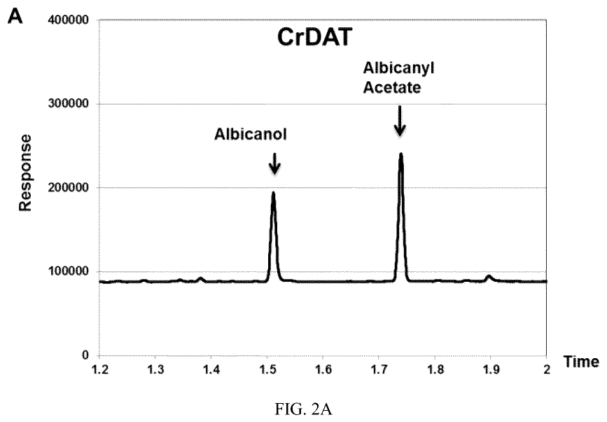

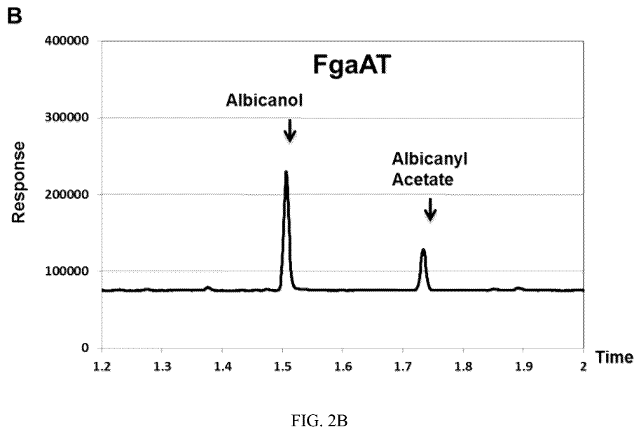

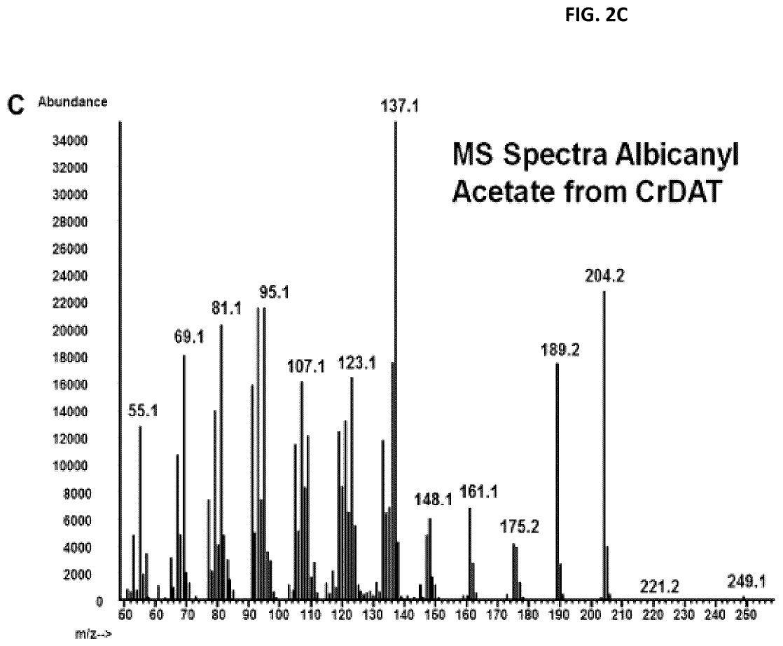

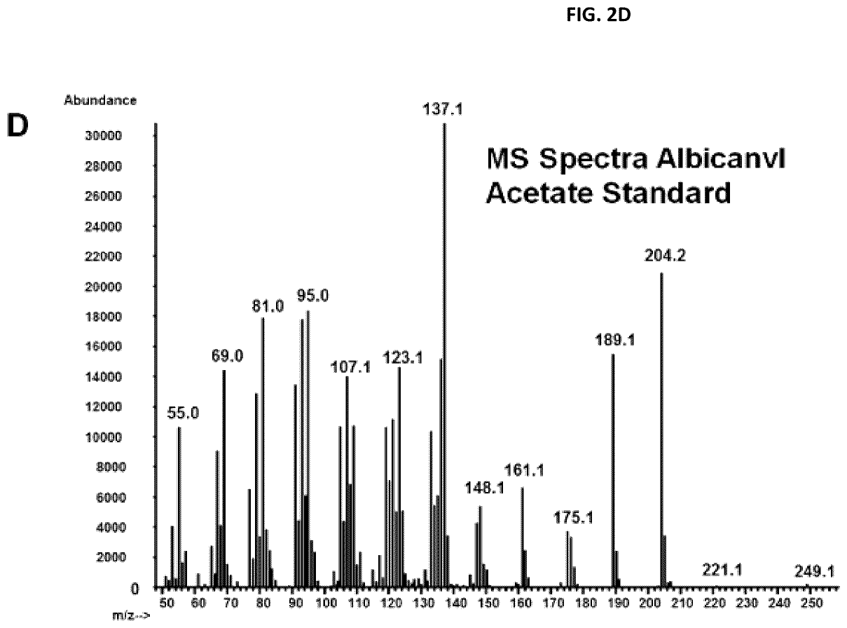

Under these experimental conditions, albicanyl acetate was detected for 9 combinations of albicanol synthase and acetyltransferases (Table 2). Remarkably, the two most active acetyltransferases (CrDAT and FgaAT) produced titers of albicanyl acetate of more than 150 mg/L under the non-optimized screening conditions. The GC-FID chromatograms are displayed in FIG. 2. Further, FIG. 2 shows that the MS spectrum of the yeast-derived albicanyl acetate is identical to a reference albicanyl acetate MS spectrum.

Interestingly, the most active acetyltransferases in our screening, CrDAT, a member of the plant BAHD-family clade 3, is involved in the biosynthesis of plant alkaloids, however, other tested acetyltransferases from the same family and clade, PsSalAT and RsVISY, and involved in plant alkaloid biosynthesis (Thebaine and Vinorine, respectively) were not active when albicanol was presented as substrate. In addition, FgaAT from Aspergillus fumigatus, also involved in the biosynthesis of alkaloids (active on Fumigaclavine B), produced significant amounts of albicanyl acetate in our screening; whereas, surprisingly, the putative Fumigaclavine B O-acetyltransferase GAO81666.1 produced one order of magnitude less albicanyl acetate than FgaAT.

It is worth to notice, that neither the acetyltransferase AstG nor any of the proteins selected base on its similarity with AstG (excluding GAO81666.1, see above) produced the Drimanyl acetylated sesquiterpene albicanyl acetate, despite the fact that AstG is involved in the biosynthesis of the modified drimane-type sesquiterpene Astellolide.

The only identified acetyltransferases in diterpene biosynthesis are those involved in the production of Paclitaxel (Taxol) and Forskolin. From the eight acetyltransferases tested from C. forskohlii only CfACT1-6 and CfACT1-8 were able to acetylate albicanol. Although the genes TcTAT and TcDBAT are capable to acetylate structurally similar intermediates in the biosynthesis of Taxol (Taxa-4 (20), 11 (12)-dien-5a-yl acetate and Baccatin III, respectively), only TcTAT produced albicanyl acetate.

To add on the, now documented, complexity to identify an acetyltransferase capable to use albicanol as acyl acceptor, only one of the five tested substrate versatile Chloramphenicol acetyltransferases was able to convert albicanol to albicanyl acetate.

TABLE 2
Acetyltransferases found able to convert albicanol to albicanyl acetate in
Saccharomyces cerevisiae

| | SEQ ID | | | |
|---|---|---|---|---|
| | NO | | | |
| | (amino | | | |
| | acid | Accession | | Titers Albicanyl |
| Name | sequence) | Number | Organism | Acetate [mg/L] |
| CrDAT | 9 | NCBI: | Catharanthus roseus | 400 |
| | | AAC99311.1 | | |
| FgaAT | 17 | NCBI: | Aspergillus fumigatus | 176 |
| | | EAL94101.1 | Af293 | |
| OAH94415.1 | 25 | NCBI: | Proteus mirabilis | 15 |
| | | OAH94415.1 | | |
| TcTAT | 11 | NCBI: | Taxus cuspidata | 12 |
| | | AAF34254.1 | | |
| CrMAT | 13 | NCBI: | Catharanthus roseus | 5 |
| | | AAO13736.1 | | |
| LiAAT-4 | 15 | NCBI: | Lavandula | 8 |
| | | AIW81432.1 | x | |
| | | | intermedia | |
| GAO81666.1 | 19 | NCBI: | Aspergillus udagawae | 10 |
| | | GAO81666.1 | | |
| CfACT1-6 | 21 | NCBI: | Plectranthus barbatus | 50 |
| | | KT382361.1 | | |
| CfACT1-8 | 23 | NCBI: | Plectranthus barbatus | 56 |
| | | KT382363.1 | | |

### Example 3

In Vivo Drimenyl Acetate Production in Saccharomyces cerevisiae Co-Expressing a Drimenol Synthase and Selected Acetyltransferase Candidates.

For the production of drimenyl acetate in Saccharomyces cerevisiae the nine selected acetyltransferases (CrDAT, FgaAT, OAH94415.1, TcTAT, CrMAT, LiAAT-4, GAO81666.1, CfACT1-6, CfACT1-8) capable to convert albicanol to albicanyl acetate, as shown in example 2, were used to evaluate the conversion of drimenol to drimenyl acetate.

Drimenyl acetate was produced in vivo in the engineered S. cerevisiae strain YST069 with increased level of endogenous FPP (see Example 2) by the co-expression of each of the selected acetyltransferase enzyme candidates with the gene encoding for the drimenol synthase XP_006461126, NCBI accession No XP_006461126, from Agaricus bisporus.

For expression of XP_006461126 and the selected acetyltransferases (CrDAT, FgaAT, OAH94415.1, TcTAT, CrMAT, LiAAT-4, GAO81666.1, CfACT1-6 and CfACT1-8) in YST069, plasmids were constructed in vivo using yeast endogenous homologous recombination as previously described in Kuijpers et al., Microb Cell Fact., 2013, 12:47. The plasmids are composed of four DNA fragments which were used for S. cerevisiae co-transformation. The fragments were:
a) The plasmid pF167 (SEQ ID NO: 1) linearized by enzymatic restriction with BsmBI. pF167 was previously constructed by in vivo assembly in yeast, it contains the yeast marker LEU2 with its own promoter and terminator, the Escherichia coli marker AmpR, the 2μ yeast origin of replication, the E. coli pUC replication origin and the sequences 5′-GCACTTGCTACACTGTCAGGATAGCTTCCGTCACATGGTGGCGATCACC GTACATCTGAG-3′ (SEQ ID NO: 2) and 5′-AGGTGCAGTTCGCGTGCAATTATAACGTCGTGGCAACTGTTATCAGTCG TACCGCGCCAT-3′ (SEQ ID NO: 3) for homologous recombination;
b) a fragment composed by the sequence 5′-GCACTTGCTACACTGTCAGGATAGCTTCCGTCACATGGTGGCGATCACC GTACATCTGAG-3′ (SEQ ID NO: 2), the terminator region of the yeast gene PGK1 and the sesquiterpene synthase XP_006461126 DNA sequence codon optimized for its expression in S. cerevisiae (SEQ ID NO: 6), this fragment was obtained by DNA synthesis (ATUM, Menlo Park, CA 94025). The bidirectional GAL1/GAL10 promoter region from yeast was added to this fragment by PCR overlap extension (Yolov and Shabarova., Nucleic Acids Res. 1990, 18 (13): 3983-6);
c) a fragment composed by 60 bp corresponding to the first nucleotides of the yeast GAL10 promoter region, one of the evaluated acetyltransferases DNA coding sequence codon optimized for its expression in S. cerevisiae and 60 bp of the yeast CYC1 terminator region, this fragment was obtained by DNA synthesis (ATUM, Menlo Park, CA 94025); and
d) a fragment composed by the terminator region of the yeast gene CYC1 and the sequence 5′-AGGTGCAGTTCGCGTGCAATTATAACGTCGTGGCAACTGTTATCAGTCG TACCGCGCCAT-3′ (SEQ ID NO: 3), this fragment was obtained by DNA synthesis (ATUM, Menlo Park, CA 94025).

Yeast transformation, screening conditions and quantification of drimenol and drimenyl acetate were performed as described in example 2 for the production of albicanol and albicanyl Acetate.

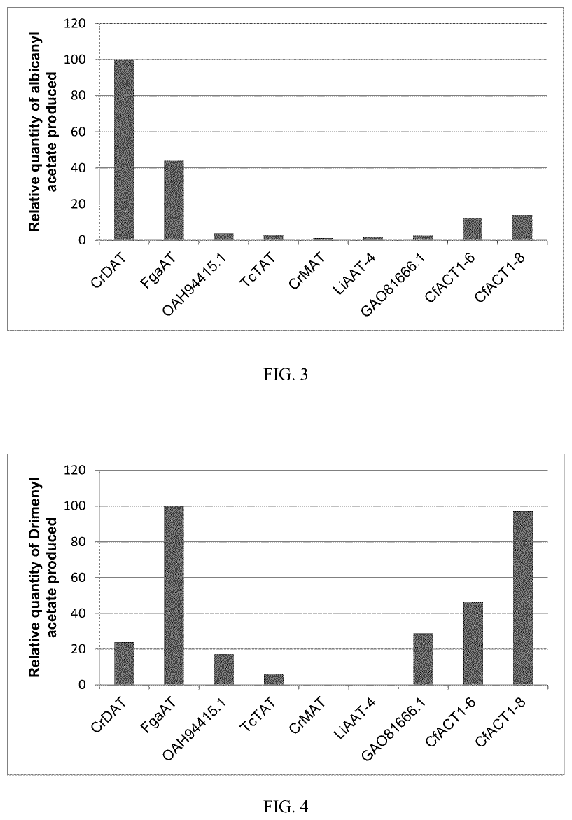

The relative quantities of albicanyl acetate (from Example 2) and drimenyl acetate are shown in FIG. 3 and FIG. 4, respectively. From the nine enzyme candidates tested, seven (CrDAT, FgaAT, OAH94415.1, TcTAT, GAO81666.1, CfACT1-6 and CfACT1-8) produced drimenyl acetate. Due to the high structural similarity between albicanol and drimenol, it was expected to find similar relative conversion rates.

Unexpectedly, the conversion of drimenol to his corresponding acetate was found to be significantly altered. In particular, CrDAT, which was found to produce the highest relative quantity of albicanyl acetate, was found to be one of the lowest producers of drimenyl acetate. By contrast, FgaAT and CfACT1-8 were found to produce the highest relative amounts ofd acetate whereas they were poorly active on albicanol. These surprising findings demonstrate the difficulty in identifying suitable acetyltransferase enzyme candidates to accept non physiological substrates.

### Example 4

Selection of Acetyltransferase Candidates for the Conversion of Drimane Sesquiterpenes into Acetylated Drimanyl Sesquiterpenes Based on Acetyltransferases Active on Drimenol and Albicanol.

### a) Experiment 1

In order to identify further acetyltransferases capable to acetylate drimane-type sesquiterpene alcohols, the amino acid sequences from the acetyltransferases CrDAT and FgaAT (shown in Example 2 and 3), which were active on albicanol and drimenol, were used in a NCBI Protein Blast search to find close protein sequence homologs. The Protein Blast search was executed using default parameters (Tatiana et al, FEMS Microbiol Lett., 1999, 174:247-250, 1999).

The plant acetyltransferase XP_008340165.2 homolog to CrDAT and the 4 fungal acetyltransferases homologs (KEY80391, PYI04555.1, XP_001276734.1, XP_024709055.1) to FgaAT were retrieved from the Blast search. They are listed in Table3.

TABLE 3
Acetyltransferases selected for the conversion of drimane sesquiterpenes
into drimanyl acetylate sesquiterpenes based on amino acid sequence
homology to either CrDAT or FgaAT

| Name | Accession Number | Organism |
|---|---|---|
| KEY80391.1 | KEY80391.1 | Aspergillus fumigatus |
| PYI04555.1 | PYI04555.1 | Aspergillus |
| | | sclerotiicarbonarius |
| XP_001276734.1 | XP_001276734.1 | Aspergillus clavatus |
| XP_024709055.1 | XP_024709055.1 | Aspergillus steynii |
| XP_008340165.2 | XP_008340165.2 | Malus domestica |

### b) Experiment 2

Additionally, extra acetyltransferases candidates were retrieved from the transcriptomes from the liverwort Bazzania trilobata and from the fern Dryopteris fragrans The liverwort Bazzania is a rich source of terpenoids including drimane sesquiterpenes; moreover, albicanyl acetate and albicanyl caffeate were reported as natural products from liverwort of the genus Bazzania (Asakawa et al, Phytochemistry, Volume 30, Issue 9, 1991, Pages 3037-3040). Similarly, several different natural products including albicanyl acetate were reported in the genus Dryopteris. (Hideyuki Ito et al. Chem. Pharm. Bull. 48 (8) 1190-1195 (2000); Froissard D et al. Nat Prod Commun. 2014 January; 9 (1): 137-40.)

The Bazzania trilobata (NCBI accession number ER364415) transcriptome was assembled using the CLC Genomic Workbench (Qiagen) resulting in a total of 22083 contigs with an average length of 1,225 base pairs. The CrDAT amino acid sequence was used to search for homologous sequences in the Bazzania trilobata transcriptome. For this search, the tBlastn algorithm was used (Altschul et al. 1990, J. Mol. Biol. 215, 403-410) with default parameters. Transcripts above an E-value of 0.001 were taken into account. 10 transcripts belonging to the plant acetyltransferase BAHD family with an amino acid sequence homology as low as 20% to CrDAT were selected.

Plant materials from D. fragrans were collected from Northern China. Fresh leaves of D. fragrans (sample ID PNLI20141074) were used for transcriptome analysis. Total RNA of D. fragrans was extracted using the RNeasy Plant Mini Kit (Cat N. 74904) from QIAGEN. The total RNA sample PNLI20141074 was processed using the NEBNext® Ultra™ RNA Library Prep Kit for Illumina (NEB, USA) and TruSeq PE Cluster Kit (Illumina, USA) and then sequenced on Illumina Miseq sequencer. An amount of 20.88 million of paired-end reads of 2×350 bp was generated. The reads were assembled using the Trinity (http://trinityrnaseq.sf.net/) software and 85753 contigs with an N50 of 1373 bp were obtained. The contigs were analysed using the EMBOSS software (http://emboss.sourceforge.net/) to get protein sequences. The amino acid sequence of the acetyltransferase ERR364415-1_contig_8546 (as obtained from the Bazzania trilobata (NCBI accession number ERR364415) transcriptome as described above) (SEQ ID NO: 144) was used to search for homologous sequences in the D. fragrans transcriptome. For this search the tBlastn algorithm was used (Altschul et al 1990, J. Mol. Biol. 215, 403-410) using standard parameters. This approach provided 20 transcripts belonging to the plant acetyltransferase BAHD family.

### Example 5

In Vivo Drimanyl Acetate Production in Saccharomyces cerevisiae by Co-Expressing Enzymes for the Production of Either Albicanol, Dirmenol or Bicyclofarnesol Together with Selected Acetyltransferase Candidates.

An extended screening of acetyltransferases to convert albicanol, drimenol and bicyclofarnesol into their corresponding acetate derivatives was performed. A total of 89 acetyltransferases, described in Table 1 of Example 1 and in Example 4, were screened in vivo in engineered Saccharomyces cerevisiae cells. Screening data generated on a preliminary number of acetyltransferases as described in Example 2 and Example 3 was not repeated. Consequently, only new combinations of acetyltransferases with Saccharomyces cerevisiae cells producing either albicanol, drimenol or bicyclofarnesol that were not tested in in Example 2 and Example 3 were screened.

The engineered S. cerevisiae strain YST069 was used for the screening of the production of albicanyl acetate and drimenyl acetate as described above in Example 2 and Example 3.

Bicyclofarnesyl acetate was produced in vivo in the engineered S. cerevisiae strain YST069 by the co-expression of each of the selected acetyltransferase enzyme candidates together with the enzymes AstC (SEQ ID NO: 138), AstI (SEQ ID NO: 140) and AstK (SEQ ID NO: 142) responsible the production of bicyclofarnesol from the astellolide biosynthesis (Yasutomo Shinohara et. al. Sci Rep. 2016, 6:32865).

For the simultaneous expression of AstC and AstI, an expression cassette containing codon optimized for S. cerevisiae versions of the genes coding for AstC (SEQ ID NO: 138) and AstI (SEQ ID NO: 140) and the bidirectional GAL1/GAL10 promoter was constructed and integrated into the genome of YST069 resulting in the new strain termed YST216. The codon optimized DNA sequences of AstC and AstI were obtained by DNA synthesis (ATUM, Menlo Park, CA 94025). The bidirectional GAL1/GAL10 promoter region from yeast was added to these genes by PCR overlap extension (Yolov and Shabarova., Nucleic Acids Res. 1990, 18 (13): 3983-6).

For expression of AstK and the evaluated acetyltransferases in YST216, plasmids were constructed in vivo using yeast endogenous homologous recombination as previously described in Kuijpers et al., Microb Cell Fact., 2013, 12:47. The plasmids are composed of four DNA fragments which were used for S. cerevisiae co-transformation. The fragments were:
a) The plasmid pF167 (SEQ ID NO: 1) linearized by enzymatic restriction with BsmBI.
b) a fragment composed by the sequence 5′-GCACTTGCTACACTGTCAGGATAGCTTCCGTCACATGGTGGCGATCACCGTACAT CTGAG-3′ (SEQ ID NO: 2), the terminator region of the yeast gene PGK1 and the AstK DNA sequence codon optimized for its expression in S. cerevisiae (SEQ ID NO: 141), this fragment was obtained by DNA synthesis (ATUM, Menlo Park, CA 94025). The bidirectional GAL1/GAL10 promoter region from yeast was added to this fragment by PCR overlap extension (Yolov and Shabarova., Nucleic Acids Res. 1990, 18 (13): 3983-6);
c) a fragment composed by 60 bp corresponding to the first nucleotides of the yeast GAL10 promoter region, one of the evaluated acetyltransferases DNA coding sequence codon optimized for its expression in S. cerevisiae and 60 bp of the yeast CYC1 terminator region, this fragment was obtained by DNA synthesis (ATUM, Menlo Park, CA 94025); and
d) a fragment composed by the terminator region of the yeast gene CYC1 and the sequence 5′-AGGTGCAGTTCGCGTGCAATTATAACGTCGTGGCAACTGTTATCAGT CGTACCGCGCCAT-3′ (SEQ ID NO: 3), this fragment was obtained by DNA synthesis (ATUM, Menlo Park, CA 94025).

YST216 was transformed with the fragments required for in vivo plasmid assembly. Yeast transformation was performed as described in example 2. Screening conditions and quantification of bicyclofarnesol and bicyclofarnesyl acetate were identical as described in example 2, with the exception of the use of 25 μL Mineral oil (2705-01, VWR International, LLC.) as organic overlay in the screening procedure.

### Results:

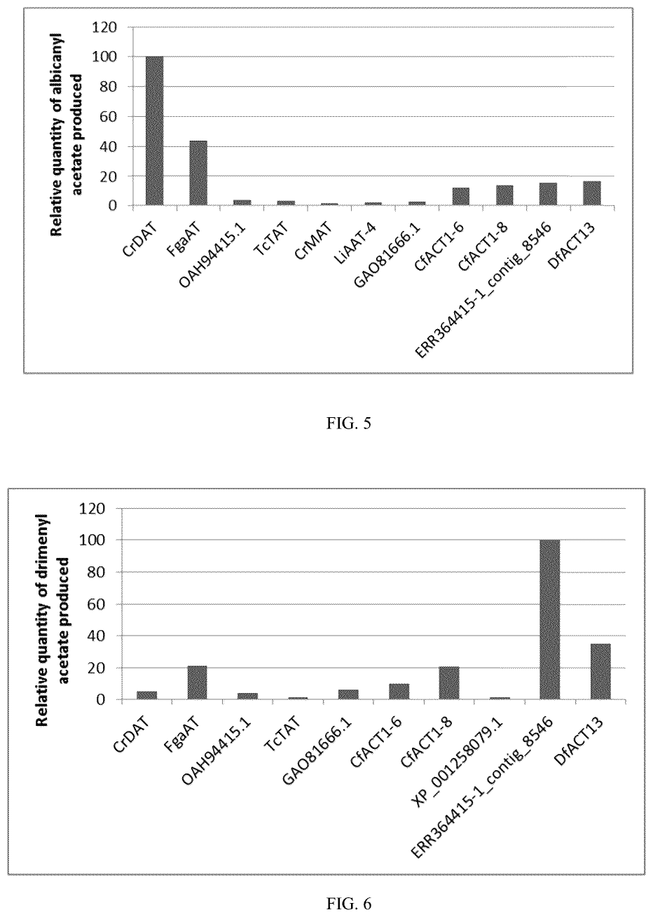

In addition to the previously described nine enzymes active on albicanol (CrDAT, FgaAT, OAH94415.1, TcTAT, CrMAT, LiAAT-4, GAO81666.1, CfACT1-6, CfACT1-8) (see Example 2), albicanyl acetate was detected, under these experimental conditions, when the acetyltransferases ERR364415-1_contig_8546 from Bazzania trilobata (SEQ ID NO: 124 or 144), and DfATC13 from Dryopteris fragrans (SEQ ID NO: 118), were used. However, their activity was lower compared to CrDAT and FgaAT. The relative quantities of albicanyl acetate are shown in FIG. 5.

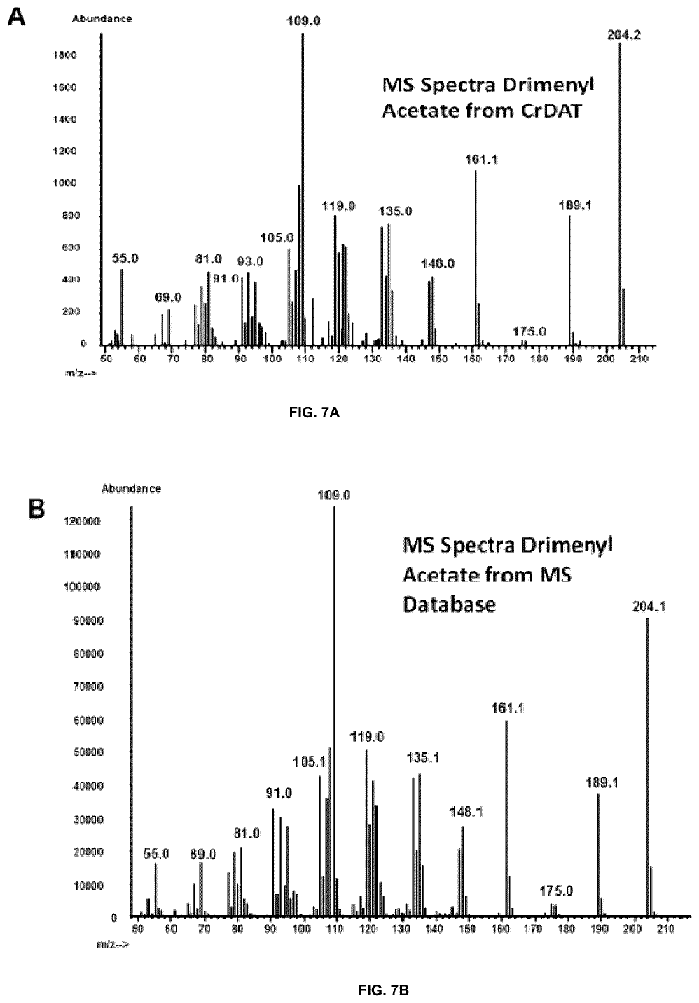

Besides the seven enzyme candidates (CrDAT, FgaAT, OAH94415.1, TcTAT, GAO81666.1, CfACT1-6 and CfACT1-8) which could convert drimenol to drimenyl acetate (see Example 3), three extra acetyltransferases, XP_001258079.1 from Aspergillus fischeri, (SEQ ID NO: 127), ERR364415-1_contig_8546 from Bazzania trilobata (SEQ ID NO: 124 or 144), and DfATC13 from Dryopteris fragrans (SEQ ID NO: 118), were found to produce drimenyl acetate from drimenol in the expanded screening. The relative quantities of drimenyl acetate obtained are shown in FIG. 6. FIG. 7 shows that the MS spectrum of the yeast-derived drimenyl acetate is identical to a reference drimenyl acetate MS spectrum. Interestingly, under these experimental conditions, ERR364415-1_contig_8546 and DfATC13 showed the highest activity of all acetyltransferases tested.

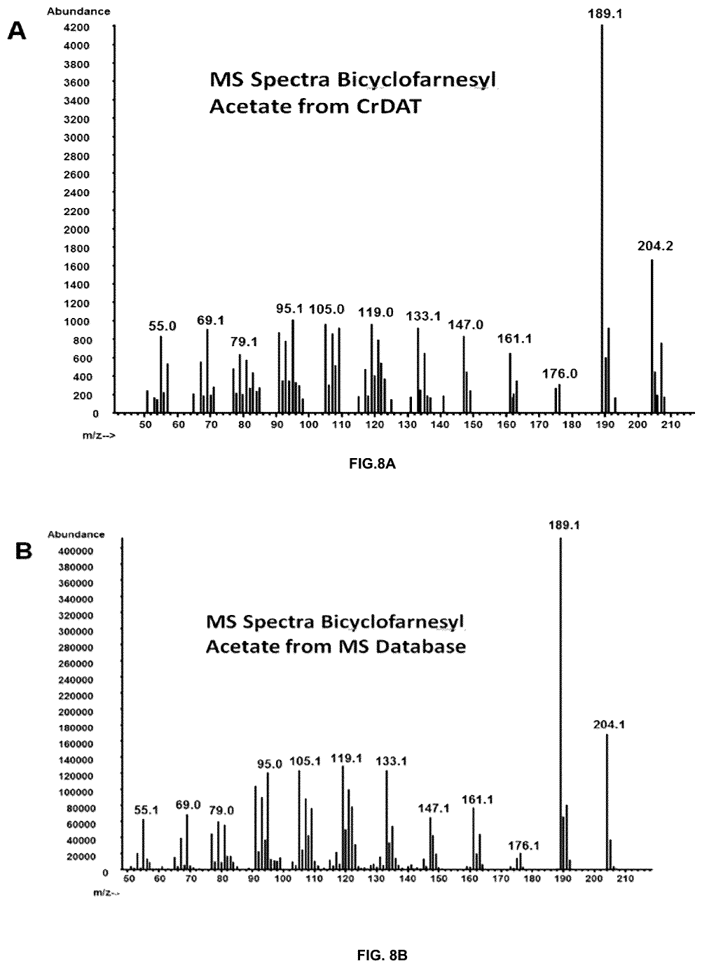

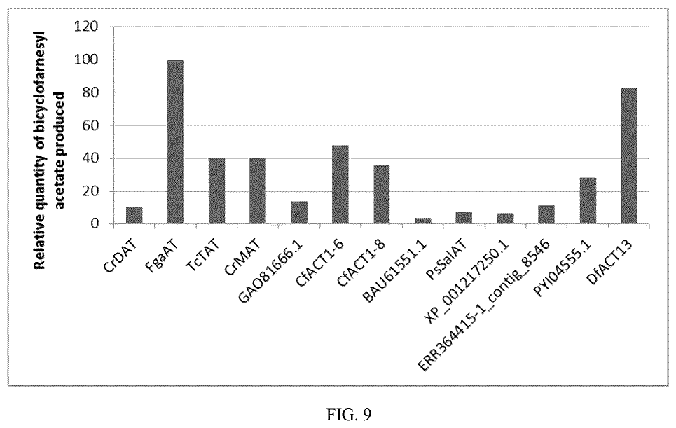

Bicyclofarnesyl acetate was detected for 13 combinations of AstC, AstI and AstK with the tested 89 acetyltransferases. FIG. 8 shows that the MS spectrum of the yeast-derived bicyclofarnesyl acetate is identical to a reference bicyclofarneyl acetate MS spectrum. The relative quantities of bicyclofarnesyl acetate are shown in FIG. 9. The acetyltransferase FgaAT from Aspergillus fumigatus and ERR364415-1_contig_8546 from Bazzania trilobata showed the highest acetylating activity with Bicyclofarnesol. Of these 13 discovered acetyltransferase, i.e. CrDAT, FgaAT, TcTAT, CrMAT, GAO81666.1, CfACT1-6, CfACT1-8 as described above in Example 2, as well as BAU61551.1 (SEQ ID NO: 133), PsSalAT (SEQ ID NO: 136), XP_001217250.1 (SEQ ID NO: 130), ERR364415-1_contig_8546 (SEQ ID NO: 124 or 144), PYI04555.1 (SEQ ID NO: 121 or 143), DfACT13 (SEQ ID NO: 118), which are active on bicyclofarnesol, nine i.e.CrDAT, FgaAT, TcTAT, CrMAT, GAO81666.1, CfACT1-6, CfACT1-8, ERR364415-1_contig_8546, DfACT13 were also found to active on albicanol and drimenol. It is worth to notice that the acetyltransferase AstG involved in the biosynthesis of the aryl acid esterified drimane-type sesquiterpene lactone, astellolide (Sci Rep. 2016, 6:32865) was not active against any drimane-type sesquiterpene alcohols tested. This shows once more the unpredictability and complexity to identify an acetyltransferase that is capable to use albicanol, drimenol or bicyclofarnesol as acyl acceptor.

The content of the documents cross-referenced is incorporated by reference.
Sequences as herein referred to are:

| SEQ ID | | | |
|---|---|---|---|
| NO | Name | Source | Type |
| 1 | Plasmid pF167 | Artificial | NA |
| 2 | Sequence for homologous recombination | Artificial | NA |
| 3 | Sequence for homologous recombination | Artificial | NA |
| 4 | XP_007369631.1 DNA sequence codon optimized | Artificial | NA |
| | for its expression in S. cerevisiae | | |
| 5 | XP_007369631.1 Amino acid sequence of | Dichomitus squalens | AA |
| | Albicanol synthase | | |
| 6 | XP_006461126 Drimeol synthase DNA sequence | Artificial | NA |
| | codon optimized for its expression in S. cerevisiae | | |
| 7 | XP_006461126 Protein sequence of Drimenol | Agaricus bisporus | AA |
| | synthase | | |
| 8 | CrDAT DNA sequence codon optimized for its | Artificial | NA |
| | expression in S. cerevisiae | | |
| 9 | CrDAT Protein sequence | Catharanthus roseus | AA |
| 10 | TcTAT DNA sequence codon optimized for its | Artificial | NA |
| | expression in S. cerevisiae | | |
| 11 | TcTAT Protein sequence | Taxus cuspidata | AA |
| 12 | CrMAT DNA sequence codon optimized for its | Artificial | NA |
| | expression in S. cerevisiae | | |
| 13 | CrMAT Protein sequence | Catharanthus roseus | AA |
| 14 | LiAAT-4 DNA sequence codon optimized for its | Artificial | NA |
| | expression in S. cerevisiae | | |
| 15 | LiAAT-4 Protein sequence | Lavandula x | AA |
| | | intermedia | |
| 16 | FgaAT DNA sequence codon optimized for its | Artificial | NA |
| | expression in S. cerevisiae | | |
| 17 | FgaAT Protein sequence | Aspergillus | AA |
| | | fumigatus Af293 | |
| 18 | GAO81666.1 DNA sequence codon optimized for | Artificial | NA |
| | its expression in S. cerevisiae | | |
| 19 | GAO81666.1 Protein sequence | Aspergillus | AA |
| | | udagawae | |
| 20 | CfACT1-6 DNA sequence codon optimized for its | Artificial | NA |
| | expression in S. cerevisiae | | |
| 21 | CfACT1-6 Protein sequence | Plectranthus | AA |
| | | barbatus | |
| 22 | CfACT1-8 DNA sequence codon optimized for its | Artificial | NA |
| | expression in S. cerevisiae | | |
| 23 | CfACT1-8 Protein sequence | Plectranthus | AA |
| | | barbatus | |
| 24 | OAH94415.1 DNA sequence codon optimized for | Artificial | NA |
| | its expression in S. cerevisiae | | |
| 25 | OAH94415.1 Protein sequence | Proteus mirabilis | AA |
| 116 | DfACT13 nativeNucleotide sequence | Dryopteris fragrans | NA |
| 117 | DfACT13 Nucleotide sequence codon optimized | Artificial | NA |
| | for its expression in S. cerevisiae. | | |
| 118 | DfACT13 Protein sequence | Dryopteris fragrans | AA |
| 119 | PYI04555.1 native Nucleotide sequence | Aspergillus | NA |
| | | sclerotiicarbonarius | |
| 120 | PYI04555.1 Nucleotide sequence codon optimized | Artificial | NA |
| | for its expression in S. cerevisiae. | | |
| 121 | PYI04555.1 Protein sequence C-terminally | Aspergillus | AA |
| | extended version of SEQ ID NO : 143 | sclerotiicarbonarius | |
| 122 | ERR364415-1_contig_8546 native Nucleotide | Bazzania trilobata | NA |
| | sequence | | |
| 123 | ERR364415-1_contig_8546 Nucleotide sequence | Artificial | NA |
| | codon optimized for its expression in S. cerevisiae | | |
| 124 | ERR364415-1_contig_8546 Protein sequence C- | Bazzania trilobata | AA |
| | terminally extended version of SEQ ID NO : 144 | | |
| 125 | XP_001258079.1 native Nucleotide sequence | Aspergillus fischeri | NA |
| 126 | XP_001258079.1 Nucleotide sequence codon | Artificial | NA |
| | optimized for its expression in S. cerevisiae | | |
| 127 | XP_001258079.1 Protein sequence | Aspergillus fischeri | AA |
| 128 | XP_001217250.1 native Nucleotide sequence | Aspergillus terreus | NA |
| 129 | XP_001217250.1 Nucleotide sequence codon | Artificial | NA |
| | optimized for its expression in S. cerevisiae | | |
| 130 | XP_001217250.1 Protein sequence | Aspergillus terreus | AA |
| 131 | BAU61551.1 native Nucleotide sequence | Penicillium | NA |
| | | simplicissimum | |
| 132 | BAU61551.1 Nucleotide sequence codon | Artificial | NA |
| | optimized for its expression in S. cerevisiae | | |
| 133 | BAU61551.1 Protein sequence | Penicillium | AA |
| | | simplicissimum | |
| 134 | PsSalAT native Nucleotide sequence | Papaver somniferum | NA |
| 135 | PsSalAT Nucleotide sequence codon optimized | Artificial | NA |
| | for its expression in S. cerevisiae. | | |
| 136 | PsSalAT Protein sequence | Papaver somniferum | AA |
| 137 | AstC Nucleotide sequence codon optimized for its | Artificial | NA |
| | expression in S. cerevisiae | | |
| 138 | AstC Protein sequence | Aspergillus oryzae | AA |
| 139 | AstI Nucleotide sequence codon optimized for its | Artificial | NA |
| | expression in S. cerevisiae | | |
| 140 | AstI Protein sequence | Aspergillus oryzae | AA |
| 141 | AstK Nucleotide sequence codon optimized for its | Artificial | NA |
| | expression in S. cerevisiae | | |
| 142 | AstK Protein sequence | Aspergillus oryzae | AA |
| 143 | PYI04555.1 Protein sequence, | Aspergillus | AA |
| | | sclerotiicarbonarius | |
| 144 | ERR364415-1_contig_8546 Protein sequence | Bazzania trilobata | AA |

NA = Nucleic Acid
AA = Amino Acid

### Listing of Sequences

Plasmid pF167
SEQ ID NO: 1
TGGTCAGCAACAACGCCGAAGAATCACTCTCGTGTTGAGAATTGCACGCCTTGACCACGACA
CGTTAAGGGATTTTGGTCATGAGATTATCAAAAAGGATCTTCACCTAGATCCTTTTAAATTA
AAAATGAAGTTTTAAATCAATCTAAAGTATATATGAGTAAACTTGGTCTGACAGTTACCAAT
GCTTAATCAGTGAGGCACCTATCTCAGCGATCTGTCTATTTCGTTCATCCATAGTTGCCTGA
CTCCCCGTCGTGTAGATAACTACGATACGGGAGGGCTTACCATCTGGCCCCAGTGCTGCAAT
GATACCGCGAGACCCACGCTCACCGGCTCCAGATTTATCAGCAATAAACCAGCCAGCCGGAA
GGGCCGAGCGCAGAAGTGGTCCTGCAACTTTATCCGCCTCCATCCAGTCTATTAATTGTTGC
CGGGAAGCTAGAGTAAGTAGTTCGCCAGTTAATAGTTTGCGCAACGTTGTTGCCATTGCTAC
AGGCATCGTGGTGTCACGCTCGTCGTTTGGTATGGCTTCATTCAGCTCCGGTTCCCAACGAT
CAAGGCGAGTTACATGATCCCCCATGTTGTGCAAAAAAGCGGTTAGCTCCTTCGGTCCTCCG
ATCGTTGTCAGAAGTAAGTTGGCCGCAGTGTTATCACTCATGGTTATGGCAGCACTGCATAA
TTCTCTTACTGTCATGCCATCCGTAAGATGCTTTTCTGTGACTGGTGAGTACTCAACCAAGT
CATTCTGAGAATAGTGTATGCGGCGACCGAGTTGCTCTTGCCCGGCGTCAATACGGGATAAT
ACCGCGCCACATAGCAGAACTTTAAAAGTGCTCATCATTGGAAAACGTTCTTCGGGGCGAAA
ACTCTCAAGGATCTTACCGCTGTTGAGATCCAGTTCGATGTAACCCACTCGTGCACCCAACT
GATCTTCAGCATCTTTTACTTTCACCAGCGTTTCTGGGTGAGCAAAAACAGGAAGGCAAAAT
GCCGCAAAAAAGGGAATAAGGGCGACACGGAAATGTTGAATACTCATACTCTTCCTTTTTCA
ATATTATTGAAGCATTTATCAGGGTTATTGTCTCATGAGCGGATACATATTTGAATGTATTT
AGAAAAATAAACAAATAGGGGTTCCGCGCACATTTTTGGCATTGGCAAAGTGCGGACTGCAT
AGTCACTGTGGTGCCGTACTTAGGGTACGCGTTCCTGAACGAAGCATCTGTGCTTCATTTTG
TAGAACAAAAATGCAACGCGAGAGCGCTAATTTTTCAAACAAAGAATCTGAGCTGCATTTTT
ACAGAACAGAAATGCAACGCGAAAGCGCTATTTTACCAACGAAGAATCTGTGCTTCATTTTT
GTAAAACAAAAATGCAACGCGAGAGCGCTAATTTTTCAAACAAAGAATCTGAGCTGCATTTT
TACAGAACAGAAATGCAACGCGAGAGCGCTATTTTACCAACAAAGAATCTATACTICTTTTT
TGTTCTACAAAAATGCATCCCGAGAGCGCTATTTTTCTAACAAAGCATCTTAGATTACTTTT
TTTCTCCTTTGTGCGCTCTATAATGCAGTCTCTTGATAACTTTTTGCACTGTAGGTCCGTTA
AGGTTAGAAGAAGGCTACTTTGGTGTCTATTTTCTCTTCCATAAAAAAAGCCTGACTCCACT
TCCCGCGTTTACTGATTACTAGCGAAGCTGCGGGTGCATTTTTTCAAGATAAAGGCATCCCC
GATTATATTCTATACCGATGTGGATTGCGCATACTTTGTGAACAGAAAGTGATAGCGTTGAT
GATTCTTCATTGGTCAGAAAATTATGAACGGTTTCTTCTATTTTGTCTCTATATACTACGTA
TAGGAAATGTTTACATTTTCGTATTGTTTTCGATTCACTCTATGAATAGTTCTTACTACAAT
TTTTTTGTCTAAAGAGTAATACTAGAGATAAACATAAAAAATGTAGAGGTCGAGTTTAGATG
CAAGTTCAAGGAGCGAAAGGTGGATGGGTAGGTTATATAGGGATATAGCACAGAGATATATA
GCAAAGAGATACTTTTGAGCAATGTTTGTGGAAGCGGTATTCGCAATATTTTAGTAGCTCGT
TACAGTCCGGTGCGTTTTTGGTTTTTTGAAAGTGCGTCTTCAGAGCGCTTTTGGTTTTCAAA
AGCGCTCTGAAGTTCCTATACTTTCTAGAGAATAGGAACTTCGGAATAGGAACTTCAAAGCG
TTTCCGAAAACGAGCGCTTCCGAAAATGCAACGCGAGCTGCGCACATACAGCTCACTGTTCA
CGTCGCACCTATATCTGCGTGTTGCCTGTATATATATATACATGAGAAGAACGGCATAGTGC
GTGTTTATGCTTAAATGCGTACTTATATGCGTCTATTTATGTAGGATGAAAGGTAGTCTAGT
ACCTCCTGTGATATTATCCCATTCCATGCGGGGTATCGTATGCTTCCTTCAGCACTACCCTT
TAGCTGTTCTATATGCTGCCACTCCTCAATTGGATTAGTCTCATCCTTCAATGCTATCATTA
TGTCACCCGCAGTTCTGTGTCGTAGTCATCAACATAGCACCTATCCTTTGGCATCTCGGTGA
GCAAAAGGCCAGCAAAAGGCCAGGAACCGTAAAAAGGCCGCGTTGCTGGCGTTTTTCCATAG
GCTCCGCCCCCCTGACGAGCATCACAAAAATCGACGCTCAAGTCAGAGGTGGCGAAACCCGA
CAGGACTATAAAGATACCAGGCGTTTCCCCCTGGAAGCTCCCTCGTGCGCTCTCCTGTTCCG
ACCCTGCCGCTTACCGGATACCTGTCCGCCTTTCTCCCTTCGGGAAGCGTGGCGCTTTCTCA
TAGCTCACGCTGTAGGTATCTCAGTTCGGTGTAGGTCGTTCGCTCCAAGCTGGGCTGTGTGC
ACGAACCCCCCGTTCAGCCCGACCGCTGCGCCTTATCCGGTAACTATCGTCTTGAGTCCAAC
CCGGTAAGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAGAAGGA
CAGTATTTGGTATCTGCGCTCTGCTGAAGCCAGTTACCTTCGGAAAAAGAGTTGGTAGCTCT
TGATCCGGCAAACAAACCACCGCTGGTAGCGGTGGTTTTTTTGTTTGCAAGCAGCAGATTAC
GCGCAGAAAAAAAGGATCTCAAGAAGATCCTTTGATCTTTTCTACGGGGTCTGACGCTCAGC
ACTTGCTACACTGTCAGGATAGCTTCCGTCACATGGTGGCGATCACCGTACATCTGAGTGAG
ACGTTAATTAAAGTAGACCGCTCACACATGGGCGGCCGCCGTCTCAAGGTGCAGTTCGCGTG
CAATTATAACGTCGTGGCAACTGTTATCAGTCGTACCGCGCCATTCGACTACGTCGTAAGGC
CGTTTCTGACAGAGTAAAATTCTTGAGGGAACTTTCACCATTATGGGAAATGCTTCAAGAAG
GTATTGACTTAAACTCCATCAAATGGTCAGGTCATTGAGTGTTTTTTATTTGTTGTATTTTT
TTTTTTTTAGAGAAAATCCTCCAATATCAAATTAGGAATCGTAGTTTCATGATTTTCTGTTA
CACCTAACTTTTTGTGTGGTGCCCTCCTCCTTGTCAATATTAATGTTAAAGTGCAATTCTTT
TTCCTTATCACGTTGAGCCATTAGTATCAATTTGCTTACCTGTATTCCTTTACTATCCTCCT
TTTTCTCCTTCTTGATAAATGTATGTAGATTGCGTATATAGTTTCGTCTACCCTATGAACAT
ATTCCATTTTGTAATTTCGTGTCGTTTCTATTATGAATTTCATTTATAAAGTTTATGTACAA
ATATCATAAAAAAAGAGAATCTTTTTAAGCAAGGATTTTCTTAACTTCTTCGGCGACAGCAT
CACCGACTTCGGTGGTACTGTTGGAACCACCTAAATCACCAGTTCTGATACCTGCATCCAAA
ACCTTTTTAACTGCATCTTCAATGGCCTTACCTTCTTCAGGCAAGTTCAATGACAATTTCAA
CATCATTGCAGCAGACAAGATAGTGGCGATAGGGTCAACCTTATTCTTTGGCAAATCTGGAG
CAGAACCGTGGCATGGTTCGTACAAACCAAATGCGGTGTTCTTGTCTGGCAAAGAGGCCAAG
GACGCAGATGGCAACAAACCCAAGGAACCTGGGATAACGGAGGCTTCATCGGAGATGATATC
ACCAAACATGTTGCTGGTGATTATAATACCATTTAGGTGGGTTGGGTTCTTAACTAGGATCA
TGGCGGCAGAATCAATCAATTGATGTTGAACCTTCAATGTAGGGAATTCGTTCTTGATGGTT
TCCTCCACAGTTTTTCTCCATAATCTTGAAGAGGCCAAAAGATTAGCTTTATCCAAGGACCA
AATAGGCAATGGTGGCTCATGTTGTAGGGCCATGAAAGCGGCCATTCTTGTGATTCTTTGCA
CTTCTGGAACGGTGTATTGTTCACTATCCCAAGCGACACCATCACCATCGTCTTCCTTTCTC
TTACCAAAGTAAATACCTCCCACTAATTCTCTGACAACAACGAAGTCAGTACCTTTAGCAAA
TTGTGGCTTGATTGGAGATAAGTCTAAAAGAGAGTCGGATGCAAAGTTACATGGTCTTAAGT
TGGCGTACAATTGAAGTTCTTTACGGATTTTTAGTAAACCTTGTTCAGGTCTAACACTACCG
GTACCCCATTTAGGACCAGCCACAGCACCTAACAAAACGGCATCAACCTTCTTGGAGGCTTC
CAGCGCCTCATCTGGAAGTGGGACACCTGTAGCATCGATAGCAGCACCACCAATTAAATGAT
TTTCGAAATCGAACTTGACATTGGAACGAACATCAGAAATAGCTTTAAGAACCTTAATGGCT
TCGGCTGTGATTTCTTGACCAACGTGGTCACCTGGCAAAACGACGATCTTCTTAGGGGCAGA
CATAGGGGCAGACATTAGAATGGTATATCCTTGAAATATATATATATATTGCTGAAATGTAA
AAGGTAAGAAAAGTTAGAAAGTAAGACGATTGCTAACCACCTATTGGAAAAAACAATAGGTC
CTTAAATAATATTGTCAACTTCAAGTATTGTGATGCAAGCATTTAGTCATGAACGCTTCTCT
ATTCTATATGAAAAGCCGGTTCCGGCCTCTCACCTTTCCTTTTTCTCCCAATTTTTCAGTTG
AAAAAGGTATATGCGTCAGGCGACCTCTGAAATTAACAAAAAATTTCCAGTCATCGAATTTG
ATTCTGTGCGATAGCGCCCCTGTGTGTTCTCGTTATGTTGAGGAAAAAAATAATGGTTGCTA
AGAGATTCGAACTCTTGCATCTTACGATACCTGAGTATTCCCACAGTTAACTGCGGTCAAGA
TATTTCTTGAATCAGGCGCCTTAGACCGCTCGGCCAAACAACCAATTACTTGTTGAGAAATA
GAGTATAATTATCCTATAAATATAACGTTTTTGAACACACATGAACAAGGAAGTACAGGACA
ATTGATTTTGAAGAGAATGTGGATTTTGATGTAATTGTTGGGATTCCATTTTTAATAAGGCA
ATAATATTAGGTATGTGGATATACTAGAAGTTCTCCTCGACCGTCGA
Sequence for homologous recombination
SEQ ID NO: 2
GCACTTGCTACACTGTCAGGATAGCTTCCGTCACATGGTGGCGATCACCGTACATCTGAG
Sequence for homologous recombination
SEQ ID NO: 3
AGGTGCAGTTCGCGTGCAATTATAACGTCGTGGCAACTGTTATCAGTCGTACCGCGCCAT
XP_007369631.1 DNA sequence of Dichomitus squalens
Albicanol synthase codon optimized for its expression
in S. cerevisiae
SEQ ID NO: 4
ATGGCTTCTATCCACAGAAGATACACTACTTTGATCTTGGACTTGGGTGACGTTTTGTTCAG
ATGGTCTCCAAAGACTGAAACTGCTATCCCACCACAACAATTGAAGGACATCTTGTCTTCTG
TTACTTGGTTCGAATACGAAAGAGGTAGATTGTCTCAAGAAGCTTGTTACGAAAGATGTGCT
GAAGAATTCAAGATCGAAGCTTCTGTTATCGCTGAAGCTTTCAAGCAAGCTAGAGGTTCTTT
GAGACCAAACGAAGAATTCATCGCTTTGATCAGAGACTTGAGAAGAGAAATGCACGGTGACT
TGACTGTTTTGGCTTTGTCTAACATCTCTTTGCCAGACTACGAATACATCATGTCTTTGTCT
TCTGACTGGACTACTGTTTTCGACAGAGTTTTCCCATCTGCTTTGGTTGGTGAAAGAAAGCC
ACACTTGGGTTGTTACAGAAAGGTTATCTCTGAAATGAACTTGGAACCACAAACTACTGTTT
TCGTTGACGACAAGTTGGACAACGTTGCTTCTGCTAGATCTTTGGGTATGCACGGTATCGTT
TTCGACAACCAAGCTAACGTTTTCAGACAATTGAGAAACATCTTCGGTGACCCAATCAGAAG
AGGTCAAGAATACTTGAGAGGTCACGCTGGTAAGTTGGAATCTTCTACTGACAACGGTTTGA
TCTTCGAAGAAAACTTCACTCAATTGATCATCTACGAATTGACTCAAGACAGAACTTTGATC
TCTTTGTCTGAATGTCCAAGAACTTGGAACTTCTTCAGAGGTGAACCATTGTTCTCTGAAAC
TTTCCCAGACGACGTTGACACTACTTCTGTTGCTTTGACTGTTTTGCAACCAGACAGAGCTT
TGGTTAACTCTGTTTTGGACGAAATGTTGGAATACGTTGACGCTGACGGTATCATGCAAACT
TACTTCGACAGATCTAGACCAAGAATGGACCCATTCGTTTGTGTTAACGTTTTGTCTTTGTT
CTACGAAAACGGTAGAGGTCACGAATTGCCAAGAACTTTGGACTGGGTTTACGAAGTTTTGT
TGCACAGAGCTTACCACGGTGGTTCTAGATACTACTTGTCTCCAGACTGTTTCTTGTTCTTC
ATGTCTAGATTGTTGAAGAGAGCTGACGACCCAGCTGTTCAAGCTAGATTGAGACCATTGTT
CGTTGAAAGAGTTAACGAAAGAGTTGGTGCTGCTGGTGACTCTATGGACTTGGCTTTCAGAA
TCTTGGCTGCTGCTTCTGTTGGTGTTCAATGTCCAAGAGACTTGGAAAGATTGACTGCTGGT
CAATGTGACGACGGTGGTTGGGACTTGTGTTGGTTCTACGTTTTCGGTTCTACTGGTGTTAA
GGCTGGTAACAGAGGTTTGACTACTGCTTTGGCTGTTACTGCTATCCAAACTGCTATCGGTA
GACCACCATCTCCATCTCCATCTGCTGCTTCTTCTTCTTTCAGACCATCTTCTCCATACAAG
TTCTTGGGTATCTCTAGACCAGCTTCTCCAATCAGATTCGGTGACTTGTTGAGACCATGGAG
AAAGATGTCTAGATCTAACTTGAAGTCTCAATAA
XP_007369631.1 Amino acid sequence of
Dichomitus squalens Albicanol synthase
SEQ ID NO: 5
MASIHRRYTTLILDLGDVLFRWSPKTETAIPPQQLKDILSSVTWFEYERGRLSQEACYERCA
EEFKIEASVIAEAFKQARGSLRPNEEFIALIRDLRREMHGDLTVLALSNISLPDYEYIMSLS
SDWTTVEDRVFPSALVGERKPHLGCYRKVISEMNLEPQTTVFVDDKLDNVASARSLGMHGIV
FDNQANVFRQLRNIFGDPIRRGQEYLRGHAGKLESSTDNGLIFEENFTQLIIYELTQDRTLI
SLSECPRTWNFFRGEPLFSETFPDDVDTTSVALTVLQPDRALVNSVLDEMLEYVDADGIMQT
YFDRSRPRMDPFVCVNVLSLFYENGRGHELPRTLDWVYEVLLHRAYHGGSRYYLSPDCFLFF
MSRLLKRADDPAVQARLRPLFVERVNERVGAAGDSMDLAFRILAAASVGVQCPRDLERLTAG
QCDDGGWDLCWFYVFGSTGVKAGNRGLTTALAVTAIQTAIGRPPSPSPSAASSSFRPSSPYK
FLGISRPASPIRFGDLLRPWRKMSRSNLKSQ
XP_006461126 Agaricus bisporus Drimeol synthase
DNA sequence codon optimized for its expression
in S. cerevisiae
SEQ ID NO: 6
ATGGCTCCACCACAAAGACCATTCACTGCTATCGTTTTCGACATCGGTGACGTTTTGTTCCA
ATGGTCTGCTACTACTAAGACTTCTATCTCTCCAAAGACTTTGAGATCTATCTTGAACTGTC
CAACTTGGTTCGACTACGAAAGAGGTAGATTGGCTGAAAACGCTTGTTACGCTGCTATCTCT
CAAGAATTCAACGTTAACCCAGACGAAGTTAGAGACGCTTTCTCTCAAGCTAGAGACTCTTT
GCAAGCTAACCACGACTTCATCTCTTTGATCAGAGAATTGAAGGCTCAAGCTAACGGTAGAT
TGAGAGTTTACGCTATGTCTAACATCTCTTTGCCAGACTGGGAAGTTTTGAGAATGAAGCCA
GCTGACTGGGACATCTTCGACCACGTTTTCACTTCTGGTGCTGTTGGTGAAAGAAAGCCAAA
CTTGGCTTTCTACAGACACGTTATCGCTGCTACTGACTTGCAACCACACCAAACTATCTTCG
TTGACGACAAGTTGGAAAACGTTTTGTCTGCTAGATCTTTGGGTTTCACTGGTATCGTTTTC
GACGAACCATCTGAAGTTAAGAGAGCTTTGAGAAACTTGATCGGTGACCCAGTTCAAAGAGG
TGGTGAATTCTTGGTTAGAAACGCTGGTAAGTTGGGTTCTATCACTAGAACTACTGCTAAGC
ACGAATCTATCCCATTGGACGAAAACTTCGCTCAATTGTTGATCTTGGAAATCACTGGTAAC
AGAGCTTTGGTTAACTTGGTTGAACACCCACAAACTTGGAACTTCTTCCAAGGTAAGGGTCA
ATTGACTACTGAAGAATTCCCATTCGACTTGGACACTACTTCTTTGGGTTTGACTATCTTGA
AGAGATCTAGAGAAATCGCTGACTCTGTTATGGACGAAATGTTGGAATACGTTGACCCAGAC
GGTATCATCCAAACTTACTTCGACCACAGAAGACCAAGATTCGACCCAGTTGTTTGTGTTAA
CGCTTTGTCTTTGTTCTACGCTTACGGTAGAGGTGAACAATTGAGATCTACTTTGACTTGGG
TTCACGAAGTTTTGTTGAACAGAGCTTACTTGGACGGTACTAGATACTACGAAACTGCTGAA
TGTTTCTTGTACTTCATGTCTAGATTGTTGGCTACTTCTGGTGACCCAGACTTGCACTCTTT
GTTGAAGCCATTGTTGAAGGAAAGAGTTCAAGAAAGAATCGGTGCTGACGGTGACTCTTTGG
CTTTGGCTATGAGAATCTTGGCTTGTGACTTCGTTGGTATCAGAGACGAAGTTGACTTGAGA
ACTTTGTTGACTTTGCAATGTGAAGACGGTGGTTGGGAAGTTGGTTGGATGTACAAGTACGG
TTCTTCTGGTATCTCTATCGGTAACAGAGGTTTGGCTACTGCTTTGGCTATCAAGGCTGTTG
ACACTATGTTCCAACCACAAATCAGATTCTCTGAATCTCCAACTGACACTTTGGTTGAAAAC
GCTATCCACAAGAGAAGACCATCTTTCTCTGAAAAGTTCTTGGGTAAGAGACCAAGATCTGG
TTCTTTCAGAAAGCCATTGCAATGGATCTTGCAAGGTTCTAAGTTGAGAAAGTCTGTTGAAA
TCGGTTCTTAA
XP_006461126 Protein sequence of
Agaricus bisporus Drimenol synthase
SEQ ID NO: 7
MAPPQRPFTAIVEDIGDVLFQWSATTKTSISPKTLRSILNCPTWEDYERGRLAENACYAAIS
QEFNVNPDEVRDAFSQARDSLQANHDFISLIRELKAQANGRLRVYAMSNISLPDWEVLRMKP
ADWDIFDHVFTSGAVGERKPNLAFYRHVIAATDLQPHQTIFVDDKLENVLSARSLGFTGIVE
DEPSEVKRALRNLIGDPVQRGGEFLVRNAGKLGSITRTTAKHESIPLDENFAQLLILEITGN
RALVNLVEHPQTWNFFQGKGQLTTEEFPFDLDTTSLGLTILKRSREIADSVMDEMLEYVDPD
GIIQTYFDHRRPRFDPVVCVNALSLFYAYGRGEQLRSTLTWVHEVLLNRAYLDGTRYYETAE
CFLYFMSRLLATSGDPDLHSLLKPLLKERVQERIGADGDSLALAMRILACDFVGIRDEVDLR
TLLTLQCEDGGWEVGWMYKYGSSGISIGNRGLATALAIKAVDTMFQPQIRFSESPTDTLVEN
AIHKRRPSFSEKFLGKRPRSGSFRKPLQWILQGSKLRKSVEIGS
CrDAT DNA sequence codon optimized for
its expression in S. cerevisiae
SEQ ID NO: 8
ATGGAAAGTGGTAAAATATCTGTAGAAACGGAAACCCTATCGAAAACCCTTATCAAGCCGTC
CTCGCCCACGCCACAGTCTCTTTCCCGTTACAACTTATCTTACAATGATCAGAACATTTATC
AGACCTGCGTATCTGTAGGGTTCTTTTACGAAAATCCTGATGGCATCGAGATATCCACGATC
CGTGAGCAGCTGCAGAACAGCCTGTCCAAAACTCTGGTCTCATACTATCCCTTCGCAGGCAA
AGTAGTGAAGAACGACTACATACATTGTAACGACGATGGCATTGAGTTCGTCGAAGTCAGAA
TTAGATGCAGGATGAATGATATACTTAAATACGAACTACGTTCTTACGCTCGTGATTTAGTC
CTGCCAAAGAGGGTTACCGTGGGCTCTGAAGATACCACCGCTATCGTGCAGCTGTCCCATTT
CGACTGTGGCGGTTTGGCCGTGGCTTTTGGTATCTCCCACAAGGTGGCCGATGGTGGTACTA
TAGCCTCTTTCATGAAGGACTGGGCGGCCTCTGCTTGCTACTTGTCCTCAAGTCACCACGTT
CCAACGCCTCTATTGGTCTCAGACAGTATTTTCCCAAGACAGGACAACATAATATGCGAACA
ATTTCCCACCTCAAAGAATTGCGTGGAGAAGACATTCATTTTCCCACCAGAAGCAATCGAAA
AGTTAAAGAGTAAGGCCGTTGAATTCGGCATTGAGAAACCGACCAGAGTAGAGGTCCTGACT
GCATTCTTATCTAGATGCGCCACCGTAGCAGGTAAGTCGGCAGCTAAGAACAACAATTGTGG
TCAAAGCCTGCCCTTTCCGGTTCTACAGGCCATTAATTTGAGGCCGATTCTAGAATTGCCAC
AGAACTCTGTGGGTAATCTAGTTTCGATCTACTTCAGCAGGACAATTAAGGAAAACGACTAC
CTAAATGAAAAGGAGTACACTAAATTGGTGATAAACGAGTTGCGTAAAGAAAAGCAAAAGAT
CAAGAATTTGAGCAGAGAGAAGTTGACCTACGTCGCCCAGATGGAAGAGTTCGTTAAGTCAC
TTAAAGAGTTCGATATCTCCAACTTCCTAGATATCGATGCCTACTTAAGCGATAGTTGGTGC
AGGTTTCCTTTCTACGACGTCGACTTCGGATGGGGAAAGCCTATCTGGGTCTGCCTATTCCA
GCCTTACATCAAGAACTGCGTTGTTATGATGGACTATCCTTTCGGTGACGATTATGGTATCG
AGGCTATTGTTTCTTTCGAGCAAGAGAAAATGTCCGCGTTCGAGAAGAACGAACAGTTACTG
CAGTTTGTGTCTAACTAA
CrDAT Protein sequence
SEQ ID NO: 9
MESGKISVETETLSKTLIKPSSPTPQSLSRYNLSYNDQNIYQTCVSVGFFYENPDGIEISTI
REQLQNSLSKTLVSYYPFAGKVVKNDYIHCNDDGIEFVEVRIRCRMNDILKYELRSYARDLV
LPKRVTVGSEDTTAIVQLSHEDCGGLAVAFGISHKVADGGTIASFMKDWAASACYLSSSHHV
PTPLLVSDSIFPRQDNIICEQFPTSKNCVEKTFIFPPEAIEKLKSKAVEFGIEKPTRVEVLT
AFLSRCATVAGKSAAKNNNCGQSLPFPVLQAINLRPILELPQNSVGNLVSIYFSRTIKENDY
LNEKEYTKLVINELRKEKQKIKNLSREKLTYVAQMEEFVKSLKEFDISNELDIDAYLSDSWC
RFPFYDVDFGWGKPIWVCLFQPYIKNCVVMMDYPFGDDYGIEAIVSFEQEKMSAFEKNEQLL
QFVSN
TcTAT DNA sequence codon optimized for
its expression in S. cerevisiae
SEQ ID NO: 10
ATGGAGAAGACGGATTTACACGTGAACCTTATCGAAAAGGTTATGGTCGGTCCAAGCCCACC
TTTGCCGAAGACTACACTACAACTATCCTCAATCGATAACCTACCTGGTGTTAGAGGCTCGA
TCTTTAATGCCTTGTTAATATATAATGCTTCACCGTCTCCCACGATGATCTCCGCTGATCCA
GCTAAACCTATCAGAGAAGCTTTGGCCAAAATCCTGGTTTACTACCCACCTTTCGCTGGCAG
GCTACGTGAGACTGAGAATGGCGATCTAGAGGTAGAGTGCACGGGAGAGGGTGCGATGTTTC
TTGAAGCGATGGCCGATAATGAATTAAGTGTCTTAGGGGATTTCGACGACAGTAACCCTAGC
TTCCAGCAATTGTTGTTCTCCCTTCCACTGGACACTAACTTCAAGGATCTATCCCTGTTAGT
CGTGCAGGTGACAAGGTTTACATGCGGCGGGTTCGTCGTTGGGGTTTCATTTCACCACGGTG
TATGCGATGGGAGAGGAGCTGCACAATTTCTGAAGGGACTAGCCGAAATGGCAAGGGGAGAA
GTTAAGTTGTCGCTGGAGCCGATCTGGAATCGTGAACTAGTTAAGCTGGACGATCCCAAGTA
TTTGCAATTCTTTCACTTCGAATTCTTGAGAGCACCATCAATCGTCGAAAAGATAGTTCAAA
CCTACTTCATCATCGATTTCGAAACTATTAACTACATAAAGCAGTCAGTAATGGAAGAATGT
AAAGAATTCTGCAGCTCTTTTGAAGTTGCTTCTGCAATGACGTGGATAGCTAGGACCAGGGC
CTTTCAAATACCCGAGAGCGAGTACGTGAAAATCCTATTTGGTATGGACATGAGGAACTCTT
TTAACCCGCCTCTGCCGTCAGGTTATTATGGAAACTCAATTGGTACGGCTTGTGCAGTGGAC
AATGTTCAAGACTTGTTGAGTGGTTCACTACTGAGGGCAATAATGATAATCAAGAAATCAAA
GGTATCACTTAACGACAACTTTAAGAGCCGTGCTGTCGTTAAGCCTTCAGAACTTGACGTAA
ACATGAACCATGAGAATGTGGTGGCATTTGCTGACTGGTCAAGGCTGGGGTTCGACGAAGTC
GATTTTGGCTGGGGAAACGCGGTGTCTGTTTCCCCGGTACAGCAACAGTCGGCACTTGCAAT
GCAGAATTACTTCCTGTTCTTGAAGCCAAGCAAGAACAAGCCCGATGGAATTAAGATTCTAA
TGTTCCTACCATTGTCCAAGATGAAATCCTTCAAAATTGAAATGGAAGCGATGATGAAGAAG
TACGTCGCCAAGGTATAA
TcTAT Protein sequence
SEQ ID NO: 11
MEKTDLHVNLIEKVMVGPSPPLPKTTLQLSSIDNLPGVRGSIFNALLIYNASPSPTMISADP
AKPIREALAKILVYYPPFAGRLRETENGDLEVECTGEGAMFLEAMADNELSVLGDEDDSNPS
FQQLLFSLPLDTNFKDLSLLVVQVTRFTCGGFVVGVSFHHGVCDGRGAAQFLKGLAEMARGE
VKLSLEPIWNRELVKLDDPKYLQFFHFEFLRAPSIVEKIVQTYFIIDFETINYIKQSVMEEC
KEFCSSFEVASAMTWIARTRAFQIPESEYVKILFGMDMRNSFNPPLPSGYYGNSIGTACAVD
NVQDLLSGSLLRAIMIIKKSKVSLNDNEKSRAVVKPSELDVNMNHENVVAFADWSRLGEDEV
DFGWGNAVSVSPVQQQSALAMQNYFLFLKPSKNKPDGIKILMFLPLSKMKSFKIEMEAMMKK
YVAKV
CrMAT DNA sequence codon optimized for
its expression in S. cerevisiae
SEQ ID NO: 12
ATGGATTCTATTACTATGGTTGAAACCGAGACATTATCGAAGACATTGATTAAGCCATCTAG
CCCAACCCCACAGAGTCTGTCACACTACAACCTGTCTTACAACGATCAGAATATATATCCAG
AGTATATATTTGCAGGCTTCTTCTACAGCAACCCGGACGGACACGAGATCTCGACTATTAGA
GAGCAGCTGCAGAACTCTCTTAGCAAGACATTAGTATCTTATTATCCATTCGCTGGTAAAGT
GGTCAAGAACGATTACATTCACTGCAATGACGACGGAATAGAATTCGTAGACGTCAGAATCC
ACTGCAGGATGAACGATATTCTTAAGCCTGAATTGAGGTCTTACGCTTCCGAGCTTATTCGT
CCGAATAGAAGTACAGTGGGGTCAGAGGACAGTACTGCGCTTGTCCAGTTATCTCACTTTGA
CTGTGGCGGGGTAGCTGTCGCATTCGGTATATCTCACAAAGTCGCAGACGCAGCGACTATTC
TGTCGTTTATAAAGGATTGGGCCGCTTCTACGTGTGACTTGTCAAGTAGTCACGATGTGTCT
ACACCAGTACTAGTATCCGATTCCATATTCCCCCGTCAAGATAACATCATCTGTGGCCAGTT
CCCTGCTTCACCGAACTGCGTGAGGAAGCGTTTCTTATTCAGCCCGGAAGCTATCGAAAGAC
TAAAATCGAAAGCCATTGAATTTGGGATCGAGAAGCCAACGAGGGTAGAGGTCCTGACAGCA
TTCTTGTGCCGTTGCGCTACCGTTGCAGGTAAATCTGCGGCCAAGAACAACAATTGTGGACA
GTCACTGCCTTTCGCTGTTATTCAAGCAGTCAACCTGAGGCCCTTACTAGAACTGCCAAAGA
ATTCCGTCGGCAACCTTATATCAATCTACTTTTCTACAATCAAAGAAAACGACACGGTGAAC
ATCGAACAGGAGTTCACAAAATTAGTGATCGGAGAGTTGAGGAAGGCTAAGGATAAGTTAAA
GAACCTGTCGCAAGAGAAGCTGAATTACGTAGCTAGAATGCAAGATTTCGCGAATTGCCTGA
AGGAATTGGACATAAGTTCATTCTTCGACATGGAAAACGTGGACATAGACGCTTATTTATTT
TCGAGCTGGTGCAGGTTCCCCTTCTACGACATCGATTTCGGTCTGGGGAAGCCAATATGGGT
CTGCATGTTTCAGCCTCACTTTAAGAATTGTATAATTTTAATGGATTATCCCTTTGGTGACG
ATTACGGCATCGAAGCCCTAATTACGTTGGAACAAGAGAAGATGCCCGCCTTCGAAAACAAC
GAGCTGCTGCTAAGCTTCGCCAGCAACTAA
CrMAT Protein sequence
SEQ ID NO: 13
MDSITMVETETLSKTLIKPSSPTPQSLSHYNLSYNDQNIYPEYIFAGFFYSNPDGHEISTIR
EQLQNSLSKTLVSYYPFAGKVVKNDYIHCNDDGIEFVDVRIHCRMNDILKPELRSYASELIR
PNRSTVGSEDSTALVQLSHFDCGGVAVAFGISHKVADAATILSFIKDWAASTCDLSSSHDVS
TPVLVSDSIFPRQDNIICGQFPASPNCVRKRFLESPEAIERLKSKAIEFGIEKPTRVEVLTA
FLCRCATVAGKSAAKNNNCGQSLPFAVIQAVNLRPLLELPKNSVGNLISIYFSTIKENDTVN
IEQEFTKLVIGELRKAKDKLKNLSQEKLNYVARMQDFANCLKELDISSFFDMENVDIDAYLF
SSWCRFPFYDIDFGLGKPIWVCMFQPHFKNCIILMDYPFGDDYGIEALITLEQEKMPAFENN
ELLLSFASN
LiAAT-4 DNA sequence codon optimized for
its expression in S. cerevisiae
SEQ ID NO: 14
ATGGCCATGATAATCACGAAGCAAATATTAAGACCTAGTTCCCCGACGCCCCAAGCCTTTAA
GAATCACAAGCTGTCCTACTTAGACCAAATACAGGCGCCTATTTACATACCTTTGTTGTTCT
TCTATAAGAACGAGGAGTCAAAATACCCAGACCAGATCTCGCAAAGATTTAAGCAGAGTTTG
TCCGAAATTTTGACAATATTCTACCCTTTGGCTGGTACGATGAGGCATAACTCGTTCGTGGA
CTGCAATGACAGGGGTGTCGAATTTGTAGAGGTCAGGGTCCATGCGAGACTAGCCCAGTTCA
TTCAAGATCCTAAGATGGAAGAGCTTAAGCAATTGATACCTGTGGATTGTATATCTCACACT
GACGATGATTTCTTATTGCTAGTCAAGATTAGCTATTTTGACTGCGGTGAGGTCGTCGTTGG
AGTCTGCATGTCGCACAAAATTGGTGACGGAATTAGTCTGGCAGCGTTTATGAACGCTTGGG
CAGCAACGTGTAGGGGAGAATCGTCTAGTGAGATCATTCACCCATCTTTTGATCTTGCTTTA
CACTTTCCGCCTAAAGACCACTTGTCTTCAGCATCCTCATTCCGTGTGGCCATAGCCCAGGA
GAACATCATGACCAAGAGGCTAGTATTTGATAGAGAAAAGTTGGAGAAGCTGCGTAAGAGAA
TCGCTGCCAGTTCTGATGGGGTGAGAGACCCTAGCAGAGTTGAAGCTGTATCTGTCTTTATT
TGGAAAAGCTTAATTGAAGCCCACAAGGCCGAGTCACACATGACTGAGACACCAGCCGTTTC
TATTGCTAGCCACGCCGTGAACTTAAGGCCTAGAACAGTCCCACAAATGGACCAAACTTTCG
GTAACTGCTACGCTCCCGCTTCGGCCGTTGTCTCCTGGGATGAAGACTACGTACATCACAGT
CGTTTGAGGGCGGCCCTTAGAGAAATCGACGACGACTACATTAATAAGGTCTTGAAAGCCGA
CAATAATTATTTAACGCAGGATCAAATTGGTGACTTGTTTAAACCAGAAAACTCAGTTCTAA
GTTCGTGGTGGCGTTTCCCAGTTTACAAGGTAGACTTTGGATGGGGTAAGCCAGTTTGGGTT
TCTACGACGACCATCCAATACATGAACTTGATTATATTTACTTCGACGCCCTCAGAAGACGG
CATAGAGGCGTGGGTAACTACTACTCACAATTTCTTCCAAGTTCTGCAGGCAAACTATAATA
AACTTGACACGTAA
LiAAT-4 Protein sequence
SEQ ID NO: 15
MAMIITKQILRPSSPTPQAFKNHKLSYLDQIQAPIYIPLLFFYKNEESKYPDQISQRFKQSL
SEILTIFYPLAGTMRHNSFVDCNDRGVEFVEVRVHARLAQFIQDPKMEELKQLIPVDCISHT
DDDFLLLVKISYFDCGEVVVGVCMSHKIGDGISLAAFMNAWAATCRGESSSEIIHPSFDLAL
HFPPKDHLSSASSFRVAIAQENIMTKRLVFDREKLEKLRKRIAASSDGVRDPSRVEAVSVFI
WKSLIEAHKAESHMTETPAVSIASHAVNLRPRTVPQMDQTFGNCYAPASAVVSWDEDYVHHS
RLRAALREIDDDYINKVLKADNNYLTQDQIGDLFKPENSVLSSWWRFPVYKVDFGWGKPVWV
STTTIQYMNLIIFTSTPSEDGIEAWVTTTHNFFQVLQANYNKLDT
FgaAT DNA sequence codon optimized for
its expression in S. cerevisiae
SEQ ID NO: 16
ATGAAGAAGCAGGTCACTTTTAAACCTTTCAGATTGTCACCAGTCGATCATAGTTTACCTAA
GGTTTACATCTTCAAGTCTCTATATTTCAGAGGGGTAGATGACACTGGTTCTCTTAGTAGAC
TACAGGATGGCATTGATCGTTTGATATCTTGTCTGCCTTTCCTGTCAGGCGAGGTGGTTCCA
TGTGCAGACATACCAGATAAGGTCGGTGTACTTCAAGTTCAGATGCCTTGCCCTTCCTTGCA
AGAAATTCCAATGCTGCTAGTCAAAAGTTATCCAAACCATACATGGCCAGCAGCTTCTACCT
CGGAAAGATGGAGAAACACCGCTTTGCTAGATCAGTCGTATAGGCCATTGCCGGATTTTATA
CCCCCATCAAAGCCTAGACCTGTTTTGAGATTTCAGGCGAACTTCCTGGCTGATGGTTTAAT
GCTATGTATGGGATATAATCATTCTGTATTCGACGGAACAGGTGCGGGGAACATTCTGGAAA
TGTTGGCTGATTGTTGTAGAGCTAATCCAAACTCCATCTTAGCACTGCCAACAAACGGTGAC
ATAGAGAGCGAGTTGAGAGGTTTACTGTCCAGTCCAGGTGTAGCAGTTGCAAATGCCTCTCA
GGAAGCCTACGCAATTAATTGTGCACACACGGAAGTAGAACCCGAACCCAGTTCAGCTATGC
TTTACTGTTGGCCTTTCTTGCTGAGTTCTGAGAAGATTGAATGTTTACAGGAAGCATGTAAT
AGTTTACTACCACATATCGTTCGTTTGTACTCTGGTACGCAGAGTTCGCTTATAAACCAAGA
TACGAACTGGCCACATATTCTTTCATCAAACGACGTGCTAACCGCTCTTTTAGCTGTTTCAA
TTGAAAAGGCTAGGGAAGCAACAGGTGCCCTTGGTCATATGAGCAGATCATTGGCTATGGCT
GTTAACCTACGTGAGCGTTTAAAGCCTATGCCAAGACACTATTTGGGTAACCTAGTTACCAC
TGTGTGGGTATCACATCACCGTCCTGCCGTTAAGGACCTTGAAACAATGGTTTTACCAGTGC
CAGCATGCAATAGGCACGAAATAGACAGAGACGACTTGTTGTGGATAACCCACGTGGCATTC
CGTATTAGATTAGGGCTGAACGCAATAAACGAAGAACATATTAGAGGTCTGATCCACTACTT
GCATTCCCAAGACGATTGGGAACAGATTGGAATACATTTCACCGATCCAATTTTCATTTCTT
CCTGGCGTCACCTTAAGGTCTATGAACTAGACTTCGGACCTACTATCGGTCATGCTGAACAC
TTTGAGATGGATGTCGGCACCACCGATGGTGTGTGTGTTGTTATGCCTGCTAACACCAGAGC
CGTCGGCAAGACTAAGAAGGCTCCTTGGGACATTAGAATCGTGTTAAACCCCGAAGTGTTAC
AGGCACTAATAGCTAGTGCCATCTTTGGTTGGGCTATGGTCAAGGACGCTTCGACATAA
FgaAT Protein sequence
SEQ ID NO: 17
MKKQVTFKPFRLSPVDHSLPKVYIFKSLYFRGVDDTGSLSRLQDGIDRLISCLPFLSGEVVP
CADIPDKVGVLQVQMPCPSLQEIPMLLVKSYPNHTWPAASTSERWRNTALLDQSYRPLPDFI
PPSKPRPVLRFQANFLADGLMLCMGYNHSVEDGTGAGNILEMLADCCRANPNSILALPINGD
IESELRGLLSSPGVAVANASQEAYAINCAHTEVEPEPSSAMLYCWPFLLSSEKIECLQEACN
SLLPHIVRLYSGTQSSLINQDTNWPHILSSNDVLTALLAVSIEKAREATGALGHMSRSLAMA
VNLRERLKPMPRHYLGNLVTTVWVSHHRPAVKDLETMVLPVPACNRHEIDRDDLLWITHVAF
RIRLGLNAINEEHIRGLIHYLHSQDDWEQIGIHFTDPIFISSWRHLKVYELDEGPTIGHAEH
FEMDVGTTDGVCVVMPANTRAVGKTKKAPWDIRIVLNPEVLQALIASAIFGWAMVKDAST
GAO81666.1 DNA sequence codon optimized for
its expression in S. cerevisiae
SEQ ID NO: 18
ATGGAAGAACATACCAGATCCTTTGAGCCATTTGACCTGGCCTGCTTAGATCATACTGTGGG
GCCGGTTTTCATGAATTTCTTCTTAAGTTTCAAGCCTGCCAAGATCGAAGAGAGTCTTATCT
CAATTGAAGAAGGTGTAACTAGGTTAGTCAACAGACTGCCATTCCTGGCCGGTGACGTTGTT
AATTCTGAAAACGTAGACGGTCGTGTTAATGTAATGAGGATTCAGCCAAGCTCGACATTGAT
TAGAGAGATTCCCATGCTACACACAAAGCACCATCCTCATCATATTTTGCCGAATACACCTA
GACAGATTAACCCAGGGTCCGACCAGGATCAAAGGTTTGCTCCCTTGGACGATTCGTACGTA
CCACCAGTGTCTTTATTACCTCTTGCTCCGGGCCCAAGGCCCGTAGTAAGATTCCAAACTAA
TGTTGTAATTGACGGGATTGTGCTTGCACTTGGGTTCCACCACTCAGTATTTGATGCTACCG
GAGTGGGTTTGTTGATCGAAATGTTGGCCACATGTTGTTCAAGCGATTGTCCTGCCCTATCA
TCCCATATTGAACTGGAAGAAGAAATAAGGCTAAGGCGTTCTGTGGACAAGATAGGTAACGG
CGCTACTGATTTAGCATCCCAAAGAGATGAAGTTCAGGACCCAAATGGCAGTATCACTGAGT
CCCCAGCTCATGTGCCTATGGGTGACAGTAGCTGGGTACCCCCTAAACTATCTGTTTACTCC
TTTAATTTATCGGCTGCCGGTTTGGCTCATTTGAAGACAGCCTGTAACAAGTTATTGCCCGC
TATCCATGCGAGTCAAAATGGTTCACCACAAAGTGCGGAATCAGAAGGTGAGAAATTACAGC
AAGAATTCGTTTCGACGAATGATGTTCTAACGGCTTTGTTGGCTACTTCCATTCACCAAGCG
AGGTCTAGAGTTACTGAAACGGAATTAGTTCCGACAAAAGCGAAACTTGCGATGGCCGTTAA
TTTGAGGGAAAGGGCCAGTTCGTTACCCAAGACTTATTTGGGGAACTCGCTAACAGTTACAG
AAGCTTTTGTGTATTCTTTAGCTGCTACCGACGGTTTCGATGGTGTCCCCGAGAGACATTAT
CATCCTGACTTGCGTAACGCCTTGCTATTAGAAATTGCTAGAGTTGCATTGCAATTGAGAAA
AGGGCTGGCAGCGATAGATGACGCGTATTTTAGGCAATTTGTGTCTAGATTGAGGGCTAATC
TAGACTGGTCACAGTTAGGTGCAAACCTGCCTGACACTATGGTGTCAAGCTGGAGACATTTG
AAAGTATATCGTTTAGACTTCGGTGCTAGATTGGGGAGAGTTGTAGAGTTCCATCCACAAAC
CGCTTTGGTTGACGGTATTTGTATCATCCAACCTGAAAGGATAGCCCATGAGGATGATTCAG
CTGATATGGCTCCAGAATCTGGGTGGGAAGTTTGTGTAACATTACAAAGCGATGCCATGGAG
TGCTTCTTAAGAGGTGGGTTGTTCACAAGTCTTTCTCAAGGTGCAATTAGAAGAGTTTAA
GAO81666.1 Protein sequence
SEQ ID NO: 19
MEEHTRSFEPFDLACLDHTVGPVEMNFFLSFKPAKIEESLISIEEGVTRLVNRLPFLAGDVV
NSENVDGRVNVMRIQPSSTLIREIPMLHTKHHPHHILPNTPRQINPGSDQDQRFAPLDDSYV
PPVSLLPLAPGPRPVVRFQTNVVIDGIVLALGFHHSVEDATGVGLLIEMLATCCSSDCPALS
SHIELEEEIRLRRSVDKIGNGATDLASQRDEVQDPNGSITESPAHVPMGDSSWVPPKLSVYS
FNLSAAGLAHLKTACNKLLPAIHASQNGSPQSAESEGEKLQQEFVSTNDVLTALLATSIHQA
RSRVTETELVPTKAKLAMAVNLRERASSLPKTYLGNSLTVTEAFVYSLAATDGEDGVPERHY
HPDLRNALLLEIARVALQLRKGLAAIDDAYFRQFVSRLRANLDWSQLGANLPDTMVSSWRHL
KVYRLDFGARLGRVVEFHPQTALVDGICIIQPERIAHEDDSADMAPESGWEVCVTLQSDAME
CFLRGGLFTSLSQGAIRRV
CfACT1-6 DNA sequence codon optimized for
its expression in S. cerevisiae
SEQ ID NO: 20
ATGAAGGTAGAACGTTTCTCAAGAAAGTTGATAAAACCCCACACGCCTACTCCCGAGAACTT
AAAGAAATATAAATTGTCTCTATTGGACAAATGCCTTGGACACGACAACTTCGCGATCGTGT
TGTTTTACGAATCTAAACCAAGAAATAAGAGTGAGCTTGAGGAATCGTTAGAGAAGGTCTTG
GTAGACTTTTATCCCTTGGCTGGGCGTCACACGATGAACGACCATATTGTCGACTGCTCGGA
TGTCGGCGCTGTCTTTGTAGAAGCCGAGGCTTTAGATGTAGAGTTGACTATGGATGAGTTAG
TCAAGAACATGGAAGCTCAAACTATACACCACTTGTTGCCAAATCAGTACTTCAGTGCAGAT
GCTCCGAACCCGCTGCTTTCCATCCAAGTTACGCACTTTCCTTCCGGTGGTTTGGCTATCGG
AATCGCAGTCTCGCACGCTGTATTTGATGGTTTCTCTTTGGGCGTGTTCGTAGCAGCATGGT
CAAAGGCGACCATGAACCCGGATAGGAAAATCAAAATAACACCGTCATTCGACTTACCATCA
CTTCTGCCCTACAAAGACGACAACTTTGGTTTGACTGCTGCTGAAATTGTCAGCCAGAGCGA
GGACATCGTAGTTAAGAGATTTATCTTCGGCAAGGAAGCCATCACGAGGTTGAGAAGTAAGC
TTAGTCCAAATAGGAACGGGAAGAAAATATCCAGGGTTAGGGTCGTTTGTGCAGTCATTGTA
AAGGCCTTGATGGGATTGGAGCGTGCCAAACACGGTAAGACGCGTGATTTCTTAATTACTCA
ATCAATTAACATGAGGGAGAGAACTAAGGCGCCGCTGCAGAAACACGCCTGCGGCAACTTAG
CAGTCTTGAGTTGCACGAGAAGAGTAGAGGCCGAGGAGATGATGGAGTTACAGAACCTAGTT
AATTTGATCGGCGACAGTACCGAAAAGGACATCGCCGACTTTGCAGAATTATTATCACCAGA
TCAAGTGGGCAGAGACATTATAATCAAGATGATGAAGTCATTCATGCAGTTCTTGGACAATG
ACATTTACAGCGTGTGCTTTACCGATTGGTCAAAGTTTGAATTTTACGAAGCGGACTTCGGC
TTCGGGAAGCCGGTTTGGATGGCAGCAGGCCCACAGAGACCCATTATCTCTACTGCAATATT
GATGTCAGACAGGGAAGGCGACGGTATCGAGGCTTGGTTACATTTGAATAAGAACGACATGT
TGATTTTCGAACAAGACGAGGAAATCAAGTTATTCACTACATAA
CfACT1-6 Protein sequence
SEQ ID NO: 21
MKVERFSRKLIKPHTPTPENLKKYKLSLLDKCLGHDNFAIVLFYESKPRNKSELEESLEKVL
VDFYPLAGRHTMNDHIVDCSDVGAVFVEAEALDVELTMDELVKNMEAQTIHHLLPNQYFSAD
APNPLLSIQVTHFPSGGLAIGIAVSHAVEDGFSLGVFVAAWSKATMNPDRKIKITPSFDLPS
LLPYKDDNFGLTAAEIVSQSEDIVVKRFIFGKEAITRLRSKLSPNRNGKKISRVRVVCAVIV
KALMGLERAKHGKTRDFLITQSINMRERTKAPLQKHACGNLAVLSCTRRVEAEEMMELQNLV
NLIGDSTEKDIADFAELLSPDQVGRDIIIKMMKSFMQFLDNDIYSVCFTDWSKFEFYEADFG
FGKPVWMAAGPQRPIISTAILMSDREGDGIEAWLHLNKNDMLIFEQDEEIKLFTT
CfACT1-8 DNA sequence codon optimized
for its expression in S. cerevisiae
SEQ ID NO: 22
ATGAAAGTCGAAAGGATTTCACGTAAATTCATCAAGCCATATACACCTACACCACAGAACCT
TAAGAAGTACAAGCTATCCTTGCTGGATAAATGCATGGGACACATGGACTTCGCTGTAGTAT
TGTTTTACGAATCAAAGCCAAGAAACAAGAATGAGCTGGAAGAATCACTAGAGAAAGTGTTA
GTCGATTTCTATCCATTGGCAGGCAGGTATACCATGAACGACCACATTGTCGATTGCAGCGA
TGAGGGCGCCGTTTTCGTTGAGGCAGAGGCCCCTAATGTTGAGCTTACAGTGGACCAGTTGG
TTAAGAACATGGAAGCCCAGACAATCCACGACTTCTTACCAGACCAATATTTTCCTGCTGAC
GCACCAAATCCGTTGCTAAGTATTCAAGTAACGCACTTCCCTTGTGGTGGTTTAGCTATCGG
GATTGTTGTTAGTCACGCGGTCTTTGATGGATTCTCATTGGGCGTATTCTTAGCCGCCTGGA
GCAAAGCTACCATGAACCCTGAGAGGAAGATCGAAATCACCCCTTCCTTCGATTTGCCTAGT
CTTCTGCCCTACAAGGATGAATCTTTCGGTTTAAATTTTAGCGAAATTGTCAAAGCTGAGAA
TATCGTAGTTAAACGTTTGAATTTCGGGAAAGAGGCTATTACGCGTTTGAGGTCCAAGCTGT
CTCCTAACCAGAATGGTAAAACCATTTCCAGAGTGAGGGTAGTCTGCGCGGTGATAGTTAAG
GCGTTGATGGGACTGGAGAGAGCAAAGACTAGAGACTTTATGATATGTCAGGGGATCAACAT
GAGAGAGAGGACGAAGGCGCCCCTGCAGAAGCATGCGTGTGGCAACCTAGCAGTTTCGTCTT
ACACTAGAAGGGTAGCGGCAGCGGAAGCAGAAGAACTGCAGTCCTTAGTGAATTTGATCGGG
GACTCTATCGAAAAGTCAATCGCAGACTACGCTGATATACTTTCGAGTGATCAAGATGGGAG
ACACATCATTTCCACTATGATGAAGAGCTTTATGCAGTTTGCTGCACCTGATATAAAAGCCA
TTTCATTTACCGACTGGTCAAAGTTCGGCTTTTACCAAGTAGATTTTGGTTTTGGTAAACCA
GTTTGGACCGGCGTCCGTCCAGAACGTCCAATCTTCTCAGCCGCGATATTGATGAGTAACAG
GGAAGGCGATGGCATCGAGGCTTGGCTTCATTTGGACAAGAACGATATGCTAATATTTGAGC
AAGACGAAGAAATTAAGTIGTTGATAACGTAA
CfACT1-8 Protein sequence
SEQ ID NO: 23
MKVERISRKFIKPYTPTPQNLKKYKLSLLDKCMGHMDFAVVLFYESKPRNKNELEESLEKVL
VDFYPLAGRYTMNDHIVDCSDEGAVEVEAEAPNVELTVDQLVKNMEAQTIHDFLPDQYFPAD
APNPLLSIQVTHFPCGGLAIGIVVSHAVEDGFSLGVFLAAWSKATMNPERKIEITPSFDLPS
LLPYKDESFGLNFSEIVKAENIVVKRLNFGKEAITRLRSKLSPNQNGKTISRVRVVCAVIVK
ALMGLERAKTRDEMICQGINMRERTKAPLQKHACGNLAVSSYTRRVAAAEAEELQSLVNLIG
DSIEKSIADYADILSSDQDGRHIISTMMKSFMQFAAPDIKAISFTDWSKFGFYQVDFGFGKP
VWTGVRPERPIFSAAILMSNREGDGIEAWLHLDKNDMLIFEQDEEIKLLITT
Optionally the C-terminal “T” may be missing
OAH94415.1 DNA sequence codon optimized for
its expression in S. cerevisiae
SEQ ID NO: 24
ATGGATACCAAGAGGGTCGGTTATACAGTCGTTGATTTGAGTCAGTGGGGCAGAAAAGAACA
CTTCGAAGCATTCCAGAGCTTCGCCCAGTGCACCTTTTCCCAGACTGTTCAATTAGACATAA
CTTCCCTGCTGAAGACTGTCAAGCAGAACGGATATAAATTCTACCCGACCTTTATCTACATA
ATAAGCCGTTTAGTGAACAAGCATGCCGAATTCCGTATGGCCATGAAGGATGGTGAACTGGT
AATTTGGGACTCCGTAAACCCTGGTTACACAATCTTTCACGAACAGACCGAAACATTTTCAT
CTCTGTGGAGCTATTACCACAAGGACATCAATCAGTTTCTTAAAACGTATTCTGAAGACATC
GCTCAGTACGGGGACGATCTGGCGTACTTCCCCAAGGAGTTTATTGAAAATATGTTCTTCGT
GTCAGCGAACCCATGGGTGAGCTTCACAAGTTTCAACTTAAACGTGGCCAATATTAATAACT
TCTTCGCACCGGTCTTTACAATCGGTAAATACTACACGCAGGGCGACAAGGTGTTGATGCCG
TTAGCTATTCAGGTCCATCATGCAGTCTGCGACGGGTTTCATGTCGGCAGGTTATTGAACGA
AATTCAGCAGTACTGCGATGAGGGATGCAAGTAA
OAH94415.1 Protein sequence
SEQ ID NO: 25
MDTKRVGYTVVDLSQWGRKEHFEAFQSFAQCTFSQTVQLDITSLLKTVKQNGYKFYPTFIYI
ISRLVNKHAEFRMAMKDGELVIWDSVNPGYTIFHEQTETFSSLWSYYHKDINQFLKTYSEDI
AQYGDDLAYFPKEFIENMFFVSANPWVSFTSFNLNVANINNFFAPVFTIGKYYTQGDKVLMP
LAIQVHHAVCDGFHVGRLLNEIQQYCDEGCK
DfACT13 native Nucleotide sequence
SEQ ID NO: 116
ATGGCCTGTGGAGGCGGCGTGAGGAATATTGAGGTGAAGGCGCAGGAGCCCGTTTTGGTGCA
GCCTTTATCTACAGCACAAAGCTCTGCCTATAATCTTCTTACAAAACTGGACCAGACGCTCG
CACAATTGGTGGTGCAGATTGTCTTTGTCTTCGATGTGAAAAACCCCGTAACCCGCCGACCC
CACGATGGCGCCGATCCTGCCAAGCTTCTGAAGGAGGCTCTGAGGAAGGTCCTTGTGCCCTT
CTACCCGCTCGCTGGGCGTCTTTGCCTCTCGCCCGATGACGGCAGTCTGTTCATTGACTGCA
ATGCTCAGGGGGTTTCCTTTGTTGAGGCCAATGCCGACGCGGATATCTCCGAACTCGGCGAC
TTCTCGCAGCCCGATTTCGCGACTCTGGGCTCCCTTGTCTTCCCCTTACCTCCTATAGCTTC
GGATGATGGTCCTCTTCTATCTGCGCAGGTGACCAGATTCAAGTGTGGAGGATTTGTGCTGG
GCTTCGTATTCCATCATTGTTTATTTGATGGATTCGCACTTTCGGAATTCCTAAATGCGTGG
GCGGAGACTGCATGCGGCGTGCCTCTTTCTACACCTCCTGTCCTCGACAGAACTTTTCCGAG
GGCGCGTTCTCCCTTGCAAATCAAGTATCCCCACACCGAGTTCCTGGAAGTCGAAGATGTCT
CTTTGACTCAAAATATCTCTAACGATGCCATCAACCGGTCTTTCTGCTTTACTTCGGCAAGT
CTAGAGATACTCAAGAAGAAAGCCTTGGAGGATGGGGTGCTATCAAAATGCACTACTTTTGA
AGCTTTATCTGGGCTGATATGGAGGGCCCGAACTAGAGCCCTGTGGAGCGATTACCCGGAGC
ACAAACTAAAGGTGCTCATTGTCGTCGACCCGAGAGCACGTTTTGAGCCTCGTGTGGTGCCA
AAAGGGTATGTGGGCAATGCGGTGCTTTTTACATGTGCTTTCGCAAGCGCAAGGGAGCTGGA
AGAAAATCCCTTGTCACATGCAGTGAAGCATGTGCAACATGCCATCGGGCGTATGACGGAGG
AGTACATGTTGTCGCAAATCGACTACATGGAGCATCAGAAGGTATGGTGCCCACCGCTAGGA
GCTAGTACATCTTTCATGACCAAATGGTCTAGGTTGGCCTTCAATATTCTAGACTTTGGGTG
GGGCAGGCCGAAGTATGTGGGGCCGGCCACGTCGCTGTCGATGGAGACGACTACTTTTGTAT
CTTATGGAAAGGGCATGAGTGTGGTGTTGGCTCTTCCTCCAGAAGCAATGCGCAAATTTGAA
AAAATCGTACATCCCTACCTCAATCCATGA
DfACT13 Nucleotide sequence codon
optimized for its expression in
S. cerevisiae.
SEQ ID NO: 117
ATGGCATGTGGCGGTGGGGTCAGGAATATCGAAGTGAAGGCTCAAGAGCCAGTCTTGGTGCA
ACCGTTAAGCACTGCTCAATCGTCAGCCTACAATTTGTTAACAAAGTTAGACCAGACTTTGG
CGCAATTGGTTGTCCAAATAGTGTTTGTTTTCGATGTGAAGAACCCTGTGACAAGAAGGCCT
CATGATGGAGCCGATCCAGCTAAGTTATTGAAAGAGGCCTTAAGGAAAGTACTAGTACCATT
CTATCCCCTTGCCGGAAGACTATGTCTTTCCCCAGACGATGGTAGTCTATTCATAGACTGCA
ACGCTCAAGGTGTTTCATTTGTCGAAGCAAACGCTGATGCCGACATCAGTGAGTTAGGTGAT
TTCTCCCAACCGGATTTTGCGACCTTAGGTTCTTTAGTCTTCCCTCTTCCACCAATTGCGTC
TGACGATGGACCATTGTTAAGTGCTCAAGTCACAAGATTCAAATGTGGTGGCTTCGTTCTAG
GTTTCGTCTTCCATCACTGTCTATTTGATGGTTTTGCCTTATCTGAATTTCTTAATGCATGG
GCTGAGACAGCGTGTGGAGTCCCGTTATCAACCCCACCTGTTTTAGATAGAACATTTCCTCG
TGCTAGATCTCCCCTGCAAATAAAGTACCCACATACAGAGTTCCTAGAGGTAGAAGATGTTT
CATTAACTCAGAACATCTCTAACGATGCTATCAACAGATCCTTCTGTTTTACATCAGCCTCT
TTGGAAATTTTGAAGAAGAAGGCGCTAGAAGACGGGGTGTTAAGCAAATGTACGACCTTCGA
AGCTCTATCTGGCTTAATATGGAGAGCACGTACCAGAGCCTTATGGAGCGACTACCCCGAGC
ACAAGTTAAAGGTCCTGATTGTCGTGGACCCTCGTGCCAGATTTGAACCGAGAGTGGTGCCA
AAAGGCTACGTAGGGAATGCAGTCTTGTTTACTTGTGCATTTGCTTCAGCCAGAGAACTAGA
AGAAAATCCATTATCTCATGCTGTTAAACACGTACAGCACGCGATCGGCCGTATGACTGAGG
AATATATGCTATCACAAATTGATTACATGGAGCACCAGAAGGTTTGGTGTCCCCCACTAGGT
GCGTCGACTTCTTTTATGACTAAGTGGTCAAGGCTTGCCTTTAACATCTTAGATTTTGGTTG
GGGTAGACCTAAGTATGTTGGTCCGGCTACTTCATTGTCTATGGAAACAACTACATTCGTCT
CCTATGGAAAGGGTATGAGCGTGGTGTTAGCCCTACCCCCAGAGGCAATGAGAAAGTTCGAA
AAGATTGTACACCCTTATTTGAACCCTTAA
DfACT13 Protein sequence
SEQ ID NO: 118
MACGGGVRNIEVKAQEPVLVQPLSTAQSSAYNLLTKLDQTLAQLVVQIVFVEDVKNPVTRRP
HDGADPAKLLKEALRKVLVPFYPLAGRLCLSPDDGSLFIDCNAQGVSFVEANADADISELGD
FSQPDFATLGSLVFPLPPIASDDGPLLSAQVTRFKCGGFVLGFVFHHCLFDGFALSEFLNAW
AETACGVPLSTPPVLDRTFPRARSPLQIKYPHTEFLEVEDVSLTQNISNDAINRSFCFTSAS
LEILKKKALEDGVLSKCTTFEALSGLIWRARTRALWSDYPEHKLKVLIVVDPRARFEPRVVP
KGYVGNAVLFTCAFASARELEENPLSHAVKHVQHAIGRMTEEYMLSQIDYMEHQKVWCPPLG
ASTSFMTKWSRLAFNILDFGWGRPKYVGPATSLSMETTTFVSYGKGMSVVLALPPEAMRKFE
KIVHPYLNP
PYI04555.1 native Nucleotide sequence
SEQ ID NO: 119
ATGGGTGCCAGCGTCTCTTTCCAGCCCTTTGTGCCCACTCCCCTGGACCATGCTATGCCCCC
AATTTACGTGTCTCAATTTCTATGCTTTCCTACCACAACTCCGCAATCCGCTATTCAAAGTC
TCCAAGTGGGAATCGAGAGATTATTCGAACGCCTGCCATTCCTGGCGGGAGAGATTCTCATC
AATGAACACACCGGAGCCATCAAAGTCCAGGCTCCCAGTGCTTTGATCCGGGAAATTCCCTA
CATGGCCCTCCGAGCCCATCCTGATCTTTACCTTCCAGCTAAGCAATGTGCAACCACACCAA
TCGAGAGACAGTTGAAGACCAACAGCCTTGATGAATCCTACCATCCACTCCCGGCGGCACTT
CCACTCTCCCAACCCCAGCCCGTCATCCGATTCCAAGCAAACACCCTCGCAGACGGCATTCT
CTTTGCAGTCAGCTATCATCATTGCATATTCGATGGCACTGGATGCGGACAGATTCTGGAAA
TGTTGGCTCAGTGCTGTTCGGCCTCCGACGATAAGATCTCCCTGCCGACTGATTGCCACACC
GATGTGCTCCTCCGCGAATACATCTCCAATCTAAGCCCTACTACCAACATCCCCCACGATTA
CACGCAAGCGTATAGCACTACGGTGCAACCGGACCCCGATGCCTCAGACCCAGACACGTCCC
CCGCCATACCCTCCTCACTCTACACAGAAGCATTCACCTTTCCCTCCCAACAAATCACCACT
CTCCGTGATGCATGCAACCACCTCTTGCCCAAATTACCCAGCACCAGCAACGCACATCCCCA
CAAACCAACACCGAATCCCCTATCATCAAATGACGTCCTCACCGCACTAATAGCCCTATGCA
TCACACGCGCCACCAACACCACCACCCCACCCCTCCAACCCAACAATCACAGTCTCTCAATG
GCCGTCAACCTCCGAACCCGCATCCAGCCCCAAGTACCAGATCACTTTCTGGGAAACTTCGC
CACGCTACTCCCAATACACTTTACCAGTCCAGTCCACACCCAACAGTCTGATCTCCTCCTCA
CCACAGAACCCCCCGACCCCGCCCTCATCCACCTAACCACCCTCGCCTCCCAAATCCGGTCC
AGCCTATCCACAGTCAACACCGATTACATCTGCGGTCTCATGACGGATCTCCGAACCCGGCG
CAATGCAGGAGAAAACAGCAGTCTTCTAATTGAAGGCATTAAGATTTCCAGTTGGAGACATC
TATCCGTCTACAAGCCGGACTTTGGCCCTGGGTTGGGGAAAATAGCGGGGTTTGAGTTCCAG
GCGGGGCTTATGGATAATTTGGTGGTGATTTTGCCTTGGAGAAATGGGGATTGGGATGTGCG
TGTTACGTTGTTGGAGAGGGATATGCGCGGGTTTAGGGAGGATCGGTTGGTTAGGTGGGCGT
TGGGGTCTGGGTAG
PYI04555.1 Nucleotide sequence codon
optimized for its expression in
S. cerevisiae, encoding C-terminally
extended protein variant of SEQ ID
NO: 143.
SEQ ID NO: 120
ATGGGTGCGTCAGTTAGTTTTCAACCTTTCGTTCCTACCCCCTTGGATCACGCCATGCCACC
GATATACGTGTCACAGTTCTTGTGTTTCCCTACTACGACGCCGCAGTCAGCAATACAATCCC
TACAGGTCGGTATTGAGCGTTTGTTTGAAAGACTACCTTTTCTAGCCGGGGAAATTCTGATA
AATGAGCACACAGGTGCAATAAAAGTTCAAGCTCCCTCTGCTCTGATTAGAGAAATTCCGTA
CATGGCCTTAAGGGCTCATCCGGATTTGTATCTGCCTGCTAAGCAATGTGCAACTACTCCAA
TAGAGAGGCAGTTGAAAACTAACAGCCTTGATGAGTCTTATCATCCATTACCTGCGGCACTA
CCATTGTCTCAACCACAACCAGTCATCAGATTCCAAGCTAATACACTTGCTGACGGAATACT
TTTCGCTGTCAGTTACCACCATTGTATATTTGATGGAACGGGCTGCGGACAGATCCTTGAAA
TGTTAGCCCAGTGTTGTTCGGCCTCAGACGATAAGATCAGCTTGCCTACGGACTGTCATACA
GATGTATTGTTGAGAGAATATATTTCGAATCTATCTCCAACCACGAATATCCCGCATGACTA
CACTCAAGCTTATTCTACTACAGTTCAACCAGATCCTGACGCATCCGATCCTGATACTAGCC
CAGCCATTCCGAGTTCGCTGTACACTGAAGCATTTACGTTTCCTTCACAACAGATTACCACA
CTAAGAGATGCGTGCAACCATTTATTGCCTAAATTACCATCTACTTCAAACGCACATCCCCA
CAAACCAACACCAAACCCGTTATCCTCAAATGACGTTTTGACGGCACTGATCGCATTGTGCA
TCACCAGAGCTACTAATACGACGACACCCCCATTACAACCAAACAACCATTCTCTTAGCATG
GCCGTCAATCTAAGGACTCGTATTCAACCCCAAGTCCCGGACCACTTTCTTGGTAATTTTGC
CACCTTGCTACCTATTCACTTCACAAGTCCAGTCCATACGCAGCAATCAGATTTATTATTGA
CTACTGAGCCGCCAGACCCAGCATTGATCCACCTTACAACCCTTGCATCGCAAATTAGGTCT
AGTTTATCGACCGTTAACACCGATTACATATGTGGCTTGATGACAGATCTGAGGACTAGGAG
AAACGCCGGTGAGAATTCCTCTTTGTTAATAGAAGGTATTAAGATTTCTTCATGGAGACATC
TTAGCGTTTACAAGCCAGATTTCGGACCTGGATTAGGGAAGATTGCAGGATTTGAGTTTCAA
GCTGGTTTAATGGACAACCTTGTTGTGATATTGCCTTGGAGAAACGGTGATTGGGACGTGCG
TGTAACTCTTCTGGAGAGAGATATGAGGGGTTTTAGGGAAGACAGATTAGTCAGATGGGCAT
TAGGTAGCGGAACAGGCCCCTTTTCCTTTGTCGATATCATGTAA
PYI04555.1 Protein sequence C-terminally
extended protein variant of SEQ ID NO: 143.
SEQ ID NO: 121
MGASVSFQPFVPTPLDHAMPPIYVSQFLCFPTTTPQSAIQSLQVGIERLFERLPFLAGEILI
NEHTGAIKVQAPSALIREIPYMALRAHPDLYLPAKQCATTPIERQLKTNSLDESYHPLPAAL
PLSQPQPVIRFQANTLADGILFAVSYHHCIFDGTGCGQILEMLAQCCSASDDKISLPTDCHT
DVLLREYISNLSPTTNIPHDYTQAYSTTVQPDPDASDPDTSPAIPSSLYTEAFTFPSQQITT
LRDACNHLLPKLPSTSNAHPHKPTPNPLSSNDVLTALIALCITRATNTTTPPLQPNNHSLSM
AVNLRTRIQPQVPDHFLGNFATLLPIHFTSPVHTQQSDLLLTTEPPDPALIHLTTLASQIRS
SLSTVNTDYICGLMTDLRTRRNAGENSSLLIEGIKISSWRHLSVYKPDFGPGLGKIAGFEFQ
AGLMDNLVVILPWRNGDWDVRVTLLERDMRGFREDRLVRWALGSGTGPFSFVDIM
ERR364415-1_contig_8546 native Nucleotide
sequence
SEQ ID NO: 122
ATGGCCCGGGCACCACCACCTCCGCCTCCTGGTCTCAGAATGAGAGACACAGTGCTCAGCAT
CGTGAAGCCGATACGGAAGACACAGCATTTGGAGACGATCGACGCAACGTTCGTCGATTTGA
TGAGAATGGACAGCTTTATACCCGTGATTTTCGCCTACAGGCCTGCGGACAAGTCCGAGGCC
GCGTACTCGCGTCTCGTGAATCGCATCAAGGAGTCGCTCCAGAAGGTTTTGGTCCCCTTTTT
CGGGTTCGCGGGCCGCTGGGTTCCAAGCAGTGGCGGGAGCAGGCGGCTCTTGTGCAACGATG
AGGGCGTTCCCTTCATTGAAGCGTTTGTGGACGAAGAGTTGGACTCGGTGGTGAAGGCTTCC
GCCGCATTCCAGCCGGTTACGGAGCTGAATGGCTTGGGCGTCCTCGGAATGGACATGACTTC
ATACGATCAAAGGATGCCACCGGAAGGTGGGCAACCTTGCGTCGTTGCTCAAGTCACACGGT
TCAAATGTGGGGGAGTGGTTCTGGGGGTGGCTTTCAATCACACTCACACTGACGGCCAGGGA
TTCTACACCTTCATGCGAGCATGGTCCGACTTCTCTCGAACCAACGGAACGGCAATCAAGGT
GGACCACAACCGGGCCCTGCCAGAACTGGCTTCCCTCTCACAGTTCTTCATCAAACAGCACG
ACCGAATAGGAGGCAAAACTTCTACCGATCGAGTCAACGATCATTGTTCTAAAGTTCCGGAA
CGGCTGGCTTTGAAAGCTTTCGAGGTTCGTGCGTCTAAGATCAAAGCCGCAAAGCTAGCAGC
CGAAGATGGAGGGGTTGGGTATGTCAGCACGGTAGATTGCATTGTGGCTCACTTATGGAAAA
CTCTTGCCAGATTGCCGCCCGTCGTGTTGGATGGGAGGGAGATTACGGTCTTCTCGCCTGTG
GAGGGGAGGAACAGATTCTTGGACCCGCCAAGACCCAATATGTGTGGAAATTGTTTTGCAGC
AATGGTGACCCCCAAAATCCCAACCCAGGAGTTGCTGGAGATGCCTCTCGCTGCAATTGCAG
GCAAGCAACGGGAGAAATTATCCACAACCCGAAGGGAGGAATGGTTTGGACAGCAAAGCTTT
AGGGAGCTGGCCTCCGCGATGAACACCAGCAAATCTGCTCTACTTATTGTGACCTCGTGGTT
CAACTTTCCCATGTATGAGATCGACTTTGGAGCTGGCAAACCATTTTTTGCATCCACTACGA
ACATGATTTCTCCTATCAACGGCGTGTGTTGTGGAGTCATTGCACCCCCAACTCCTGGGAGC
TGCTCCTCCATTGCCACTCTGTACATTTTGTGCCTTCCCGCGGTACTAGAGGCTCTTGAAAA
TGTTCCAGATTTCCTATCCTTCTTCGTTCCTCACCCAAATCACAAAGATAACTCGCAATAG
ERR364415-1 contig 8546 Nucleotide sequence
codon optimized for its expression in
S. cerevisiae, encoding C-terminally
extended protein variant of SEQ ID NO: 144.
SEQ ID NO: 123
ATGGCCAGGGCTCCACCCCCACCACCACCGGGCTTGCGTATGAGAGATACGGTCCTATCAAT
CGTCAAACCTATAAGAAAGACTCAACACCTAGAGACAATTGATGCAACATTCGTAGATTTAA
TGAGAATGGACTCATTTATCCCTGTGATCTTCGCTTATAGACCTGCAGATAAGAGCGAGGCT
GCGTACTCTAGATTAGTTAATAGGATAAAAGAAAGCTTACAGAAAGTACTAGTACCATTCTT
TGGATTTGCTGGAAGGTGGGTGCCTTCTTCCGGTGGCTCAAGGCGTCTGCTATGCAATGACG
AAGGTGTCCCTTTTATTGAGGCGTTTGTAGACGAAGAACTGGACTCTGTCGTTAAGGCTTCA
GCTGCCTTCCAACCTGTAACTGAACTTAATGGTCTAGGTGTGTTGGGTATGGATATGACTAG
TTATGATCAAAGAATGCCCCCTGAAGGCGGTCAACCGTGCGTCGTAGCTCAGGTAACGAGAT
TTAAATGCGGCGGTGTGGTATTGGGTGTAGCATTCAACCATACCCATACTGACGGGCAGGGC
TTTTATACTTTTATGCGTGCATGGTCGGATTTCAGTAGAACGAATGGTACCGCAATTAAAGT
CGATCACAACAGGGCACTACCCGAATTAGCATCTTTGAGTCAGTTCTTTATTAAGCAGCATG
ATAGAATTGGTGGGAAAACTTCCACCGATAGAGTCAATGACCACTGTAGCAAAGTCCCTGAA
AGACTAGCACTAAAGGCTTTTGAAGTTAGGGCGTCCAAAATCAAGGCAGCAAAACTAGCCGC
AGAAGATGGTGGTGTAGGCTACGTCTCGACGGTGGATTGTATTGTTGCTCATCTATGGAAGA
CACTAGCTCGTTTGCCACCAGTTGTGTTAGACGGTCGTGAGATCACTGTGTTTAGCCCAGTA
GAAGGCAGAAATAGGTTTCTTGATCCGCCCCGTCCGAACATGTGTGGTAATTGTTTCGCTGC
AATGGTAACCCCTAAAATCCCAACACAAGAATTGTTAGAGATGCCATTAGCCGCCATTGCCG
GCAAGCAGAGAGAGAAACTATCTACCACGAGACGTGAAGAGTGGTTCGGACAGCAATCATTC
AGGGAGTTGGCTTCAGCTATGAATACTTCTAAATCAGCTTTGTTAATCGTGACATCTTGGTT
TAACTTCCCGATGTATGAAATCGATTTTGGTGCCGGTAAGCCGTTCTTCGCTAGTACGACTA
ATATGATTTCTCCTATAAATGGAGTTTGTTGTGGTGTCATAGCCCCGCCCACCCCCGGTTCC
TGTTCATCCATAGCGACATTATACATTTTATGTTTACCAGCCGTGTTAGAAGCTCTTGAAAA
TGTCCCAGATTTCCTTTCGTTCTTCGTACCGCATCCAAACCATAAAGACAACAGCCAAACAG
GCCCCTTTTCCTTTGTCGATATCATGTAA
ERR364415-1_contig_8546 Protein sequence
C-terminally extended protein variant of
SEQ ID NO: 144.
SEQ ID NO: 124
MARAPPPPPPGLRMRDTVLSIVKPIRKTQHLETIDATFVDLMRMDSFIPVIFAYRPADKSEA
AYSRLVNRIKESLQKVLVPFFGFAGRWVPSSGGSRRLLCNDEGVPFIEAFVDEELDSVVKAS
AAFQPVTELNGLGVLGMDMTSYDQRMPPEGGQPCVVAQVTRFKCGGVVLGVAFNHTHTDGQG
FYTFMRAWSDFSRINGTAIKVDHNRALPELASLSQFFIKQHDRIGGKTSTDRVNDHCSKVPE
RLALKAFEVRASKIKAAKLAAEDGGVGYVSTVDCIVAHLWKTLARLPPVVLDGREITVESPV
EGRNRFLDPPRPNMCGNCFAAMVTPKIPTQELLEMPLAAIAGKQREKLSTTRREEWFGQQSF
RELASAMNTSKSALLIVTSWFNFPMYEIDFGAGKPFFASTINMISPINGVCCGVIAPPTPGS
CSSIATLYILCLPAVLEALENVPDELSFFVPHPNHKDNSQTGPFSEVDIM
XP_001258079.1 native Nucleotide sequence
SEQ ID NO: 125
ATGACCGTGACCATCAGTTTCGAGCCATATGTGGGCTCCTCTGTCGATGCTCTAAGCATCCC
TCTCTATCTTCGATGTCAACTCGTCTTCAAACTTTCTAAGCCACTTGCTGCGGTGCCTCTGC
TTGAGTCTGGAGTTAATCGTCTTGTACAAGCGTTACCCTTCCTCTCGGGCGAGTTCACGGCC
GTGCCAGCATCCGACGGTGGGAAAGAAATTCTTCTCGTTCGCCCTGTGCTCAACTTCGAGCT
CAGCCGTATACTCAAGATCAAGTACCATGAAACATCCCTACGACATGTATGCAAACAGATGA
ACAGGCCAAGCAGCCAGGGTGGTGACCTTCCGCATGAGCCATACATGCCCTACCCACGACTT
CCAGATCCTTCACGCCCTCAACCCATCGTCGGGTTCCAAGTCAACGTTCACACGGATGGCAT
CATTCTCTCCGTTGCTACGCATCACTGTTCCTTTGACGCAACAGGGATGGGATCAATCGTCC
AAAACCTCGCGGCTTGTTGCCGTTCTCCTCCGAGCGACGAGCCTGACTTGACCACGTCGCCA
GCCCAGGAAGCAGAAGCAAGAAAAGTCCTCTCGCAAGTCCGCGAGACGCCTTTTGATCCAAA
GATGTTCCCGGAGTACAGACCCTTGGACAGTATGCTGTCCTATTACAAAGGCGTCCAGTCAG
CGCTCCAGGGTCGTCAAACCACTATCGTCAATCGATGTTTCACAATCGCCGCCGACAAGATC
AACGCGCTCAAAAGGCGCTGCAATCAACTGATACCGGAAATGGTGAAGAAGTATGGGCTTTC
AACTGAGGATGCCATTGGGAGCGCCTGGGTCTCGAGTAATGATGTTGTTGCTGCCCTCTTGT
GGACATGTATCAATCGAGCGCGATATCCCGAAATCCGCGAGCGCAGCGTTCACCAGCTCCCA
CCAGACCTCCTACATGCGACATCTAGCCTGGGTGTGCCAGTGAACGTTCGCTCTCGACTGTC
GCCGCCCTTACCCAAATCGACTTTAGGTAACGCCGTGTGTCTTCTCCGGGAGAAGGTCCCGC
TGCAATTTTTCGCTTTGCCTAGTCATGCCAACATGGAGGCCACTTCTAGCGTTTGCGCAGAC
CATTCCGGAGACGACGAATGGGCCTTGTCCTTCTGTCGAGTCGCCTACGGACTCAGAGCGAA
GCTGAACGCAATTGATGACGACTATATCCGCGACTATATCTCCTACGTGCAAAAGTCTCCGT
GCCATCTGTCAGTGACACTGGATACAGAGAACCTGTACCTCAGTAACTGGCGCGAGATCGGT
GTGTATGATGCTGATTTTGGAGGCATGCTGGGCAAGCCGCTACGGATGAGAGCTCCGGATGG
ATACACCGATGGCCTGATTTTTGTGATGGCGCAGCGGAGCGAAGATAAGTCTGCACCGTGGG
AGTTTAATATCTCGCTGGAGGCATCGACAATGAAGCGTATTGTGCATGATCCCCTCTGGTGC
AAGTATGTTGAGCTGGATGCGTTCTGGCATGGAGAAGAATGA
XP_001258079.1 Nucleotide sequence
codon optimized for its expression
in S. cerevisiae.
SEQ ID NO: 126
ATGACCGTCACGATTTCATTCGAACCATATGTGGGATCTAGCGTGGACGCGCTGTCCATACC
CTTGTATCTAAGGTGCCAACTGGTGTTCAAATTATCCAAGCCTCTTGCAGCCGTGCCCCTGC
TAGAATCTGGGGTAAACCGTCTAGTACAAGCATTACCATTCTTGTCTGGTGAATTTACTGCT
GTCCCTGCAAGCGATGGTGGGAAGGAAATCTTACTTGTTAGACCAGTCTTAAATTTCGAACT
AAGTCGTATACTGAAGATCAAATACCACGAAACATCCTTAAGACACGTATGCAAGCAGATGA
ACAGACCATCCTCGCAAGGTGGTGACTTGCCACACGAACCGTATATGCCATACCCCAGGTTA
CCAGATCCATCTAGGCCTCAACCAATCGTGGGTTTTCAAGTCAATGTCCACACTGACGGTAT
AATCCTGAGTGTAGCAACTCACCATTGCAGTTTTGACGCCACGGGGATGGGAAGTATTGTAC
AGAACTTGGCCGCATGCTGTAGATCTCCGCCATCGGACGAGCCAGATTTAACTACAAGCCCT
GCTCAAGAAGCAGAAGCTAGGAAGGTCCTGAGCCAAGTTAGAGAAACACCATTCGACCCAAA
GATGTTTCCCGAATATAGGCCCTTAGACTCTATGTTATCTTATTACAAAGGTGTCCAGTCTG
CTTTGCAGGGTCGTCAAACTACTATCGTTAACAGATGTTTCACTATCGCTGCTGATAAGATA
AACGCCTTAAAGAGGAGATGTAACCAACTTATTCCGGAAATGGTAAAGAAATACGGATTGAG
TACAGAAGATGCTATCGGTTCTGCATGGGTCTCTTCTAATGACGTGGTTGCCGCACTTTTGT
GGACCTGTATTAATCGTGCTAGATACCCAGAGATTAGAGAAAGAAGTGTACATCAGCTTCCA
CCAGACTTGTTGCATGCTACATCTTCATTGGGTGTACCTGTTAATGTTAGATCTAGACTTTC
ACCACCCTTACCCAAATCCACTCTTGGGAATGCGGTCTGCCTGCTAAGGGAGAAGGTACCTT
TGCAATTCTTCGCTCTTCCGAGTCACGCAAACATGGAAGCAACCAGTTCAGTTTGCGCTGAC
CATTCAGGGGATGATGAATGGGCGTTGAGCTTTTGTAGGGTAGCTTATGGTCTGCGTGCAAA
ATTGAACGCGATCGATGATGATTACATAAGGGATTACATTAGTTATGTACAGAAGTCGCCCT
GTCATCTTTCAGTTACACTAGATACTGAGAACTTATACCTGTCTAATTGGAGAGAAATAGGC
GTCTACGATGCAGACTTCGGTGGTATGCTAGGGAAACCATTGCGTATGAGAGCTCCGGATGG
CTACACTGACGGTTTGATTTTCGTTATGGCCCAAAGATCTGAAGACAAGTCAGCTCCGTGGG
AATTCAACATATCCCTTGAGGCTTCTACAATGAAAAGGATTGTACATGATCCGCTGTGGTGT
AAATATGTTGAATTGGATGCCTTTTGGCATGGGGAAGAGTAA
XP_001258079.1 Protein sequence
SEQ ID NO: 127
MTVTISFEPYVGSSVDALSIPLYLRCQLVFKLSKPLAAVPLLESGVNRLVQALPFLSGEFTA
VPASDGGKEILLVRPVLNFELSRILKIKYHETSLRHVCKQMNRPSSQGGDLPHEPYMPYPRL
PDPSRPQPIVGFQVNVHTDGIILSVATHHCSFDATGMGSIVQNLAACCRSPPSDEPDLTTSP
AQEAEARKVLSQVRETPFDPKMFPEYRPLDSMLSYYKGVQSALQGRQTTIVNRCFTIAADKI
NALKRRCNQLIPEMVKKYGLSTEDAIGSAWVSSNDVVAALLWTCINRARYPEIRERSVHQLP
PDLLHATSSLGVPVNVRSRLSPPLPKSTLGNAVCLLREKVPLQFFALPSHANMEATSSVCAD
HSGDDEWALSFCRVAYGLRAKLNAIDDDYIRDYISYVQKSPCHLSVTLDTENLYLSNWREIG
VYDADFGGMLGKPLRMRAPDGYTDGLIFVMAQRSEDKSAPWEFNISLEASTMKRIVHDPLWC
KYVELDAFWHGEE
XP_001217250.1 native Nucleotide sequence
SEQ ID NO: 128
ATGGCAACCTTCGACCATATCGAGGATGTCATCGGCCAGCTACCTATGCTGAAGAGCTACAC
CCATATCTTGCTGTGCTTTCCCCTCGCTGAGAGCCAACTCAATGAAGCCATCGAGAGCCTTG
AATCTGCTGTACGTCAGGTTATAAAAACCTTTTCGTTCTTGGCTGGCAAAGTAGTCAACGAA
GGCAAGGGACCCAACAGCTCGGGTACTTTCAGGGTCGCTCCCTGTGAGACGTGGGAGTCCCC
AGATCATCAATTTGTGCGAGTCGTTGATCGCTCTTTCATGCTGGCCTCCTACGATGAGATCC
GCGGAGCACAGGCACCTGCTTCCATGCTCCCAGGAAGTCAACTCGGGTATCGGGTGGCTTTT
CCAGCGCATTATCACGAGACAGAGGACGATCCCGCGCCGGTCCTGGACATTCAGTGCAATTT
GATACGGGGCGGACTGCTACTTGACATCGCTGCCCAACACAACATTATCGATGCGAGCGGTA
TCTTTCAAATTGCCAGCTTGATCGCTCTTTCCATGCGAGGCGAGTCAATTCCTGAGGATGTC
ATCAAGGAAGGGAACCGTGATCGACGCAATATCATTCCACTACTAGAGGCAGATGAGCCTCT
TCTTGATCACAGCGAGCTCAAAGCCAGCAGCGCGGTGCAAAATCCGCCGCCCGTCAACTTCC
TCCAGGGGTATAAATGGCAAATCTTCAAGCTCTCTGCCGAGGTGTTAACTCGAATTACCGCT
GAAGGACGACGACAGCCACAGGAGTTTGTCCCCTCCGTCACATTTGTCTCGGCAAACGATTG
CTTGACAGCCTTTCTGTGGCAACGGGTGATAGCCATGCGCCTGAAGCGGCTCCATACGCCCG
AGGCCGTATCCAAACTGAGCCGTGCTGTTGATCTTCGGCGGGCGATGGGCATTACCCCAGCA
TACATGGGCCATATGATTCGTGTCGCAAATACTAGTCTCACTTTTCAAGAAATTGTGGCATG
CTCTTTATCCAGGCTTGCATCTCTGCTCCGCAAGAGCATCATCGATGTCAGCCAGCCGTATG
CGATTCGGAGCTACGTGACCTTTATTGCAAATGAGACGGATAAATCAAAGATTGCGTATGCG
GGTGCTTTCAATCCTTGCACCGACATGTCGTGCTCGTCCATTGCACACATCACTGCTCCTGA
ATTCGGTCGTTTGGGAGCGCCTGACTTTATAAGGAGGCCTACCTACGGGCCTCTGCCGTGCT
GCACCTATGTCGCTCCCGATAAGAACGATGGAGCTTTGGATCAAAACCAGGCATGGTCAGAC
GTTGTGAAGCGCATCGGTTGA
XP_001217250.1 Nucleotide sequence
codon optimized for its expression
in S. cerevisiae.
SEQ ID NO: 129
ATGGCCACCTTCGACCACATTGAGGACGTGATAGGACAATTGCCCATGCTAAAGAGCTACAC
ACATATTTTACTTTGCTTTCCGTTGGCAGAATCTCAACTGAACGAGGCGATTGAATCTTTGG
AGTCAGCAGTCAGACAAGTAATTAAGACGTTTTCATTCCTTGCAGGTAAGGTTGTTAATGAA
GGCAAAGGTCCGAATTCCTCTGGTACGTTCCGTGTTGCTCCGTGTGAAACTTGGGAATCTCC
TGATCATCAATTTGTTCGTGTTGTAGATAGGAGCTTTATGTTGGCCTCGTATGATGAGATCC
GTGGTGCTCAAGCCCCCGCTTCCATGTTACCTGGGTCACAACTGGGGTATAGGGTAGCATTC
CCAGCCCATTATCATGAAACTGAAGACGATCCAGCTCCAGTTCTAGATATCCAGTGTAATTT
GATAAGAGGTGGTCTGCTTCTAGATATAGCGGCCCAACACAACATAATTGATGCTTCCGGGA
TATTTCAAATCGCTAGTCTGATCGCCTTAAGCATGAGAGGAGAAAGTATCCCTGAAGATGTT
ATCAAAGAAGGAAACAGAGATAGAAGGAATATCATTCCGTTATTGGAAGCCGATGAGCCTTT
ATTAGACCATAGTGAATTGAAGGCATCCAGCGCCGTTCAGAACCCGCCACCAGTTAATTTCT
TGCAAGGTTATAAATGGCAGATTTTCAAACTGTCCGCTGAGGTATTGACCCGTATTACTGCC
GAAGGACGTAGACAACCACAAGAATTTGTGCCCTCAGTGACCTTTGTGTCCGCAAACGACTG
CTTGACTGCTTTCTTGTGGCAGCGTGTGATAGCTATGAGGCTAAAGAGATTGCATACCCCCG
AGGCCGTTTCCAAGCTATCTAGAGCCGTGGATTTAAGAAGGGCCATGGGTATAACCCCTGCA
TACATGGGCCATATGATAAGAGTCGCAAACACATCCTTAACCTTTCAAGAAATTGTCGCATG
TAGTTTATCACGTCTTGCGAGCTTACTTAGAAAATCTATCATTGATGTGTCCCAACCATATG
CGATCAGATCATACGTCACCTTCATAGCAAACGAAACAGACAAATCTAAGATAGCCTATGCC
GGGGCATTTAACCCGTGTACTGATATGTCATGTTCATCCATAGCTCATATTACGGCACCGGA
GTTTGGTAGGTTAGGGGCGCCTGACTTCATTAGGAGACCCACTTATGGCCCACTACCTTGCT
GTACTTACGTAGCTCCTGACAAGAATGACGGTGCATTGGATCAGAATCAAGCATGGTCAGAT
GTCGTAAAGAGGATAGGTTAA
XP_001217250.1 Protein sequence
SEQ ID NO: 130
MATFDHIEDVIGQLPMLKSYTHILLCFPLAESQLNEAIESLESAVRQVIKTFSFLAGKVVNE
GKGPNSSGTFRVAPCETWESPDHQFVRVVDRSFMLASYDEIRGAQAPASMLPGSQLGYRVAF
PAHYHETEDDPAPVLDIQCNLIRGGLLLDIAAQHNIIDASGIFQIASLIALSMRGESIPEDV
IKEGNRDRRNIIPLLEADEPLLDHSELKASSAVQNPPPVNFLQGYKWQIFKLSAEVLTRITA
EGRRQPQEFVPSVTFVSANDCLTAFLWQRVIAMRLKRLHTPEAVSKLSRAVDLRRAMGITPA
YMGHMIRVANTSLTFQEIVACSLSRLASLLRKSIIDVSQPYAIRSYVTFIANETDKSKIAYA
GAFNPCTDMSCSSIAHITAPEFGRLGAPDFIRRPTYGPLPCCTYVAPDKNDGALDQNQAWSD
VVKRIG
BAU61551.1 native Nucleotide sequence
SEQ ID NO: 131
ATGTCCAAGCCCTTATTCGAAGCGTATCCTCTCACAGGGCTTGATCATACGATTCCTCCATG
TTATGTTCGCTTCCTTCTAACTTTCCCCGTGCCGGATGTGGCATTGGCTGTCAATCAGCTGC
AAAAGGGAGCTGAAAATTTAATCGAGAAACTTCCTTTCCTGGCCGGATATTTGGCTTCATGC
GAGACCCCCGGCGTACGCCCAGGGCAGCTCGAGATTCGACCTCCCGCTGGGGAAAGGAGACC
TGTCTGCCTCGTCGCACATCACTCGAACTCCTATCTCGCAGATTCCAGTGCGACGTCGACGA
CGGAACAGCTGGGCACCGCCAACGAGAACTATCTCCCTGTCCCGTTCTTCCCGGAGCTAGAC
AAGCCGGTGCCCATCTTCCGGGTTAAGGTGAATGCCATGACGGACGGCATCATTCTGGGATT
TGCGTTCCACCATAGCGTGATCGATGCCACCGGGATGGGCACCATTGTCCGGGACTTTGCCA
GATGCTGCCGTGGCCCTGATGGCGGTCCCCTGGAAATCAGTCTGGAGTCTCAGCAGGACTCT
AGAGAGAAGCTGAGACACTCCGGAGGACCTCCCGATCCGCGGTTTGACCACAATGGGGAGTA
CCCTCTCGTGGCGTCTCTGCCCGCCGACCTCGAAGCCATGAAGCAGGTCTTGATCCAGACGG
CCCGTCTCATGTCAACGCAGTATTTCCGCATCCCTGCCAGCCTAGTTAACACGCTAAAGGAA
TCCTGCAATCGGATGCTTCGGGAATCACCAGCGCTCAGGGACGAAGGGGAGAATCCATGGAT
TTCGAGCAACGATCTGGTGGTGTCGCTGTTGTGGCTGTGTCTGAATCGCGTTCGGTATCCTG
AAGATAATACCAACGTCATTCCTCCTTCCGATTCCTCGGTCTGCATGGCCGTGAATATCCGA
GGGCGTTTGCAGTCGCCCATTGATCCAGGATACGTTGGCAACGCCATCGTCCTTCTCCGGGA
GAGCGTTGGCATGAATGCTTTTCTGCATAAACCGGGCGACGATGATCCCCTGGGCGCCCAAT
GTTACGAAACAGCGAAACGGCTAGGCCGAGAAGCGTGGGAAGCAGCCCTGGTGCGCATCGCC
CTGGCCATCCGCCGCAAGCTCAACACCATAAACGCGAGTTACGTGCGCAGTGTTATATCCTA
TCTGGAGGACGTGCCCGACCTGTCCACTGTGGCGTTTGGCCAGACGGACTACCACATCAGCA
GCTGGCGGGATATTGGCGTCTACGAGGCTGATTTTGGTGGCCACATGGGCCATCCCAGCGAA
ATGCGAGTACCAGATGGGATGGTCGATGGCATGTTTTACATCTTACCTCGAAGGCAGGGAAC
ACACCCTTGCTGGGAGATCCATGTTACTATCCACCAGGACACAATGAAGCGACTCATTGCAG
ACCCTGTGTGGGCACGATATACAGTGAGAAAGCCTTCATCACTCTGCCGGGATGAATGA
BAU61551.1 Nucleotide sequence codon
optimized 1 for its expression in
S. cerevisiae.
SEQ ID NO: 132
ATGTCAAAGCCTCTGTTTGAAGCATACCCCCTAACTGGTCTAGATCATACTATACCCCCTTG
TTACGTCAGGTTCTTATTAACTTTCCCAGTTCCAGATGTAGCCCTTGCTGTTAACCAATTAC
AGAAAGGCGCCGAAAATTTAATAGAGAAATTGCCTTTTCTTGCAGGGTACCTAGCTAGCTGT
GAAACACCTGGCGTAAGACCGGGGCAGTTAGAAATCAGGCCACCAGCAGGTGAAAGAAGACC
AGTGTGTTTGGTCGCACATCACAGTAACTCGTACCTTGCGGATTCTTCTGCAACAAGTACGA
CGGAGCAATTGGGAACAGCTAACGAAAATTACCTTCCCGTACCATTCTTTCCAGAGTTGGAC
AAACCTGTTCCAATATTCCGTGTCAAAGTTAACGCTATGACAGATGGGATTATCTTGGGGTT
CGCTTTCCACCATAGTGTAATAGATGCTACGGGAATGGGTACAATAGTTAGGGACTTCGCCA
GGTGCTGCAGAGGTCCTGATGGTGGGCCCTTAGAGATAAGTCTAGAAAGCCAACAAGATTCG
AGAGAAAAGCTGAGGCACTCAGGCGGTCCGCCAGATCCCAGATTCGATCATAACGGAGAATA
CCCATTGGTGGCCTCACTGCCAGCGGACTTAGAAGCTATGAAACAAGTTTTAATCCAAACAG
CGAGGCTGATGAGTACACAATACTTTAGAATACCTGCTAGCCTTGTGAACACTTTAAAAGAG
TCATGTAATAGAATGCTTCGTGAATCCCCTGCACTGAGGGATGAAGGTGAAAACCCGTGGAT
TAGTTCTAACGATTTAGTAGTGAGTCTACTGTGGCTTTGTTTGAACAGGGTGAGGTACCCCG
AAGACAATACAAATGTGATTCCACCCTCTGACAGTTCTGTTTGCATGGCTGTAAATATAAGA
GGGAGATTACAGTCGCCGATCGATCCAGGTTATGTTGGTAATGCTATTGTATTATTAAGAGA
ATCTGTTGGAATGAATGCCTTTCTACATAAACCTGGTGATGACGACCCGCTTGGTGCCCAAT
GTTACGAGACAGCTAAAAGACTTGGAAGAGAAGCATGGGAAGCAGCATTAGTCAGGATTGCT
TTGGCAATTAGGCGTAAGTTGAACACTATTAATGCTTCCTATGTCAGATCAGTTATTAGCTA
CTTAGAGGATGTGCCCGATCTATCAACCGTTGCCTTCGGGCAAACTGATTATCATATCTCCA
GTTGGAGAGACATTGGAGTTTACGAGGCTGACTTTGGTGGTCATATGGGCCATCCATCTGAG
ATGAGAGTCCCTGACGGGATGGTCGATGGTATGTTTTACATACTACCTAGAAGACAAGGTAC
TCACCCATGTTGGGAAATTCATGTGACTATACACCAGGATACCATGAAAAGACTGATCGCTG
ATCCGGTTTGGGCAAGATATACCGTTAGAAAGCCTAGTTCTTTGTGCAGGGACGAGTAA
BAU61551.1 Protein sequence
SEQ ID NO: 133
MSKPLFEAYPLTGLDHTIPPCYVRFLLTFPVPDVALAVNQLQKGAENLIEKLPFLAGYLASC
ETPGVRPGQLEIRPPAGERRPVCLVAHHSNSYLADSSATSTTEQLGTANENYLPVPFFPELD
KPVPIFRVKVNAMTDGIILGFAFHHSVIDATGMGTIVRDFARCCRGPDGGPLEISLESQQDS
REKLRHSGGPPDPRFDHNGEYPLVASLPADLEAMKQVLIQTARLMSTQYFRIPASLVNTLKE
SCNRMLRESPALRDEGENPWISSNDLVVSLLWLCLNRVRYPEDNTNVIPPSDSSVCMAVNIR
GRLQSPIDPGYVGNAIVLLRESVGMNAFLHKPGDDDPLGAQCYETAKRLGREAWEAALVRIA
LAIRRKLNTINASYVRSVISYLEDVPDLSTVAFGQTDYHISSWRDIGVYEADFGGHMGHPSE
MRVPDGMVDGMFYILPRRQGTHPCWEIHVTIHQDTMKRLIADPVWARYTVRKPSSLCRDE
PsSalAT native Nucleotide sequence
SEQ ID NO: 134
ATGGCAACAATGTATAGTGCTGCTGTTGAAGTGATCTCTAAGGAAACCATTAAACCCACAAC
TCCAACCCCATCTCAACTTAAAAACTTCAATCTGTCACTTCTCGATCAATGTTTTCCTTTAT
ATTATTATGTTCCAATCATTCTTTTCTACCCAGCCACCGCCGCTAATAGTACCGGTAGCAGT
AACCATCATGATGATCTTGACTTGCTTAAGAGTTCTCTTTCCAAAACACTAGTTCACTTTTA
TCCAATGGCTGGTAGGATGATAGACAATATTCTGGTCGACTGTCATGACCAAGGGATTAACT
TTTACAAAGTTAAAATTAGAGGTAAAATGTGTGAGTTCATGTCGCAACCGGATGTGCCACTA
AGCCAGCTTCTTCCCTCTGAAGTTGTTTCCGCGAGTGTCCCTAAGGAAGCACTGGTGATCGT
TCAAGTGAACATGTTTGACTGTGGTGGAACAGCCATTTGTTCGAGTGTATCACATAAGATTG
CCGATGCAGCTACAATGAGTACGTTCATTCGTAGTTGGGCAAGCACCACTAAAACATCTCGT
AGTGGGGGTTCAACTGCTGCCGTTACAGATCAGAAATTGATTCCTTCTTTCGACTCGGCATC
TCTATTCCCACCTAGTGAACGATTGACATCTCCATCAGGGATGTCAGAGATACCATTTTCCA
GTACCCCAGAGGATACAGAAGATGATAAAACTGTCAGCAAGAGATTTGTGTTCGATTTTGCA
AAGATAACATCTGTACGTGAAAAGTTGCAAGTATTGATGCATGATAACTACAAAAGCCGCAG
GCAAACAAGGGTTGAGGTGGTTACTTCTCTAATATGGAAGTCCGTGATGAAATCCACTCCAG
CCGGTTTTTTACCAGTGGTACATCATGCCGTGAACCTTAGAAAGAAAATGGACCCACCATTA
CAAGATGTTTCATTCGGAAATCTATCTGTAACTGTTTCGGCGTTCTTACCAGCAACAACAAC
GACAACAACAAATGCGGTCAACAAGACAATCAATAGTACGAGTAGTGAATCACAAGTGGTAC
TTCATGAGTTACATGATTTTATAGCTCAGATGAGGAGTGAAATAGATAAGGTCAAGGGTGAT
AAAGGTAGCTTGGAGAAAGTCATTCAAAATTTTGCTTCTGGTCATGATGCTTCAATAAAGAA
AATCAATGATGTTGAAGTGATAAACTTTTGGATAAGTAGCTGGTGCAGGATGGGATTATACG
AGATTGATTTTGGTTGGGGAAAGCCAATTTGGGTAACAGTTGATCCAAATATCAAGCCGAAC
AAGAATTGTTTTTTCATGAATGATACGAAATGTGGTGAAGGAATAGAAGTTTGGGCGAGCTT
TCTTGAGGATGATATGGCTAAGTTCGAGCTTCACCTAAGTGAAATCCTTGAATTGATTTGA
PsSalAT Nucleotide sequence codon optimized
for its expression in S. cerevisiae.
SEQ ID NO: 135
ATGGCAACAATGTACTCAGCTGCAGTTGAGGTTATATCTAAGGAAACGATAAAACCAACCAC
TCCAACCCCAAGCCAATTGAAGAATTTCAATTTATCTTTATTAGACCAGTGCTTTCCCTTGT
ACTACTATGTCCCCATCATCTTGTTCTACCCTGCGACTGCTGCAAACTCCACTGGTTCCTCG
AACCACCATGATGATCTAGATCTTCTGAAGAGCTCCCTTAGCAAGACACTTGTTCACTTCTA
CCCTATGGCCGGTAGGATGATCGATAACATATTGGTTGACTGCCACGATCAGGGTATCAATT
TCTATAAAGTTAAAATCAGGGGCAAGATGTGTGAATTCATGTCTCAGCCTGATGTGCCACTG
TCGCAGCTGCTACCTAGTGAAGTGGTATCCGCATCTGTCCCAAAAGAGGCCTTGGTCATAGT
CCAAGTTAATATGTTCGATTGCGGTGGGACGGCCATCTGCTCGTCGGTCAGTCATAAGATCG
CAGACGCCGCAACCATGTCAACATTTATTAGATCTTGGGCGAGTACCACCAAAACTTCAAGG
TCTGGCGGGTCAACCGCCGCTGTTACTGACCAGAAGTTGATTCCTAGCTTTGATTCGGCAAG
CTTATTCCCACCTTCCGAAAGGTTGACTTCACCAAGCGGGATGTCTGAGATACCATTTTCTT
CAACCCCTGAAGATACCGAGGACGACAAAACAGTTAGCAAAAGATTCGTGTTTGACTTTGCA
AAGATAACATCTGTTAGAGAAAAGCTTCAGGTATTAATGCACGACAACTACAAAAGCAGGCG
TCAGACCAGGGTTGAGGTCGTAACGAGCCTGATCTGGAAGAGTGTCATGAAGTCAACACCAG
CTGGGTTCCTTCCCGTCGTGCATCATGCGGTAAATTTGAGGAAGAAGATGGACCCACCATTG
CAGGATGTCTCCTTCGGCAACCTGAGTGTTACTGTCTCAGCATTCTTGCCCGCCACAACTAC
CACCACCACAAATGCCGTTAATAAGACAATAAATTCAACTTCATCGGAGAGTCAGGTGGTGC
TACATGAATTGCACGATTTTATCGCACAAATGAGAAGTGAGATAGACAAAGTTAAAGGCGAT
AAGGGTAGCCTAGAAAAGGTGATTCAGAACTTTGCCTCTGGTCATGACGCTTCAATAAAGAA
AATAAATGACGTAGAGGTCATAAATTTCTGGATATCTTCATGGTGCAGAATGGGCTTGTATG
AGATCGACTTCGGCTGGGGCAAGCCGATTTGGGTTACAGTTGACCCTAATATCAAGCCAAAT
AAGAATTGCTTCTTTATGAACGACACAAAATGCGGAGAAGGAATAGAGGTATGGGCAAGCTT
CCTGGAAGACGACATGGCCAAGTTTGAATTGCACTTATCGGAGATATTGGAATTGATCTAA
PsSalAT Protein sequence
SEQ ID NO: 136
MATMYSAAVEVISKETIKPTTPTPSQLKNFNLSLLDQCFPLYYYVPIILFYPATAANSTGSS
NHHDDLDLLKSSLSKTLVHFYPMAGRMIDNILVDCHDQGINFYKVKIRGKMCEFMSQPDVPL
SQLLPSEVVSASVPKEALVIVQVNMEDCGGTAICSSVSHKIADAATMSTFIRSWASTTKTSR
SGGSTAAVTDQKLIPSFDSASLFPPSERLTSPSGMSEIPFSSTPEDTEDDKTVSKREVFDFA
KITSVREKLQVLMHDNYKSRRQTRVEVVTSLIWKSVMKSTPAGFLPVVHHAVNLRKKMDPPL
QDVSFGNLSVTVSAFLPATTTTTTNAVNKTINSTSSESQVVLHELHDFIAQMRSEIDKVKGD
KGSLEKVIQNFASGHDASIKKINDVEVINFWISSWCRMGLYEIDFGWGKPIWVTVDPNIKPN
KNCFFMNDTKCGEGIEVWASFLEDDMAKFELHLSEILELI
AstC Nucleotide sequence codon optimized
for its expression in S. cerevisiae.
SEQ ID NO: 137
ATGACTAAGATCAACCCATACAAGGGTATCTTGGTTGAATTGAAGGACATCGTTTTCACTTC
TTCTTCTGACCAAATCAAGTTGCCAATCAACACTTTCAAGTCTATCTTGTGTTGTGGTGCTA
CTGCTCAATACCAATGTGGTAAGATCAACAGAGCTCAATACTACTCTAGATTGGCTAGAGAC
TTCGCTTTGTCTTTGGCTGACGTTACTGCTTTGTTCGACACTGTTCAAGCTACTATCAGACC
AGAAGAATCTTTCTTGGCTTTCTTGGCTGAATTGAAGTCTAGATTCGGTGAACAATTGAAGT
TGTACGCTGTTGCTAACATGTCTAGAGAAGACTACGCTATGTTGAAGTCTTTGCCAATCGAC
TGGTCTTTGTTCGACGGTGTTTTCTTGTCTGCTGACTTGGGTATGAGAAAGCCAGAATTGAG
ATTCTTCAGACACGTTTTGGAATCTATCTCTATGAAGCCAGAAGACACTATCTTGGTTGACA
ACGACACTGACAACATCTTGTGTGCTTTGTCTATGGGTTTGAAGGGTATCTTGTTCGGTTCT
ACTTCTGTTCCACAAGCTTTGACTAACTTGTTGGAATACGACCACATCTCTAGAGCTGAACA
ATTCTTGAGATCTCACGCTAAGTCTTTGCACTCTGTTACTCACACTGGTGTTACTATCAGAG
AAAACTTCGCTCAATTGTTGATCTTGGAAGCTACTGGTGACATCGACTTGGTTGAATTGGAA
TACCACCCAACTACTTGGAACTACTTCATCGGTACTCCAGTTTTGACTCAAACTGAATTCCC
ACACGACTTGGACACTACTTCTTTGGCTACTACTGTTTTGGACAGACCAAAGGACATCGCTA
ACGAAATCATGGACGAAATGTTGAAGTACAGATCTGACGACGACTTGATGTTGACTTTCTTC
ACTGACTTCAAGAACAGAGTTGACCCAGTTGTTTGTTGTAACGTTTTGTCTTTGTTCTACAA
GTACGGTAGAGGTCACGAATTGCACCACACTTTGGCTTGGGTTAGACAAGTTTTGATCAGAA
GAGCTTACATCAACGGTACTGCTTTCTACCCAATGCCAGAAGCTTTCTTGTACTTCTTCTTC
AGATTCTTGCAACACATCACTCACTTGCCACAATTGTACGACGGTTTGAAGGTTTTGTTGAA
GGAAAGATTGCAAGAAAGAGTTGGTGTTCCAGTTGACCCAATCTCTTTGTCTATGAGATTGA
TCGCTTGTAACGGTGTTGGTATCCACGACAGAATGGGTTTGAACGCTTTGTTGTCTATGCAA
AACCCAGACGGTTCTTGGGACTTGGGTACTATGTACCACTACGCTTCTAAGAGATTGCCAAT
CGGTAACCAAGGTGTTTCTACTGCTATGGCTATCAAGGCTATCAAGCAATGTCAAGCTAACC
AATGTGCTGGTATCTAA
AstC Protein sequence
SEQ ID NO: 138
MTKINPYKGILVELKDIVFTSSSDQIKLPINTFKSILCCGATAQYQCGKINRAQYYSRLARD
FALSLADVTALFDTVQATIRPEESFLAFLAELKSRFGEQLKLYAVANMSREDYAMLKSLPID
WSLFDGVFLSADLGMRKPELRFFRHVLESISMKPEDTILVDNDTDNILCALSMGLKGILFGS
TSVPQALTNLLEYDHISRAEQFLRSHAKSLHSVTHTGVTIRENFAQLLILEATGDIDLVELE
YHPTTWNYFIGTPVLTQTEFPHDLDTTSLATTVLDRPKDIANEIMDEMLKYRSDDDLMLTFF
TDFKNRVDPVVCCNVLSLFYKYGRGHELHHTLAWVRQVLIRRAYINGTAFYPMPEAFLYFFE
RFLQHITHLPQLYDGLKVLLKERLQERVGVPVDPISLSMRLIACNGVGIHDRMGLNALLSMQ
NPDGSWDLGTMYHYASKRLPIGNQGVSTAMAIKAIKQCQANQCAGI
AstI Nucleotide sequence codon optimized
for its expression in S. cerevisiae.
SEQ ID NO: 139
ATGACTAGACAATCTCACTACCAAGCTATCATCTTGGACTTGGGTAACGTTGTTTTCGAATG
GGACACTTCTCAAAACCCACCAACTGCTGCTCCAAACCAAATCTCTTTGTTGAGAACTTCTA
TGAAGTCTCCAGTTTACCACTCTTACGAAAGAGGTCAATTGTCTACTGAAGAATGTCACAGA
TTGTTGGGTGAATCTTTGCACGTTGACCCAGGTCAAATCAAGGAAGCTTTCGACTTGGCTAG
ACAATCTTTGAGATCTAACCCAGCTTTGTTGGACTTCATCAGACAATTGAAGCAAACTAGAG
GTGTTGCTGTTTACGCTATGTCTAACATCCCACAAGCTGAAATCGAATACTTGAAGGAATCT
AGAGCTGGTGACATGGAAGTTTTCGACGAAGTTTTCGCTTCTGGTTACGTTGGTTCTAGAAA
GCCAGAAACTGAATTCTACAGAAGAGTTATGGGTGAAATCGGTTTGAAGGCTGAAAGAGTTG
TTTTCGTTGACGACAAGGAAGAAAACGTTGACGTTGCTAGAGGTTTGGGTTTGTACGGTGTT
TGTTTCGGTGGTGTTGAAGAATTGAGAGGTCACTTGTTGGGTATCTAA
AstI Protein sequence
SEQ ID NO: 140
MTRQSHYQAIILDLGNVVFEWDTSQNPPTAAPNQISLLRTSMKSPVYHSYERGQLSTEECHR
LLGESLHVDPGQIKEAFDLARQSLRSNPALLDFIRQLKQTRGVAVYAMSNIPQAEIEYLKES
RAGDMEVEDEVFASGYVGSRKPETEFYRRVMGEIGLKAERVVFVDDKEENVDVARGLGLYGV
CFGGVEELRGHLLGI
AstK Nucleotide sequence codon optimized
for its expression in S. cerevisiae.
SEQ ID NO: 141
ATGTGTACTACTTTCAAGGCTGCTATCTTCGACATGGGTGGTGTTTTGTTCACTTGGAACCC
AATCGTTGACACTCAAGTTTCTTTGAAGGACTTGGGTACTATCATCAACTCTGAAACTTGGG
AACAATTCGAAAGAGGTAAGATCGAACCAGACGACTGTTACCACCAATTGGGTTCTCAAATC
GGTTTGCCAGGTTCTGAAATCGCTGCTACTTTCAGACAAACTACTGGTTGTTTGAGACCAGA
CGCTAGAATGACTTCTTTGTTGAGAGAATTGAAGGGTCAAGGTGTTGCTGTTTACATGATGA
CTAACATCCCAGCTCCAGACTTCCACCAATTGAGAGAAATGCACTACGAATGGGACTTGTTC
GACGGTATCTTCGCTTCTGCTTTGGAAGGTATGAGAAAGCCAGACTTGGAATTCTACGAACA
CGTTTTGAAGCAAATCGACACTTCTGCTGCTGAAACTATCTTCGTTGACGACAAGTTGGAAA
ACGTTATCGCTGCTCAAGCTGTTGGTATGGTTGGTTTGCACTTGACTGACTCTTTGGCTACT
TGTATGGAATTGAGACAATTGGTTGGTTGTTAA
AstK Protein sequence
SEQ ID NO: 142
MCTTFKAAIFDMGGVLFTWNPIVDTQVSLKDLGTIINSETWEQFERGKIEPDDCYHQLGSQI
GLPGSEIAATFRQTTGCLRPDARMTSLLRELKGQGVAVYMMTNIPAPDFHQLREMHYEWDLE
DGIFASALEGMRKPDLEFYEHVLKQIDTSAAETIFVDDKLENVIAAQAVGMVGLHLTDSLAT
CMELRQLVGC
PYI04555.1 Protein sequence not
C-terminally extended.
SEQ ID NO: 143
MGASVSFQPFVPTPLDHAMPPIYVSQFLCFPTTTPQSAIQSLQVGIERLFERLPFLAGEILI
NEHTGAIKVQAPSALIREIPYMALRAHPDLYLPAKQCATTPIERQLKINSLDESYHPLPAAL
PLSQPQPVIRFQANTLADGILFAVSYHHCIFDGTGCGQILEMLAQCCSASDDKISLPTDCHT
DVLLREYISNLSPTTNIPHDYTQAYSTTVQPDPDASDPDTSPAIPSSLYTEAFTFPSQQITT
LRDACNHLLPKLPSTSNAHPHKPTPNPLSSNDVLTALIALCITRATNTTTPPLQPNNHSLSM
AVNLRTRIQPQVPDHFLGNFATLLPIHFTSPVHTQQSDLLLTTEPPDPALIHLTTLASQIRS
SLSTVNTDYICGLMTDLRTRRNAGENSSLLIEGIKISSWRHLSVYKPDFGPGLGKIAGFEFQ
AGLMDNLVVILPWRNGDWDVRVTLLERDMRGFREDRLVRWALGSG
ERR364415-1 contig 8546 Protein sequence
not C-terminally extended.
SEQ ID NO: 144
MARAPPPPPPGLRMRDTVLSIVKPIRKTQHLETIDATFVDLMRMDSFIPVIFAYRPADKSEA
AYSRLVNRIKESLQKVLVPFFGFAGRWVPSSGGSRRLLCNDEGVPFIEAFVDEELDSVVKAS
AAFQPVTELNGLGVLGMDMTSYDQRMPPEGGQPCVVAQVTRFKCGGVVLGVAFNHTHTDGQG
FYTFMRAWSDFSRINGTAIKVDHNRALPELASLSQFFIKQHDRIGGKTSTDRVNDHCSKVPE
RLALKAFEVRASKIKAAKLAAEDGGVGYVSTVDCIVAHLWKTLARLPPVVLDGREITVESPV
EGRNRFLDPPRPNMCGNCFAAMVTPKIPTQELLEMPLAAIAGKQREKLSTTRREEWFGQQSF
RELASAMNTSKSALLIVTSWFNFPMYEIDFGAGKPFFASTINMISPINGVCCGVIAPPTPGS
CSSIATLYILCLPAVLEALENVPDFLSFFVPHPNHKDNSQ

Further sequences applicable in the invention are listed below. The “previous” SEQ ID NOs refer to those as used in the respective patent document.
1. Albicanyl diphosphate synthases as described in PCT/CN2018/088902 (filed May 29, 2018) are:
DfHAD, DfHAD-9 (V274A) and DfHAD-8 (K532R) of Dryopteris fragrans

| Previous | | | Current |
|---|---|---|---|
| SEQ ID | | | SEQ ID |
| NO | Name | Type | NO |
| 1 | DfHAD | NA | 26 |
| 2 | DfHAD | AA | 27 |
| 3 | DfHAD-8 (K532R) | NA | 28 |
| 4 | DfHAD-8 (K532R) | AA | 29 |
| 5 | DfHAD-9 (V274A) | NA | 30 |
| 6 | DfHAD-9 (V274A) | AA | 31 |
| 7 | DfHAD codon optimized for E. coli | NA | 32 |
| 8 | DfHAD codon optimized for Tobacco | NA | 33 |
| 9 | DfHAD-8 (K532R) codon optimized for E. coli | NA | 34 |
| 10 | DfHAD-9 (V274A) codon optimized for E. coli | NA | 35 |
| 17 | DfHAD-His-GST | NA | 36 |
| 18 | DfHAD-His-GST | AA | 37 |

NA = Nucleic Acid
AA = Amino Acid

2. Drimane synthases (i.e. albicanol synthases or drimenol synthases) as described in PCT/EP2018/064344 (filed May 31, 2018) are

| Previous | | | Current | Current |
|---|---|---|---|---|
| SEQ ID | | | SEQ | SEQ ID |
| NO | Name or NCBI | | ID NO | NO |
| (AA) | accession number | Source | (AA) | (NA) |
| 1 | CvTps1 | Cryptoporus volvatus | 38 | 39, 40, 41 |
| 5 | LoTps1 | Laricifomes officinalis | 42 | 43, 44, 45, |
| | | | | 95 |
| 9 | OCH93767.1 | Obba rivulosa | 46 | 47,48 |
| 12 | EMD37666.1 | Gelatoporia | 49 | 50, 51, 96 |
| | | subvermispora | | |
| 15 | EMD37666-B | Gelatoporia | 52 | 53 |
| | | subvermispora | | |
| 17 | XP_001217376.1 | Aspergillus terreus | 54 | 55, 56,97 |
| 20 | OJJ98394.1 | Aspergillus aculeatus | 57 | 58, 59 |
| 23 | GAO87501.1 | Aspergillus udagawae | 60 | 61, 62 |
| 26 | XP_008034151.1 | Trametes versicolor | 63 | 64, 65 |
| 29 | XP_007369631.1 | Dichomitus squalens | 66 | 67, 68, 93 |
| 32 | ACg006372 | Antrodia cinnamomea | 69 | 70,71 |
| 35 | KIA75676.1 | Aspergillus ustus | 72 | 73, 74 |
| 38 | XP_001820867.2 | Aspergillus oryzae | 75 | 76, 77 |
| 41 | CEN60542.1 | Aspergillus calidoustus | 78 | 79,80 |
| 44 | XP_009547469.1 | Heterobasidion | 81 | 82,83 |
| | | irregulare | | |
| 47 | KLO09124.1 | Schizopora paradoxa | 84 | 85,86 |
| 50 | OJI95797.1 | Aspergillus versicolor | 87 | 88, 89 |
| 63 | XP_006461126 | Agaricus bisporus | 90 | 91, 92, 94 |

AA = Amino Acid

3. Drimenol synthases as described in WO2015/169871 are

| Previous | | | Current | Current |
|---|---|---|---|---|
| SEQ ID | | | SEQ ID | SEQ ID |
| NO | | | NO | NO |
| (AA) | Name | Source | (AA) | (NA) |
| 2 | DITps589 | Drimys lanceolota | 99 | 98, 100 |
| 8 | SCH51_3228_11 | Drimys winteri | 105 | 104, 106 |
| 5 | SCH51_3228_9 | Drimys winteri | 102 | 101,103 |
| 11 | SCH51_998_28 | Drimys winteri | 108 | 107, 109 |
| 14 | SCH52_13163_6 | Drimys lanceolota | 111 | 110, 112 |

4. Drimenol synthases as described in WO 2015/176959 are
VaTPS3 (SEQ ID NO:6) of Valeriana amurensis

| Previous | | | Current | Current |
|---|---|---|---|---|
| SEQ ID | | | SEQ ID | SEQ ID |
| NO | | | NO | NO |
| (AA) | Name | Source | (AA) | (NA) |
| 6 | VaTPS3 | Valeriana amurensis | 114 | 113, 115 |

What is claimed is:

1. A biocatalytic method of producing a drimanyl acetate compound comprising the steps of
(1) contacting in the presence of an acetyl group donor a drimanyl alcohol with a polypeptide having acetyl transferase activity of the enzyme class EC 2.3.1 capable of transferring an acetyl group from said acetyl group donor to said drimanyl alcohol to obtain a drimanyl acetate; and
(2) optionally isolating a drimanyl acetate compound from the reaction product of step (1),
wherein the method is performed in vivo in cell culture or in vitro in a liquid reaction medium, under conditions conducive to the production of at least one drimanyl acetate.

2. The method of claim 1, performed in a recombinant host cell or a recombinant non-human host organism capable of functionally expressing,
a) at least one acetyl transferase selected from the group consisting of
polypeptides comprising an amino acid sequence selected from the group consisting of SEQ ID NO: 9, 17, 118, 124, 144, 23, 21, 11, 19, 13, 15, 25, 121, 143, 127, 130, 133, and 136; and
polypeptides having acetyl transferase activity and comprising an amino acid sequence showing a degree of sequence identity of at least 40% to at least one of said amino acid sequences selected from the group consisting of SEQ ID NO: 9, 17, 118, 124, 144, 23, 21, 11, 19, 13, 15, 25, 121, 143, 127, 130, 133, and 136; optionally
b) at least one polypeptide having the ability to convert the non-cyclic sesquiterpene precursor FPP to at least one drimanyl alcohol in one or more enzymatic steps; and optionally
c) at least one enzyme selected from enzymes involved in the mevalonate pathway.

3. The method of claim 2, wherein said non-human host cell or host organism is selected from the group consisting of a prokaryotic or eukaryotic microorganism, and a cell derived therefrom.

4. The method of claim 1, further comprising as step (3) the processing of the drimanyl acetate of step (1) or of step (2) to obtain a derivative using chemical or biocatalytic synthesis or a combination of both.

5. The method of claim 1, which comprises providing a non-human host organism or host cell with
a) at least one nucleic acid comprising a nucleic acid sequence encoding at least one polypeptide having acetyl transferase activity capable of transferring an acetyl group from an acetyl group donor to drimanyl alcohol, optionally stably integrated into the genome; optionally
b) at least one nucleic acid comprising a nucleic acid sequence encoding at least one polypeptide having drimanyl alcohol synthase activity capable of producing a drimanyl alcohol from a non-cyclic sesquiterpene precursor, optionally stably integrated into the genome; and optionally
c) at least one nucleic acid comprising a nucleic acid sequence encoding at least one polypeptide involved in the biosynthetic pathway for producing said non-cyclic sesquiterpene precursor, optionally stably integrated into the genome.

6. A method of using the acetyltransferase as defined in claim 1, the method comprising using the acetyltransferase for preparing odorants, flavour or fragrance ingredients; or as insect/pest control.

7. The method of claim 1, further comprising, prior to step 1) the enzymatic synthesis of said drimanyl alcohol compound from a non-cyclic sesquiterpene precursor.

8. The method of claim 1, further comprising, prior to step 1) the biocatalytic formation of said drimanyl alcohol compound,
wherein said enzymatic synthesis of said drimanyl alcohol is catalysed by one or more polypeptides having the ability to convert said non-cyclic sesquiterpene precursor to at least one drimanyl alcohol in one or more enzymatic steps,
wherein said at least one drimanyl alcohol is produced in a single or more enzymatic steps from FPP,
wherein said at least one drimanyl alcohol is produced by an enzymatic conversion of FPP, catalysed by
a) a polypeptide having drimane sesquiterpene synthase activity forming said drimanyl alcohol; or
b) a combination of a polypeptide having drimanyl phosphate synthase activity forming at least one drimanyl phosphate intermediate, and a polypeptide having phosphatase activity converting said at least one drimanyl phosphate intermediate to at least one drimanyl alcohol,
wherein said combination of polypeptides comprises an albicanyl diphosphate synthase activity and a phosphatase.

9. The method of claim 3, wherein said non-human host cell or host organism is selected from the group consisting of bacterial, fungal and plant cells and plants.


SEQUENCE LISTING (144 sequences)

SEQ ID NO: 1 - DNA, 5689 bp, synthetic construct
  misc_feature 1..5689: note = Plasmid pF167
  source 1..5689: mol_type = other DNA
tggtcagcaacaacgccgaagaatcactctcgtgttgagaattgcacgccttgaccacgacacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttttggcattggcaaagtgcggactgcatagtcactgtggtgccgtacttagggtacgcgttcctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcattatgtcacccgcagttctgtgtcgtagtcatcaacatagcacctatcctttggcatctcggtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagcacttgctacactgtcaggatagcttccgtcacatggtggcgatcaccgtacatctgagtgagacgttaattaaagtagaccgctcacacatgggcggccgccgtctcaaggtgcagttcgcgtgcaattataacgtcgtggcaactgttatcagtcgtaccgcgccattcgactacgtcgtaaggccgtttctgacagagtaaaattcttgagggaactttcaccattatgggaaatgcttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcga

SEQ ID NO: 2 - DNA, 60 bp, synthetic construct
  misc_feature 1..60: note = Sequence for homologous recombination
  source 1..60: mol_type = other DNA
gcacttgctacactgtcaggatagcttccgtcacatggtggcgatcaccgtacatctgag

SEQ ID NO: 3 - DNA, 60 bp, synthetic construct
  misc_feature 1..60: note = Sequence for homologous recombination
  source 1..60: mol_type = other DNA
aggtgcagttcgcgtgcaattataacgtcgtggcaactgttatcagtcgtaccgcgccat

SEQ ID NO: 4 - DNA, 1584 bp, synthetic construct
  misc_feature 1..1584: note = Dichomitus squalens Albicanol synthase codon optimized for its expression in S. cerevisiae
  source 1..1584: mol_type = other DNA
  CDS 1..1584
atggcttctatccacagaagatacactactttgatcttggacttgggtgacgttttgttcagatggtctccaaagactgaaactgctatcccaccacaacaattgaaggacatcttgtcttctgttacttggttcgaatacgaaagaggtagattgtctcaagaagcttgttacgaaagatgtgctgaagaattcaagatcgaagcttctgttatcgctgaagctttcaagcaagctagaggttctttgagaccaaacgaagaattcatcgctttgatcagagacttgagaagagaaatgcacggtgacttgactgttttggctttgtctaacatctctttgccagactacgaatacatcatgtctttgtcttctgactggactactgttttcgacagagttttcccatctgctttggttggtgaaagaaagccacacttgggttgttacagaaaggttatctctgaaatgaacttggaaccacaaactactgttttcgttgacgacaagttggacaacgttgcttctgctagatctttgggtatgcacggtatcgttttcgacaaccaagctaacgttttcagacaattgagaaacatcttcggtgacccaatcagaagaggtcaagaatacttgagaggtcacgctggtaagttggaatcttctactgacaacggtttgatcttcgaagaaaacttcactcaattgatcatctacgaattgactcaagacagaactttgatctctttgtctgaatgtccaagaacttggaacttcttcagaggtgaaccattgttctctgaaactttcccagacgacgttgacactacttctgttgctttgactgttttgcaaccagacagagctttggttaactctgttttggacgaaatgttggaatacgttgacgctgacggtatcatgcaaacttacttcgacagatctagaccaagaatggacccattcgtttgtgttaacgttttgtctttgttctacgaaaacggtagaggtcacgaattgccaagaactttggactgggtttacgaagttttgttgcacagagcttaccacggtggttctagatactacttgtctccagactgtttcttgttcttcatgtctagattgttgaagagagctgacgacccagctgttcaagctagattgagaccattgttcgttgaaagagttaacgaaagagttggtgctgctggtgactctatggacttggctttcagaatcttggctgctgcttctgttggtgttcaatgtccaagagacttggaaagattgactgctggtcaatgtgacgacggtggttgggacttgtgttggttctacgttttcggttctactggtgttaaggctggtaacagaggtttgactactgctttggctgttactgctatccaaactgctatcggtagaccaccatctccatctccatctgctgcttcttcttctttcagaccatcttctccatacaagttcttgggtatctctagaccagcttctccaatcagattcggtgacttgttgagaccatggagaaagatgtctagatctaacttgaagtctcaataa

SEQ ID NO: 5 - protein, 527 aa, synthetic construct
  REGION 1..527: note = Synthetic Construct
MASIHRRYTTLILDLGDVLFRWSPKTETAIPPQQLKDILSSVTWFEYERGRLSQEACYERCAEEFKIEASVIAEAFKQARGSLRPNEEFIALIRDLRREMHGDLTVLALSNISLPDYEYIMSLSSDWTTVFDRVFPSALVGERKPHLGCYRKVISEMNLEPQTTVFVDDKLDNVASARSLGMHGIVFDNQANVFRQLRNIFGDPIRRGQEYLRGHAGKLESSTDNGLIFEENFTQLIIYELTQDRTLISLSECPRTWNFFRGEPLFSETFPDDVDTTSVALTVLQPDRALVNSVLDEMLEYVDADGIMQTYFDRSRPRMDPFVCVNVLSLFYENGRGHELPRTLDWVYEVLLHRAYHGGSRYYLSPDCFLFFMSRLLKRADDPAVQARLRPLFVERVNERVGAAGDSMDLAFRILAAASVGVQCPRDLERLTAGQCDDGGWDLCWFYVFGSTGVKAGNRGLTTALAVTAIQTAIGRPPSPSPSAASSSFRPSSPYKFLGISRPASPIRFGDLLRPWRKMSRSNLKSQ

SEQ ID NO: 6 - DNA, 1623 bp, synthetic construct
  misc_feature 1..1623: note = Agaricus bisporus Drimeol synthase sequence codon optimized for its expression in S. cerevisiae
  source 1..1623: mol_type = other DNA
  CDS 1..1623
atggctccaccacaaagaccattcactgctatcgttttcgacatcggtgacgttttgttccaatggtctgctactactaagacttctatctctccaaagactttgagatctatcttgaactgtccaacttggttcgactacgaaagaggtagattggctgaaaacgcttgttacgctgctatctctcaagaattcaacgttaacccagacgaagttagagacgctttctctcaagctagagactctttgcaagctaaccacgacttcatctctttgatcagagaattgaaggctcaagctaacggtagattgagagtttacgctatgtctaacatctctttgccagactgggaagttttgagaatgaagccagctgactgggacatcttcgaccacgttttcacttctggtgctgttggtgaaagaaagccaaacttggctttctacagacacgttatcgctgctactgacttgcaaccacaccaaactatcttcgttgacgacaagttggaaaacgttttgtctgctagatctttgggtttcactggtatcgttttcgacgaaccatctgaagttaagagagctttgagaaacttgatcggtgacccagttcaaagaggtggtgaattcttggttagaaacgctggtaagttgggttctatcactagaactactgctaagcacgaatctatcccattggacgaaaacttcgctcaattgttgatcttggaaatcactggtaacagagctttggttaacttggttgaacacccacaaacttggaacttcttccaaggtaagggtcaattgactactgaagaattcccattcgacttggacactacttctttgggtttgactatcttgaagagatctagagaaatcgctgactctgttatggacgaaatgttggaatacgttgacccagacggtatcatccaaacttacttcgaccacagaagaccaagattcgacccagttgtttgtgttaacgctttgtctttgttctacgcttacggtagaggtgaacaattgagatctactttgacttgggttcacgaagttttgttgaacagagcttacttggacggtactagatactacgaaactgctgaatgtttcttgtacttcatgtctagattgttggctacttctggtgacccagacttgcactctttgttgaagccattgttgaaggaaagagttcaagaaagaatcggtgctgacggtgactctttggctttggctatgagaatcttggcttgtgacttcgttggtatcagagacgaagttgacttgagaactttgttgactttgcaatgtgaagacggtggttgggaagttggttggatgtacaagtacggttcttctggtatctctatcggtaacagaggtttggctactgctttggctatcaaggctgttgacactatgttccaaccacaaatcagattctctgaatctccaactgacactttggttgaaaacgctatccacaagagaagaccatctttctctgaaaagttcttgggtaagagaccaagatctggttctttcagaaagccattgcaatggatcttgcaaggttctaagttgagaaagtctgttgaaatcggttcttaa

SEQ ID NO: 7 - protein, 540 aa, synthetic construct
  REGION 1..540: note = Synthetic Construct
MAPPQRPFTAIVFDIGDVLFQWSATTKTSISPKTLRSILNCPTWFDYERGRLAENACYAAISQEFNVNPDEVRDAFSQARDSLQANHDFISLIRELKAQANGRLRVYAMSNISLPDWEVLRMKPADWDIFDHVFTSGAVGERKPNLAFYRHVIAATDLQPHQTIFVDDKLENVLSARSLGFTGIVFDEPSEVKRALRNLIGDPVQRGGEFLVRNAGKLGSITRTTAKHESIPLDENFAQLLILEITGNRALVNLVEHPQTWNFFQGKGQLTTEEFPFDLDTTSLGLTILKRSREIADSVMDEMLEYVDPDGIIQTYFDHRRPRFDPVVCVNALSLFYAYGRGEQLRSTLTWVHEVLLNRAYLDGTRYYETAECFLYFMSRLLATSGDPDLHSLLKPLLKERVQERIGADGDSLALAMRILACDFVGIRDEVDLRTLLTLQCEDGGWEVGWMYKYGSSGISIGNRGLATALAIKAVDTMFQPQIRFSESPTDTLVENAIHKRRPSFSEKFLGKRPRSGSFRKPLQWILQGSKLRKSVEIGS

SEQ ID NO: 8 - DNA, 1320 bp, synthetic construct
  misc_feature 1..1320: note = CrDAT codon optimized for its expression in S. cerevisiae
  source 1..1320: mol_type = other DNA
  CDS 1..1320
atggaaagtggtaaaatatctgtagaaacggaaaccctatcgaaaacccttatcaagccgtcctcgcccacgccacagtctctttcccgttacaacttatcttacaatgatcagaacatttatcagacctgcgtatctgtagggttcttttacgaaaatcctgatggcatcgagatatccacgatccgtgagcagctgcagaacagcctgtccaaaactctggtctcatactatcccttcgcaggcaaagtagtgaagaacgactacatacattgtaacgacgatggcattgagttcgtcgaagtcagaattagatgcaggatgaatgatatacttaaatacgaactacgttcttacgctcgtgatttagtcctgccaaagagggttaccgtgggctctgaagataccaccgctatcgtgcagctgtcccatttcgactgtggcggtttggccgtggcttttggtatctcccacaaggtggccgatggtggtactatagcctctttcatgaaggactgggcggcctctgcttgctacttgtcctcaagtcaccacgttccaacgcctctattggtctcagacagtattttcccaagacaggacaacataatatgcgaacaatttcccacctcaaagaattgcgtggagaagacattcattttcccaccagaagcaatcgaaaagttaaagagtaaggccgttgaattcggcattgagaaaccgaccagagtagaggtcctgactgcattcttatctagatgcgccaccgtagcaggtaagtcggcagctaagaacaacaattgtggtcaaagcctgccctttccggttctacaggccattaatttgaggccgattctagaattgccacagaactctgtgggtaatctagtttcgatctacttcagcaggacaattaaggaaaacgactacctaaatgaaaaggagtacactaaattggtgataaacgagttgcgtaaagaaaagcaaaagatcaagaatttgagcagagagaagttgacctacgtcgcccagatggaagagttcgttaagtcacttaaagagttcgatatctccaacttcctagatatcgatgcctacttaagcgatagttggtgcaggtttcctttctacgacgtcgacttcggatggggaaagcctatctgggtctgcctattccagccttacatcaagaactgcgttgttatgatggactatcctttcggtgacgattatggtatcgaggctattgtttctttcgagcaagagaaaatgtccgcgttcgagaagaacgaacagttactgcagtttgtgtctaactaa

SEQ ID NO: 9 - protein, 439 aa, synthetic construct
  REGION 1..439: note = Synthetic Construct
MESGKISVETETLSKTLIKPSSPTPQSLSRYNLSYNDQNIYQTCVSVGFFYENPDGIEISTIREQLQNSLSKTLVSYYPFAGKVVKNDYIHCNDDGIEFVEVRIRCRMNDILKYELRSYARDLVLPKRVTVGSEDTTAIVQLSHFDCGGLAVAFGISHKVADGGTIASFMKDWAASACYLSSSHHVPTPLLVSDSIFPRQDNIICEQFPTSKNCVEKTFIFPPEAIEKLKSKAVEFGIEKPTRVEVLTAFLSRCATVAGKSAAKNNNCGQSLPFPVLQAINLRPILELPQNSVGNLVSIYFSRTIKENDYLNEKEYTKLVINELRKEKQKIKNLSREKLTYVAQMEEFVKSLKEFDISNFLDIDAYLSDSWCRFPFYDVDFGWGKPIWVCLFQPYIKNCVVMMDYPFGDDYGIEAIVSFEQEKMSAFEKNEQLLQFVSN

SEQ ID NO: 10 - DNA, 1320 bp, synthetic construct
  misc_feature 1..1320: note = TcTAT codon optimized for its expression in S. cerevisiae
  source 1..1320: mol_type = other DNA
  CDS 1..1320
atggagaagacggatttacacgtgaaccttatcgaaaaggttatggtcggtccaagcccacctttgccgaagactacactacaactatcctcaatcgataacctacctggtgttagaggctcgatctttaatgccttgttaatatataatgcttcaccgtctcccacgatgatctccgctgatccagctaaacctatcagagaagctttggccaaaatcctggtttactacccacctttcgctggcaggctacgtgagactgagaatggcgatctagaggtagagtgcacgggagagggtgcgatgtttcttgaagcgatggccgataatgaattaagtgtcttaggggatttcgacgacagtaaccctagcttccagcaattgttgttctcccttccactggacactaacttcaaggatctatccctgttagtcgtgcaggtgacaaggtttacatgcggcgggttcgtcgttggggtttcatttcaccacggtgtatgcgatgggagaggagctgcacaatttctgaagggactagccgaaatggcaaggggagaagttaagttgtcgctggagccgatctggaatcgtgaactagttaagctggacgatcccaagtatttgcaattctttcacttcgaattcttgagagcaccatcaatcgtcgaaaagatagttcaaacctacttcatcatcgatttcgaaactattaactacataaagcagtcagtaatggaagaatgtaaagaattctgcagctcttttgaagttgcttctgcaatgacgtggatagctaggaccagggcctttcaaatacccgagagcgagtacgtgaaaatcctatttggtatggacatgaggaactcttttaacccgcctctgccgtcaggttattatggaaactcaattggtacggcttgtgcagtggacaatgttcaagacttgttgagtggttcactactgagggcaataatgataatcaagaaatcaaaggtatcacttaacgacaactttaagagccgtgctgtcgttaagccttcagaacttgacgtaaacatgaaccatgagaatgtggtggcatttgctgactggtcaaggctggggttcgacgaagtcgattttggctggggaaacgcggtgtctgtttccccggtacagcaacagtcggcacttgcaatgcagaattacttcctgttcttgaagccaagcaagaacaagcccgatggaattaagattctaatgttcctaccattgtccaagatgaaatccttcaaaattgaaatggaagcgatgatgaagaagtacgtcgccaaggtataa

SEQ ID NO: 11 - protein, 439 aa, synthetic construct
  REGION 1..439: note = Synthetic Construct
MEKTDLHVNLIEKVMVGPSPPLPKTTLQLSSIDNLPGVRGSIFNALLIYNASPSPTMISADPAKPIREALAKILVYYPPFAGRLRETENGDLEVECTGEGAMFLEAMADNELSVLGDFDDSNPSFQQLLFSLPLDTNFKDLSLLVVQVTRFTCGGFVVGVSFHHGVCDGRGAAQFLKGLAEMARGEVKLSLEPIWNRELVKLDDPKYLQFFHFEFLRAPSIVEKIVQTYFIIDFETINYIKQSVMEECKEFCSSFEVASAMTWIARTRAFQIPESEYVKILFGMDMRNSFNPPLPSGYYGNSIGTACAVDNVQDLLSGSLLRAIMIIKKSKVSLNDNFKSRAVVKPSELDVNMNHENVVAFADWSRLGFDEVDFGWGNAVSVSPVQQQSALAMQNYFLFLKPSKNKPDGIKILMFLPLSKMKSFKIEMEAMMKKYVAKV

SEQ ID NO: 12 - DNA, 1332 bp, synthetic construct
  misc_feature 1..1332: note = CrMAT codon optimized for its expression in S. cerevisiae
  source 1..1332: mol_type = other DNA
  CDS 1..1332
atggattctattactatggttgaaaccgagacattatcgaagacattgattaagccatctagcccaaccccacagagtctgtcacactacaacctgtcttacaacgatcagaatatatatccagagtatatatttgcaggcttcttctacagcaacccggacggacacgagatctcgactattagagagcagctgcagaactctcttagcaagacattagtatcttattatccattcgctggtaaagtggtcaagaacgattacattcactgcaatgacgacggaatagaattcgtagacgtcagaatccactgcaggatgaacgatattcttaagcctgaattgaggtcttacgcttccgagcttattcgtccgaatagaagtacagtggggtcagaggacagtactgcgcttgtccagttatctcactttgactgtggcggggtagctgtcgcattcggtatatctcacaaagtcgcagacgcagcgactattctgtcgtttataaaggattgggccgcttctacgtgtgacttgtcaagtagtcacgatgtgtctacaccagtactagtatccgattccatattcccccgtcaagataacatcatctgtggccagttccctgcttcaccgaactgcgtgaggaagcgtttcttattcagcccggaagctatcgaaagactaaaatcgaaagccattgaatttgggatcgagaagccaacgagggtagaggtcctgacagcattcttgtgccgttgcgctaccgttgcaggtaaatctgcggccaagaacaacaattgtggacagtcactgcctttcgctgttattcaagcagtcaacctgaggcccttactagaactgccaaagaattccgtcggcaaccttatatcaatctacttttctacaatcaaagaaaacgacacggtgaacatcgaacaggagttcacaaaattagtgatcggagagttgaggaaggctaaggataagttaaagaacctgtcgcaagagaagctgaattacgtagctagaatgcaagatttcgcgaattgcctgaaggaattggacataagttcattcttcgacatggaaaacgtggacatagacgcttatttattttcgagctggtgcaggttccccttctacgacatcgatttcggtctggggaagccaatatgggtctgcatgtttcagcctcactttaagaattgtataattttaatggattatccctttggtgacgattacggcatcgaagccctaattacgttggaacaagagaagatgcccgccttcgaaaacaacgagctgctgctaagcttcgccagcaactaa

SEQ ID NO: 13 - protein, 443 aa, synthetic construct
  REGION 1..443: note = Synthetic Construct
MDSITMVETETLSKTLIKPSSPTPQSLSHYNLSYNDQNIYPEYIFAGFFYSNPDGHEISTIREQLQNSLSKTLVSYYPFAGKVVKNDYIHCNDDGIEFVDVRIHCRMNDILKPELRSYASELIRPNRSTVGSEDSTALVQLSHFDCGGVAVAFGISHKVADAATILSFIKDWAASTCDLSSSHDVSTPVLVSDSIFPRQDNIICGQFPASPNCVRKRFLFSPEAIERLKSKAIEFGIEKPTRVEVLTAFLCRCATVAGKSAAKNNNCGQSLPFAVIQAVNLRPLLELPKNSVGNLISIYFSTIKENDTVNIEQEFTKLVIGELRKAKDKLKNLSQEKLNYVARMQDFANCLKELDISSFFDMENVDIDAYLFSSWCRFPFYDIDFGLGKPIWVCMFQPHFKNCIILMDYPFGDDYGIEALITLEQEKMPAFENNELLLSFASN

SEQ ID NO: 14 - DNA, 1254 bp, synthetic construct
  misc_feature 1..1254: note = LiAAT-4 DNA sequence codon optimized for its expression in S. cerevisiae
  source 1..1254: mol_type = other DNA
  CDS 1..1254
atggccatgataatcacgaagcaaatattaagacctagttccccgacgccccaagcctttaagaatcacaagctgtcctacttagaccaaatacaggcgcctatttacatacctttgttgttcttctataagaacgaggagtcaaaatacccagaccagatctcgcaaagatttaagcagagtttgtccgaaattttgacaatattctaccctttggctggtacgatgaggcataactcgttcgtggactgcaatgacaggggtgtcgaatttgtagaggtcagggtccatgcgagactagcccagttcattcaagatcctaagatggaagagcttaagcaattgatacctgtggattgtatatctcacactgacgatgatttcttattgctagtcaagattagctattttgactgcggtgaggtcgtcgttggagtctgcatgtcgcacaaaattggtgacggaattagtctggcagcgtttatgaacgcttgggcagcaacgtgtaggggagaatcgtctagtgagatcattcacccatcttttgatcttgctttacactttccgcctaaagaccacttgtcttcagcatcctcattccgtgtggccatagcccaggagaacatcatgaccaagaggctagtatttgatagagaaaagttggagaagctgcgtaagagaatcgctgccagttctgatggggtgagagaccctagcagagttgaagctgtatctgtctttatttggaaaagcttaattgaagcccacaaggccgagtcacacatgactgagacaccagccgtttctattgctagccacgccgtgaacttaaggcctagaacagtcccacaaatggaccaaactttcggtaactgctacgctcccgcttcggccgttgtctcctgggatgaagactacgtacatcacagtcgtttgagggcggcccttagagaaatcgacgacgactacattaataaggtcttgaaagccgacaataattatttaacgcaggatcaaattggtgacttgtttaaaccagaaaactcagttctaagttcgtggtggcgtttcccagtttacaaggtagactttggatggggtaagccagtttgggtttctacgacgaccatccaatacatgaacttgattatatttacttcgacgccctcagaagacggcatagaggcgtgggtaactactactcacaatttcttccaagttctgcaggcaaactataataaacttgacacgtaa

SEQ ID NO: 15 - protein, 417 aa, synthetic construct
  REGION 1..417: note = Synthetic Construct
MAMIITKQILRPSSPTPQAFKNHKLSYLDQIQAPIYIPLLFFYKNEESKYPDQISQRFKQSLSEILTIFYPLAGTMRHNSFVDCNDRGVEFVEVRVHARLAQFIQDPKMEELKQLIPVDCISHTDDDFLLLVKISYFDCGEVVVGVCMSHKIGDGISLAAFMNAWAATCRGESSSEIIHPSFDLALHFPPKDHLSSASSFRVAIAQENIMTKRLVFDREKLEKLRKRIAASSDGVRDPSRVEAVSVFIWKSLIEAHKAESHMTETPAVSIASHAVNLRPRTVPQMDQTFGNCYAPASAVVSWDEDYVHHSRLRAALREIDDDYINKVLKADNNYLTQDQIGDLFKPENSVLSSWWRFPVYKVDFGWGKPVWVSTTTIQYMNLIIFTSTPSEDGIEAWVTTTHNFFQVLQANYNKLDT

SEQ ID NO: 16 - DNA, 1485 bp, synthetic construct
  misc_feature 1..1485: note = FgaAT codon optimized for its expression in S. cerevisiae
  source 1..1485: mol_type = other DNA
  CDS 1..1485
atgaagaagcaggtcacttttaaacctttcagattgtcaccagtcgatcatagtttacctaaggtttacatcttcaagtctctatatttcagaggggtagatgacactggttctcttagtagactacaggatggcattgatcgtttgatatcttgtctgcctttcctgtcaggcgaggtggttccatgtgcagacataccagataaggtcggtgtacttcaagttcagatgccttgcccttccttgcaagaaattccaatgctgctagtcaaaagttatccaaaccatacatggccagcagcttctacctcggaaagatggagaaacaccgctttgctagatcagtcgtataggccattgccggattttatacccccatcaaagcctagacctgttttgagatttcaggcgaacttcctggctgatggtttaatgctatgtatgggatataatcattctgtattcgacggaacaggtgcggggaacattctggaaatgttggctgattgttgtagagctaatccaaactccatcttagcactgccaacaaacggtgacatagagagcgagttgagaggtttactgtccagtccaggtgtagcagttgcaaatgcctctcaggaagcctacgcaattaattgtgcacacacggaagtagaacccgaacccagttcagctatgctttactgttggcctttcttgctgagttctgagaagattgaatgtttacaggaagcatgtaatagtttactaccacatatcgttcgtttgtactctggtacgcagagttcgcttataaaccaagatacgaactggccacatattctttcatcaaacgacgtgctaaccgctcttttagctgtttcaattgaaaaggctagggaagcaacaggtgcccttggtcatatgagcagatcattggctatggctgttaacctacgtgagcgtttaaagcctatgccaagacactatttgggtaacctagttaccactgtgtgggtatcacatcaccgtcctgccgttaaggaccttgaaacaatggttttaccagtgccagcatgcaataggcacgaaatagacagagacgacttgttgtggataacccacgtggcattccgtattagattagggctgaacgcaataaacgaagaacatattagaggtctgatccactacttgcattcccaagacgattgggaacagattggaatacatttcaccgatccaattttcatttcttcctggcgtcaccttaaggtctatgaactagacttcggacctactatcggtcatgctgaacactttgagatggatgtcggcaccaccgatggtgtgtgtgttgttatgcctgctaacaccagagccgtcggcaagactaagaaggctccttgggacattagaatcgtgttaaaccccgaagtgttacaggcactaatagctagtgccatctttggttgggctatggtcaaggacgcttcgacataa

SEQ ID NO: 17 - protein, 494 aa, synthetic construct
  REGION 1..494: note = Synthetic Construct
MKKQVTFKPFRLSPVDHSLPKVYIFKSLYFRGVDDTGSLSRLQDGIDRLISCLPFLSGEVVPCADIPDKVGVLQVQMPCPSLQEIPMLLVKSYPNHTWPAASTSERWRNTALLDQSYRPLPDFIPPSKPRPVLRFQANFLADGLMLCMGYNHSVFDGTGAGNILEMLADCCRANPNSILALPTNGDIESELRGLLSSPGVAVANASQEAYAINCAHTEVEPEPSSAMLYCWPFLLSSEKIECLQEACNSLLPHIVRLYSGTQSSLINQDTNWPHILSSNDVLTALLAVSIEKAREATGALGHMSRSLAMAVNLRERLKPMPRHYLGNLVTTVWVSHHRPAVKDLETMVLPVPACNRHEIDRDDLLWITHVAFRIRLGLNAINEEHIRGLIHYLHSQDDWEQIGIHFTDPIFISSWRHLKVYELDFGPTIGHAEHFEMDVGTTDGVCVVMPANTRAVGKTKKAPWDIRIVLNPEVLQALIASAIFGWAMVKDAST

SEQ ID NO: 18 - DNA, 1548 bp, synthetic construct
  misc_feature 1..1548: note = GAO81666.1 codon optimized for its expression in S. cerevisiae
  source 1..1548: mol_type = other DNA
  CDS 1..1548
atggaagaacataccagatcctttgagccatttgacctggcctgcttagatcatactgtggggccggttttcatgaatttcttcttaagtttcaagcctgccaagatcgaagagagtcttatctcaattgaagaaggtgtaactaggttagtcaacagactgccattcctggccggtgacgttgttaattctgaaaacgtagacggtcgtgttaatgtaatgaggattcagccaagctcgacattgattagagagattcccatgctacacacaaagcaccatcctcatcatattttgccgaatacacctagacagattaacccagggtccgaccaggatcaaaggtttgctcccttggacgattcgtacgtaccaccagtgtctttattacctcttgctccgggcccaaggcccgtagtaagattccaaactaatgttgtaattgacgggattgtgcttgcacttgggttccaccactcagtatttgatgctaccggagtgggtttgttgatcgaaatgttggccacatgttgttcaagcgattgtcctgccctatcatcccatattgaactggaagaagaaataaggctaaggcgttctgtggacaagataggtaacggcgctactgatttagcatcccaaagagatgaagttcaggacccaaatggcagtatcactgagtccccagctcatgtgcctatgggtgacagtagctgggtaccccctaaactatctgtttactcctttaatttatcggctgccggtttggctcatttgaagacagcctgtaacaagttattgcccgctatccatgcgagtcaaaatggttcaccacaaagtgcggaatcagaaggtgagaaattacagcaagaattcgtttcgacgaatgatgttctaacggctttgttggctacttccattcaccaagcgaggtctagagttactgaaacggaattagttccgacaaaagcgaaacttgcgatggccgttaatttgagggaaagggccagttcgttacccaagacttatttggggaactcgctaacagttacagaagcttttgtgtattctttagctgctaccgacggtttcgatggtgtccccgagagacattatcatcctgacttgcgtaacgccttgctattagaaattgctagagttgcattgcaattgagaaaagggctggcagcgatagatgacgcgtattttaggcaatttgtgtctagattgagggctaatctagactggtcacagttaggtgcaaacctgcctgacactatggtgtcaagctggagacatttgaaagtatatcgtttagacttcggtgctagattggggagagttgtagagttccatccacaaaccgctttggttgacggtatttgtatcatccaacctgaaaggatagcccatgaggatgattcagctgatatggctccagaatctgggtgggaagtttgtgtaacattacaaagcgatgccatggagtgcttcttaagaggtgggttgttcacaagtctttctcaaggtgcaattagaagagtttaa

SEQ ID NO: 19 - protein, 515 aa, synthetic construct
  REGION 1..515: note = Synthetic Construct
MEEHTRSFEPFDLACLDHTVGPVFMNFFLSFKPAKIEESLISIEEGVTRLVNRLPFLAGDVVNSENVDGRVNVMRIQPSSTLIREIPMLHTKHHPHHILPNTPRQINPGSDQDQRFAPLDDSYVPPVSLLPLAPGPRPVVRFQTNVVIDGIVLALGFHHSVFDATGVGLLIEMLATCCSSDCPALSSHIELEEEIRLRRSVDKIGNGATDLASQRDEVQDPNGSITESPAHVPMGDSSWVPPKLSVYSFNLSAAGLAHLKTACNKLLPAIHASQNGSPQSAESEGEKLQQEFVSTNDVLTALLATSIHQARSRVTETELVPTKAKLAMAVNLRERASSLPKTYLGNSLTVTEAFVYSLAATDGFDGVPERHYHPDLRNALLLEIARVALQLRKGLAAIDDAYFRQFVSRLRANLDWSQLGANLPDTMVSSWRHLKVYRLDFGARLGRVVEFHPQTALVDGICIIQPERIAHEDDSADMAPESGWEVCVTLQSDAMECFLRGGLFTSLSQGAIRRV

SEQ ID NO: 20 - DNA, 1284 bp, synthetic construct
  misc_feature 1..1284: note = CfACT1-6 DNA sequence codon optimized for its expression in S. cerevisiae
  source 1..1284: mol_type = other DNA
  CDS 1..1284
atgaaggtagaacgtttctcaagaaagttgataaaaccccacacgcctactcccgagaacttaaagaaatataaattgtctctattggacaaatgccttggacacgacaacttcgcgatcgtgttgttttacgaatctaaaccaagaaataagagtgagcttgaggaatcgttagagaaggtcttggtagacttttatcccttggctgggcgtcacacgatgaacgaccatattgtcgactgctcggatgtcggcgctgtctttgtagaagccgaggctttagatgtagagttgactatggatgagttagtcaagaacatggaagctcaaactatacaccacttgttgccaaatcagtacttcagtgcagatgctccgaacccgctgctttccatccaagttacgcactttccttccggtggtttggctatcggaatcgcagtctcgcacgctgtatttgatggtttctctttgggcgtgttcgtagcagcatggtcaaaggcgaccatgaacccggataggaaaatcaaaataacaccgtcattcgacttaccatcacttctgccctacaaagacgacaactttggtttgactgctgctgaaattgtcagccagagcgaggacatcgtagttaagagatttatcttcggcaaggaagccatcacgaggttgagaagtaagcttagtccaaataggaacgggaagaaaatatccagggttagggtcgtttgtgcagtcattgtaaaggccttgatgggattggagcgtgccaaacacggtaagacgcgtgatttcttaattactcaatcaattaacatgagggagagaactaaggcgccgctgcagaaacacgcctgcggcaacttagcagtcttgagttgcacgagaagagtagaggccgaggagatgatggagttacagaacctagttaatttgatcggcgacagtaccgaaaaggacatcgccgactttgcagaattattatcaccagatcaagtgggcagagacattataatcaagatgatgaagtcattcatgcagttcttggacaatgacatttacagcgtgtgctttaccgattggtcaaagtttgaattttacgaagcggacttcggcttcgggaagccggtttggatggcagcaggcccacagagacccattatctctactgcaatattgatgtcagacagggaaggcgacggtatcgaggcttggttacatttgaataagaacgacatgttgattttcgaacaagacgaggaaatcaagttattcactacataa

SEQ ID NO: 21 - protein, 427 aa, synthetic construct
  REGION 1..427: note = Synthetic Construct
MKVERFSRKLIKPHTPTPENLKKYKLSLLDKCLGHDNFAIVLFYESKPRNKSELEESLEKVLVDFYPLAGRHTMNDHIVDCSDVGAVFVEAEALDVELTMDELVKNMEAQTIHHLLPNQYFSADAPNPLLSIQVTHFPSGGLAIGIAVSHAVFDGFSLGVFVAAWSKATMNPDRKIKITPSFDLPSLLPYKDDNFGLTAAEIVSQSEDIVVKRFIFGKEAITRLRSKLSPNRNGKKISRVRVVCAVIVKALMGLERAKHGKTRDFLITQSINMRERTKAPLQKHACGNLAVLSCTRRVEAEEMMELQNLVNLIGDSTEKDIADFAELLSPDQVGRDIIIKMMKSFMQFLDNDIYSVCFTDWSKFEFYEADFGFGKPVWMAAGPQRPIISTAILMSDREGDGIEAWLHLNKNDMLIFEQDEEIKLFTT

SEQ ID NO: 22 - DNA, 1272 bp, synthetic construct
  misc_feature 1..1272: note = CfACT1-8 codon optimized for its expression in S. cerevisiae
  source 1..1272: mol_type = other DNA
  CDS 1..1272
atgaaagtcgaaaggatttcacgtaaattcatcaagccatatacacctacaccacagaaccttaagaagtacaagctatccttgctggataaatgcatgggacacatggacttcgctgtagtattgttttacgaatcaaagccaagaaacaagaatgagctggaagaatcactagagaaagtgttagtcgatttctatccattggcaggcaggtataccatgaacgaccacattgtcgattgcagcgatgagggcgccgttttcgttgaggcagaggcccctaatgttgagcttacagtggaccagttggttaagaacatggaagcccagacaatccacgacttcttaccagaccaatattttcctgctgacgcaccaaatccgttgctaagtattcaagtaacgcacttcccttgtggtggtttagctatcgggattgttgttagtcacgcggtctttgatggattctcattgggcgtattcttagccgcctggagcaaagctaccatgaaccctgagaggaagatcgaaatcaccccttccttcgatttgcctagtcttctgccctacaaggatgaatctttcggtttaaattttagcgaaattgtcaaagctgagaatatcgtagttaaacgtttgaatttcgggaaagaggctattacgcgtttgaggtccaagctgtctcctaaccagaatggtaaaaccatttccagagtgagggtagtctgcgcggtgatagttaaggcgttgatgggactggagagagcaaagactagagactttatgatatgtcaggggatcaacatgagagagaggacgaaggcgcccctgcagaagcatgcgtgtggcaacctagcagtttcgtcttacactagaagggtagcggcagcggaagcagaagaactgcagtccttagtgaatttgatcggggactctatcgaaaagtcaatcgcagactacgctgatatactttcgagtgatcaagatgggagacacatcatttccactatgatgaagagctttatgcagtttgctgcacctgatataaaagccatttcatttaccgactggtcaaagttcggcttttaccaagtagattttggttttggtaaaccagtttggaccggcgtccgtccagaacgtccaatcttctcagccgcgatattgatgagtaacagggaaggcgatggcatcgaggcttggcttcatttggacaagaacgatatgctaatatttgagcaagacgaagaaattaagttgttgataacgtaa

SEQ ID NO: 23 - protein, 423 aa, synthetic construct
  REGION 1..423: note = Synthetic Construct
MKVERISRKFIKPYTPTPQNLKKYKLSLLDKCMGHMDFAVVLFYESKPRNKNELEESLEKVLVDFYPLAGRYTMNDHIVDCSDEGAVFVEAEAPNVELTVDQLVKNMEAQTIHDFLPDQYFPADAPNPLLSIQVTHFPCGGLAIGIVVSHAVFDGFSLGVFLAAWSKATMNPERKIEITPSFDLPSLLPYKDESFGLNFSEIVKAENIVVKRLNFGKEAITRLRSKLSPNQNGKTISRVRVVCAVIVKALMGLERAKTRDFMICQGINMRERTKAPLQKHACGNLAVSSYTRRVAAAEAEELQSLVNLIGDSIEKSIADYADILSSDQDGRHIISTMMKSFMQFAAPDIKAISFTDWSKFGFYQVDFGFGKPVWTGVRPERPIFSAAILMSNREGDGIEAWLHLDKNDMLIFEQDEEIKLLIT

SEQ ID NO: 24 - DNA, 654 bp, synthetic construct
  misc_feature 1..654: note = OAH94415.1 codon optimized for its expression in S. cerevisiae
  source 1..654: mol_type = other DNA
  CDS 1..654
atggataccaagagggtcggttatacagtcgttgatttgagtcagtggggcagaaaagaacacttcgaagcattccagagcttcgcccagtgcaccttttcccagactgttcaattagacataacttccctgctgaagactgtcaagcagaacggatataaattctacccgacctttatctacataataagccgtttagtgaacaagcatgccgaattccgtatggccatgaaggatggtgaactggtaatttgggactccgtaaaccctggttacacaatctttcacgaacagaccgaaacattttcatctctgtggagctattaccacaaggacatcaatcagtttcttaaaacgtattctgaagacatcgctcagtacggggacgatctggcgtacttccccaaggagtttattgaaaatatgttcttcgtgtcagcgaacccatgggtgagcttcacaagtttcaacttaaacgtggccaatattaataacttcttcgcaccggtctttacaatcggtaaatactacacgcagggcgacaaggtgttgatgccgttagctattcaggtccatcatgcagtctgcgacgggtttcatgtcggcaggttattgaacgaaattcagcagtactgcgatgagggatgcaagtaa

SEQ ID NO: 25 - protein, 217 aa, synthetic construct
  REGION 1..217: note = Synthetic Construct
MDTKRVGYTVVDLSQWGRKEHFEAFQSFAQCTFSQTVQLDITSLLKTVKQNGYKFYPTFIYIISRLVNKHAEFRMAMKDGELVIWDSVNPGYTIFHEQTETFSSLWSYYHKDINQFLKTYSEDIAQYGDDLAYFPKEFIENMFFVSANPWVSFTSFNLNVANINNFFAPVFTIGKYYTQGDKVLMPLAIQVHHAVCDGFHVGRLLNEIQQYCDEGCK

SEQ ID NO: 26 - DNA, 1755 bp, Dryopteris fragrans
  source 1..1755: mol_type = genomic DNA
  CDS 1..1755
atggagttctctgcctctgctcctcctcctaggctagccagtgtcataatattggagcctctcggcttcctcctcacaccacactactcctctcagcttcccaaaaagctgctccgtcgcctgttgtgcactagaatctggcacaggtatcagcgaggccgccttcgcctgcgtgacgctgctatgctgctcgcccagctcccattcctagctgtgtctgatcacccctgggctctggacaatctcgcaagcctgctccgccccacagctgtgcgtgcggtgccatggatgctgctgctgctcgacttcctacgagacgagctccatctgaaggtagtctgcgcgaccaactcctccccagaagagctgcaagagctgcgccaccagtttccggccctctttgccaaggtcgatgccaccgtttcttcaggcgaggagggcgtgggcaagccgtccgtgcgcttcctgcaggctgcgttggacaaagccggtgtccacgcgcagcaaaccttgtatcttgactcttttgacagcttggagaccatcatggctgcacgctctcttggcatgcatgcactatctgtagagccatgccacattgatgagctcaccgccagggcctcttccggccagctaagagatgcacagcttataaggcgtattgtgtgcgccatgcacgggccagcagtatctgcagttgtgtcgggcagtatcacatcgtccggcccacagacagcaaagatcgaggaattgccaacagctgctgatagtcatctccgcagcgcagctctcacttctgctcagcagtttttcctcaaagttattgctccacatcgtcctgagaagccattcgtccagcttccatctctcacctcggagggcatccgaatatacgacacctttgcacagtttgtcatagccgacctgctcgacgacacccgcttcctacccatgcaatctcctcctcccaatgggctcatcacctttgttaacccaagcgcgtaccttgctgatgatataaagaatggcaacagccatattgtcccgggtgtgcaattttacgcatccgatgcgtgcactctcatcgacatcccacatgacctagacaccacctccgttggcttgtcagtactgcacaagtttggaaaggtggacaaggacacactcaacaaagtgctagacagaatgctcgagcaagtgagtgaagacgacggcattctgcaggtgtattttgatgtggagcgtccgcgcatcgatccagttgtggtggcaaacacggtgtttctgttccacttgggaaagagagggcatgaggtggcgaggagtgagaagtttgtggagagtgtgctgctgcagagggcatacgaagaagggacgttgtattacaacctgggggaagcatttttggtgagtgtggcgaggctggtgcacgagtttaaggagcactttacaaggagcggcatgaggagggcactggaggagaggctaagagagcgggcaagggcgggcatgcaagagagggatgatgcgctggcgctagccatgcgcattcgtgcatgcgctttgtgtggcctggccggagagggcctcacaaaagcagcagagcaggagcttttgcgcctgcagtgcaagtccaagggctgttgggggtgccaccctttctatcgcaatggcagtaatgtgctcagctggatcggcagtgaggcccttaccactgcttacgctattgctgcgctacagcccattgatatttaa

SEQ ID NO: 27 - protein, 584 aa, Dryopteris fragrans
  REGION 1..584: note = DfHAD_Dryopteris fragrans
MEFSASAPPPRLASVIILEPLGFLLTPHYSSQLPKKLLRRLLCTRIWHRYQRGRLRLRDAAMLLAQLPFLAVSDHPWALDNLASLLRPTAVRAVPWMLLLLDFLRDELHLKVVCATNSSPEELQELRHQFPALFAKVDATVSSGEEGVGKPSVRFLQAALDKAGVHAQQTLYLDSFDSLETIMAARSLGMHALSVEPCHIDELTARASSGQLRDAQLIRRIVCAMHGPAVSAVVSGSITSSGPQTAKIEELPTAADSHLRSAALTSAQQFFLKVIAPHRPEKPFVQLPSLTSEGIRIYDTFAQFVIADLLDDTRFLPMQSPPPNGLITFVNPSAYLADDIKNGNSHIVPGVQFYASDACTLIDIPHDLDTTSVGLSVLHKFGKVDKDTLNKVLDRMLEQVSEDDGILQVYFDVERPRIDPVVVANTVFLFHLGKRGHEVARSEKFVESVLLQRAYEEGTLYYNLGEAFLVSVARLVHEFKEHFTRSGMRRALEERLRERARAGMQERDDALALAMRIRACALCGLAGEGLTKAAEQELLRLQCKSKGCWGCHPFYRNGSNVLSWIGSEALTTAYAIAALQPIDI

SEQ ID NO: 28 - DNA, 1755 bp, Dryopteris fragrans
  source 1..1755: mol_type = genomic DNA
  CDS 1..1755
atggagttctctgcctctgctcctcctcctaggctagccagtgtcataatattggagcctctcggcttcctcctcacaccacactactcctctcagcttcccaaaaagctgctccgtcgcctgttgtgcactagaatctggcacaggtatcagcgaggccgccttcgcctgcgtgacgctgctatgctgctcgcccagctcccattcctagctgtgtctgatcacccctgggctctggacaatctcgcaagcctgctccgccccacagctgtgcgtgcggtgccatggatgctgctgctgctcgacttcctacgagacgagctccatctgaaggtagtctgcgcgaccaactcctccccagaagagctgcaagagctgcgccaccagtttccggccctctttgccaaggtcgatgccaccgtttcttcaggcgaggagggcgtgggcaagccgtccgtgcgcttcctgcaggctgcgttggacaaagccggtgtccacgcgcagcaaaccttgtatcttgactcttttgacagcttggagaccatcatggctgcacgctctcttggcatgcatgcactatctgtagagccatgccacattgatgagctcaccgccagggcctcttccggccagctaagagatgcacagcttataaggcgtattgtgtgcgccatgcacgggccagcagtatctgcagttgtgtcgggcagtatcacatcgtccggcccacagacagcaaagatcgaggaattgccaacagctgctgatagtcatctccgcagcgcagctctcacttctgctcagcagtttttcctcaaagttattgctccacatcgtcctgagaagccattcgtccagcttccatctctcacctcggagggcatccgaatatacgacacctttgcacagtttgtcatagccgacctgctcgacgacacccgcttcctacccatgcaatctcctcctcccaatgggctcatcacctttgttaacccaagcgcgtaccttgctgatgatataaagaatggcaacagccatattgtcccgggtgtgcaattttacgcatccgatgcgtgcactctcatcgacatcccacatgacctagacaccacctccgttggcttgtcagtactgcacaagtttggaaaggtggacaaggacacactcaacaaagtgctagacagaatgctcgagcaagtgagtgaagacgacggcattctgcaggtgtattttgatgtggagcgtccgcgcatcgatccagttgtggtggcaaacacggtgtttctgttccacttgggaaagagagggcatgaggtggcgaggagtgagaagtttgtggagagtgtgctgctgcagagggcatacgaagaagggacgttgtattacaacctgggggaagcatttttggtgagtgtggcgaggctggtgcacgagtttaaggagcactttacaaggagcggcatgaggagggcactggaggagaggctaagagagcgggcaagggcgggcatgcaagagagggatgatgcgctggcgctggccatgcgcattcgtgcatgcgctttgtgtggcctggccggagagggcctcacaagagcagcagagcaggagctactgcgcctgcagtgcaagtccaagggctgttgggggtgccaccctttctatcgcaatggcagtaatgtgctcagctggatcggcagtgaggcccttaccactgcttacgctattgctgcgctacagcccattgatatttaa

SEQ ID NO: 29 - protein, 584 aa, Dryopteris fragrans
  REGION 1..584: note = DfHAD-8(K532R)_Dryopteris fragrans
MEFSASAPPPRLASVIILEPLGFLLTPHYSSQLPKKLLRRLLCTRIWHRYQRGRLRLRDAAMLLAQLPFLAVSDHPWALDNLASLLRPTAVRAVPWMLLLLDFLRDELHLKVVCATNSSPEELQELRHQFPALFAKVDATVSSGEEGVGKPSVRFLQAALDKAGVHAQQTLYLDSFDSLETIMAARSLGMHALSVEPCHIDELTARASSGQLRDAQLIRRIVCAMHGPAVSAVVSGSITSSGPQTAKIEELPTAADSHLRSAALTSAQQFFLKVIAPHRPEKPFVQLPSLTSEGIRIYDTFAQFVIADLLDDTRFLPMQSPPPNGLITFVNPSAYLADDIKNGNSHIVPGVQFYASDACTLIDIPHDLDTTSVGLSVLHKFGKVDKDTLNKVLDRMLEQVSEDDGILQVYFDVERPRIDPVVVANTVFLFHLGKRGHEVARSEKFVESVLLQRAYEEGTLYYNLGEAFLVSVARLVHEFKEHFTRSGMRRALEERLRERARAGMQERDDALALAMRIRACALCGLAGEGLTRAAEQELLRLQCKSKGCWGCHPFYRNGSNVLSWIGSEALTTAYAIAALQPIDI

SEQ ID NO: 30 - DNA, 1755 bp, Dryopteris fragrans
  source 1..1755: mol_type = genomic DNA
  CDS 1..1755
atggagttctctgcctctgctcctcctcctaggctagccagtgtcataatattggagcctctcggcttcctcctcacaccacactactcctctcagcttcccaaaaagctgctccgtcgcctgttgtgcactagaatctggcacaggtatcagcgaggccgccttcgcctgcgtgacgctgctatgctgctcgcccagctcccattcctagctgtgtctgatcacccctgggctctggacaatctcgcaagcctgctccgccccacagctgtgcgtgcggtgccatggatgctgctgctgctcgacttcctacgagacgagctccatctgaaggtagtctgcgcgaccaactcctccccagaagagctgcaagagctgcgccaccagtttccggccctctttgccaaggtcgatgccaccgtttcttcaggcgaggagggcgtgggcaagccgtccgtgcgcttcctgcaggctgcgttggacaaagccggtgtccacgcgcagcaaaccttgtatcttgactcttttgacagcttggagaccatcatggctgcacgctctcttggcatgcatgcactatctgtagagccatgccacattgatgagctcaccgccagggcctcttccggccagctaagagatgcacagcttataaggcgtattgtgtgcgccatgcacgggccagcagtatctgcagttgtgtcgggcagtatcacatcgtccggcccacagacagcaaagatcgaggaattgccaacagctgctgatagtcatctccgcagcgcagctctcacttctgctcagcagtttttcctcaaagctattgctccacatcgtcctgagaagccattcgtccagcttccatctctcacctcggagggcatccgaatatacgacacctttgcacagtttgtcatagccgacctgctcgacgacacccgcttcctacccatgcaatctcctcctcccaatgggctcatcacctttgttaacccaagcgcgtaccttgctgatgatataaagaatggcaacagccatattgtcccgggtgtgcaattttacgcatctgatgcgtgcactctcatcgacatcccacatgacctagacaccacctccgttggcttgtcagtactgcacaagtttggaaaggtggacaaggacacactcaacaaagtgctagacagaatgctggagcaagtgagtgaagacgacggcattctccaggtgtattttgatgtggagcgtccgcgcatcgatccagttgtggtggcaaacacggtgtttctgttccacttgggaaagagagggcatgaggtggcgaggagtgagaagtttgtggagagtgtgctgctgcagagggcatacgaagaagggacgttgtattacaacctgggggaagcatttttggtgagtgtggcgaggctggtgcacgagtttaaggagcactttacaaggagcggcatgaggagggcactggaggagaggctaagagagcgggcaagggcgggcatgcaagagagggatgatgcgctggcgctggccatgcgcattcgtgcatgcgctttgtgtggcctggccggagagggcctcacaaaagcagcagagcaggagctactgcgcctgcagtgcaagtccaagggctgttgggggtgccaccctttctatcgcaatggcagtaatgtgctcagctggatcggcagtgaggcccttaccactgcttacgctattgctgcgctacagcccattgatatttaa

SEQ ID NO: 31 - protein, 584 aa, Dryopteris fragrans
  REGION 1..584: note = DfHAD-9(V274A)_Dryopteris fragrans
MEFSASAPPPRLASVIILEPLGFLLTPHYSSQLPKKLLRRLLCTRIWHRYQRGRLRLRDAAMLLAQLPFLAVSDHPWALDNLASLLRPTAVRAVPWMLLLLDFLRDELHLKVVCATNSSPEELQELRHQFPALFAKVDATVSSGEEGVGKPSVRFLQAALDKAGVHAQQTLYLDSFDSLETIMAARSLGMHALSVEPCHIDELTARASSGQLRDAQLIRRIVCAMHGPAVSAVVSGSITSSGPQTAKIEELPTAADSHLRSAALTSAQQFFLKAIAPHRPEKPFVQLPSLTSEGIRIYDTFAQFVIADLLDDTRFLPMQSPPPNGLITFVNPSAYLADDIKNGNSHIVPGVQFYASDACTLIDIPHDLDTTSVGLSVLHKFGKVDKDTLNKVLDRMLEQVSEDDGILQVYFDVERPRIDPVVVANTVFLFHLGKRGHEVARSEKFVESVLLQRAYEEGTLYYNLGEAFLVSVARLVHEFKEHFTRSGMRRALEERLRERARAGMQERDDALALAMRIRACALCGLAGEGLTKAAEQELLRLQCKSKGCWGCHPFYRNGSNVLSWIGSEALTTAYAIAALQPIDI

SEQ ID NO: 32 - DNA, 1755 bp, synthetic construct
  misc_feature 1..1755: note = Codon-optimized sequence of DfHAD by Genscript genetic codon frequency of E. coli
  source 1..1755: mol_type = other DNA
atggagttcagcgcgagcgctccgccgccgcgtctggcgagcgtgatcattctggaaccgctgggttttctgctgaccccgcactacagcagccagctgccgaagaaactgctgcgtcgtctgctgtgcacccgtatctggcaccgttatcagcgtggccgtctgcgtctgcgtgacgcggcgatgctgctggcgcaactgccgttcctggcggttagcgaccacccgtgggcgctggataacctggcgagcctgctgcgtccgaccgcggttcgtgcggtgccgtggatgctgctgctgctggactttctgcgtgatgagctgcacctgaaagtggtttgcgcgaccaacagcagcccggaggaactgcaggaactgcgtcaccaattcccggcgctgtttgcgaaggttgacgcgaccgtgagcagcggcgaggaaggtgttggcaaaccgagcgtgcgtttcctgcaagcggcgctggataaggcgggcgtgcacgcgcagcaaaccctgtacctggacagctttgatagcctggagaccatcatggcggcgcgtagcctgggtatgcacgcgctgagcgttgagccgtgccacattgacgaactgaccgcgcgtgcgagcagcggtcagctgcgtgatgcgcaactgatccgtcgtattgtttgcgcgatgcacggtccggctgtgagcgcggtggttagcggtagcatcaccagcagcggtccgcagaccgcgaaaattgaggaactgccgaccgcggcggacagccacctgcgtagcgcggcgctgaccagcgcgcagcaattctttctgaaagtgattgcgccgcaccgtccggagaagccgttcgttcaactgccgagcctgaccagcgaaggtatccgtatttatgacaccttcgcgcagtttgtgatcgcggatctgctggacgatacccgtttcctgccgatgcaaagcccgccgccgaacggcctgattacctttgttaacccgagcgcgtacctggcggacgatatcaaaaacggtaacagccacattgttccgggcgtgcagttctatgcgagcgacgcgtgcaccctgatcgatattccgcacgacctggataccaccagcgttggtctgagcgtgctgcacaagtttggcaaagttgacaaggataccctgaacaaggtgctggatcgtatgctggagcaagttagcgaagacgatggtatcctgcaagtttactttgacgtggagcgtccgcgtattgatccggtggttgtggcgaacaccgtgttcctgtttcacctgggtaaacgtggccacgaagttgcgcgtagcgagaagttcgttgaaagcgtgctgctgcagcgtgcgtacgaggaaggcaccctgtactataacctgggcgaagcgtttctggttagcgtggcgcgtctggtgcacgagttcaaagaacactttacccgtagcggtatgcgtcgtgcgctggaggaacgtctgcgtgagcgtgcgcgtgcgggtatgcaagaacgtgacgatgcgctggcgctggcgatgcgtatccgtgcgtgcgcgctgtgcggtctggcgggcgagggtctgaccaaggcggcggagcaggaactgctgcgtctgcaatgcaagagcaaaggttgctggggctgccacccgttctaccgtaacggtagcaacgttctgagctggatcggcagcgaagcgctgaccaccgcgtatgcgattgcggcgctgcagccgatcgacatttaa

SEQ ID NO: 33 - DNA, 1755 bp, synthetic construct
  misc_feature 1..1755: note = Codon-optimized sequence of DfHAD by Genscript genetic codon frequency of tobacco
  source 1..1755: mol_type = other DNA
atggaattttctgcttcagctccacctccaagacttgcttcagttattattcttgagcctttgggatttcttttgactccacattactcttcacaattgcctaagaaacttttgagaaggcttttgtgtacaagaatttggcataggtaccaaaggggtaggcttagattgagggatgctgctatgcttttggctcaacttccatttttggctgtttcagatcatccttgggctcttgataatttggcttctcttttgagaccaactgctgttagggctgttccttggatgcttttgcttttggattttcttagagatgaacttcatttgaaggttgtttgcgctactaattcttcaccagaagagcttcaagagttgaggcatcaatttcctgctttgtttgctaaggttgatgctacagtttcttcaggagaagagggagttggtaaaccatctgttagatttcttcaagctgctttggataaggctggtgttcatgctcaacaaactctttatttggattctttcgattcacttgaaacaattatggctgctaggtcattgggaatgcatgctctttctgttgaaccatgtcatattgatgagttgactgctagagcttcttcaggacaattgagggatgctcaacttattagaaggattgtttgcgctatgcatggtcctgctgtttcagctgttgtttctggatcaattacttcttcaggtccacaaacagctaaaattgaagagcttcctactgctgctgattctcatttgagatcagctgctcttacatctgctcaacaatttttccttaaagttattgctccacatagacctgaaaagccatttgttcaacttccttctttgacttcagagggaatcaggatctatgatacattcgctcaattcgttatcgctgatcttttggatgatactaggtttttgccaatgcaatcacctccacctaatggtcttatcacattcgttaacccttctgcttatttggctgatgatattaaaaatggtaactcacatattgttccaggtgttcaattttacgcttctgatgcttgtactttgattgatattcctcatgatcttgatactacatctgttggactttcagttttgcataagttcggtaaagttgataaggatacacttaataaggttttggatagaatgcttgaacaagtttcagaggatgatggaatccttcaagtttacttcgatgttgaaagacctaggattgatccagttgttgttgctaacactgtttttcttttccatttgggaaaaagaggtcatgaggttgctagatcagaaaagtttgttgagtctgttcttttgcaaagagcttacgaagagggaactttgtattacaatcttggtgaagcttttcttgtttctgttgctagacttgttcatgagtttaaggagcattttacaaggtctggaatgagaagggctttggaagagagacttagggaaagagctagggctggtatgcaagagagagatgatgctcttgctttggctatgagaattagggcttgtgctctttgcggtttggctggagaaggtcttacaaaggctgctgaacaagagcttttgagattgcaatgcaagtctaaaggatgttggggttgccatccattctacaggaatggttctaacgttttgtcatggattggttctgaggctcttactacagcttacgctattgctgctcttcaacctattgatatttga

SEQ ID NO: 34 - DNA, 1755 bp, synthetic construct
  misc_feature 1..1755: note = Codon-optimized sequence of DfHAD-8(K532R) by Genscript genetic codon frequency of E. coli
  source 1..1755: mol_type = other DNA
atggagttcagcgcgagcgctccgccgccgcgtctggcgagcgtgatcattctggaaccgctgggttttctgctgaccccgcactacagcagccagctgccgaagaaactgctgcgtcgtctgctgtgcacccgtatctggcaccgttatcagcgtggccgtctgcgtctgcgtgacgcggcgatgctgctggcgcaactgccgttcctggcggttagcgaccacccgtgggcgctggataacctggcgagcctgctgcgtccgaccgcggttcgtgcggtgccgtggatgctgctgctgctggactttctgcgtgatgagctgcacctgaaagtggtttgcgcgaccaacagcagcccggaggaactgcaggaactgcgtcaccaattcccggcgctgtttgcgaaggttgacgcgaccgtgagcagcggcgaggaaggtgttggcaaaccgagcgtgcgtttcctgcaagcggcgctggataaggcgggcgtgcacgcgcagcaaaccctgtacctggacagctttgatagcctggagaccatcatggcggcgcgtagcctgggtatgcacgcgctgagcgttgagccgtgccacattgacgaactgaccgcgcgtgcgagcagcggtcagctgcgtgatgcgcaactgatccgtcgtattgtttgcgcgatgcacggtccggctgtgagcgcggtggttagcggtagcatcaccagcagcggtccgcagaccgcgaaaattgaggaactgccgaccgcggcggacagccacctgcgtagcgcggcgctgaccagcgcgcagcaattctttctgaaagtgattgcgccgcaccgtccggagaagccgttcgttcaactgccgagcctgaccagcgaaggtatccgtatttatgacaccttcgcgcagtttgtgatcgcggatctgctggacgatacccgtttcctgccgatgcaaagcccgccgccgaacggcctgattacctttgttaacccgagcgcgtacctggcggacgatatcaaaaacggtaacagccacattgttccgggcgtgcagttctatgcgagcgacgcgtgcaccctgatcgatattccgcacgacctggataccaccagcgttggtctgagcgtgctgcacaagtttggcaaagttgacaaggataccctgaacaaggtgctggatcgtatgctggagcaagttagcgaagacgatggtatcctgcaagtttactttgacgtggagcgtccgcgtattgatccggtggttgtggcgaacaccgtgttcctgtttcacctgggtaaacgtggccacgaagttgcgcgtagcgagaagttcgttgaaagcgtgctgctgcagcgtgcgtacgaggaaggcaccctgtactataacctgggcgaagcgtttctggttagcgtggcgcgtctggtgcacgagttcaaagaacactttacccgtagcggtatgcgtcgtgcgctggaggaacgtctgcgtgagcgtgcgcgtgcgggtatgcaagaacgtgacgatgcgctggcgctggcgatgcgtatccgtgcgtgcgcgctgtgcggtctggcgggcgagggtctgacccgggcggcggagcaggaactgctgcgtctgcaatgcaagagcaaaggttgctggggctgccacccgttctaccgtaacggtagcaacgttctgagctggatcggcagcgaagcgctgaccaccgcgtatgcgattgcggcgctgcagccgatcgacatttaa

SEQ ID NO: 35 - DNA, 1755 bp, synthetic construct
  misc_feature 1..1755: note = Codon-optimized sequence of DfHAD-9(V274A) by Genscript genetic codon frequency of E. coli
  source 1..1755: mol_type = other DNA
atggagttcagcgcgagcgctccgccgccgcgtctggcgagcgtgatcattctggaaccgctgggttttctgctgaccccgcactacagcagccagctgccgaagaaactgctgcgtcgtctgctgtgcacccgtatctggcaccgttatcagcgtggccgtctgcgtctgcgtgacgcggcgatgctgctggcgcaactgccgttcctggcggttagcgaccacccgtgggcgctggataacctggcgagcctgctgcgtccgaccgcggttcgtgcggtgccgtggatgctgctgctgctggactttctgcgtgatgagctgcacctgaaagtggtttgcgcgaccaacagcagcccggaggaactgcaggaactgcgtcaccaattcccggcgctgtttgcgaaggttgacgcgaccgtgagcagcggcgaggaaggtgttggcaaaccgagcgtgcgtttcctgcaagcggcgctggataaggcgggcgtgcacgcgcagcaaaccctgtacctggacagctttgatagcctggagaccatcatggcggcgcgtagcctgggtatgcacgcgctgagcgttgagccgtgccacattgacgaactgaccgcgcgtgcgagcagcggtcagctgcgtgatgcgcaactgatccgtcgtattgtttgcgcgatgcacggtccggctgtgagcgcggtggttagcggtagcatcaccagcagcggtccgcagaccgcgaaaattgaggaactgccgaccgcggcggacagccacctgcgtagcgcggcgctgaccagcgcgcagcaattctttctgaaagcgattgcgccgcaccgtccggagaagccgttcgttcaactgccgagcctgaccagcgaaggtatccgtatttatgacaccttcgcgcagtttgtgatcgcggatctgctggacgatacccgtttcctgccgatgcaaagcccgccgccgaacggcctgattacctttgttaacccgagcgcgtacctggcggacgatatcaaaaacggtaacagccacattgttccgggcgtgcagttctatgcgagcgacgcgtgcaccctgatcgatattccgcacgacctggataccaccagcgttggtctgagcgtgctgcacaagtttggcaaagttgacaaggataccctgaacaaggtgctggatcgtatgctggagcaagttagcgaagacgatggtatcctgcaagtttactttgacgtggagcgtccgcgtattgatccggtggttgtggcgaacaccgtgttcctgtttcacctgggtaaacgtggccacgaagttgcgcgtagcgagaagttcgttgaaagcgtgctgctgcagcgtgcgtacgaggaaggcaccctgtactataacctgggcgaagcgtttctggttagcgtggcgcgtctggtgcacgagttcaaagaacactttacccgtagcggtatgcgtcgtgcgctggaggaacgtctgcgtgagcgtgcgcgtgcgggtatgcaagaacgtgacgatgcgctggcgctggcgatgcgtatccgtgcgtgcgcgctgtgcggtctggcgggcgagggtctgaccaaggcggcggagcaggaactgctgcgtctgcaatgcaagagcaaaggttgctggggctgccacccgttctaccgtaacggtagcaacgttctgagctggatcggcagcgaagcgctgaccaccgcgtatgcgattgcggcgctgcagccgatcgacatttaa

SEQ ID NO: 36 - DNA, 2496 bp, synthetic construct
  misc_feature 1..2496: note = Codon-optimized sequence of DfHAD-6His-GST by Genscript genetic codon frequency of E. coli
  source 1..2496: mol_type = other DNA
  CDS 1..2496
atgtctggttctcatcatcatcatcatcatagcagcggtatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgggccacacaggccatagatctgacgacgacgacaagcatatggagttcagcgcgagcgctccgccgccgcgtctggcgagcgtgatcattctggaaccgctgggttttctgctgaccccgcactacagcagccagctgccgaagaaactgctgcgtcgtctgctgtgcacccgtatctggcaccgttatcagcgtggccgtctgcgtctgcgtgacgcggcgatgctgctggcgcaactgccgttcctggcggttagcgaccacccgtgggcgctggataacctggcgagcctgctgcgtccgaccgcggttcgtgcggtgccgtggatgctgctgctgctggactttctgcgtgatgagctgcacctgaaagtggtttgcgcgaccaacagcagcccggaggaactgcaggaactgcgtcaccaattcccggcgctgtttgcgaaggttgacgcgaccgtgagcagcggcgaggaaggtgttggcaaaccgagcgtgcgtttcctgcaagcggcgctggataaggcgggcgtgcacgcgcagcaaaccctgtacctggacagctttgatagcctggagaccatcatggcggcgcgtagcctgggtatgcacgcgctgagcgttgagccgtgccacattgacgaactgaccgcgcgtgcgagcagcggtcagctgcgtgatgcgcaactgatccgtcgtattgtttgcgcgatgcacggtccggctgtgagcgcggtggttagcggtagcatcaccagcagcggtccgcagaccgcgaaaattgaggaactgccgaccgcggcggacagccacctgcgtagcgcggcgctgaccagcgcgcagcaattctttctgaaagtgattgcgccgcaccgtccggagaagccgttcgttcaactgccgagcctgaccagcgaaggtatccgtatttatgacaccttcgcgcagtttgtgatcgcggatctgctggacgatacccgtttcctgccgatgcaaagcccgccgccgaacggcctgattacctttgttaacccgagcgcgtacctggcggacgatatcaaaaacggtaacagccacattgttccgggcgtgcagttctatgcgagcgacgcgtgcaccctgatcgatattccgcacgacctggataccaccagcgttggtctgagcgtgctgcacaagtttggcaaagttgacaaggataccctgaacaaggtgctggatcgtatgctggagcaagttagcgaagacgatggtatcctgcaagtttactttgacgtggagcgtccgcgtattgatccggtggttgtggcgaacaccgtgttcctgtttcacctgggtaaacgtggccacgaagttgcgcgtagcgagaagttcgttgaaagcgtgctgctgcagcgtgcgtacgaggaaggcaccctgtactataacctgggcgaagcgtttctggttagcgtggcgcgtctggtgcacgagttcaaagaacactttacccgtagcggtatgcgtcgtgcgctggaggaacgtctgcgtgagcgtgcgcgtgcgggtatgcaagaacgtgacgatgcgctggcgctggcgatgcgtatccgtgcgtgcgcgctgtgcggtctggcgggcgagggtctgaccaaggcggcggagcaggaactgctgcgtctgcaatgcaagagcaaaggttgctggggctgccacccgttctaccgtaacggtagcaacgttctgagctggatcggcagcgaagcgctgaccaccgcgtatgcgattgcggcgctgcagccgatcgacatttaa

SEQ ID NO: 37 - protein, 831 aa, synthetic construct
  REGION 1..831: note = Synthetic Construct
MSGSHHHHHHSSGMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLGHTGHRSDDDDKHMEFSASAPPPRLASVIILEPLGFLLTPHYSSQLPKKLLRRLLCTRIWHRYQRGRLRLRDAAMLLAQLPFLAVSDHPWALDNLASLLRPTAVRAVPWMLLLLDFLRDELHLKVVCATNSSPEELQELRHQFPALFAKVDATVSSGEEGVGKPSVRFLQAALDKAGVHAQQTLYLDSFDSLETIMAARSLGMHALSVEPCHIDELTARASSGQLRDAQLIRRIVCAMHGPAVSAVVSGSITSSGPQTAKIEELPTAADSHLRSAALTSAQQFFLKVIAPHRPEKPFVQLPSLTSEGIRIYDTFAQFVIADLLDDTRFLPMQSPPPNGLITFVNPSAYLADDIKNGNSHIVPGVQFYASDACTLIDIPHDLDTTSVGLSVLHKFGKVDKDTLNKVLDRMLEQVSEDDGILQVYFDVERPRIDPVVVANTVFLFHLGKRGHEVARSEKFVESVLLQRAYEEGTLYYNLGEAFLVSVARLVHEFKEHFTRSGMRRALEERLRERARAGMQERDDALALAMRIRACALCGLAGEGLTKAAEQELLRLQCKSKGCWGCHPFYRNGSNVLSWIGSEALTTAYAIAALQPIDI

SEQ ID NO: 38 - protein, 526 aa, Cryptoporus volvatus
  REGION 1..526: note = MISC_FEATURE - CvTps1 protein
MTTIHRRHTTLILDLGDVLFRWSPKTETAIPPRQLKEILTSVTWFEYERGQISQTECYERCAAEFKVDPLVIAEAFKQARESLRPNKAFIALIRELRHQMHGDLTVLALSNISLPDYEYIMSLSSDWATVFNRVFPSALVGERKPHLGCYRKVISEMSLEPQTTVFVDDKLDNVASARSLGMHGIVFDNEANVFRQLRNIFGNPVSRGQGYLRKHAGKLESSTDNGLTFEENFTQLIIYEVTQDRSLITLSECPRTWNFFRGQPLFSESFPDDVDTTSVALTVLQPDRALVDSILDQMLEYVDADGIMQTYFDSSRPRIDPFVCVNVLSLFYANGRGRELPHTLEWVYEVLLHRAYHGGSRYYLSPDCFLFFMSRLLKRANDSALQARFRPLFMERVKERVGAAGDSMDLAFRILAAATIGVHCPQDLERLAAAQCEDGGWDMCWFYAFGSTGIKAGNRGLTTALAVAAIRTALGRPPSPSPSNISSSSKLDAPNSFLGIPRPTSPIRFGELFRSWRKNKPTAKSQ

SEQ ID NO: 39 - DNA, 1956 bp, Cryptoporus volvatus
  misc_feature 1..1956: note = CvTps1 transcript (including non-coding sequences)
  source 1..1956: mol_type = genomic DNA
catcccgccttttgagcatggcacacaaacagcctttaaggagctccttggttgcctagtcatgcctccacctgccccctcctcactcatcccctcgcatcctaaaacatgaccacgattcaccgtcggcacaccactctcatcttggacctcggcgacgtcctcttccgctggtcaccaaagaccgagaccgccatcccccctcggcagcttaaggagatacttacctccgtcacctggttcgagtacgaacgaggccagatatcccaaacagaatgttacgaacgatgcgctgcagaattcaaagtcgaccccttagtgatcgctgaagccttcaagcaagctcgcgagtcattacggcccaacaaagcgttcatcgccttgattcgcgaacttcgccatcaaatgcatggagacctcacggtcctcgccctttccaacatttccctccccgattacgaatatatcatgtctctgagctcggattgggcaaccgtcttcaatcgcgtattcccttctgcacttgttggcgagcgaaaaccccatctggggtgctaccgcaaggtcatttcggagatgagcttggaaccccagacaaccgtatttgtcgatgataagctagacaacgtcgcctctgctcgctcacttggcatgcacggcatcgtattcgacaacgaagccaatgtcttccggcaactgcgcaatatcttcgggaatccggttagccgcggtcaaggctatcttcgcaagcatgccggaaagcttgagtcttctaccgacaatggcttgacctttgaggagaacttcacccagctcatcatctacgaggtgacacaagacaggagtctcatcacgctctcagaatgtccccgtacctggaatttctttcgaggtcaaccgctcttctcggagtctttcccggatgatgtggacacaacatccgtggcattgacagtactacaacccgatagagcgctcgttgattctattctagaccaaatgcttgaatatgttgacgccgacggcatcatgcagacatacttcgacagctcgcgaccacgcatagacccttttgtttgcgtcaatgtgctttctctgttctacgcaaacggccggggtcgggagctccctcacacactggagtgggtctatgaagtactcctgcatcgcgcctaccatggaggctcacgttactacctatcaccggactgctttttattcttcatgagccgcttgctcaagcgcgccaacgactcggccctccaggctcggttccgcccactgttcatggagagagtgaaagaacgagtaggggcagccggagactcaatggacctggccttccgcatcctcgccgcggctaccattggcgtccattgcccccaagatctagaaagattggccgccgcgcaatgcgaggacggtggatgggacatgtgctggttctacgcgttcgggtcgacaggtatcaaggcgggcaaccgcggcctcaccacggcccttgccgtcgcagctatacgaaccgccctcgggcgccccccctctcccagcccctccaacatctcgtcgtcgtcgaagctcgacgctcccaacagcttcttgggcatcccgcgcccaaccagccccattcgctttggcgaacttttccgttcctggcgaaagaacaaaccgaccgcaaaatctcaatgaatctcaggttctcgtgctctcgtgctatcttcgtacttatgctactcgacattacccgtcgctgtctacaatgatacgggtactttgatgaaactgtagatgtatttgtatcatattgacctccatccatagtcacctagctactgttcgtgttatcacctgttgctgttatatgatacaagatgcccaaacgagaatgtagaaatgttccgtacacttgtgtacctgtgatgaagctacataggccttcaatcgatcacttggtcc

SEQ ID NO: 40 - DNA, 1581 bp, Cryptoporus volvatus
  misc_feature 1..1581: note = cDNA
  source 1..1581: mol_type = genomic DNA
atgaccacgattcaccgtcggcacaccactctcatcttggacctcggcgacgtcctcttccgctggtcaccaaagaccgagaccgccatcccccctcggcagcttaaggagatacttacctccgtcacctggttcgagtacgaacgaggccagatatcccaaacagaatgttacgaacgatgcgctgcagaattcaaagtcgaccccttagtgatcgctgaagccttcaagcaagctcgcgagtcattacggcccaacaaagcgttcatcgccttgattcgcgaacttcgccatcaaatgcatggagacctcacggtcctcgccctttccaacatttccctccccgattacgaatatatcatgtctctgagctcggattgggcaaccgtcttcaatcgcgtattcccttctgcacttgttggcgagcgaaaaccccatctggggtgctaccgcaaggtcatttcggagatgagcttggaaccccagacaaccgtatttgtcgatgataagctagacaacgtcgcctctgctcgctcacttggcatgcacggcatcgtattcgacaacgaagccaatgtcttccggcaactgcgcaatatcttcgggaatccggttagccgcggtcaaggctatcttcgcaagcatgccggaaagcttgagtcttctaccgacaatggcttgacctttgaggagaacttcacccagctcatcatctacgaggtgacacaagacaggagtctcatcacgctctcagaatgtccccgtacctggaatttctttcgaggtcaaccgctcttctcggagtctttcccggatgatgtggacacaacatccgtggcattgacagtactacaacccgatagagcgctcgttgattctattctagaccaaatgcttgaatatgttgacgccgacggcatcatgcagacatacttcgacagctcgcgaccacgcatagacccttttgtttgcgtcaatgtgctttctctgttctacgcaaacggccggggtcgggagctccctcacacactggagtgggtctatgaagtactcctgcatcgcgcctaccatggaggctcacgttactacctatcaccggactgctttttattcttcatgagccgcttgctcaagcgcgccaacgactcggccctccaggctcggttccgcccactgttcatggagagagtgaaagaacgagtaggggcagccggagactcaatggacctggccttccgcatcctcgccgcggctaccattggcgtccattgcccccaagatctagaaagattggccgccgcgcaatgcgaggacggtggatgggacatgtgctggttctacgcgttcgggtcgacaggtatcaaggcgggcaaccgcggcctcaccacggcccttgccgtcgcagctatacgaaccgccctcgggcgccccccctctcccagcccctccaacatctcgtcgtcgtcgaagctcgacgctcccaacagcttcttgggcatcccgcgcccaaccagccccattcgctttggcgaacttttccgttcctggcgaaagaacaaaccgaccgcaaaatctcaatga

SEQ ID NO: 41 - DNA, 1581 bp, synthetic construct
  misc_feature 1..1581: note = codon optimized cDNA
  source 1..1581: mol_type = other DNA
atgactacgatccaccgccgccatactacgctgatcctggacctgggtgatgttctgttccgctggtccccgaaaaccgaaaccgcaattccgcctcgtcagctgaaagaaatcttgaccagcgttacctggttcgagtatgagcgtggccaaattagccagaccgaatgctacgagcgttgtgctgccgagtttaaagttgatccgctggttattgccgaagcgtttaaacaagcgcgtgaaagcctgcgtccgaacaaagcgtttatcgcgttgatccgtgagttgcgccaccagatgcatggtgacctgacggtcctggcactgagcaacattagcctgcctgattatgagtacattatgtcgctgagctccgattgggcgacggtctttaatcgcgtgtttccgagcgcactggtgggtgagcgtaagccacacctgggttgctaccgcaaggtcatcagcgagatgtctctggagccgcagaccacggttttcgtcgatgacaaactggacaatgtcgcaagcgctcgtagcctgggcatgcatggcatcgtgttcgacaacgaagcgaacgtttttcgtcagctgcgtaatatcttcggtaacccggttagccgcggtcaaggttacttgcgtaaacacgccggtaaactggaatctagcacggataatggtctgaccttcgaagagaacttcactcaattaattatttacgaagtcacgcaagaccgcagcctgatcaccctgagcgagtgcccgcgtacctggaacttcttccgcggtcaaccactgttttctgagagctttccggacgacgtggacaccacctctgtggcgttgaccgttctgcagccggatcgtgcgttggtggatagcatcctggaccagatgttggaatatgttgacgcggatggtattatgcaaacctactttgattcatcccgtccgcgcattgacccgttcgtgtgcgtgaatgtcctgagcctgttctacgccaatggcagaggccgcgagctgccacacacgctggaatgggtctatgaagttctgctgcaccgtgcgtaccacggcggtagccgttattacctgagcccggactgtttcctgttctttatgagccgtctgctgaagcgcgcgaatgactcggcgctgcaggcccgttttcgcccgcttttcatggaacgtgtgaaagagcgtgtgggcgcagccggcgatagcatggacctggcgttccgcattctggccgctgcaaccatcggcgttcattgcccacaagatctggagcgtctggcagcagcgcagtgcgaagatggtggctgggatatgtgttggttttatgcgtttggcagcacgggtatcaaggctggcaaccgcggtctgaccaccgcgttggctgtcgccgcaattcgtaccgcgctgggtcgtccgccttccccgagcccgagcaatatttctagctccagcaaactggacgcgccgaactccttcctgggcatcccgcgtccgaccagcccgatccgtttcggtgaactgtttcgtagctggcgtaagaacaagccgaccgcgaaaagccagtaa

SEQ ID NO: 42 - protein, 521 aa, Laricifomes officinalis
  REGION 1..521: note = MISC_FEATURE - LoTps1 protein
MYTALILDLGDVLFSWSSTTNTTIPPRQLKEILSSPAWFEYERGRITQAECYERVSAEFSLDATAVAEAFRQARDSLRPNDKFLTLIRELRQQSHGELTVLALSNISLPDYEFIMALDSKWTSVFDRVFPSALVGERKPHLGAFRQVLSEMNLDPHTTVFVDDKLDNVVSARSLGMHGVVFDSQDNVFRMLRNIFGDPIHRGRDYLRQHAGRLETSTDAGVVFEENFTQLIIYELTNDKSLITTSNCARTWNFFRGKPLFSASFPDDMDTTSVALTVLRLDHALVNSVLDEMLKYVDADGIMQTYFDHTRPRMDPFVCVNVLSLFHEQGRGHELPNTLEWVHEVLLHRAYIGGSRYYLSADCFLFFMSRLLQRITDPSVLGRFRPLFIERVRERVGATGDSIDLAFRIIAASTVGIQCPRDLESLLAAQCEDGGWDLCWFYQYGSTGVKAGNRGLTTALAIKAIDSAIARPPSPALSVASSSKSEIPKPIQRSLRPLSPRRFGGFLMPWRRSQRNGVAVSS

SEQ ID NO: 43 - DNA, 2291 bp, Laricifomes officinalis
  misc_feature 1..2291: note = LoTps1 transcript (including non-coding sequence)
  source 1..2291: mol_type = genomic DNA
gcgtctgctgcggtctctcaccgcgccgagcgacgggaagcggaggctttttgatgcagccagctcagcgccatcctctcacgcagggggtttgatccagatctgatcgcctccgggttctcatctagaacgcacggcggctcccaggaagttctatcgaccctctgcgcgctggtcggcggcacgatgtggctacaccagtcccaatcatatctcacacccagcaccatcatctcgggcctcttcgtcatgtaaccctcccaagcctatttttcagggcgttccccctcaccggcgcgcttcttaaagaatcccgaaatgtatacggctcttatccttgacctcggcgacgttctgttctcttggtcgtcgacgaccaacacgactattccccctcggcagctaaaggagatcctctcatctcctgcctggtttgagtacgagcgtggtcgcataacgcaagccgaatgctacgagcgtgtcagcgccgagttcagcctagacgccaccgccgtcgcggaagcattccggcaagctcgcgactccttgcgcccgaacgacaagttcctcacgttaattcgcgagcttcgacaacaatctcatggggagctcacggtgcttgcgctgtccaacatatcccttcccgactatgaattcatcatggccctcgactcgaagtggacttctgtctttgaccgcgtcttcccttctgccctcgtgggcgaacggaagccacaccttggagcgtttcgccaggttctgtccgagatgaatcttgacccgcacacaactgtgttcgtcgatgacaagctggacaatgtcgtctccgcacggtccctcgggatgcacggcgtcgtgttcgactcccaagacaatgtctttcggatgctgagaaacatctttggcgatcccattcatcggggacgtgactatctccgacagcacgccggacgtctggagacctccacggatgccggtgtggtcttcgaagagaatttcacgcaactcatcatctacgaactgacgaatgacaagtctctcatcacgacatcaaactgtgctcgtacttggaatttctttcgtgggaagcctttgttctcagcatcgttccctgacgacatggacacgacctcggttgccttgactgtattacgtttagaccacgccctcgtgaactcggttttggacgagatgctaaagtatgtcgacgcagacggcatcatgcagacctacttcgaccatacacgcccacgcatggatccatttgtctgcgtcaatgtgctctcgttgtttcacgaacaaggtcgtggccacgagcttccgaacaccctcgaatgggtccatgaggtcctcctccaccgcgcgtacatcgggggctcgcggtactacctctccgcggactgcttcctctttttcatgagccgcctcctgcagcgcatcaccgacccgtccgtccttggccgcttccgtccactattcatagagcgcgttcgggagcgtgtaggtgcgaccggggactccatcgatctcgcattccgcatcatcgccgcgtccacagtaggcatccagtgtccacgcgacttggaaagtctcctcgccgcacagtgtgaagacggtggctgggacctgtgctggttctaccagtacggatcgaccggtgtcaaggcgggcaaccgcgggctcaccaccgctctggcgatcaaagctattgactccgccattgcgaggccaccttcgcctgccctctcagtcgcttcgtcgtccaaatcggagataccgaaacccatacaacggtcccttaggccccttagcccccgccggtttggcggtttcctgatgccgtggcgcaggtcacagcgcaatggcgtggcggtctctagttgaacacttgacccttgacacttcgctttgcactgcctgctcccctgccaatcctcccctacgatcgtatcatccctctcttgccctcgcctccccctcgtaccccctctcatggggtgccatttgtagatatgtacgtagcgtgatgtagcggtactcggatcgttctcgtactcgtcttgctctgccgtcgcttccagcccgtgctgttctctcgttcaggctattcgttggttacgcgtatatcgtaatagaccgccccggttcctcgcctacagacactcgcccgtctcgccacggactcggctacggattcagactacatgagtggcagttatcacacgcagatccctccttggtcgttctgtagtacccacatatgtaattgtaccagtccactgttgcagatc

SEQ ID NO: 44 - DNA, 1566 bp, Laricifomes officinalis
  misc_feature 1..1566: note = cDNA
  source 1..1566: mol_type = genomic DNA
atgtatacggctcttatccttgacctcggcgacgttctgttctcttggtcgtcgacgaccaacacgactattccccctcggcagctaaaggagatcctctcatctcctgcctggtttgagtacgagcgtggtcgcataacgcaagccgaatgctacgagcgtgtcagcgccgagttcagcctagacgccaccgccgtcgcggaagcattccggcaagctcgcgactccttgcgcccgaacgacaagttcctcacgttaattcgcgagcttcgacaacaatctcatggggagctcacggtgcttgcgctgtccaacatatcccttcccgactatgaattcatcatggccctcgactcgaagtggacttctgtctttgaccgcgtcttcccttctgccctcgtgggcgaacggaagccacaccttggagcgtttcgccaggttctgtccgagatgaatcttgacccgcacacaactgtgttcgtcgatgacaagctggacaatgtcgtctccgcacggtccctcgggatgcacggcgtcgtgttcgactcccaagacaatgtctttcggatgctgagaaacatctttggcgatcccattcatcggggacgtgactatctccgacagcacgccggacgtctggagacctccacggatgccggtgtggtcttcgaagagaatttcacgcaactcatcatctacgaactgacgaatgacaagtctctcatcacgacatcaaactgtgctcgtacttggaatttctttcgtgggaagcctttgttctcagcatcgttccctgacgacatggacacgacctcggttgccttgactgtattacgtttagaccacgccctcgtgaactcggttttggacgagatgctaaagtatgtcgacgcagacggcatcatgcagacctacttcgaccatacacgcccacgcatggatccatttgtctgcgtcaatgtgctctcgttgtttcacgaacaaggtcgtggccacgagcttccgaacaccctcgaatgggtccatgaggtcctcctccaccgcgcgtacatcgggggctcgcggtactacctctccgcggactgcttcctctttttcatgagccgcctcctgcagcgcatcaccgacccgtccgtccttggccgcttccgtccactattcatagagcgcgttcgggagcgtgtaggtgcgaccggggactccatcgatctcgcattccgcatcatcgccgcgtccacagtaggcatccagtgtccacgcgacttggaaagtctcctcgccgcacagtgtgaagacggtggctgggacctgtgctggttctaccagtacggatcgaccggtgtcaaggcgggcaaccgcgggctcaccaccgctctggcgatcaaagctattgactccgccattgcgaggccaccttcgcctgccctctcagtcgcttcgtcgtccaaatcggagataccgaaacccatacaacggtcccttaggccccttagcccccgccggtttggcggtttcctgatgccgtggcgcaggtcacagcgcaatggcgtggcggtctctagttga

SEQ ID NO: 45 - DNA, 1566 bp, synthetic construct
  misc_feature 1..1566: note = codon optimized cDNA
  source 1..1566: mol_type = other DNA
atgtacacggcgctgattttggatttgggtgatgttctgtttagctggagctcaacgactaacaccaccattccgccgcgtcagctgaaagaaatcttgagctccccggcgtggttcgagtacgagcgtggccgtatcacccaggcagagtgttatgagcgtgtcagcgcagagtttagcctggatgcgacggccgtggctgaggcttttcgtcaggcacgtgatagcctgcgtccgaacgacaaatttctgaccctgatccgtgagctgcgtcaacagagccacggtgaattgaccgttctggccttgtctaacatcagcctgccggattacgaatttattatggcactggactcgaagtggaccagcgtgtttgatcgtgtgttcccgagcgccctggtgggcgaacgcaagccgcacctgggcgcgttccgccaagtcctgtccgagatgaatttggacccgcataccaccgtttttgtggacgacaaactggacaatgttgtcagcgcacgcagcctgggtatgcacggtgtcgtgttcgacagccaagacaatgtttttcgtatgctgcgtaacattttcggtgacccaattcaccgcggtcgtgactatctgcgccagcacgctggtcgtcttgaaacgtccaccgatgcgggcgttgtgttcgaagagaacttcacccaactgatcatttacgaactgaccaacgataagagcctgatcaccacctctaattgcgcccgcacctggaacttcttccgcggcaaacctctgttctccgcgagctttccggacgatatggacactacgtcggtagcgctgaccgtgctgcgtctggaccatgcgctggtgaatagcgttctggatgaaatgctgaaatacgtcgatgctgacggtattatgcagacctactttgatcatacgcgtcctcgtatggacccgttcgtttgcgtcaatgtgctgagcctgtttcacgagcaaggtcgcggtcatgaactgccgaatacgctggaatgggtgcatgaagtcctgctgcaccgtgcgtatatcggtggcagccgctattatctgagcgcggattgtttcctgttctttatgagccgtctgttgcaacgtattaccgacccgagcgttttaggtagatttcgcccgctgttcatcgagcgtgttcgcgagcgcgttggcgcgactggcgacagcatcgacctggcattccgtatcatcgcggccagcacggtcggcattcaatgcccgcgtgacctggagtctctgctggcagcacagtgcgaagatggtggctgggatctgtgttggttttaccagtacggcagcacgggtgttaaggccggtaaccgtggtctgaccacggcgttggcgatcaaagcgattgacagcgccatcgcgcgtccgccaagcccggccctgtccgttgcaagctccagcaagagcgagattccgaagccgattcagcgtagcctccgcccgttgagcccgcgtcgcttcggtggcttcctgatgccgtggcgtcgtagccaacgcaatggtgtcgcggtgagctcttaa

SEQ ID NO: 46 - protein, 527 aa, Obba rivulosa
  REGION 1..527: note = MISC_FEATURE - OCH93767.1 protein
MSAAVRYTTLILDLGDVLFTWSPKTKTSISPRILKEILNSATWYEYERGSITQHECYERVGVEFGIAPSEIHNAFKQARDSMESNDELIALVRELKEQSDGELLVFALSNISLPDYEYVLTKPADWSIFDKVFPSALVGERKPHLGIYKHVIAETGVDPRTTVFVDDKIDNVLSARSLGMHGIVFDKHEDVMRALRNIFGDPVRRGREYLRRNARKLESITDHGVAFGENFTQLLILELTSDASLVTLPDRPRTWNFFRGKPLFSEAFPDDLDTTSLALTVLKRDAATVSSVMDEMLKYRDADGIMQTYFDNGRQRLDPFVNANVLTLFYANGRGHELDQSLSWVREVLLYRAYLGGSRYYPSADCFLYFISRLFACTSDPVLHHQLKPLFVERVHERIGVQGDALELAFRLLVCASFNISNQPDMRKLLEMQCQDGGWDGGNLYRFGTTGLKVTNRGLTTAAAVQAIEATQLRPPSPAFSVESPKSPVTPVTPMLEIPALGLSISRPSSPLLGYFKLPWKKSAEVH

SEQ ID NO: 47 - DNA, 1584 bp, Obba rivulosa
  misc_feature 1..1584: note = cDNA
  source 1..1584: mol_type = genomic DNA
atgtccgcagcagttcggtacacgaccctcatcctcgaccttggcgacgtcttgttcacttggtcaccgaagacgaagaccagcatctcgcctcgtattctgaaggagatcctgaattccgcgacctggtatgagtacgagcgcggtagtatcactcagcacgaatgttacgaacgcgttggcgtggagttcggtattgcgccgagcgagatccacaacgcgttcaagcaggctcgggactctatggagtcgaatgacgagctgatcgcccttgttcgggaactgaaggagcagtcagatggagagcttctcgtcttcgcattatcgaacatctcactgccggactacgaatacgtcctgacgaagcccgcggactggtccatcttcgacaaagtctttccttccgctctcgtcggcgagcgcaagccccatctcggcatctacaaacacgtcatcgcagagacgggcgttgatccgcgaacaaccgtcttcgtggacgacaagatcgacaatgtgctttcggcgcggtcgctcggtatgcacggcattgtcttcgacaaacacgaagacgtaatgcgcgctctgcgaaacattttcggtgaccccgtgcgaagaggacgagaatatttgcgtcgaaatgcaaggaaattggaatccatcacagatcacggcgtcgccttcggggagaacttcacccagcttctgatcctcgaacttactagtgatgcgtccctcgttactctccctgatcgtcctcggacatggaattttttccgagggaagccgctcttttcggaggccttccccgatgaccttgatactacttccttggcactcactgtcctgaaaagagatgccgccactgtatcgtccgtgatggacgagatgctgaaatacagggacgcggacggcatcatgcagacatacttcgacaacggtcggcaacgactcgatccgttcgtcaacgccaacgttttgaccctcttctacgccaacggtcgcggacacgagctggatcagagcctcagctgggttcgcgaagtcttgctctaccgcgcttacctcggcggttcccgctactacccctccgccgactgcttcctatatttcatcagccgcctcttcgcctgcaccagcgacccggtcctccatcatcaacttaagcccctctttgttgagcgtgtgcacgagcggataggagtgcagggcgacgcgctggagctcgccttccgcctgcttgtatgcgcgagcttcaacatctcgaaccagcctgacatgcgcaagctgctcgagatgcagtgccaggacggaggctgggatggcggaaacctgtatcgtttcggcaccacgggcctcaaggtcacgaaccggggtctgaccaccgcagcagccgtgcaagccatcgaggcgacgcagctgcgtccaccatcaccggcgttctctgtcgagtcgcctaagagcccggtgacgccggtgacgcccatgctggagattccagcgctgggtctcagcatctcgcggccctccagtcctctgttggggtatttcaagctcccgtggaagaagtcagccgaggttcattga

SEQ ID NO: 48 - DNA, 1584 bp, synthetic construct
  misc_feature 1..1584: note = codon optimized cDNA
  source 1..1584: mol_type = other DNA
atgtctgcagctgttcgttatactactctgatcctggatttgggcgatgttctgttcacctggtccccgaaaaccaagacctctatcagcccacgtatcctgaaagaaatcctgaacagcgcgacctggtacgagtatgagcgtggcagcatcacccagcacgagtgctacgagcgtgttggcgtcgaatttggtattgcgccgagcgagattcacaacgcgttcaaacaagcccgcgacagcatggaatccaacgacgaactgattgctctggtgcgtgagctgaaagaacagagcgatggtgagctgctggtctttgccctgagcaatatctctctgccggattacgaatacgttctgaccaaaccagcggactggtcaatcttcgataaagtctttccgagcgctttggtcggtgagcgtaaaccgcatctgggtatttacaaacacgttattgcggaaaccggtgttgacccgagaacgaccgtttttgttgacgataagattgacaacgtcctgagcgcacgcagcctgggtatgcatggtattgtctttgataaacacgaagatgtgatgcgtgctctgcgcaatatctttggcgacccggtgcgtcgcggtcgtgagtatttgcgccgcaacgcgcgcaaattggagtccattaccgatcatggtgtcgcatttggtgagaatttcacccagctcctgattctggaactgaccagcgacgcgtccctggtgacgctgccggatcgtccgcgtacgtggaacttcttccgcggcaagccgctgtttagcgaagcgttcccggatgacctggacaccacgagcctggcactgacggtgctgaaacgcgatgcagcaactgtgagctccgtcatggacgaaatgctgaagtaccgcgacgcggatggcatcatgcagacgtatttcgacaacggtcgtcagcgtctggacccgtttgtcaacgccaatgttctgacgctgttttacgcgaatggccgtggtcatgaactggaccagagcttatcatgggtgcgtgaagtgctgctgtatcgcgcctatctgggtggcagccgctactatccgagcgcggactgttttctgtacttcattagccgcttgttcgcctgcaccagcgatccggttctgcatcaccaactgaagccattgttcgtcgagcgtgtgcacgagcgtattggtgttcagggcgacgcactggaactggcgttccgtctgttggtgtgtgcgagcttcaacattagcaatcagccggatatgcgtaagctgctggaaatgcaatgccaagatggcggctgggacggtggtaatctgtaccgttttggcaccaccggtttaaaagtgacgaatcgtggtttgaccaccgctgcggccgttcaagcaattgaagcaacgcaactgcgtccgccgagcccagcatttagcgtagagtcgcctaagagcccggttacgccggtgacgccgatgctggaaatcccggcgctgggtctgtctatcagccgtccgtcgagcccgctgctgggctatttcaagttgccgtggaagaaaagcgccgaagtgcactaa

SEQ ID NO: 49 - protein, 533 aa, Gelatoporia subvermispora
  REGION 1..533: note = MISC_FEATURE - EMD37666.1 protein
MSAAAQYTTLILDLGDVLFTWSPKTKTSIPPRTLKEILNSATWYEYERGRISQDECYERVGTEFGIAPSEIDNAFKQARDSMESNDELIALVRELKTQLDGELLVFALSNISLPDYEYVLTKPADWSIFDKVFPSALVGERKPHLGVYKHVIAETGIDPRTTVFVDDKIDNVLSARSVGMHGIVFEKQEDVMRALRNIFGDPVRRGREYLRRNAMRLESVTDHGVAFGENFTQLLILELTNDPSLVTLPDRPRTWNFFRGNGGRPSKPLFSEAFPDDLDTTSLALTVLQRDPGVISSVMDEMLNYRDPDGIMQTYFDDGRQRLDPFVNVNVLTFFYTNGRGHELDQCLTWVREVLLYRAYLGGSRYYPSADCFLYFISRLFACTNDPVLHHQLKPLFVERVQEQIGVEGDALELAFRLLVCASLDVQNAIDMRRLLEMQCEDGGWEGGNLYRFGTTGLKVTNRGLTTAAAVQAIEASQRRPPSPSPSVESTKSPITPVTPMLEVPSLGLSISRPSSPLLGYFRLPWKKSAEVH

SEQ ID NO: 50 - DNA, 1602 bp, Gelatoporia subvermispora
  misc_feature 1..1602: note = cDNA
  source 1..1602: mol_type = genomic DNA
atgtccgcggcagctcaatacacgaccctcattctcgaccttggcgacgtcctgttcacctggtcaccgaaaaccaagacgagcatcccccctcggactctgaaggagattctcaattccgcgacatggtatgagtatgagcgcggccgcatctctcaggacgaatgttacgaacgcgttggcacggagttcggaatcgcgcctagcgaaatcgacaacgcgttcaagcaagctcgggattccatggaatccaacgacgaactgatcgcccttgttcgggaactcaagacgcagttggacggcgaactccttgtcttcgcactctcaaatatctcgttgcctgactacgagtacgtcctcacgaaaccggccgactggtccatcttcgacaaggtcttcccttccgccctcgtgggcgagcgcaagccgcacctcggcgtttacaagcacgtcattgcagaaacgggcattgatccgcgaaccaccgttttcgtggacgacaagatcgacaacgtgctctcagcgcggtctgtaggtatgcatgggatcgttttcgagaagcaggaagacgtaatgcgcgctctccgaaacatcttcggagacccggttcggcgagggcgcgagtacttgcgccgtaatgccatgaggcttgaatcggttacagaccatggtgtggcgtttggcgagaacttcacacaactccttatcctcgaactaacgaacgatccctccctcgttacgctccctgatcgtcctcgaacatggaatttcttccgaggtaacgggggacgaccaagcaaaccattattctcggaggccttccccgatgacttggacactacttcactagcgttgactgtcctccaaagagatcccggcgtcatctcttctgtgatggacgaaatgttgaactacagggatccggacggcattatgcagacatacttcgacgatggtcggcaaagactcgatccatttgtcaatgtcaatgtcttaacgttcttctacaccaacggacgtggtcatgaactggaccaatgccttacatgggtccgcgaagttttgctctatcgcgcctatctcggcggctcacgttattacccctccgccgactgctttctctacttcatcagccgccttttcgcatgcacgaatgaccccgtgctacaccaccaactcaaaccgctcttcgtcgagcgcgtgcaggagcaaatcggcgtggagggcgatgcgctcgagttggcgttccgattgctcgtctgtgcaagcctggacgtccaaaacgcgatcgacatgcgcaggctgctcgagatgcaatgcgaagatggcggctgggagggcgggaacctttataggtttggcacgaccgggctcaaggtgactaaccggggcctgacgactgcagcggccgtacaggccatcgaggcgtcccaacggcgcccaccatcaccgtccccctccgtcgaatctacaaagagcccaataacccctgtgacgcccatgctggaggtcccctcgctcggcctgagcatctcgaggccgtccagccctttactcggctacttcaggctcccgtggaagaagtcggccgaagtacactga

SEQ ID NO: 51 - DNA, 1602 bp, synthetic construct
  misc_feature 1..1602: note = codon optimized cDNA
  source 1..1602: mol_type = other DNA
atgtctgcggcggctcaatacacgactttgattctggatctgggtgatgttctgttcacttggtccccgaaaaccaagaccagcatccctccgcgtaccctgaaagaaatcctgaatagcgctacctggtatgagtacgagcgtggtcgcatttcccaagacgagtgttacgaacgtgtgggcaccgagttcggcattgcgccgagcgagattgacaacgcgttcaaacaagcgcgcgattcgatggaaagcaatgatgaactgatcgcactggtccgtgagctgaaaacgcagctggacggtgagctgctggttttcgcactgtccaatattagcctgccggattacgaatacgtcttgaccaaaccggcggactggagcatctttgacaaagtgttccctagcgccttggtgggcgagcgtaagccgcatctgggcgtttataaacacgttattgcggaaacgggcattgatccgcgcacgacggttttcgtggacgacaagattgacaatgtgttaagcgcacgcagcgtcggtatgcatggtatcgtgtttgagaaacaagaagatgtcatgcgtgcactgcgtaacatctttggtgatccggtccgtcgtggtcgtgagtatctgcgtagaaacgcaatgcgtctggagtccgtgaccgaccacggcgtggcgtttggtgagaactttacccagttgctgattctggaattgacgaacgacccgagcctggtcaccctgcctgatcgtccgcgtacctggaacttttttcgcggcaatggtggccgcccgagcaagccgctgttcagcgaagcgttcccggatgatctggataccacgagcctggcgctgaccgtgctgcagcgcgacccgggtgttatcagcagcgttatggacgaaatgctgaattaccgtgacccggacggtatcatgcagacttatttcgatgacggtcgccaacgcttggacccatttgtgaacgtcaatgttctgacctttttctatacgaacggccgtggtcacgaactggaccagtgtctgacgtgggtgcgtgaagtcctcttgtatcgtgcgtaccttggtggctcacgctactacccatcggcggattgcttcctgtacttcatctctcgtctgtttgcgtgtaccaatgacccggtgctgcaccatcagctgaagccactgtttgttgagcgtgtccaagagcaaattggtgtcgagggtgatgcactggaactggcttttcgtctgctggtctgcgccagcctggatgtccagaatgccatcgacatgcgccgtctgctggaaatgcagtgcgaagatggcggttgggagggtggtaacctctaccgcttcggcaccacgggcctgaaagttaccaaccgcggtctgacgaccgcagccgccgttcaagcgatcgaagcgagccaacgccgtccgccgagcccgagcccgtctgtagagagcacgaaaagcccgattaccccggtgaccccgatgctggaagttccaagcctgggcttatctatcagccgtccgtccagcccgctgctgggttatttccgtttgccgtggaagaaaagcgcagaagtgcactaa

SEQ ID NO: 52 - protein, 527 aa, Gelatoporia subvermispora
  REGION 1..527: note = MISC_FEATURE - EMD37666-B protein
MSAAAQYTTLILDLGDVLFTWSPKTKTSIPPRTLKEILNSATWYEYERGRISQDECYERVGTEFGIAPSEIDNAFKQARDSMESNDELIALVRELKTQLDGELLVFALSNISLPDYEYVLTKPADWSIFDKVFPSALVGERKPHLGVYKHVIAETGIDPRTTVFVDDKIDNVLSARSVGMHGIVFEKQEDVMRALRNIFGDPVRRGREYLRRNAMRLESVTDHGVAFGENFTQLLILELTNDPSLVTLPDRPRTWNFFRGKPLFSEAFPDDLDTTSLALTVLQRDPGVISSVMDEMLNYRDPDGIMQTYFDDGRQRLDPFVNVNVLTFFYTNGRGHELDQCLTWVREVLLYRAYLGGSRYYPSADCFLYFISRLFACTNDPVLHHQLKPLFVERVQEQIGVEGDALELAFRLLVCASLDVQNAIDMRRLLEMQCEDGGWEGGNLYRFGTTGLKVTNRGLTTAAAVQAIEASQRRPPSPSPSVESTKSPITPVTPMLEVPSLGLSISRPSSPLLGYFRLPWKKSAEVH

SEQ ID NO: 53 - DNA, 1584 bp, synthetic construct
  misc_feature 1..1584: note = codon optimized cDNA
  source 1..1584: mol_type = other DNA
atgtctgcggctgctcaatatactactttgattctggatctgggcgacgttctgttcacgtggagcccgaaaaccaagaccagcattccaccgcgtaccctgaaggagatcctcaatagcgcgacttggtacgagtatgagcgtggccgcatcagccaagacgagtgctacgaacgcgtcggtacggaatttggcattgcaccaagcgagattgacaatgcgtttaaacaagcgcgtgacagcatggaaagcaatgacgaactgatcgcgctggtccgtgagctgaaaacccagctggatggtgagctgttggtgtttgcgctgtcgaacatctctctgccggactacgagtatgttctgaccaaaccggcggattggagcatttttgataaagtgtttccgagcgcgctggttggtgagcgcaagccgcacctgggtgtgtacaaacacgttattgcagagactggcatcgacccgcgtacgacggttttcgttgacgacaagatcgataacgttctgagcgcacgtagcgtcggtatgcacggtattgttttcgaaaaacaagaagatgttatgcgcgcactgcgtaatatcttcggcgatccggtcagacgtggccgtgagtatctgcgccgcaatgcgatgcgtctggaatcggtgaccgatcatggtgtcgcctttggcgagaatttcacccagctgctgattttagagctgaccaatgatcctagcctggtgacgctgccggatcgtccgcgtacctggaactttttccgcggcaagccgttgttctccgaagccttcccggacgacctggacacgaccagcctggcgctgaccgtgctgcaacgtgatccgggtgtgatctcttccgtaatggacgaaatgctgaactaccgtgacccggacggtatcatgcagacctattttgacgacggtcgtcagcgtctggacccgtttgtgaacgtgaatgtcctgacgttcttttacaccaatggtcgcggtcacgaactggatcagtgtctgacctgggtccgcgaagtgctgctgtatcgtgcatacctgggtggcagccgttattacccgagcgccgattgctttctgtactttatcagccgtctgttcgcgtgcacgaacgatccggttctgcatcaccagctgaagccgttatttgttgagcgcgttcaggaacaaattggtgtcgagggtgatgcgctggaattggcattccgcctgttggtctgcgccagccttgatgtccagaacgccattgacatgcgtcgcttgctcgaaatgcagtgtgaggacggcggttgggagggtggcaacctgtaccgtttcggtacgaccggcctgaaagtcacgaaccgtggtctgacgacggcagctgcggtgcaagcaattgaagccagccaacgtcgtccgccatccccgtcaccgagcgttgagtccaccaagagcccgattacccctgtgaccccgatgcttgaagttccgagcctgggtctgagcatctcccgtcctagcagcccgctgttgggttacttccgcctgccgtggaagaaaagcgctgaggtgcattaa

SEQ ID NO: 54 - protein, 486 aa, Aspergillus terreus
  REGION 1..486: note = MISC_FEATURE - Protein
MAITKGPVKALILDFSNVLCSWKPPSNVAVPPQILKMIMSSDIWHDYECGRYSREDCYARVADRFHISAADMEDTLKQARKSLQVHHETLLFIQQVKKDAGGELMVCGMTNTPRPEQDVMHSINAEYPVFDRIYISGLMGMRKPSICFYQRVMEEIGLSGDAIMFIDDKLENVIAAQSVGIRGVLFQSQQDLRRVVLNFLGDPVHRGLQFLAANAKKMDSVTNTGDTIQDNFAQLLILELAQDRELVKLQAGKRTWNYFIGPPKLTTATFPDDMDTTSMALSVLPVAEDVVSSVLDEMLKFVTDDGIFMTYFDSSRPRVDPVVCINVLGVFCRHNRERDVLPTFHWIRDILINRAYLSGTRYYPSPDLFLFFLARLCLAVRNQSLREQLVLPLVDRLRERVGAPGEAVSLAARILACRSFGIDSARDMDSLRGKQCEDGGWPVEWVYRFASFGLNVGNRGLATAFAVRALESPYGESAVKVMRRIV

SEQ ID NO: 55 - DNA, 1461 bp, Aspergillus terreus
  misc_feature 1..1461: note = cDNA
  source 1..1461: mol_type = genomic DNA
atggctatcaccaagggtccagttaaggcgcttattcttgacttttccaatgttctctgctcgtggaagcctcccagcaatgttgcggtgccgccccagatactcaaaatgatcatgtcctctgacatatggcatgactacgagtgcggacggtactcgagagaggactgctatgccagagtggcagaccgttttcatatcagcgccgcggacatggaagacacgctgaaacaggcgcgcaagagcctgcaggttcaccatgagacactgttgtttatccagcaagtcaagaaggatgccgggggcgagttgatggtgtgtgggatgaccaacacgccccggccagagcaagacgtaatgcattcaatcaacgcggagtatcctgtgtttgataggatatatatatccggtctcatgggcatgaggaagccgagcatctgcttctaccagcgggtgatggaggagattggcctatcaggcgatgcgatcatgtttatagatgacaagttggagaatgtcatcgccgcccagtcggtagggatccgaggcgttctatttcagagtcagcaagatctccgtcgggttgtattaaatttcttgggcgatccggtccatcgcggcctgcagttcctagcggccaatgcgaaaaagatggatagtgtgaccaacaccggcgatactatccaagataattttgctcagctcctcatcttggagctggcccaggacagggaattggtgaagcttcaggctggaaaaaggacttggaattacttcatagggcctcccaagctcacaacagccacgttccccgatgacatggacaccacatctatggctctctcggtccttcctgtggccgaggatgtggtctcttctgtcctggatgagatgcttaaattcgtcaccgatgacggtatctttatgacttacttcgattcctcgcgccctcgagtcgacccagtcgtatgtatcaacgtcttgggtgttttctgcaggcataaccgagagcgagacgtccttccaacgttccattggattcgagacatcctgatcaaccgggcatatctctcgggcacccgatactacccatcgcccgatttgtttttgtttttccttgcacgcctctgcctggcagtccggaatcagagcctacgggaacaacttgtcttgcctctggtagaccgactgcgtgagcgggtgggcgcacctggagaagcggtctcattggcagcgcggatccttgcctgccgtagctttggtatcgacagtgcgagagacatggacagcttgaggggaaaacaatgcgaggatggcggctggccagtggagtgggtttaccggtttgcctctttcggcctgaacgtaggcaatcggggtcttgctactgccttcgcggtcagggcgctcgaaagcccctatggtgagtcggcggtgaaggttatgagacgcatcgtctga

SEQ ID NO: 56 - DNA, 1461 bp, synthetic construct
  misc_feature 1..1461: note = codon optimized cDNA
  source 1..1461: mol_type = other DNA
atggcaatcactaagggcccagttaaagcgctgattcttgatttttctaacgttctgtgtagctggaagccgccgagcaatgttgcggtcccgcctcaaattctgaagatgattatgtcgagcgacatctggcatgattatgagtgtggccgttacagccgtgaggactgctacgcccgtgttgctgaccgttttcatatcagcgcagcggacatggaagataccctgaaacaggcacgtaagtccctgcaagtgcaccacgaaacgctgctgttcatccaacaggtgaagaaagacgcgggtggtgagctgatggtttgcggcatgaccaacacgccgcgtccggaacaagacgtgatgcattccatcaatgctgagtatccggtgttcgaccgtatttacattagcggcctgatgggcatgcgtaaaccgagcatttgtttctaccaacgcgtaatggaagagattggtctgagcggtgacgccatcatgttcattgacgataaactggaaaatgtgattgccgcacagagcgtgggtatccgcggtgtgctgttccaaagccagcaagatctgcgtcgtgtcgtgctgaactttctgggcgatccggtccaccgtggtctgcagttcttggcggcgaacgcaaagaaaatggacagcgtcacgaataccggcgacactatccaagacaatttcgcacagctgttgatcttagagctggcgcaggatcgcgaattggtgaaattgcaggccggtaaacgtacctggaactactttattggtccgccgaagctgaccacggcgacgtttccggatgatatggacacgaccagcatggcgctgtcggtgctgcctgtcgcggaagatgtcgtgagctctgttctggacgagatgctgaagttcgtgaccgatgatggtatctttatgacctatttcgactctagccgtccgcgtgtcgatccggttgtctgcattaatgtgttgggtgttttctgccgccacaatcgtgagcgcgacgtgttgccgacctttcactggattcgtgatattctgatcaaccgcgcatatctgagcggcacgcgctattacccgtccccggatctgtttctgtttttcctggctcgtctgtgcctggccgttcgcaaccagagcctgcgcgaacaactggttctcccgctggttgatcgtctgcgcgagcgtgttggtgctccgggtgaggctgtgagcctggcggcacgtatcctggcgtgccgtagcttcggtatcgactcagcccgcgacatggactccttgcgtggcaaacagtgtgaagatggtggttggccggtcgaatgggtctatcgcttcgcgagctttggtctgaacgttggcaaccgtggtttggccaccgcgtttgcggttagagcgctggagtccccatacggcgagagcgcagttaaggttatgcgccgtatcgtgtaa

SEQ ID NO: 57 - protein, 483 aa, Aspergillus aculeatus
MPSVKALVLDFAGVLCSWTPPAESPLSPAQLKQLMSSEIWFEYERGRYSEEECYAKLVERFSISAADMASTMEQARQSLELNHAVLQLVSEIRKRNPGLKVYGMTNTPHAEQDCVNRIVNSYPVFDHVYLSGLVGMRKPDLGFYRFVLAETGLRPDEVVFVDDKTENVLVAQSVGMHGVVFQNVTDFKQQIINVTGDPVSRGLRYLRSNAKSLLTVTSNNSVIHENFAQLLILELTGDRDLIELEPWDRTWNYFIGVPQSPTSTFPNDLDTTSIALSVLPIHKDVVADVMDEIMLLLDNDGIVPTYFDPTRPRVDPVVCVNVLSLFAQNGRESELLATFNWVLDVLRHRAYLQGTRYYISPDAFLYFLARLSVFLRMSPLRARLMPLLEERVYERIGAHGDAISLAMRIYTCKLLGMSNMLDERALRDMQCEDGGFPTSWVYRFGSTGVKIGNRGLTTALAIKAIEMPLASLWKSWGLTTDIR

SEQ ID NO: 58 - DNA, 1452 bp, Aspergillus aculeatus
  misc_feature 1..1452: note = cDNA
  source 1..1452: mol_type = genomic DNA
atgccctccgtcaaagcactggtcctggacttcgccggagttctatgctcatggaccccgccagccgagagcccgctctccccagcccagctcaaacaactcatgtcctccgagatatggttcgaatacgagcgcgggagatattccgaagaagaatgttatgcgaagctcgtcgaacggttctccatcagcgctgcggacatggcttccaccatggaacaggcccgtcagagcctggaactgaaccacgccgtacttcagcttgtcagcgagataaggaagcggaaccccgggctcaaagtttatggcatgacgaacacgccccatgcggaacaggattgtgtgaatcgcatcgtgaacagctatcctgttttcgaccatgtgtatctctccgggctcgttgggatgcgcaaaccagatcttggattctatcggtttgttctcgcagagaccgggttgaggcctgacgaggtcgtgttcgtcgacgacaaaacggagaatgtgttggtcgcgcagtccgtggggatgcacggcgtggtgttccagaacgttacggatttcaagcagcagatcataaacgtgacgggagaccctgtctctcggggcttgaggtatctccgctcgaatgcaaagagcctcctcactgtgactagcaataactccgtgatccacgaaaactttgcgcagttgctgattctggagctgacgggcgaccgagacttgatcgaactcgagccttgggatcgaacatggaactacttcatcggggttcctcagtcgccgacgagcaccttccccaacgacctggacaccacctctatcgcgctctcggtccttcccattcataaggacgtcgttgccgatgtgatggacgagattatgcttctcctagacaacgacgggatagtcccaacatattttgatcccactcgccctcgagtcgacccagtcgtgtgtgtgaatgtactcagcctgtttgcccaaaacggccgagaatccgagttactcgccaccttcaactgggtgctggacgtgctgcgacatagagcctacctgcagggcacgagatattacatcagtccggacgccttcttgtactttctagccagactctcggtctttctgaggatgagtccactccgcgctcggctaatgcctctcctggaagaaagagtgtatgagcgaattggtgcccatggcgacgccatttcgctggctatgcggatctatacgtgtaagctgctcgggatgtcgaatatgctcgatgaaagagcattgcgggacatgcagtgtgaggatggcggcttccctacaagttgggtctatagatttggatcgaccggagtgaagattgggaacagggggttgactactgcacttgcaataaaggccattgagatgcctctcgcttcgctttggaagtcgtggggattgacgactgacattcgataa

SEQ ID NO: 59 - DNA, 1452 bp, synthetic construct
  misc_feature 1..1452: note = codon optimized cDNA
  source 1..1452: mol_type = other DNA
atgccgtcggttaaagcgttggttctggattttgcgggtgtgttgtgttcttggactccaccggcggaaagcccgttgtccccagcgcagctgaagcagctgatgagcagcgagatctggtttgagtatgagcgtggccgctatagcgaagaagagtgttatgcaaaattggtggagcgtttctctatctcggccgcagatatggcgagcacgatggaacaggcccgtcaatcgctggagttgaaccacgccgtgctgcaattagtttccgagattcgtaaacgtaatccgggcttaaaggtttacggtatgactaataccccgcatgcagagcaagattgtgtgaaccgtattgtcaatagctatccggtttttgatcatgtctacctgagcggtctggtgggtatgcgcaaaccggatctgggcttttaccgtttcgttctggcagagactggtctgcgcccggatgaagtcgtgttcgttgacgacaagaccgaaaatgtcctggtggctcaatccgttggcatgcatggtgtggtgttccaaaatgtaaccgacttcaaacaacagattatcaatgtcacgggtgatcctgtcagccgtggtttgcgctacttgcgttccaacgcgaagtctctgctcactgttaccagcaataacagcgttatccatgagaatttcgcgcagctgctgatcctggaactgacgggcgaccgtgacctgattgaactggaaccgtgggaccgtacgtggaactactttatcggcgtgccgcaaagcccgaccagcacctttccgaacgacctggatacgaccagcattgccctgagcgttctgccgattcacaaagatgtggttgcggacgtgatggatgagattatgctgctgctggacaatgacggtattgtcccgacctacttcgatccaacccgtccgcgtgttgatcctgttgtgtgcgtcaacgttctgagcctgttcgcacagaacggtcgcgagtccgaattgctggcgacgttcaactgggttttggacgttctgagacaccgtgcgtatttgcagggtacgcgctattatatcagcccggatgcctttctgtattttctggcgcgcctgtctgtgtttctgcgtatgtctccgttgcgcgctcgtctgatgccgctgctggaagaacgcgtttatgagcgtatcggcgcacacggcgatgctattagcctggcgatgcgcatttacacctgtaagctgctgggcatgagcaatatgctggacgagcgtgcactgcgtgacatgcagtgtgaagatggtggtttcccaaccagctgggtgtaccgttttggtagcacgggcgtgaaaattggtaaccgtggcttgacgaccgcactggccattaaggccatcgaaatgccgctggccagcctttggaaaagctggggcctgaccaccgatattcgctaa

SEQ ID NO: 60 - protein, 485 aa, Aspergillus udagawae
MTRQKSPQYKAIIFDLGDVFFTWDAPKDTAVLPNLFKKMLTSPTWSDYERGKLSEESCYERLAEQFDVDSSEIARSLRKAQQSLTTDAAIVSLISEIRALAGHIAIYAMSNISAPAYAAVLQTQPEMGIFDGVFPSGCYGTRKPELLFYKKVLQEIAVPPNQIIFIDDQLENVVSAQSTGMHGIVYTGAGELSRQLRNLVLDPVQRGREFLRRNAGALYSICETGQVIRENFSQLLILEATGDRSLVNLEYQQRSWNFFQGGPPSTSETFPDDVDTTSIALMILPADDNTVNSVLGEISEVANDEGIVNTYFDQTRQRIDPAVCVNVLRLFYTYGRGATLPLTLQWVSDVLEHRAHLHGTRYYPSPEVFLYFVSQLCRFSKREPTLQLLETLLTDRLKERIQVKADTLSLAMRILACLSVGISQVEVDVRELLALQCKDGSWEPGSFYRFGSSKMNVGNRGLTTALATRAVELYQGTRIRSKGTE

SEQ ID NO: 61 - DNA, 1458 bp, Aspergillus udagawae
  misc_feature 1..1458: note = cDNA
  source 1..1458: mol_type = genomic DNA
atgacccgacagaaatcgcctcaatacaaagcaatcatctttgacctaggggatgtctttttcacctgggacgcccccaaagacactgctgtcttgcccaacctcttcaagaaaatgcttacctcgccaacctggtcagattacgagcgcggcaagttgagcgaagaaagctgctacgagagactggccgaacagtttgacgttgactcgtcggaaatcgcgcgcagcttaaggaaagcacagcagtctcttaccacagacgcagcaatcgtgagcctgatatcagagatcagagcgttggccggacatattgccatctacgccatgtccaacatttccgccccagcttatgcagctgtgctccagactcagcccgaaatgggcatctttgacggagtgttcccgtctggatgctatgggacgaggaagccggagctgttgttctataagaaagtcttgcaggagattgcagtgccgccaaatcagatcatctttattgatgatcagctagagaatgtagtttctgcgcagtcaacaggtatgcacggcattgtctacaccggtgcgggtgagctcagtcgacagctcagaaatctggtgttggaccctgtacaaaggggtcgagagtttctacggcgcaatgctggggcattgtatagtatctgcgagactggtcaagtcatccgggaaaacttctcgcagctgctcatcctagaggcgacgggtgatagaagcctggtcaaccttgaatatcagcagcggagctggaatttctttcaaggaggtcccccttctacgtcggaaacattcccagatgatgtcgacacaacatccattgccttgatgattctccctgccgatgataacacagtcaactcggttctcggcgagatttccgaggtagctaatgacgagggcattgtaaatacgtactttgaccagacccgacagcgaatcgacccagcagtctgcgtcaatgtcctccgtctcttttatacctacggccggggcgccactctcccattgaccctccagtgggtgtccgacgttcttgagcatcgtgcgcacttacatggtacgcgatactaccccagcccggaggttttcctctactttgtcagtcaactctgccggttctccaagagggaaccgacgctgcagctgctggagacgttgctcacggatcgcctcaaggagcgcattcaggtcaaggcagacactctgtcactggctatgcggatcctggcatgcttgtctgtgggtatatcacaagttgaagtggatgtccgagagctgctcgccttgcaatgcaaggatggatcgtgggaacccggctcgttttaccggtttgggtcgtccaagatgaacgttggtaatcgaggtcttacgactgcgttggcgactagggcggttgagttgtaccaggggactagaatacgctctaagggcaccgagtag

SEQ ID NO: 62 - DNA, 1458 bp, synthetic construct
  misc_feature 1..1458: note = codon optimized cDNA
  source 1..1458: mol_type = other DNA
atgactcgccaaaaaagccctcaatacaaagcaattatcttcgatctgggtgacgttttcttcacctgggatgcgccgaaagatacggccgtactgccgaacctgttcaagaaaatgctgacctcgccgacctggagcgactatgagcgtggtaagctgtctgaggaaagctgttacgaacgcttggccgagcaatttgacgtggacagcagcgagatcgcgcgtagcctccgtaaagcgcagcaaagcctgacgaccgacgcagccatcgtgagcctgatcagcgagatccgcgcattggcgggtcacattgctatctatgctatgtctaacatttctgcgccagcatacgcagcggtgttacagacccagccggaaatgggtatctttgatggtgtttttccgagcggctgctatggtacgcgtaaaccggaactgctgttttacaaaaaagtgcttcaagaaattgcggttccgccgaatcagattatcttcattgacgatcagctggaaaacgtcgtcagcgcacagtccacgggcatgcatggcattgtttacaccggtgccggtgagctgagccgtcaactgcgtaatctggtcctggacccggtgcagcgtggtcgtgagttcctgcgccgtaatgctggcgccctgtacagcatttgtgagactggccaagttatccgtgagaacttcagccagctgctgattctggaagcaaccggcgatcgttcgctggtgaacctggagtatcaacaacgttcctggaacttctttcagggtggccctccatccacgagcgaaacttttccggatgatgttgacacgacctcaatcgcgctgatgattttaccggcggacgataataccgtcaatagcgtcctgggtgaaatcagcgaagtcgcgaatgacgagggcattgtgaatacctatttcgatcagacccgccaacgtatcgatccggccgtgtgtgtcaacgtgttgcgcctgttttacacctatggtcgtggcgctacgctgccgttgaccctgcaatgggttagcgacgtgctggagcaccgtgcgcatctgcacggcacccgctactatccgtccccagaggttttcctgtactttgtctctcagctgtgccgtttttccaagcgcgaaccgaccctgcagctgctggaaacgctgttgaccgacagactgaaggaacgcatccaagttaaggcagatacgctgagcttggcaatgcgtattttggcgtgcctgagcgtgggcatcagccaggttgaggttgacgtccgcgaactgctggcgctgcagtgcaaggacggtagctgggagccgggtagcttctaccgtttcggtagcagcaagatgaatgtcggtaaccgcggtctgacgaccgctttggcgacccgtgcggttgagctgtaccagggtacgcgtattcgtagcaagggcaccgagtaa

SEQ ID NO: 63 - protein, 524 aa, Trametes versicolor
MASPHRRYTTLILDLGDVLFSWSSKTNTPIPPKKLKEILSSLTWFEYERGRISQAECYDRVSSEFSLDAATIAEAFQQARDSLRPNEEFLALIRELRQQTHGQLTVLALSNISLPDYEYIMALDSDWTSVFDRVFPSALVGERKPHLGAYRRVISEMHLDPETTVFVDDKLDNVVSARSLGMHGVVFDSQENVFQTLRNIFGDPIHRGRDYLRRHAGRLETSTDAGVVFEENFTQLIIYELTNDKSLITTSDCPRTWNFFRGKPLFSASFPDDVDTTSVALTVLRPPRTLVNSILDEMLEYVDADGIMQTYFDHSRPRMDPFVCVNVLSLFYEYGRGQDLPKTLEWVYEVLLHRAYIGGSRYYMSADCFLFFMSRLLQRITDPAVLNRLRPLFVERMHERVSAPGDSMELAFRILAGSSVGIQFPRDLEKLLAAQCADGGWDLCWFYQYGSTGVKAGNRGLTTALAIKAIESAIARPPSPALSAVSSSKLEVPKPILQRPLSPRRLGDFLMPWRRAQREVAVSS

SEQ ID NO: 64 - DNA, 1575 bp, Trametes versicolor
  misc_feature 1..1575: note = cDNA
  source 1..1575: mol_type = genomic DNA
atggcttcacctcaccgcaggtatacgacactcatcctagacctgggcgacgtcctcttctcttggtcatccaagaccaacacacctatccctcccaagaagctgaaggagatcctctcgtccctgacctggttcgagtacgagcgcggtcggatatcacaggccgagtgctatgaccgggtcagctccgagttcagtcttgacgctgccaccatcgcagaagcgttccagcaggctcgcgactctctgcgaccgaacgaagagttcctggcgttgattcgcgaactccgccaacaaacgcatggtcagcttaccgtcctcgcgctctcgaacatctcactccccgactatgaatacatcatggctctcgactcggactggacgtcggtcttcgaccgcgtcttcccttctgccctcgtcggcgagcgcaagccacatctgggggcgtaccgccgtgtcatctctgagatgcacctagacccagaaacgaccgtctttgtggacgacaagctggacaacgtggtgtccgcgcgatcgctcgggatgcacggcgtggtcttcgactcccaggagaacgtcttccagacgctgaggaatatcttcggcgacccgatacatcgcggacgtgactatctccgcaggcatgccggtcgtctggagacatctacggacgccggcgttgtcttcgaggaaaactttacgcagctcatcatctacgaactaacaaatgacaaatccctcatcacgacatcagactgtccccgcacttggaacttcttccgcgggaagcccttgttctcggcctcgtttcccgacgatgtggacacgacgtcggttgccctgacagtgttgcgcccaccccgcacgcttgtcaactcgatcttggacgagatgctagagtatgtcgacgccgacggcatcatgcagacctacttcgaccactcgcgcccgcggatggatccgttcgtctgtgtcaacgtcctgtcgctgttctacgagtacggccggggacaggacctcccgaagaccctcgaatgggtatacgaggttctgctgcaccgcgcctacatcggcggctcgcggtactacatgtccgcggactgcttcctcttcttcatgagccgccttctccaacgtatcaccgacccagccgtcctgaaccgcctccgcccgttgttcgtcgagcgcatgcacgaacgtgtcagcgcaccgggcgactccatggagctcgcgttccgcatcctcgctggctcgtccgtcggcatccagttcccacgtgacctggagaagctcctcgccgcgcagtgcgccgacggcggctgggacctgtgctggttctaccagtatgggtccaccggcgtgaaggcaggcaaccgcgggctcaccaccgcgctcgccatcaaggctatcgagagcgctatcgcgcgccctccgtcccccgctctatcagctgtatcgtcgtcgaaactggaagtgccgaaaccaattctccagcgtcccctcagcccgcgccggcttggcgacttcctgatgccctggaggagagcacagcgcgaggtcgcggtttccagctag

SEQ ID NO: 65 - DNA, 1575 bp, synthetic construct
  misc_feature 1..1575: note = codon optimized cDNA
  source 1..1575: mol_type = other DNA
atggctagcccgcaccgtcgctatactactctgattctggatttgggtgatgttttgtttagctggagcagcaaaaccaatacgcctattccgccgaaaaagctgaaagaaatcctgtctagcctgacctggttcgagtacgagcgcggtcgcatttctcaagccgagtgctatgaccgtgtgagctctgagtttagcctggacgcagcgaccattgcagaggcattccaacaggctcgtgactcgctgcgcccgaacgaagaatttctggcgttgattcgtgagctgcgccagcagacccacggccaactcaccgttctggcactgagcaacatctccctgccggattacgagtacatcatggctctggatagcgattggaccagcgtctttgatagagttttcccgagcgcgctggttggtgagcgtaagccgcatctgggtgcttaccgtcgtgtcattagcgagatgcatctggacccggagactacggtgtttgtggacgacaaactggacaacgttgtctccgcgcgcagcctgggtatgcacggcgtcgtttttgactcacaagaaaatgttttccagacgctgcgtaacattttcggtgaccctatccaccgtggccgcgactatttgcgtcgtcatgccggtcgtttggaaaccagcaccgacgcgggcgttgtttttgaagaaaacttcacccagctgatcatctacgaactgacgaatgacaagagcctgatcaccacgagcgattgtccgcgcacctggaacttcttccgtggtaagccgctgtttagcgcgtccttcccagacgatgtcgatacgacttcggtggccctgaccgttctgcgcccaccgcgcaccctggtaaacagcatcctggacgaaatgttagaatacgtcgatgcggatggtattatgcagacctatttcgaccacagccgtccgcgcatggacccgtttgtgtgtgtgaatgtgttgagcctgttctatgagtacggccgtggtcaagatctgccaaaaaccctggaatgggtctacgaagtccttctgcatcgtgcctacatcggtggctcccgttattacatgagcgcagattgctttttgttctttatgtctcgtctgctgcagcgcatcacggaccctgccgtgctgaatcgtctgcgtccgctgttcgtggagcgtatgcacgagcgcgtgtctgccccgggtgacagcatggaactggcgttccgtatcctggcgggcagcagcgtgggtattcaatttccgcgtgatttggagaaactgctggctgcgcagtgtgcggacggtggctgggatctgtgctggttttatcaatacggtagcaccggcgttaaggccggcaatcgtggcctgacgacggcactggcaattaaggccattgagtccgcgattgcgcgtccgccgagcccggcattgagcgcggtcagcagcagcaaactggaagtgccgaagccgatcttgcagcgtccactgagcccgcgtcgtctgggtgacttcctgatgccgtggcgccgtgcgcaacgcgaagtcgcggttagctcctaa

SEQ ID NO: 66 - protein, 527 aa, Dichomitus squalens; identical to SEQ ID NO: 5

SEQ ID NO: 67 - DNA, 1584 bp, Dichomitus squalens
  misc_feature 1..1584: note = cDNA
  source 1..1584: mol_type = genomic DNA
atggcctcaatccaccgtcgatacactactctcatcctcgacctcggcgacgtactctttcgttggtctccaaagactgagaccgccattccacctcaacaactcaaggatatcctctcctctgtcacctggtttgagtacgaacgcggcagactatcccaggaagcatgctacgagcgctgcgccgaggagttcaagatagaggcctcggtcattgcagaagcctttaagcaggctcgcgggtcactgcggcccaacgaggagttcatcgccttgatccgtgacctccgccgtgagatgcacggtgaccttaccgttcttgccctctccaacatctccctccccgactacgaatacatcatgtcgctaagctcagattggacgaccgtcttcgatcgcgtattcccctctgcactcgttggcgagcgcaagcctcatctgggatgctatcgcaaggtcatctcggagatgaacctagaacctcagacgactgtgttcgtggatgacaagcttgacaacgtcgcgtctgctcgctcacttggtatgcacggcatcgtgtttgacaaccaagccaacgtcttccgccaactccgcaatatcttcggagaccccatccgccgtggccaagagtatctccgtgggcatgctggcaaactcgagtcttcgaccgacaacgggttgatcttcgaggagaacttcacacagctgatcatctacgagttgacgcaagacaggactctcatctcgctttcagaatgtcctcgtacttggaatttcttccgaggcgaaccgctattctcggagaccttcccggatgatgtcgacacaacatctgtggcgttgacggtattgcaaccggacagagcactggtcaactccgttctagacgagatgctggagtatgtcgacgccgatggcatcatgcagacatacttcgatcgttcacgaccacgcatggaccccttcgtctgcgtgaacgtactctccctgttctacgagaacggtcgtggtcacgagctccctcgcacattggactgggtctacgaggtgctcctccatcgcgcgtaccacggcggttcgcgttattacctgtcgcccgactgctttctattcttcatgagccgcctactcaagcgcgcagacgatccagcagtccaggctcggctccgcccgctcttcgtcgagcgggtgaacgagcgagtaggcgccgctggcgactcgatggacctcgccttccgcatcctcgccgcagcgtctgttggcgtccagtgcccccgcgatctggaaaggttgactgccgggcaatgcgacgacggtggatgggacctctgctggttctacgtgttcggctcgacgggcgtgaaggcgggcaaccgcggcctcacaacggccctcgctgtcacggccatacagacggccatcggacgccccccttcgcccagtccctccgcggcctcctcgtctttcagacctagttccccttacaaattcctaggcatttcgcgcccagctagccccattcgctttggcgacttacttcgcccatggcggaagatgagcaggtcgaacttgaagtctcaatga

SEQ ID NO: 68 - DNA, 1584 bp, synthetic construct
  misc_feature 1..1584: note = codon optimized cDNA
  source 1..1584: mol_type = other DNA
atggcaagcattcatcgtcgctatactacgctgattctggacctgggtgatgttttgttccgctggagcccgaaaaccgagactgcgattcctccgcaacaactgaaagacatcctgagcagcgtcacctggttcgagtacgagcgtggccgtctgagccaagaggcttgctacgagcgttgcgccgaagagttcaagattgaagccagcgtgattgcggaagcgttcaaacaagcgcgtggtagcctgcgtccgaacgaagaatttatcgcactgatccgtgatctgcgtcgcgagatgcatggtgacctgaccgttctggctctgagcaatatctcgttgccggattacgagtatattatgtctctgagcagcgactggacgacggtctttgatcgtgtgttcccgtcagctctggtgggcgagcgtaaaccgcacttgggttgctatcgcaaggtcatcagcgagatgaacctggaacctcagaccacggtctttgtggacgataaactggataatgtcgcaagcgcgcgtagcctgggtatgcacggtatcgtgtttgataatcaagcgaatgtgtttcgccagctgcgtaatattttcggtgatccaatccgtcgcggtcaagagtatctgcgtggccatgccggtaaattggagagcagcacggacaatggtttgatctttgaagagaacttcacccagctgatcatttatgaactgacccaggaccgcacgttgatcagcctgtcggagtgtccgcgtacctggaacttcttccgtggcgagccgttgttttctgaaaccttcccggacgacgtggacaccacgtccgttgcactgacggttctgcaaccggatcgcgcactggttaacagcgtgctggacgaaatgctggaatatgtcgatgcggatggcatcatgcagacgtatttcgaccgctcgcgtccgcgtatggacccgtttgtttgcgtcaacgtactgagcctgttttacgagaacggtcgtggtcacgaactgccgcgcactctggattgggtgtacgaagtcctgctccaccgcgcctaccacggtggttcccgttactacctgagcccggactgtttcttgttttttatgagccgtctgctgaaacgtgcagacgacccagcggttcaggcgagattgcgtccgctgtttgtggaacgcgttaacgaacgtgttggcgcggccggtgatagcatggacctggcgtttcgcattctggccgcagcgagcgtgggtgtgcagtgtccgcgcgacctggagcgtctgaccgctggtcaatgcgatgatggcggctgggatctgtgttggttctacgttttcggcagcaccggcgttaaggccggtaatcgtggtctgaccacggcgctggcagtcaccgcgatccagaccgccatcggccgtccgcctagcccgagcccgtccgcggcaagctccagcttccgcccgagcagcccgtacaagtttctgggtattagccgtccggcgtccccaattcgcttcggtgaccttctgcgtccgtggcgtaaaatgtctcgctctaacctgaagtcccagtaa

SEQ ID NO: 69 - protein, 495 aa, Antrodia cinnamomea
MRRNVLNKATHSQSPLKPNITTLIFDLGDVLLTWSDSTPKSPLPPKIVKGILRSLTWFEYEKGNLTESQTYGQVAQEFGVDASEVKASFEAARDSLKSNPMLLQLIRSLKDSGHVIYAMSNISAPDWEFLKTRADLSDWALFDRVFPSAEAHDRKPNIGFYQHVINETGLNPSNTVFVDDRIENVVSARSAGMHGIVFDDINNVIRQLKNLCEDPIHRARSFLYANKKCLNTVSTDGTIVSENFSQLLILEAIGDESLVDFVRHEGRFNFFQGEAKLIMTNHYPDDFDTTSIGLTVVPYIDDKTRNRVMDEILAYQSEDGIVLVYFDHKRPRIDPVVCVNVLTLFYRYGRGHQLQKTLDWVEQVLINRACASGTFYYATEEQFLFFLSRLIQSSPDVRQRLEGVFKRRVVERFGADGDALAMAMRIHTAASVGLVDHVDLDKLFALQQNDGSWRDSAFYRFPSARQLASNDGLTTAIAIQAIQAAERLREDGNVL

SEQ ID NO: 70 - DNA, 1488 bp, Antrodia cinnamomea
  misc_feature 1..1488: note = cDNA
  source 1..1488: mol_type = genomic DNA
atgaggcgaaacgtactcaacaaagcaacacattctcagtcaccattgaagcccaacatcacgacgctcatatttgacttgggcgacgtacttctcacgtggtccgactcaacacctaaatctccactgcccccaaaaattgtcaagggaatactacgttcactgacctggtttgagtacgagaaagggaacttgacagagtcccagacctacgggcaagttgctcaggaatttggagtggatgcttccgaagtcaaagcttccttcgaagcagctcgcgactcgctcaagagcaacccaatgcttctccagttgatccgtagcctcaaagactctggccacgtcatttacgcaatgtctaacatatctgctcccgactgggaatttttgaagacgcgggcagacctctcagattgggctctttttgacagagtcttcccttctgccgaagcgcatgaccgcaagccgaacattggtttctatcagcacgtcataaacgagactggtctgaacccgtccaacactgtctttgtcgatgacaggatcgagaatgttgtatccgcacgctcagcaggaatgcacgggatcgtgtttgacgacataaataatgtgatccgacagttgaaaaacctctgcgaggatccgattcaccgcgcacgatcttttctttatgcaaataagaagtgtttgaatacggttagcacagatggcacaattgtgagcgagaacttctcgcaattgttgatccttgaggccattggcgacgaaagcctagtcgactttgtgaggcatgagggccgattcaacttcttccagggggaggccaaactcatcatgacgaatcactaccccgatgatttcgatactacatccataggtttaaccgttgttccatatattgacgacaagactagaaatagagttatggatgagatcctggcctaccaaagcgaagacggcattgtgctggtatactttgaccacaagcgccccaggattgatcctgttgtctgtgtcaatgtcctcaccctcttctataggtatggccgtgggcaccagcttcaaaagacactggattgggtcgaacaggtcctgatcaaccgtgcgtgtgcgtccggcacgttctattacgcaacagaggaacaattcctctttttcctctcccgcctgatccaaagctctccggacgtacgacagcggttggaaggggtctttaaaagaagagtagtcgagcggtttggtgcagacggcgacgctctcgctatggcgatgcgcattcacaccgcggcgagcgtgggcctcgttgaccatgtcgatcttgacaagctgttcgcattgcagcaaaatgacggttcttggagagacagcgctttctacagatttccgtcggccaggcaactggctagtaacgacggcttgacgactgcaatcgctattcaggccattcaagctgcggagaggctcagggaggatgggaacgtgctttga

SEQ ID NO: 71 - DNA, 1488 bp, synthetic construct
  misc_feature 1..1488: note = codon optimized cDNA
  source 1..1488: mol_type = other DNA
atgcgccgtaatgtcctgaacaaagcaacccatagccagtcaccgttgaaaccgaatatcaccacgctgatttttgacttgggcgatgtcctgctgacctggagcgacagcactccgaaatctccgttgccgccgaagatcgtcaagggcatcctgcgtagcctgacttggttcgagtacgaaaagggcaatttgaccgaaagccaaacgtatggtcaggtcgcgcaagaatttggtgtggatgcctctgaagtgaaggccagctttgaggctgcgcgtgatagcttgaaatcgaatccgatgctgctgcagctgattcgcagcctgaaagattccggtcacgtgatctacgccatgagcaacatcagcgcgcctgattgggaatttctgaaaacccgcgctgacctgtctgactgggccctgtttgaccgcgtgttcccgtctgccgaggcacatgaccgcaaaccgaacattggcttttaccaacacgtgatcaatgaaacgggtctgaatccatccaataccgtgttcgttgacgaccgtattgaaaacgttgttagcgcacgtagcgctggtatgcacggtatcgttttcgatgacattaacaacgtcattcgccagctgaagaatctgtgcgaggacccaattcaccgtgcacgttcctttttgtatgcgaacaaaaagtgcctgaataccgtgagcaccgatggtacgatcgtcagcgagaactttagccagcttctgattctggaagccattggtgacgagtccctggtagacttcgtccgccatgagggccgttttaacttcttccagggtgaggcaaagctgatcatgaccaatcactacccggacgatttcgataccacgagcattggtctgaccgttgtcccgtatatcgatgacaaaacgcgtaatcgtgtgatggatgaaatcctggcgtatcagtccgaggatggtatcgttctggtgtacttcgatcacaagcgtccgcgcattgacccggtcgtttgtgtgaacgttctgacgctgttctaccgctatggtcgtggccatcaactgcagaaaaccctggactgggttgagcaagtcctgattaatcgtgcgtgtgcgagcggcacgttctactacgcgaccgaagaacagttcctgtttttcctgagccgtctgattcagtcgagccctgacgtgcgccaacgtctggaaggcgtgttcaagcgtcgtgtcgttgagcgctttggtgcggacggtgatgccctggcaatggcgatgcgtatccataccgcagcgagcgttggcctggtggaccacgtggatctggataagctgttcgcgctgcaacagaacgacggtagctggcgcgatagcgcgttttatcgttttccgagcgcgcgtcaactcgcgagcaacgacggcttgaccacggcaattgctattcaggccatccaagcggctgagagattacgtgaggatggtaacgttctgtaa

SEQ ID NO: 72 - protein, 543 aa, Aspergillus ustus
MVRALILDLGDVLFNWDAPKSTPVSRKTLSQMLHSDIWGEYECGQLTEPESYKALASRYSCQAQDVADTFYLARESLRLDATFKTFLQDLKQRANGSLRVYGMSNISQPDYEVLLSKADDLSLFDKIFPSGHVGMRKPDLAFFRHVLREISTASEDIVFVDDNLENVTSARSLGMQGIVFRDKEDVQRQLRNLFGSPAERGREYLSINKTKLQSVTTTNIPILDNFGQLLILEATRDPDLVSMHPGQRTWNFFIGSPTLTTDAFPDDMDTTSLGLSIIPPSPEIAASVMDEIVTRLNKDGIVPTYFDSTRPRVDPIVCVNVLTLFAKYGREDELSGTIAWVRDVLYHRAYLAGTRYYASPEAFLFFFTRFTRNLRPGPRKQELTALLSQRLQERNKTPVDALALSMRIIACLTLGIESPADDVATLTGMQCGDGGWPACVIYKYGAGGLGITNRGVSTAFAVKAITTTPLAVQPEVSVSAGAGGSSRPVGADAAAVSLRPRWRAVVQSLHPLSRVGGLVAVIFAALHFNLAWLYNVSLASRIV

SEQ ID NO: 73 - DNA, 1632 bp, Aspergillus ustus
  misc_feature 1..1632: note = cDNA
  source 1..1632: mol_type = genomic DNA
atggtccgcgcactgattctcgatctcggcgacgtcctcttcaactgggacgccccaaagtcaacccccgtttcccgcaagacactcagccagatgctgcatagcgacatctggggcgaatacgaatgtggccaactgacagagccggaaagctacaaggcgcttgccagccgctattcttgccaggctcaagatgttgcagataccttctatctagcccgcgaatcgctgaggctcgatgcgaccttcaagaccttcctgcaggacttgaagcagagggccaacggctcacttcgcgtatatgggatgtccaacatctcccagcccgattatgaggtcctgctgtccaaggcggatgacttgagcctgtttgacaagatcttcccatccggccacgtcgggatgcgtaagcctgaccttgcgttttttcgacatgtcctgcgtgagatctcgacggccagcgaggatattgtgtttgttgacgacaacctggagaacgtgacatctgcccggtctctgggcatgcaggggattgtctttcgcgacaaggaggatgtacagagacagctgcggaacctctttggcagtcctgctgaacgtggaagggagtatttgtccatcaacaagacaaagctccagagcgtcacgacgaccaatatccccattctcgacaactttggccagctccttatcctcgaagccaccagagacccagacctggtgtccatgcatcctggacagaggacctggaactttttcatcggatctccaactctgacaacggacgccttcccagacgatatggacaccacctcacttggcctttctattatacccccaagtcccgagattgcagcgtccgtgatggatgagattgtgacccgcctgaacaaggacggcattgtcccaacatattttgacagcaccagaccccgcgtcgacccgatcgtctgcgtcaacgttctcaccctcttcgctaaatacggccgcgaagacgagctgtccgggaccatagcctgggtgcgcgatgtgctgtatcacagggcctaccttgcagggaccagatactacgcatccccagaagcattccttttcttcttcacgcgcttcacccgaaacctgcgcccgggcccgcgcaagcaggagctcacggcgctgctgtcccagcgcctgcaggagcgcaacaagacgcccgttgacgcacttgcgctctcgatgcggattattgcgtgcctcacgctgggtattgaatcccccgctgacgacgtggctaccctcacgggcatgcagtgtggggatggcgggtggccggcctgtgtcatctacaagtacggcgccggtgggctggggatcacgaacaggggggtctcgaccgcgtttgctgtcaaggcaatcactactactcctttggcggtgcagcctgaagttagtgtcagcgcaggtgcaggaggcagcagtcgccctgtgggtgccgatgctgctgcagtctcgctccgcccgagatggcgagctgttgtgcagagtctccatccgctctctcgggttggtgggttggtggccgtcatttttgctgcactgcatttcaacttggcctggctttataatgtgtcccttgctagtaggatcgtttag

SEQ ID NO: 74 - DNA, 1632 bp, synthetic construct
  misc_feature 1..1632: note = codon optimized cDNA
  source 1..1632: mol_type = other DNA
atggttcgtgcattgattttggatttgggtgatgtgttgtttaactgggatgcgcctaagagcaccccggtttcccgcaagactctgagccaaatgctgcactcggatatttggggcgagtacgagtgtggtcaactgactgagccggagtcctataaagccctggcgagccgctatagctgccaggcgcaagatgtcgctgacaccttttacctggcgcgtgagagcctgcgtctggacgcaacgtttaagaccttcctgcaagatctgaagcaacgcgccaacggttctctgcgtgtctatggtatgagcaatatcagccagccggattacgaagtcctgctgagcaaagctgacgatctcagcctgtttgacaaaatctttccgtcgggtcacgttggtatgagaaagcctgacctggcgtttttccgtcacgttctgcgtgagatcagcacggctagcgaagatattgtgtttgttgacgacaatttggaaaacgtcacgtctgcacgctccctgggtatgcaaggcatcgtctttcgtgataaggaagatgtccagcgccagctgcgcaatctgttcggttccccggcagagcgcggtcgtgagtatctgagcattaataagaccaaactgcagagcgtgaccaccaccaatatcccgattctggacaacttcggtcagttgctgatcctggaagctacccgtgacccggatttagtcagcatgcatccaggccaacgtacgtggaacttcttcattggcagcccgaccttgacgaccgacgcgtttccggacgatatggacacgacttctctgggcctgagcatcatcccgccgagcccggaaattgcagcaagcgttatggacgaaatcgtcacccgtctgaataaagatggtattgtgccgacctacttcgacagcacgcgtccacgtgtggacccgatcgtctgcgttaacgtcctgaccttgtttgcgaaatatggtcgtgaagatgaactgagcggcacgattgcgtgggtccgcgacgttctgtatcatcgcgcatacctggcgggcacgcgctactacgcgtccccagaggccttcctgttcttctttacgcgtttcacccgcaatctgcgtccgggtccgcgtaaacaagaacttacggcgctgctgagccagcgtctgcaggaacgcaacaagacgccggttgacgctctggccctgagcatgcgtatcatcgcctgtctgaccctgggcattgagagcccggcagacgacgtggccaccctgaccggtatgcagtgtggtgatggtggctggccggcgtgcgtgatctacaaatatggtgcgggtggcttgggtatcacgaatcgtggcgttagcactgccttcgcggtgaaagcgattacgaccaccccgctggcagtgcagccagaagtcagcgtcagcgctggtgccggcggctccagccgcccggttggtgcggatgcggcagcggttagcttgcgtccgcgttggcgtgcggttgtgcagagcctgcatccgctgagccgcgtgggtggcctggttgccgtgatcttcgcggcactgcactttaacctggcgtggctgtacaacgtaagcctggctagccgtattgtgtaa

SEQ ID NO: 75 - protein, 477 aa, Aspergillus oryzae
MTRWKSSQYQAIIFDLGGVILTWDLPEDTVISAQIFKRMLTSQTWSDYERGNLSENGCYQRLAEDFGIDSADIAHTVRQARESLVTDTAIMNIISEIRAGANHIAIFAMSNISQPDYAALLLDHRGMCSFDRVFPSGCYGTRKPELSFYNKVLREIDTPPENVIFVDDQLENVISAQSIGIHGIAYTNAAELGRQLRNLIFDPVERGREFLRRNAGEFHSITETDQIVRENFSQLLILEATGDKSLVSLEYHQKSWNFFQGNPILTTETFPDDVDTTSLALMTLPTDTKTANLLLDQILGLVNADEIVTTYFDQTRERIDPVVCVNVLRLFCTYGRGIALPLTLQWVYDVLAHRAYINGTRYYTSPESFLYFVGQLCRFSTGVLALRPLETLLIDRLKERLQVKADPLSLAMRILTCLSVGVSQVEVDLRELLSMQCEDGSWEHCPFTRYGLSKVSIGNRGLTTAFVVKAVEMCRGS

SEQ ID NO: 76 - DNA, 1434 bp, Aspergillus oryzae
  misc_feature 1..1434: note = cDNA
  source 1..1434: mol_type = genomic DNA
atgactcgatggaaatcgtcccaataccaagcaattatctttgacctaggcggtgtcattttaacatgggacctcccggaagacactgtgatatcggcccagatctttaagagaatgctcacatcgcagacatggtcagattatgagcgcggaaatctcagcgaaaatggttgctaccagaggttggccgaggattttggcattgactctgccgacattgcacataccgttagacaagcacgggaatcccttgtcactgataccgctatcatgaacattatatctgagatcagagctggggctaaccatattgctatcttcgctatgtcgaacatctcccaaccagattatgcggctctgctccttgatcatcgcgggatgtgcagttttgaccgggtgttcccatctggatgctacgggacaaggaaaccagagctctcattctataacaaagtcttgcgggagattgacacgccaccggaaaacgtcatctttgtcgatgatcagctggaaaatgtgatctctgcgcagtccattggcatacacgggattgcctatacgaatgctgctgaactcggtcgacagcttaggaacctaatatttgaccctgtagagaggggtagggaattcttacggcgcaatgctggagagttccatagcatcactgaaaccgatcaaattgttcgggaaaatttctcacagttgctcattctagaagcgactggtgataagagtctggtatctcttgaatatcaccagaagagctggaatttcttccaaggaaaccctattctcacgacagagacattcccagatgatgttgacacaacatctcttgccttgatgactctacctacagacacaaaaactgcaaatttgttactcgaccagattttggggctagtcaacgctgatgaaatcgtaacaacatactttgaccagacccgagaacggatcgatccagtagtctgcgtcaatgtccttcgtctcttttgcacctacggccggggcattgcgctccctttgactcttcagtgggtgtacgacgtcctcgctcatcgggcatatataaacggtacacgttactacacaagtcccgaaagcttcctatacttcgtcggtcaactttgtcgattctcaacaggggtactggcacttcggccgctggaaacgttgcttatagatcgtctcaaggaacgtcttcaggtcaaagcagatcctctatcactcgctatgcggatcttgacctgtttgtccgttggtgtgtctcaagttgaagtcgatctccgagagttgctctcgatgcagtgtgaagatggctcgtgggaacattgtccattcacccggtatggtttgtccaaagtgagcattggcaatcggggccttacaactgcttttgtggtcaaggcggttgaaatgtgtcgaggcagttag

SEQ ID NO: 77 - DNA, 1434 bp, synthetic construct
  misc_feature 1..1434: note = codon optimized cDNA
  source 1..1434: mol_type = other DNA
atgactcgttggaaaagctctcaatatcaggcaatcattttcgatctgggcggtgttattctgacctgggacttgccggaagatacggttatctccgcgcaaatctttaagcgtatgctgaccagccagacctggtccgattatgagcgcggtaatctgagcgagaacggctgctatcaacgtttggcggaagatttcggcatcgatagcgccgatattgcccacaccgtccgtcaggcacgtgagtccctggtgaccgacaccgccatcatgaatatcatctccgagatccgtgcaggcgcgaaccacatcgcaattttcgcgatgagcaacatctcacagccggattacgctgcgctgctgctggaccatcgcggtatgtgcagctttgaccgcgtctttccgagcggttgttacggcacccgtaagcctgagctgagcttctacaataaagtgctgcgtgaaattgacaccccgccggaaaatgttattttcgttgacgatcaattggaaaatgtgattagcgcgcaaagcattggtattcatggcattgcgtatacgaatgccgcggaactgggccgccagctgagaaacctgatcttcgatccggtggagcgcggtcgtgagttcctgcgtcgtaacgctggtgagtttcactctattacggaaacggaccagattgtgcgcgagaacttcagccagctgctgattctggaagcgaccggtgacaaaagcctggttagcctggaataccaccaaaagtcgtggaacttcttccaaggtaacccaatcctgacgacggaaaccttcccggacgatgttgacactactagcctggctctgatgacgctgccgacggacaccaagaccgcgaatctgttgctggaccagattctgggtttggttaatgccgatgaaattgtgactacgtacttcgaccagacccgtgagcgtatcgatccagtggtctgtgtgaatgtcctgcgcctgttctgtacgtacggccgcggcatcgcgctgccgctgaccctgcaatgggtctacgatgtgctggcgcaccgcgcatacattaacggtacgcgttattacaccagcccggagagctttctgtattttgtcggtcagctctgtcgttttagcaccggtgtgctggcactgcgtccgctggagactctgctgattgatcgtctgaaagagcgcctgcaagttaaagctgacccgctgagcctggcaatgcgcatccttacgtgcttatctgtcggtgtcagccaggttgaagtggacttgcgtgagttgttgagcatgcagtgcgaggacggtagctgggagcattgcccgttcacccgctacggcctgagcaaggtttccatcggtaaccgtggcctgaccacggcgtttgtggttaaagccgtcgagatgtgccgtggcagctaa

SEQ ID NO: 78 - protein, 528 aa, Aspergillus calidoustus
MVRALILDLGDVLFNWDAPASTPISRKTLGQMLHSEIWGEYERGHLTEDEAYNALAKRYSCEAKDVAHTFVLARESLRLDTKFKTFLQTLKQNANGSLRVYGMSNISKPDFEVLLGKADDWTLFDKIFPSGHVGMRKPDLAFFRYVLKDISTPVEDVVFVDDNLDNVTSARSLGMRSVLFHKKDEVQRQLTNIFGSPAERGLEYLSANKTNLQSATTTDIPIQDNFGQLLILEATEDPSLVRMEPGKRTWNFFIGSPSLTTDTFPDDLDTTSLALSIVPTSPDVVNSVIDEIISRRDKDGIVPTYFDNTRPRVDPIVCVNVLSMFAKYGREHDLPATVAWVRDVLYHRAYLGGTRYYGSAEAFLFFFTRFVRNLRPGTLKQDLHALLSERVRERLNTPVDALALSMRIQACHALGFDAPADIATLITMQDEDGGWPAAVIYKYGAGGLGITNRGVSTAFAVKAITGSPVKTETNIGGDGARAVSAMSSLEARRLQPISSVGDWVRFIIASLHVHLAWLWNVLLLSKVV

SEQ ID NO: 79 - DNA, 1587 bp, Aspergillus calidoustus
  misc_feature 1..1587: note = cDNA
  source 1..1587: mol_type = genomic DNA
atggtccgcgcactcatcctcgatctcggcgatgtcctcttcaactgggacgcgcctgcgtccacccccatttcacgcaagaccctcggccagatgctgcatagtgagatctggggtgagtatgaacgtggccatttgacagaagacgaggcatacaacgcactcgcgaagcggtattcctgcgaggccaaggatgtcgcacatacctttgtcctggcacgagaatcgctgcggctcgacacgaaattcaaaacgtttctgcagactctaaagcagaatgccaacggctcccttcgtgtctatggcatgtcgaatatatcgaaaccggatttcgaagtcctgctgggcaaggccgatgactggactctgtttgacaagatcttcccctctggccatgtcggtatgcgcaagccagatcttgccttcttccgctatgtgctcaaggacatttcaacgcctgtcgaggatgtggtgtttgttgacgataacctggacaacgtgacgagtgctcggtctctgggcatgcgcagcgtcctctttcataagaaagacgaggtccagcgacagctcaccaacatctttggcagccctgctgagcggggcttggagtatctctccgccaacaagacgaatctgcagagtgctaccacgacagatatcccaatccaggataactttggccaacttctgattctcgaggccactgaagacccatcgctggtccgcatggagcccggtaagcgaacctggaatttcttcatcggttctccatccctcacaaccgacaccttccccgacgatctcgacaccacatcccttgccctctccatcgtacccacaagccccgacgtcgtcaactcggtcatcgacgagattatcagccgtcgcgacaaggacggtatcgtcccgacttacttcgacaacacccgcccccgcgtggacccaatcgtctgcgtaaacgtcctctccatgttcgcaaagtacggccgcgagcacgacctccccgcaacagttgcgtgggtccgcgacgtcttgtatcatcgagcatacctcggcggaacacggtactacgggtcagctgaggccttcctcttcttcttcactcgcttcgttcgcaacctccgaccgggaactctcaagcaggatctacacgcattgctatcagagcgcgtgcgcgagcgactcaatacccccgtcgacgcactcgccctgtcaatgcgcatccaggcctgtcatgcgctgggctttgacgcccccgcagacattgcgacgctcatcacaatgcaggacgaggacggcgggtggccggcagccgtcatctacaagtacggggccggggggttggggatcacgaaccggggtgtttcgactgcgtttgccgtaaaggcgattacagggtcgcccgtgaagactgaaaccaacataggcggcgatggagctcgcgctgtctcggccatgtcctccttggaggcgaggaggctacagccgatctcgtcggttggggactgggtgcggtttatcattgcgtcgttgcatgtccatctggcttggctttggaatgttttgcttttgagcaaggttgtttga

SEQ ID NO: 80 - DNA, 1587 bp, synthetic construct
  misc_feature 1..1587: note = codon optimized cDNA
  source 1..1587: mol_type = other DNA
atggttcgtgcgttgattttggatttgggtgatgtgttgtttaattgggacgcccctgcaagcactccgatcagccgtaagaccctgggccagatgctgcattccgagatttggggtgagtatgagcgtggtcacctgaccgaagatgaagcgtacaacgcgctggcaaagcgctacagctgcgaggcaaaagacgtggcgcatacttttgttttggcgcgtgaaagcctgcgcctggataccaagtttaagacttttctgcagaccctgaaacagaacgcgaacggctcgctgcgtgtttatggtatgtccaatatcagcaaaccggattttgaagtgctgctgggtaaagctgacgactggaccttgttcgacaagatcttcccgagcggtcatgtcggtatgcgcaaaccggacctggctttctttcgttacgtgctgaaagacatcagcaccccggttgaggatgttgtgtttgttgacgataacctggataatgtgacgtctgcccgttccctgggtatgcgtagcgtcctgttccacaaaaaagacgaagtccaacgtcagctgaccaacattttcggtagccctgctgagcgcggtctggagtatctgtccgcgaacaagaccaatctgcaaagcgcaaccaccaccgacatccctatccaagacaactttggtcaattactgattctggaagccaccgaagatccgagcctggtacgcatggaaccgggcaagcgtacctggaatttcttcattggctctccgagcctgacgacggataccttcccggatgacctggacacgacgagcctcgcactgtccatcgtgccgaccagcccagatgttgttaatagcgtgatcgatgagatcatcagccgtcgcgacaaggacggtattgtgccgacgtactttgataacacgcgcccgcgtgtggacccgattgtttgtgttaacgttctgtctatgttcgcgaaatatggccgtgagcacgatctgccggcgacggtcgcgtgggtccgcgacgtcctctatcatcgcgcatacctgggtggcaccagatactacggtagcgcggaagccttccttttcttctttacgcgctttgtgcgtaatctgcgtccgggcacgctgaaacaagatctgcacgcgttgctgagcgagcgtgtccgtgagcgcctgaataccccggtggatgcgctggcgctgagcatgcgcattcaggcttgccacgcactgggctttgacgccccagctgacatcgcgacgctgattaccatgcaagatgaagatggtggctggccggcggcagttatctacaaatatggtgcgggtggcctgggcattacgaaccgtggtgtgtccacggcattcgcggtgaaggcaatcacgggtagcccggttaaaaccgaaaccaacatcggcggcgacggtgcccgtgcagtgtcggccatgagcagcctggaagcccgtcgtttgcagccgatttctagcgtcggcgactgggtccgtttcatcatcgcatcactgcacgtccacctggcgtggctgtggaatgtcctgctgctgagcaaagtcgtttaa

SEQ ID NO: 81 - protein, 531 aa, Heterobasidion irregulare
MSMIPRCSNLILDIGDVLFTWSPKTSTSISPRTMKSILSSTTWHQYETGHISQGDCYRLIGNQFSIDPQEVGLAFQQARDSLQPNVDFIHFIRALKAESHGTLRVFAMSNISQPDYAVLRTKDADWAVFDDIFTSADAGVRKPHLGFYKLVLGKIGADPNDTVFVDDKGDNVLSARSLGLHGIVFDSMDNVKRALRYLISDPIRRGREFLQARAGHLESETNTGIEIGDNFAQLLILEATKDRTLVNYMDHPNKWNFFRDQPLLTTEEFPFDLDTTSIGTLATQRDDGTANLVMDEMLQYRDEDGIIQTYFDHERPRIDPIVCVNVLSLFYSRGRGSELAPTLEWVRGVLKHRAYLDGTRYYETGECFLFFLSRLLQSTKDAALHASLKSLFAERVKERIGAPGDALALAMRILACAAVGVRDEIDLRSLLPLQCEDGGWEAGWVYKYGSSGVKIGNRGLTTALALNAIEAVEGRRTRPKSGKISRVSRHSEVAAAPRSSTSSHRSNRSISRTFQAYFKASWTSMKQVAVA

SEQ ID NO: 82 - DNA, 1596 bp, Heterobasidion irregulare
  misc_feature 1..1596: note = cDNA
  source 1..1596: mol_type = genomic DNA
atgtccatgatacccagatgctcgaatctcatcctcgacatcggggatgttctcttcacatggtctccgaagacgtccacttcgatctccccccgcaccatgaagagcatactgtcatcgacgacctggcaccaatacgagaccgggcacatttcacagggcgactgctaccgcctcataggcaaccagttctccatcgatcctcaggaagtcggacttgcattccaacaagctcgggactcattgcagcctaatgttgacttcattcacttcatccgcgccctcaaggcggaatcacacgggacgctgcgcgtcttcgctatgtccaacatctctcagcccgattacgcagttcttcggactaaggacgccgactgggccgtttttgacgatatattcacgtctgcagatgctggggttcgaaagccacaccttgggttctacaagttggtactcggaaagatcggcgccgatccaaacgataccgtcttcgtcgatgacaagggggacaatgtcctctctgcacggtctctcggccttcatggaatcgtctttgacagtatggacaacgtcaagcgagccctgcgctacttgatcagcgaccccatacggcgaggacgagagtttctccaagcgcgagccggccatttggagtcggagaccaatacgggcatcgaaatcggtgataattttgcccagctccttattctcgaggccacgaaggataggacactcgtcaattatatggaccatccgaacaaatggaatttcttccgagatcaaccgctcctcacaacggaggagttccctttcgatctcgatacgacatctattggaacgcttgcgacgcagcgcgatgatgggactgccaatctagtaatggatgagatgcttcagtaccgtgatgaggatggcataatacaaacatatttcgatcatgaacgaccgaggatagatcccatcgtctgtgtcaacgtcttgagccttttctactcccggggtcgtggttcggagctagcaccgacactagagtgggtgcgtggtgtcctcaagcaccgcgcgtatctcgatggaacgcgatactacgagacaggcgaatgcttccttttcttcctcagccggctcttgcaatcaaccaaggacgccgccttgcacgcatcgttgaaatctttgttcgccgaacgggtcaaggagcgcataggggcaccaggggacgcgctggcgctggcgatgcgtatactggcatgcgcagcagtgggcgtgcgggacgagatcgatcttcgatcactattacctctgcagtgcgaggatggggggtgggaggcaggctgggtgtacaagtatgggtcttcgggagtcaagatcggcaatcgtggcctcacgactgcgcttgcgctcaatgccatcgaggctgtggagggacgtcgcacgaggccgaagtcgggtaagatcagccgagtcagccgtcattctgaggtcgcagcagcgccacggtcttccaccagcagtcatcgttctaatcgctcgatctcaaggacattccaggcgtacttcaaggcgtcgtggacatcgatgaaacaggtggccgtggcgtga

SEQ ID NO: 83 - DNA, 1596 bp, synthetic construct
  misc_feature 1..1596: note = codon optimized cDNA
  source 1..1596: mol_type = other DNA
atgagcatgattccacgttgtagcaatctgattctcgacatcggtgatgtgttgtttacgtggagcccgaaaaccagcaccagcattagcccgcgtaccatgaaatctatcctgagctctaccacctggcatcaatatgagactggccacatcagccagggtgattgctaccgcctgatcggtaatcagttctccatcgacccgcaagaggtcggtttggccttccagcaagccagagacagcctgcaaccgaatgttgatttcatccatttcattcgtgccctgaaagctgagtcgcacggcaccctgcgcgtttttgcgatgagcaatatcagccaacctgactatgcagtcctgcgtacgaaagacgcggactgggctgtttttgatgatatcttcacgagcgcggatgctggtgttcgtaaaccgcacctgggtttttataaactggtcttaggcaagattggcgcggaccctaacgacaccgtttttgtggatgataagggtgacaacgtcctctctgcacgttccctgggtctgcacggtatcgtttttgattcaatggacaacgtgaagcgcgcactgcgctacctgattagcgacccgatccgccgcggccgtgaatttctgcaggcccgtgcgggtcacctggagtccgaaacgaacacgggtattgagattggtgataatttcgcgcaattgctgatcctggaagcgaccaaagatcgtactctggtgaactacatggaccacccgaacaagtggaacttcttccgtgaccagccgctgctgaccaccgaagaatttccgttcgacctggacacgaccagcattggcacgctggccacccaacgtgacgatggtacggcgaatctggtaatggacgaaatgttgcagtatcgtgacgaagatggcatcattcagacctatttcgatcatgagcgcccgcgtattgatccgattgtttgtgtgaatgtgctgtctctgttctacagccgtggccgtggctctgagttggcgccgacgctggaatgggtgcgcggtgtgttgaaacatcgtgcgtacctggatggtacgcgttattacgagactggtgagtgtttcctgtttttcctgagccgtctgctgcagagcaccaaagacgcagccctgcacgcgagcctgaagtccctgtttgcagagcgtgttaaagagcgcatcggtgcgccgggcgatgctctggcgctggctatgcgcatcctggcgtgcgccgctgttggtgtgcgcgatgaaattgatttgcgtagcctgctgccgctgcaatgcgaagatggcggctgggaagcgggctgggtctacaaatacggcagcagcggtgtgaagattggcaatcgcggtcttaccacggcgctggcattgaatgctatcgaagccgttgagggccgtcgcacccgcccaaagtccggtaagatcagccgtgttagccgtcatagcgaagtcgcagcggcaccgcgttcctcgacgagcagccaccgtagcaaccgtagcattagccgcaccttccaggcatattttaaagcgagctggaccagcatgaaacaagtcgcagtggcgtaa

SEQ ID NO: 84 - protein, 518 aa, Schizopora paradoxa
MSIHGSSMSSYSSTVPSMTSSPASTSTPSSPASSIHEIGPVPEARRKGQCNALIFDLGDVLFTWSAETKTTISPKLLKKILNSLTWFEYEKGNIGEQEAYDAVAKEFGVPSSEVGAAFQCARDSLQSNPRLVSLIRELKSQYDLKVYAMSNISAPDWEVLRTKATPEEWAMFDRVFTSAAARERKPNLGFYRQVVEATGVDPARSVFVDDKLDNVISARSVGLNAIIFDSFENVARQLKNYVADPIGRAEAWLRDNAKKMLSITDAGVVVYENFGQMLILEATGDRSLVDYVEYPRLFNFFQGNGVFTTESFPCDLDSTSIGLTVTNHVDEKTRHSVMDEMLTYKNEDGIIATYFDATRPRIDPVVCANVLTFFYKNGRGEELNETLDWVYDILLHRAYLDGTRYYFGSDTFLFFLSRLLSESPSVYARFAPVFQERVKERMGATGDAMSLAMRIIAAATVKIQDRVDCDALLQTQEDDGGFPIGWMYKYGATGMLLGNKGLSTALAIQAIKAVESFP

SEQ ID NO: 85 - DNA, 1557 bp, Schizopora paradoxa
  misc_feature 1..1557: note = cDNA
  source 1..1557: mol_type = genomic DNA
atgtcgattcacggttcttctatgtcctcctattcctcgactgtgccgtcaatgacttcctctcccgcgtccacttctactccgtcgtctcctgcatcgtcgatccatgagattggtcctgtcccagaagctcgacgaaagggacagtgcaacgcgctgatcttcgacctcggagacgtcctcttcacctggtcggcagagactaagaccaccatttccccgaaactcctgaaaaagatccttaactccttaacatggttcgaatacgagaagggaaacatcggggagcaggaggcgtatgacgcagtcgcaaaggagtttggcgtcccgtcgtccgaggtcggggccgctttccagtgcgcgcgcgattcgctacagagcaatccccgcctcgtctcgctcatccgtgagctgaagtcgcaatatgatctcaaggtgtacgccatgtccaacatctctgcgccggactgggaagtcctaaggacgaaggcgacccctgaggagtgggcaatgtttgaccgcgtcttcacgagcgcggccgcgcgcgagcgtaagccaaacctcggattctacagacaggttgttgaggcgaccggcgtcgaccccgctcgctccgtgttcgtcgacgataaactcgacaatgtcatctctgcgcgttcagtcggattaaatgcgatcatcttcgactcatttgagaacgtcgcccggcagctcaaaaactatgtcgctgatcctatcggacgggcggaggcgtggttgcgcgataacgcaaagaagatgttgtcaattacggatgccggggtggtcgtatacgagaatttcggccagatgctgatcttggaggcaacaggcgataggtcgcttgtggactacgtcgagtaccctcgtctcttcaacttcttccaaggcaatggcgtctttacgaccgagtcattcccttgcgaccttgattcgacttccatcggcttaaccgtcacgaaccacgtcgatgagaaaacaaggcacagcgtcatggatgagatgctgacctacaaaaatgaggatggtatcattgcgacttactttgatgccacgcgtccccgaattgaccccgtcgtctgcgccaatgtcttgacgttcttctacaagaacggccgaggggaggagctcaatgaaacacttgactgggtctacgacatcctccttcatcgcgcgtacctcgatggcacacgctattatttcggctcagacaccttcctcttcttcctttctcgacttctctccgaatcgccatccgtttacgcccgtttcgctccggtgttccaggagagagtcaaggagcgcatgggggcgacgggagatgcgatgtcccttgcgatgcgcatcatcgcggccgcaactgtcaagatccaagaccgagtcgactgcgacgctctgctgcagacgcaggaagacgacggtggattcccgataggttggatgtacaagtacggggcgaccgggatgcttctgggtaacaagggcttgtcgacagctctggcaatccaagctatcaaagcggtcgaatctttcccttga

SEQ ID NO: 86 - DNA, 1557 bp, synthetic construct
  misc_feature 1..1557: note = codon optimized cDNA
  source 1..1557: mol_type = other DNA
atgtcgattcacggtagcagcatgtcgtcttatagcagcacggttccatctatgactagcagcccggcttccacgagcacgccgtccagcccggccagcagcatccacgaaatcggcccggtccctgaggcgcgtcgcaagggccaatgcaatgcactgatcttcgacctgggtgatgttctgtttacctggagcgcagaaaccaagaccacgatcagcccgaagctgctgaaaaagattctgaacagcttgacctggtttgagtatgagaaaggcaacatcggtgaacaagaagcctatgacgccgttgcgaaagagttcggtgtgccgagctctgaggttggcgctgcgtttcaatgtgcgcgtgactccctgcaaagcaatccgcgtttggttagcctgattcgtgagctgaagtcccagtacgacctgaaagtgtacgctatgagcaatattagcgcgccagactgggaagtgctgcgtactaaagcgaccccggaagagtgggcaatgttcgatcgtgtctttacttctgcggcggcgcgtgagcgtaagccgaacttgggcttttaccgccaagtcgtggaagcaaccggtgtcgatccggcgcgtagcgttttcgtcgatgataaactggacaatgtgatcagcgcgcgctctgtcggtctgaacgctattatcttcgactccttcgaaaacgtcgcccgtcagctgaagaattacgtcgcagacccgattggtcgcgctgaggcgtggctgcgcgacaacgcaaagaaaatgctgagcatcaccgatgcgggtgttgtggtttacgagaattttggccagatgctgatcctggaagctaccggtgaccgtagcctggtggactatgtggagtatccgcgcctctttaacttcttccagggtaacggcgtttttacgaccgagagctttccatgcgatctggacagcaccagcatcggtctgactgtgaccaatcatgtggacgaaaagactcgccacagcgtcatggacgaaatgctgacctacaaaaatgaagatggtattattgcgacgtactttgacgcgacgcgcccgcgcattgaccctgttgtctgtgccaatgttctgaccttcttctacaaaaacggtcgtggtgaagaattgaacgaaaccctggattgggtgtacgacattctgctgcatcgcgcgtatctggacggtacgcgttattatttcggctccgatacgttcctgtttttcctgagccgtctgctgagcgagtctccgagcgtttacgcgcgttttgccccggtgtttcaagagcgcgtgaaagagcgtatgggcgcgaccggtgatgcgatgagcctggccatgcgtatcattgcagcagcaaccgtaaagatccaggatcgtgtggattgcgacgcactgttgcagacccaagaagatgatggcggtttcccgattggttggatgtacaaatatggtgcgaccggtatgttgctgggcaacaaaggcctgagcacggccctggcgatccaggcaattaaagccgtcgagtcgttcccgtaa

SEQ ID NO: 87 - protein, 507 aa, Aspergillus versicolor
MGSTKALVVDFGNVLCTWTPPRELSIPPKKLKQIMSSDIWLDYERGIYKSEDECYLAVATRFGVSPSDLSSVMKKARESLQPNTATLNHLSHLKKTQPGLRIYGLTNTPLPEQSSVRSIAQEWPIFDHIYISGILGMRKPDIGCYRLVLRKIGLPAESVVFIDDSPENILAAQSLGVHSILFQSHDQLSRQLGNVLGDPIQRGHNFLLSNAKQMNSTTDKGVIIRDNFAQLLIIELTQNPDLVALETWDRTWNFFIGPPQLTTESFPNDLDTTSIALSVLPVDKEVVWSVMDEMLTFTNADGIFMTYFDRSRPRVDPVVCTNVLNLFCMHGRESEVAATFDWVLDVLRNSAYLSGSRYYSSPDCFLYFLSRLSCVVRDGTRRRELKSLLKQQVSQRIGADGDSVSLATRLLASNILGITNGRDRSRLLALQETDGGWPAGWVYKFGSSGVQIGNRGLSTALALKSIERQKGPVEAISSEPEAWWPSLRLDRLLNVWPFIDWKGYSPS

SEQ ID NO: 88 - DNA, 1524 bp, Aspergillus versicolor
  misc_feature 1..1524: note = cDNA
  source 1..1524: mol_type = genomic DNA
atgggttccaccaaggctcttgttgttgactttgggaatgttttgtgtacctggacaccacccagggagttatccatcccgcccaagaagctgaaacaaatcatgtcttctgacatttggctcgactatgaacggggtatctataagtcggaggacgagtgctacttggcggttgcaactcgcttcggcgtctctcccagcgacctctcctcggtgatgaaaaaggcccgcgagagcctgcaaccaaacaccgcaaccctgaatcatctgtctcatctcaaaaagacccagcctggcctcaggatatacggtttgaccaacacccctctcccagaacaaagcagtgtacgatccatcgcccaggaatggcctatcttcgaccatatctacatatcaggcatcctcggaatgcgcaagccggacattggctgctacaggctggtgctgcgaaagattgggcttccagcggagtccgtggtcttcattgatgattcacccgagaacatcctggccgcgcagtcactgggagtacacagcatactgttccaaagccacgaccagctctctcgtcagcttggcaatgtgctgggtgatccaatccagcggggccataacttcctactctcgaacgcaaagcaaatgaatagtacgaccgacaagggagttattatccgggacaactttgcgcaactgctgatcatcgagctgacgcagaacccagaccttgtggcgttagaaacatgggaccgtacctggaatttttttattggacctccacaattgacaactgaaagctttcccaatgatcttgacactacctccatcgctctctcggttcttccggttgacaaagaagtggtatggtctgtgatggacgagatgctaacgtttaccaatgcggatgggatttttatgacctatttcgaccgatcacgccctcgagttgatccggtagtttgcaccaatgtcctgaatcttttctgcatgcatggacgggaaagcgaagttgcagccacatttgactgggtgctggacgttcttcgaaattcggcctatttatcaggatccagatactattcttcgcctgattgctttctatactttctttcacggctgagctgtgtggtccgagacggcacgcgacgcagggagctcaagtcactgttgaaacaacaagtgagccagcgtattggcgctgatggtgattccgtctctctcgccactaggctacttgcatcgaacattttaggaatcacaaatggccgtgatcgctccaggcttcttgctctgcaggaaactgacggtggatggcctgctgggtgggtttataaattcggaagctcgggggtacagattggcaatcgggggctcagtacagccttggcgttaaaatcaattgagcgtcagaaggggcctgttgaggcgatatccagtgagccagaagcgtggtggccatccctcaggcttgaccgacttctcaacgtttggcctttcatcgactggaagggatattcgccgagttga

SEQ ID NO: 89 - DNA, 1524 bp, synthetic construct
  misc_feature 1..1524: note = codon optimized cDNA
  source 1..1524: mol_type = other DNA
atgggttctacgaaagcgttggttgttgattttggtaatgttctgtgcacttggacgccaccacgtgaattgtccatcccgccgaagaaactgaagcaaatcatgagcagcgacatttggctggactatgagcgtggtatctacaaatcggaagatgagtgctacctggcagttgcgacgcgctttggtgtcagcccgtccgacctgagctccgttatgaaaaaagcccgtgagagcctgcagccgaataccgcaacgctgaaccacttgagccatctgaagaaaacccagcctggccttcgtatctacggcctgacgaacaccccgttgccggaacagagctcagtccgtagcattgcgcaggaatggccgatttttgaccacatctacattagcggcatcttgggtatgcgcaaaccggatattggttgttaccgtctggttctgcgtaagatcggtctgccagcggagtccgtcgtattcatcgacgacagcccggagaacattctggcagctcaatcgttgggtgtccatagcatcctgttccagtcccacgatcagctgagccgtcagctgggcaatgtgctgggtgatccgattcagcgcggtcacaacttcctcctgtccaacgcgaagcaaatgaacagcaccaccgataagggtgtgattatccgcgacaacttcgcccagctgctgattattgagctgacccaaaatccggatctggttgcgctggagacttgggaccgtacgtggaatttctttattggtccgccgcaactgaccaccgagagctttccgaacgacctggacaccacgagcattgccctgagcgtgttgccggtggataaagaagtcgtttggtctgtgatggatgagatgctgaccttcaccaacgcagacggcatcttcatgacctatttcgatcgtagccgtccgcgtgttgacccggtcgtttgtaccaatgtcctgaatctgttttgcatgcatggtcgcgagagcgaagtggccgcgacgttcgactgggtgctggacgtgctgcgcaacagcgcgtacctgagcggttcccgttattacagcagcccggattgttttctgtatttcctgtctcgtctgagctgcgtcgtccgtgatggcacgcgtcgtcgtgaactgaaaagcctgctgaagcaacaagtttctcaacgtatcggcgctgacggtgattccgtcagcctggccacccgtttgctggcgagcaacatcctgggcattactaacggtcgtgaccgcagccgtctgctggcattgcaagaaaccgatggtggctggcctgcaggctgggtctataagtttggtagcagcggcgtgcaaattggcaatcgcggtctgagcaccgcgctggctctgaagtctatcgagcgccagaaaggtccggtggaagcaatcagcagcgagccggaagcgtggtggcctagcttacgcttggaccgcttgctgaatgtttggccatttatcgactggaagggctactccccgagctaa

SEQ ID NO: 90 - protein, 540 aa, Agaricus bisporus; identical to SEQ ID NO: 7

SEQ ID NO: 91 - DNA, 1808 bp, Agaricus bisporus
  misc_feature 1..1808: note = cDNA
  source 1..1808: mol_type = genomic DNA
atggctccgcctcagcgaccctttactgcgattgtctttgacatcggggatgttctattccaatggtctgcaaccaccaaaacctctatctcaccaaagacactccgctctattctcaactgtccgacatggtttgactatgaacgtggacgcctggcagaaaacgcttgttatgccgctatctcacaagaattcaacgtcaacccagacgaagttcgcgacgctttcagccaagcgcgcgactctctccaagcaaaccacgacttcatcagtctcatccgtgagctgaaggcacaagcaaatggtcgtttacgtgtgtacgccatgtcgaacatatctcttcctgattgggaagtgctgcggatgaaacctgctgattgggatattttcgaccacgtcttcacatccggtgcggttggggaacgcaagcccaatctcgccttttatcgccatgttatcgcggccaccgatctgcagcctcatcagacaatatttgttgacgataagctggagaatgttctctcagcacgttccctcgggttcacaggcatcgtgtttgacgagccctccgaggtcaaacgtgcgcttcgtaacctcattggggatcctgttcaacgaggaggtgaattcttggttcggaatgccggaaagcttggctctatcacaaggactactgcaaagcacgagtcaatccccctcgacgagaattttgctcagcttcttattctcgagataacggggaacaggtgcgttagcttcttgtagggtcttctgtcgtaatactaaattttttctggtgtttagggctttggtcaacctcgttgagcatcctcaaacgtggaatttcttccaaggtgcgctgctaaaataaacatccagttgcgtttcgaagctcattgtgggcgtcccgtcacaggcaagggccagctgacaacagaagaatttccattcgatctcgatacaacttctcttggtctcacgatcctcaagcgaagcagggaaatcgccgattcagtcatggatgaaatgctggagtatgtcgatcctgatggtatcattcaggcaagtttcatttatcggcttgagaaaataaagacaaaaacgttctgatggggggatgtttctagacgtatttcgatcatcggagaccacgttttgatccagtcgtgtgtgtcaatgcattaagcctcttctatgcttacggccgcggggagcaactgcggtcgactttgacatgggtacatgaagtccttctcaatcgagcctacttggatggcacacggtactacgaaacagccgaatgcttcctctatttcatgagccgacttctcgccacttcaggcgaccctgaccttcactcccttcttaaacctcttctcaaagaacgggtgcaagaacgcattggagctgatggagactctcttgcactcgcaatgcgtattctcgcctgtgatttcgtcggaatcagagatgaagtggatttacgcacacttctgactttgcaatgtgaagatggaggttgggaagtgggttggatgtacaagtatggatcttccggtatcagtatcggaaatcgtggactggccaccgcgctcgctatcaaggccgtcgacacgatgtttcaaccccaaattcggttctctgaatcacccacagatactttggttgaaaacgctatccacaaacgccgtccctcattttccgaaaaattcctcggcaaacgtcctcgcagcggatcgttcaggaaacctttacagtggatactgcaaggttccaagcttcgcaaatctgtcgaaataggaagctaa

SEQ ID NO: 92 - DNA, 1623 bp, Agaricus bisporus
  misc_feature 1..1623: note = optimized cDNA
  source 1..1623: mol_type = genomic DNA
atggcaccaccgcaacgtccgttcactgcaattgttttcgatattggcgatgttttgttccaatggtctgcgaccacgaaaaccagcattagcccgaaaaccctgcgcagcattctgaattgtccgacctggtttgattatgagcgcggccgtctggcggaaaatgcgtgttacgctgcgatcagccaagaatttaacgtcaacccggacgaagttcgcgacgccttcagccaagcgcgcgacagcctgcaggcgaatcacgacttcatcagcctgattcgtgagctgaaagctcaggcgaacggtcgtctgcgtgtctacgccatgtctaatatcagcctgccggattgggaagtcctgcgtatgaagccagccgattgggacatctttgaccatgtatttaccagcggtgcggtgggtgagcgcaagccgaacctggccttttatcgtcacgtcatcgcggccacggatctgcagccgcaccagacgatcttcgtggatgacaaactggaaaacgtgctgtctgcgcgctcgctgggcttcacgggtatcgtgttcgacgagccaagcgaagtcaaacgtgcgctgcgtaatctgatcggcgacccggtgcagcgtggtggcgagttcctggttcgtaatgctggcaaactgggttctatcacccgtacgaccgcaaaacatgagagcatcccgctggatgagaattttgcacaactgttgattctggaaattactggtaaccgcgcactggtcaatctggttgagcacccgcagacgtggaacttcttccagggtaagggccagctgacgaccgaagaatttccttttgacctggatacgacgagcctgggtctgacgatcctgaagcgtagccgcgagattgccgactccgtcatggacgaaatgttggaatacgtggaccctgacggcatcattcagacctacttcgatcatcgtcgcccgcgctttgacccggttgtttgcgttaatgccctgagcctgttctatgcatacggccgtggtgagcaactgcgttccaccttgacctgggtgcacgaagttctgttgaaccgtgcgtatttggatggtacgcgttactatgaaacggccgagtgctttctgtatttcatgtcccgtctgctggcaaccagcggtgacccggatctgcattccctgctgaagccgttgctgaaggaacgcgtgcaagagcgcatcggcgctgacggtgacagcctggcgctggcgatgcgcattttggcatgtgattttgttggcatccgtgatgaagtggatctgcgtaccctgctgaccttacagtgcgaggatggcggttgggaagtgggctggatgtacaaatacggtagcagcggtattagcattggtaaccgtggtctggcaaccgcattggcgatcaaagctgttgacaccatgtttcaaccgcaaatccgtttcagcgagagcccgaccgacactctggtggagaacgcgattcacaagcgccgcccgagcttttcagagaaatttttaggtaagcgtccgcgttccggttcgttccgtaaaccgctgcaatggattctgcagggcagcaagctgcgcaagagcgtcgagatcggtagctaa

SEQ ID NO: 93 - DNA, 1584 bp, synthetic construct; identical to SEQ ID NO: 4
  misc_feature 1..1584: note = XP_007369631.1 optimized cDNA for S. cerevisiae expression
  source 1..1584: mol_type = other DNA

SEQ ID NO: 94 - DNA, 1623 bp, synthetic construct; identical to SEQ ID NO: 6
  misc_feature 1..1623: note = XP_006461126 optimized cDNA for S. cerevisiae expression
  source 1..1623: mol_type = other DNA

SEQ ID NO: 95 - DNA, 1566 bp, synthetic construct
  misc_feature 1..1566: note = LoTps1 Optimized cDNA for S. cerevisiae expression
  source 1..1566: mol_type = other DNA
atgtacactgctttgatcttggacttgggtgacgttttgttctcttggtcttctactactaacactactatcccaccaagacaattgaaggaaatcttgtcttctccagcttggttcgaatacgaaagaggtagaatcactcaagctgaatgttacgaaagagtttctgctgaattctctttggacgctactgctgttgctgaagctttcagacaagctagagactctttgagaccaaacgacaagttcttgactttgatcagagaattgagacaacaatctcacggtgaattgactgttttggctttgtctaacatctctttgccagactacgaattcatcatggctttggactctaagtggacttctgttttcgacagagttttcccatctgctttggttggtgaaagaaagccacacttgggtgctttcagacaagttttgtctgaaatgaacttggacccacacactactgttttcgttgacgacaagttggacaacgttgtttctgctagatctttgggtatgcacggtgttgttttcgactctcaagacaacgttttcagaatgttgagaaacatcttcggtgacccaatccacagaggtagagactacttgagacaacacgctggtagattggaaacttctactgacgctggtgttgttttcgaagaaaacttcactcaattgatcatctacgaattgactaacgacaagtctttgatcactacttctaactgtgctagaacttggaacttcttcagaggtaagccattgttctctgcttctttcccagacgacatggacactacttctgttgctttgactgttttgagattggaccacgctttggttaactctgttttggacgaaatgttgaagtacgttgacgctgacggtatcatgcaaacttacttcgaccacactagaccaagaatggacccattcgtttgtgttaacgttttgtctttgttccacgaacaaggtagaggtcacgaattgccaaacactttggaatgggttcacgaagttttgttgcacagagcttacatcggtggttctagatactacttgtctgctgactgtttcttgttcttcatgtctagattgttgcaaagaatcactgacccatctgttttgggtagattcagaccattgttcatcgaaagagttagagaaagagttggtgctactggtgactctatcgacttggctttcagaatcatcgctgcttctactgttggtatccaatgtccaagagacttggaatctttgttggctgctcaatgtgaagacggtggttgggacttgtgttggttctaccaatacggttctactggtgttaaggctggtaacagaggtttgactactgctttggctatcaaggctatcgactctgctatcgctagaccaccatctccagctttgtctgttgcttcttcttctaagtctgaaatcccaaagccaatccaaagatctttgagaccattgtctccaagaagattcggtggtttcttgatgccatggagaagatctcaaagaaacggtgttgctgtttcttcttaa

SEQ ID NO: 96 - DNA, 1602 bp, synthetic construct
  misc_feature 1..1602: note = EMD37666.1 optimized cDNA for S. cerevisiae expression
  source 1..1602: mol_type = other DNA
atgtctgctgctgctcaatacactactttgatcttggacttgggtgacgttttgttcacttggtctccaaagactaagacttctatcccaccaagaactttgaaggaaatcttgaactctgctacttggtacgaatacgaaagaggtagaatctctcaagacgaatgttacgaaagagttggtactgaattcggtatcgctccatctgaaatcgacaacgctttcaagcaagctagagactctatggaatctaacgacgaattgatcgctttggttagagaattgaagactcaattggacggtgaattgttggttttcgctttgtctaacatctctttgccagactacgaatacgttttgactaagccagctgactggtctatcttcgacaaggttttcccatctgctttggttggtgaaagaaagccacacttgggtgtttacaagcacgttatcgctgaaactggtatcgacccaagaactactgttttcgttgacgacaagatcgacaacgttttgtctgctagatctgttggtatgcacggtatcgttttcgaaaagcaagaagacgttatgagagctttgagaaacatcttcggtgacccagttagaagaggtagagaatacttgagaagaaacgctatgagattggaatctgttactgaccacggtgttgctttcggtgaaaacttcactcaattgttgatcttggaattgactaacgacccatctttggttactttgccagacagaccaagaacttggaacttcttcagaggtaacggtggtagaccatctaagccattgttctctgaagctttcccagacgacttggacactacttctttggctttgactgttttgcaaagagacccaggtgttatctcttctgttatggacgaaatgttgaactacagagacccagacggtatcatgcaaacttacttcgacgacggtagacaaagattggacccattcgttaacgttaacgttttgactttcttctacactaacggtagaggtcacgaattggaccaatgtttgacttgggttagagaagttttgttgtacagagcttacttgggtggttctagatactacccatctgctgactgtttcttgtacttcatctctagattgttcgcttgtactaacgacccagttttgcaccaccaattgaagccattgttcgttgaaagagttcaagaacaaatcggtgttgaaggtgacgctttggaattggctttcagattgttggtttgtgcttctttggacgttcaaaacgctatcgacatgagaagattgttggaaatgcaatgtgaagacggtggttgggaaggtggtaacttgtacagattcggtactactggtttgaaggttactaacagaggtttgactactgctgctgctgttcaagctatcgaagcttctcaaagaagaccaccatctccatctccatctgttgaatctactaagtctccaatcactccagttactccaatgttggaagttccatctttgggtttgtctatctctagaccatcttctccattgttgggttacttcagattgccatggaagaagtctgctgaagttcactaa

SEQ ID NO: 97 - DNA, 1461 bp, synthetic construct
  misc_feature 1..1461: note = XP_001217376.1 optimized cDNA for S. cerevisiae expression
  source 1..1461: mol_type = other DNA
atggctatcactaagggtccagttaaggctttgatcttggacttctctaacgttttgtgttcttggaagccaccatctaacgttgctgttccaccacaaatcttgaagatgatcatgtcttctgacatctggcacgactacgaatgtggtagatactctagagaagactgttacgctagagttgctgacagattccacatctctgctgctgacatggaagacactttgaagcaagctagaaagtctttgcaagttcaccacgaaactttgttgttcatccaacaagttaagaaggacgctggtggtgaattgatggtttgtggtatgactaacactccaagaccagaacaagacgttatgcactctatcaacgctgaatacccagttttcgacagaatctacatctctggtttgatgggtatgagaaagccatctatctgtttctaccaaagagttatggaagaaatcggtttgtctggtgacgctatcatgttcatcgacgacaagttggaaaacgttatcgctgctcaatctgttggtatcagaggtgttttgttccaatctcaacaagacttgagaagagttgttttgaacttcttgggtgacccagttcacagaggtttgcaattcttggctgctaacgctaagaagatggactctgttactaacactggtgacactatccaagacaacttcgctcaattgttgatcttggaattggctcaagacagagaattggttaagttgcaagctggtaagagaacttggaactacttcatcggtccaccaaagttgactactgctactttcccagacgacatggacactacttctatggctttgtctgttttgccagttgctgaagacgttgtttcttctgttttggacgaaatgttgaagttcgttactgacgacggtatcttcatgacttacttcgactcttctagaccaagagttgacccagttgtttgtatcaacgttttgggtgttttctgtagacacaacagagaaagagacgttttgccaactttccactggatcagagacatcttgatcaacagagcttacttgtctggtactagatactacccatctccagacttgttcttgttcttcttggctagattgtgtttggctgttagaaaccaatctttgagagaacaattggttttgccattggttgacagattgagagaaagagttggtgctccaggtgaagctgtttctttggctgctagaatcttggcttgtagatctttcggtatcgactctgctagagacatggactctttgagaggtaagcaatgtgaagacggtggttggccagttgaatgggtttacagattcgcttctttcggtttgaacgttggtaacagaggtttggctactgctttcgctgttagagctttggaatctccatacggtgaatctgctgttaaggttatgagaagaatcgtttaa

SEQ ID NO: 98 - DNA, 1680 bp, Drimys lanceolata
  misc_feature 1..1680: note = DlTps589 wild type DNA sequence
  source 1..1680: mol_type = genomic DNA
atggatcttattaatccctccccagcggcttccaccctccctctcccagttgatggagattcagaagttgttaggcgatctgccgggtttcatccgactatctggggcgatcacttcctctcctacaagcccgatccaaagaaaatagatgcatggaataaaagggttgaagagctgaaggaagaagtgaagaagatattaagcaatgcaaaagggacggtggaagagctgaatttgattgatgatctcgtacaccttgggattagttatcattttgagaaggagattgatgatgctctacaacacatctttgatacccatcttgatgattttcctaaggatgatctatatgtcgccgctctccgatttggcgtcttaaggaaacaggggcaccgtgtttctccagatgtattcaaaaaattcaaagatgagcaggggaatttcaaggcagagttgagcaccgatgcgaaaggtttgctatgtttaaatgatgtggcttatctcagcacaagaggggaagatatcttggatgaagccattcctttcactgaggagcaccttaggtcttgtattagccatgtagattctcatatggcagcaaaaattgaacattctctcgagcttccccttcatcatcgcataccaaggctagagaacaggcactacatctcagtctatgaaggagacaaggaaaggaacgaagttgtccttgagcttgccaatttagatttcaatctgattcaaatcttgcaccaaagagagctgagagacatcacaatgtggtggaaggagattgaccttgcagcaaagctgccttttattagggataggttggtggagtgctactactggatcatgggggtctattttgaaccaatatactcgagggctagggttttttccaccaaaatgacaatgttggtctcagttgtggacgacatatatgatgtgtatgctaccgaggatgagcttcaactattcactgatgccatctataggtgggatgctgatgacattgatcagctgcctcagtacttgaaagatgcttttatggtactctacaacactgtgaagactctagaagaagaacttgaaccagaaggaaactcttatcgtggattctatgtaaaagatgcaatgaaggttttggcaagggattactttgtggagcacaaatggtataacagaaaaattgtgccatccgtagaggaatacttgaaaatttcttgcatcagtgtggccgttcatatggctacagttcactgtattgctgggatgtatgaaattgcaaccaaagaggcattcgaatggttgatgactgagcccaaacttgttattgatgcatctctgattggtcgtctccttgatgacatgcagtccacctcgtttgagcaacagagaggccacgtgtcatcagcagtacagtgttacatggctgaatatggtgtaacagcggaagaagcatgtgaaaagctccgagatatggctgcaattgcttggaaagatgtgaacgaggcatgccttaggcccacggttttccctatgcctatccttttgccttctatcaacttggcacgtgtggcagaagtcatctacctacgtggagatggatacacgcacgctgggggtgagaccaagaaacacatcacggccatgcttgttaagccaattgaagtctga

SEQ ID NO: 99 - protein, 559 aa, Drimys lanceolata
MDLINPSPAASTLPLPVDGDSEVVRRSAGFHPTIWGDHFLSYKPDPKKIDAWNKRVEELKEEVKKILSNAKGTVEELNLIDDLVHLGISYHFEKEIDDALQHIFDTHLDDFPKDDLYVAALRFGVLRKQGHRVSPDVFKKFKDEQGNFKAELSTDAKGLLCLNDVAYLSTRGEDILDEAIPFTEEHLRSCISHVDSHMAAKIEHSLELPLHHRIPRLENRHYISVYEGDKERNEVVLELANLDFNLIQILHQRELRDITMWWKEIDLAAKLPFIRDRLVECYYWIMGVYFEPIYSRARVFSTKMTMLVSVVDDIYDVYATEDELQLFTDAIYRWDADDIDQLPQYLKDAFMVLYNTVKTLEEELEPEGNSYRGFYVKDAMKVLARDYFVEHKWYNRKIVPSVEEYLKISCISVAVHMATVHCIAGMYEIATKEAFEWLMTEPKLVIDASLIGRLLDDMQSTSFEQQRGHVSSAVQCYMAEYGVTAEEACEKLRDMAAIAWKDVNEACLRPTVFPMPILLPSINLARVAEVIYLRGDGYTHAGGETKKHITAMLVKPIEV

SEQ ID NO: 100 - DNA, 1680 bp, synthetic construct
  misc_feature 1..1680: note = Codon optimized DNA sequence of DlTps589 from D. lanceolata
  source 1..1680: mol_type = other DNA
atggacctgattaacccgagccctgctgcatccaccctgccactgccagtcgatggtgatagcgaagttgtgcgccgtagcgcgggtttccatccgaccatctggggtgaccactttctgtcttataagccggacccgaaaaagattgatgcgtggaacaagcgtgttgaggaactgaaagaagaggtcaaaaagattttgagcaatgcgaaaggcacggttgaggaactgaatttgattgacgacctggtacacctgggtattagctatcactttgagaaagaaatcgacgacgcgctgcagcatatcttcgatacgcacctggatgatttcccgaaagatgacctctacgtggctgcgctgcgttttggcgtcctgcgtaagcaaggccatcgtgtcagcccggacgtctttaagaaattcaaagacgagcaaggcaacttcaaagcggagctgtcaaccgatgcaaagggcctgttgtgcctgaacgatgtggcgtacctgagcacccgtggtgaggatatcctggacgaagcgatcccgttcacggaagaacatttgcgctcgtgcattagccacgttgatagccacatggcagcgaagattgagcactctctggagctgccgctgcaccatcgcattccgcgtttagagaatcgccattacatctccgtgtacgagggtgacaaagagcgtaatgaagtcgttctggagttggctaacttggactttaatcttatccagatcctgcaccagcgcgagctgcgcgacatcacgatgtggtggaaagaaattgatctggccgcaaagctgccgtttattcgtgaccgtctggtggagtgttactattggattatgggcgtgtacttcgagccgatctacagccgtgcgcgcgtgtttagcaccaagatgaccatgctggttagcgtggtggatgacatctatgatgtctacgctacggaagatgagttgcagctgtttaccgacgccatttacagatgggacgccgatgacattgatcaactgccgcaatatctgaaagacgcctttatggttctgtacaacaccgtcaaaaccctggaagaagaactggagccggaaggtaactcttatcgtggtttctacgttaaagatgcgatgaaagttctggcgcgtgactatttcgttgagcataagtggtacaatcgtaagatcgtcccgtccgttgaagagtacttgaagattagctgtatcagcgtcgcagtccacatggcgaccgtgcactgtatcgccggcatgtatgagatcgccacgaaagaagcattcgagtggctgatgaccgagccgaaactggtgattgacgcaagcctgattggtcgcctgctggacgatatgcagagcacgagctttgagcagcagcgcggtcatgttagctccgcagttcaatgctacatggctgagtacggtgtgactgccgaagaagcatgcgagaagctgcgtgatatggcggccattgcgtggaaagatgtgaatgaagcatgcctgcgcccgaccgttttcccgatgccgattttactgcctagcatcaacctggcacgtgtggcggaagttatctatctgcgtggcgacggttatacgcacgcgggtggtgagactaagaagcacatcaccgcgatgctggtcaagccgatcgaagtgtaa

SEQ ID NO: 101 - DNA, 1656 bp, Drimys winteri
  misc_feature 1..1656: note = SCH51_3228_9 wild type DNA sequence
  source 1..1656: mol_type = genomic DNA
atggcttccaccctccctctcccagcttatggagattcagaagttgttaggcgatctgccgggtttcatccgacgatctggggcgatcacttcctctcctacaagcctgatccaacgaaaatagatgaatggaataaaagggttgaagagctgaaggaagaagtgaagaagatattaagcaatgcaaaagggacagtggaagagctgaatttgcttgatgatctcgtacaccttgggattagttatcattttgagaaggagattgatgatgctttacaacaaatctttgatacccatcttgatgtttttcctaaggatgatctatatgccaccgctctccgatttggcgtcttaaggaaacaggggcaccgtgtttctccagatgtattcaaaaaattcaaagatgagcaggggaatttcaaggcagagttgagcaccgatgcgaagggtttgctatgtttatatgatgtggcttatctcagcacaagaggggaagatatcttggatgaagccattcctttcactaaggagcaccttaggtcttgtattagccatgtcgattctcatatggcagcaaaaattgagcattctctagagcttccccttcatcatcgcataccaaggctagagaacaggcactacatctcagtctatgaaggagacaaggaaaggaatgaagttgtccttgagcttgccaaattagatttcaatctgattcaaatcttgcaccaaagagagctgagggacatcacaacgtggtggaaggagattgaccttgcagcaaagctaccttttattagggataggttggtggagtgctactattggatcatgggagtctattttgaaccaatatactcaagggctagagttttttcgaccaaaatgacaatcttggtctcagttgtggacgacatatatgatgtatatgctacagaggatgagctccaacttttcactgatgcaatctataggtgggatgctgaggacattgagcagcttccacagtacttgaaagatgcttttcttgtactctataacactgtgaaggacctagaagaggaattggaaccagaaggaaactcttatcgtggatactatgtaaaagatgcgatgaaggttttggcaagggattactttgtggagcacaaatggtataacagaaaaattgtgccatcagtagaggactacctgcgaatttcttgcattagtgttgccgttcatatggccacagttcattgtattgctgggatgtatgaaattgcaaccaaagaggcattcgaatggttgaagacggaacctaaacttgttatagatgcatcactgattgggcgtctcctcgatgacatgcagtccacctcgtttgagcaacagagaggtcatgtgtcatcagcggtacagtgttacatgatccaatatggggtatcacacgaagaagcgtgtgagaagttgcgagaaatggctgcaattgcgtggaaagatgtaaaccaagcatgccttaggcccactgttttccctatgcctattcttctgccctccatcaaccttgcacgtgtggcagaagtgatttacctacgcggagatggatatacacatgcgggtggtgagaccaaaaaacatatcacggccatgcttgttgatccaatcaaagtctga

SEQ ID NO: 102 - protein, 551 aa, Drimys winteri
MASTLPLPAYGDSEVVRRSAGFHPTIWGDHFLSYKPDPTKIDEWNKRVEELKEEVKKILSNAKGTVEELNLLDDLVHLGISYHFEKEIDDALQQIFDTHLDVFPKDDLYATALRFGVLRKQGHRVSPDVFKKFKDEQGNFKAELSTDAKGLLCLYDVAYLSTRGEDILDEAIPFTKEHLRSCISHVDSHMAAKIEHSLELPLHHRIPRLENRHYISVYEGDKERNEVVLELAKLDFNLIQILHQRELRDITTWWKEIDLAAKLPFIRDRLVECYYWIMGVYFEPIYSRARVFSTKMTILVSVVDDIYDVYATEDELQLFTDAIYRWDAEDIEQLPQYLKDAFLVLYNTVKDLEEELEPEGNSYRGYYVKDAMKVLARDYFVEHKWYNRKIVPSVEDYLRISCISVAVHMATVHCCAGMDEIATKEAFEWLKTEPKLVIDASLIGRLLDDMQSTSFEQQRGHVSSAVQCYMIQYGVSHEEACEKLREMAAIAWKDVNQACLRPTVFPMPILLPSINLARVAEVIYLRGDGYTHAGGETKKHITAMLVDPIKV

SEQ ID NO: 103 - DNA, 1656 bp, synthetic construct
  misc_feature 1..1656: note = Codon optimized DNA sequence of SCH51-3228-9
  source 1..1656: mol_type = other DNA
atggcaagcaccctgccgctgcctgcctatggtgatagcgaagttgttcgtcgtagcgcaggttttcatccgaccatttggggtgatcattttctgagctataaaccggatccgaccaaaattgatgaatggaataaacgtgtcgaagaactgaaagaagaagtgaaaaaaatcctgagcaatgccaaaggcaccgttgaggaactgaatctgctggatgatctggttcatctgggtatcagctatcactttgagaaagaaatcgatgatgcactgcagcagatttttgatacccatctggatgttttcccgaaagatgatctgtatgcaaccgcactgcgttttggtgttctgcgtaaacagggtcatcgtgttagtccggatgtgttcaaaaaattcaaagatgaacagggcaacttcaaagcagaactgagcaccgatgcaaaaggtctgctgtgtctgtatgatgttgcatatctgagcacccgtggtgaagatattctggatgaagcaattccgtttaccaaagaacatctgcgtagctgtattagccatgttgatagccacatggcagcgaaaattgaacatagcctggaactgcctctgcatcaccgtattccgcgtctggaaaatcgtcactatattagcgtttatgagggcgataaagaacgcaatgaagttgtgctggaactggcaaaactggattttaacctgattcagattctgcatcagcgtgaactgcgtgatattaccacctggtggaaagaaattgatctggcagcaaaactgccgtttattcgtgatcgtctggttgaatgctattattggattatgggcgtgtatttcgaaccgatttatagccgtgcacgtgtttttagcaccaaaatgaccattctggttagcgtggtggatgatatctatgatgtttatgccaccgaagatgaactgcagctgtttaccgatgccatttatcgttgggatgcagaagatattgaacagctgccgcagtatctgaaagatgcatttctggttctgtacaacaccgtgaaagatctggaagaagaactggaaccggaaggtaatagctatcgtggttattatgttaaagatgccatgaaagttctggcacgcgattattttgttgagcacaaatggtataaccgcaaaattgttccgagcgtggaagattatctgcgtattagctgcattagcgttgcagttcacatggcaaccgttcattgttgtgcaggtatggatgaaattgcaaccaaagaagcatttgagtggctgaaaaccgaaccgaaactggttattgatgcaagcctgattggtcgtctgctggacgatatgcagagcaccagctttgaacagcagcgtggtcatgttagcagcgcagttcagtgttatatgattcagtatggtgttagccatgaagaagcatgcgaaaaactgcgcgaaatggcagcaattgcatggaaagatgttaatcaggcatgtctgcgtccgaccgtttttccgatgccgattctgctgccgagcattaatctggcacgtgttgccgaagttatctatctgcgtggtgatggttatacccatgccggtggtgaaaccaaaaaacatattaccgcaatgctggtcgatccgattaaagtttaa

SEQ ID NO: 104 - DNA, 1656 bp, Drimys winteri
  misc_feature 1..1656: note = SCH51_3228_11 wild type DNA sequence
  source 1..1656: mol_type = genomic DNA
atggcttccaccctccctctcccagcttatggagattcagaagttgttaggcgatctgccgggtttcatccgacgatctggggcgatcacttcctctcctacaagcctgatccaacgaaaatagatgaatggaataaaagggttgaagagctgaaggaagaagtgaagaagatattaagcaatgcaaaagggacagtggaagagctgaatttgcttgatgatctcgtacaccttgggattagttatcattttgagaaggagattgatgatgctttacaacaaatctttgatacccatcttgatgtttttcctaaggatgatctatatgccaccgctctccgatttggcgtcttaaggaaacaggggcaccgtgtttctccagatgtattcaaaaaattcaaagatgagcaggggaatttcaaggcagagttgagcaccgatgcgaagggtttgctatgtttatatgatgtggcttatctcagcacaagaggggaagatatcttggatgaagccattcctttcactaaggagcaccttaggtcttgtattagccatgtcgattctcatatggcagcaaaaattgagcattctctagagcttccccttcatcatcgcataccaaggctagagaacaggcactacatctcagtctatgaaggagacaaggaaaggaatgaagttgtccttgagcttgccaaattagatttcaatctgattcaaatcttgcaccaaagagagctgagggacatcacaatgtggtggaaggagattgaccttgcagcaaagctaccttttattagagataggttggtggagtgctactactggatcatgggggtctattttgaaccaatatactccagggctagggttttttccactaaaatgacaatcttggtctcagttgtggacgacatatatgatgtctatgctacggaggatgagcttcaactattcactgatgcaatctataggtgggatgctgatgacattgatcagctgcctcagtacttgaaagatgcttttatggtactctataacactgtgaagactctagaagaagaacttgaaccagaaggaaactcttatcgtggatactacgtaaaagatgcaatgaaggttttggcaagagattactttgtggaacacaaatggtataacagacaaattgtgccatccgtagaggaatacttgaaaatttcttgcattagtgtggctgttcatatggctacagttcattgtattgctgggatgtatgaaattgctaccaaagaggcattcgaatggttgaagactgaacccaaacttgttatcgatgcatctctgatcggtcgtcttcttgatgacatgcagtctacctcgtttgagcaacaaagagggcacgtgtcatcagcagtacagtgttacatggcccaatatggagtaacagcagaagaagcatgtgaaaagctacgagaaatggctgcaattgcttggaaagatgtgaatgaagcatgccttaggcccacggtattccctatgcctatcctcttgccttctatcaacttggcacgtgtggcagaagtgatctacctacgtggagatggatacacgcacgctgggggtgagaccaaaaaacacatcacggccatgcttgttaagccaattgaagtctga

SEQ ID NO: 105 - protein, 551 aa, Drimys winteri
MASTLPLPAYGDSEVVRRSAGFHPTIWGDHFLSYKPDPTKIDEWNKRVEELKEEVKKILSNAKGTVEELNLLDDLVHLGISYHFEKEIDDALQQIFDTHLDVFPKDDLYATALRFGVLRKQGHRVSPDVFKKFKDEQGNFKAELSTDAKGLLCLYDVAYLSTRGEDILDEAIPFTKEHLRSCISHVDSHMAAKIEHSLELPLHHRIPRLENRHYISVYEGDKERNEVVLELAKLDFNLIQILHQRELRDITMWWKEIDLAAKLPFIRDRLVECYYWIMGVYFEPIYSRARVFSTKMTILVSVVDDIYDVYATEDELQLFTDAIYRWDADDIDQLPQYLKDAFMVLYNTVKTLEEELEPEGNSYRGYYVKDAMKVLARDYFVEHKWYNRQIVPSVEEYLKISCISVAVHMATVHCIAGMYEIATKEAFEWLKTEPKLVIDASLIGRLLDDMQSTSFEQQRGHVSSAVQCYMAQYGVTAEEACEKLREMAAIAWKDVNEACLRPTVFPMPILLPSINLARVAEVIYLRGDGYTHAGGETKKHITAMLVKPIEV

SEQ ID NO: 106 - DNA, 1656 bp, synthetic construct
  misc_feature 1..1656: note = Codon optimized DNA sequence of SCH51-3228-11
  source 1..1656: mol_type = other DNA
atggcatctactcttccactgccggcttatggtgattctgaggttgttcgtcgttccgcgggttttcaccctaccatctggggcgatcactttctgtcctataagccagacccgaccaagattgacgagtggaataagcgtgtcgaggaactgaaagaagaagtgaaaaagatcctgtccaacgcaaaaggtactgtcgaggagctgaatctgctggatgacctggtgcatctgggcatcagctatcacttcgaaaaggaaattgacgacgctttgcagcaaatttttgatacgcacctggacgtctttccgaaagatgacctgtatgcgaccgcgctgcgctttggtgtgctgcgtaaacagggtcatcgcgtgtctcctgatgtgttcaagaaatttaaagatgaacagggcaatttcaaggccgagttgagcacggacgccaaaggtttgctctgcctgtacgacgttgcatatctgagcacccgtggtgaagatatcctggacgaagcgattccgttcaccaaggaacatctgcgctcgtgcatttcccatgtagatagccacatggcggccaagatcgagcacagcctggagctgcctttgcaccatcgtattccgcgcctggagaatcgccattacattagcgtctatgagggtgacaaagagcgcaacgaagtcgtgttagagctggcgaagctggacttcaacctgattcaaattctgcatcaacgcgagctgcgcgacattaccatgtggtggaaagagattgatctggcagcgaagctgccgttcatccgcgatcgtctggttgagtgctactactggatcatgggcgtctacttcgagccgatctacagccgcgctcgtgtgttttcgacgaagatgaccatcctggttagcgttgttgatgacatttatgacgtttacgcgaccgaagatgaactgcagctgtttacggacgcaatctaccgttgggacgcggatgatatcgaccagctgccgcaatacttgaaagatgcgttcatggttttgtacaacaccgtcaaaacgctggaagaagaactggagccggaaggcaacagctaccgtggttactatgttaaagatgcgatgaaagttctggcgcgcgactacttcgtcgagcacaagtggtataaccgtcagattgtgccgagcgtcgaggaatacctgaagattagctgtatcagcgttgccgttcacatggcaacggtgcactgcatcgccggtatgtacgagattgcgacgaaagaagccttcgaatggttgaaaaccgagccgaagctggttatcgacgccagcctgatcggtcgtttgctggacgacatgcaaagcacgagcttcgagcagcagcgcggccatgtgagcagcgctgttcagtgttatatggcgcaatatggcgtgaccgcagaagaagcgtgcgagaagctgcgtgagatggcagcaattgcgtggaaagatgtgaatgaagcctgtctgcgtccgactgtgtttccgatgccgatcctgctgccgagcattaacctggcgcgtgtggcagaggtcatctatctgcgtggtgacggttacacccacgcgggtggcgaaaccaagaaacatatcaccgcaatgctggttaagccgattgaagtgtaa

SEQ ID NO: 107 - DNA, 1677 bp, Drimys winteri
  misc_feature 1..1677: note = SCH51_998_28 wild type DNA sequence
  source 1..1677: mol_type = genomic DNA
atggatcttagtacttcacctgttctttcttcctccccccttccggtggaagacggaaaaaatccggccgttcgccgttcagctggatttcaccccagtatttggggtgatcatttcctctcctacactgaagatcacaagaagctggatgcatggagcgaaaggactcaagtgttgaaggaagaggtgaggagaattttaatcaatgccaaggggtcactagaagagttggatttgttggatgcaatccaacgccttggggtgaaatatcactttgagaaagagattgaagaggcattacaccatatttatgttgcagaaactcatgtttctactgatgacttatattccgtttctctccggtttcgacttcttagacaacaagggtacaatgtatctgctgatgtatttaaaaagttcaaagatgagaggggcaacttcaaggcaagcttaagtactgatgccagggggttgctaagcttgtatgaagctgcatttctcagcatacgaggagatgatatcttagatgaagccataactttcacaagagagcagcttaagtcttctatgacccatgttgatgcccctcttgccaaacaaatagcccatgccttagaggtaccagcgcacaagcgcatacaaagactagagaacattcgctacctcacaatctaccaagaagagaaaggaaggaatgatgtgttgcttgagcttgccaagttggatttcaatatcttacaacaattgcataagaaagaactgagagaccttacaaagtggtggaaggacacagacgttgcaggaaagctacctttcatcagagataggttggtggaatgctattattggatcttgggtgtgtattatgagccagaatactccagagctagaattttttctaccaaaatgacaatcatggtctcagttgttgatgacatatatgacgtatatgctactgaagatgagctccaactattcactgatgcaatctataggtgggatctggagggcctagatcaactcccacagttcttgaaagactgttttcttgtactctatgacaccgtcaaggaattagaagacgaactagaaccggaaggaaaatcctatcgtggatactatgtaaaggatgcgatgaaggttttggctagagattacttcgttgagcacaaatggtataacagaaacatagtgccaagtgtagaagaatatctccgtgtttcttgcatcagtgttgcagtccatatggctaacgtccattgctgtgctgggatgggagatgtaatgagcaaagaggcattcgaatggttgaagagtgaaccaaaggttgtaatggatgcatcactaattggccgactgctcgatgacatgcagtccaccgagtttgagcaaaagagaggccatgttgcatcggctgtccaatgttacatgaatgagtatggagtgacttacaaagaagcgtgtgaaaagctgcatgaaatggctgcccttgcatggaaagacgtaaaccaggcttgccttaaaccaactgttttccctctccctgtatttatgcctgcaatcaaccttgcgcgagtggctgaagtcatctaccttcgtggagatgggtatactcattcaggaggagagactaaagaaaatatcacgttgatgcttgtcaatccaatctctgtgtga

SEQ ID NO: 108 - protein, 558 aa, Drimys winteri
MDLSTSPVLSSSPLPVEDGKNPAVRRSAGFHPSIWGDHFLSYTEDHKKLDAWSERTQVLKEEVRRILINAKGSLEELDLLDAIQRLGVKYHFEKEIEEALHHIYVAETHVSTDDLYSVSLRFRLLRQQGYNVSADVFKKFKDERGNFKASLSTDARGLLSLYEAAFLSIRGDDILDEAITFTREQLKSSMTHVDAPLAKQIAHALEVPAHKRIQRLENIRYLTIYQEEKGRNDVLLELAKLDFNILQQLHKKELRDLTKWWKDTDVAGKLPFIRDRLVECYYWILGVYYEPEYSRARIFSTKMTIMVSVVDDIYDVYATEDELQLFTDAIYRWDLEGLDQLPQFLKDCFLVLYDTVKELEDELEPEGKSYRGYYVKDAMKVLARDYFVEHKWYNRNIVPSVEEYLRVSCISVAVHMANVHCCAGMGDVMSKEAFEWLKSEPKVVMDASLIGRLLDDMQSTEFEQKRGHVASAVQCYMNEYGVTYKEACEKLHEMAALAWKDVNQACLKPTVFPLPVFMPAINLARVAEVIYLRGDGYTHSGGETKENITLMLVNPISV

SEQ ID NO: 109 - DNA, 1677 bp, synthetic construct
  misc_feature 1..1677: note = Codon optimized DNA sequence of SCH51-998-28
  source 1..1677: mol_type = other DNA
atggatctgagcaccagtccggttctgagcagctcaccgctgccggttgaagatggtaaaaatccggcagttcgtcgtagcgcaggttttcatccgagcatttggggtgatcattttctgagctataccgaggatcacaaaaaactggatgcatggtcagaacgtacccaggttctgaaagaagaagtgcgtcgtattctgattaatgcaaaaggtagcctggaagaactggatctgctggatgcaattcagcgtctgggtgttaaatatcactttgagaaagaaatcgaagaagccctgcatcatatttatgttgcagaaacccatgtgtcaaccgatgatctgtatagcgttagcctgcgttttcgtctgctgcgtcagcagggttataatgttagcgcagatgtgttcaaaaaattcaaagatgaacgcggtaacttcaaagcaagcctgagcaccgatgcacgtggtctgctgagcctgtatgaagcagcatttctgagcattcgtggtgatgatattctggatgaagcaattacctttacccgtgaacagctgaaaagcagcatgacccatgttgatgcaccgctggcaaaacaaattgcacatgcactggaagttccggcacataaacgtattcagcgcctggaaaatattcgctatctgaccatttaccaagaagagaaaggtcgtaacgatgttctgctggaactggccaaactggattttaacattctgcagcagctgcataaaaaagaactgcgtgatctgaccaaatggtggaaagataccgatgttgcaggtaaactgccgtttattcgtgatcgtctggttgaatgctattattggattctgggcgtttattatgagccggaatatagccgtgcacgtatttttagcaccaaaatgaccattatggttagcgtggtggatgacatctatgatgtttatgcaaccgaagatgaactgcagctgtttaccgatgcaatttatcgttgggatctggaaggtctggatcagctgccgcagttcctgaaagattgttttctggttctgtatgataccgtgaaagaactggaagatgagctggaaccggaaggtaaaagctatcgtggttattatgttaaagatgccatgaaagttctggcacgcgattattttgttgagcacaaatggtataaccgcaatattgttccgagcgtggaagaatatctgcgtgttagctgtattagcgttgcagttcacatggcaaatgttcattgttgtgcaggtatgggtgatgtgatgagcaaagaagcatttgaatggctgaaaagtgaaccgaaagttgttatggatgccagcctgattggtcgcctgctggacgatatgcagagcaccgaatttgaacagaaacgtggtcatgttgcaagcgcagttcagtgttatatgaatgaatatggcgtgacctataaagaggcatgcgaaaaactgcatgaaatggcagcactggcatggaaagatgttaatcaggcatgtctgaaaccgaccgtttttccgctgcctgtttttatgcctgcaattaatctggcacgtgttgccgaagttatttacctgcgtggggatggttatacccatagcggtggtgaaaccaaagaaaacattaccctgatgctggttaatccgattagcgtttaa

SEQ ID NO: 110 - DNA, 1680 bp, Drimys lanceolata
  source 1..1680: mol_type = genomic DNA
atggatgttctaattccctcccctgtggcttccactctccctctgcccgaagatggaaacttggacgtcgttcgcagatccgccgggtttcatccgacggtctggggcgatcacttcctcgcttactcgcccgatccaaccaaaatagatgcttggactaaaagagttgaagagctgaagcaagaagtgaagaggattctaagcaatgtgaaagggtcactggaagagctgaacttgcttgatgctatccaacaccttgggattggttatcattttgagaaagagattgatgatgctttacaactaatctttgattcccatattgatgcttttcctactgatgatctatatgtggctgccctccgatttagcctactaaggcgacaagggcactgtgtttcttcagatgtattcaaaaaattcaaagatgagcaggggaatttcaaggcagagctgagcaccgatgcgaaaggtttgctgagtctctatgacgcggcgtatctcagtgtaagaggggaagatatattggatgaggccattcctttcactagggagcaccttaggacttgtattagccatgtagattctcatttggcagcaaaaattgagcattctctagagcttcccctgcatcatcgcataccaaggctagagaacaggcactacatctcagtgtacgaaggagagaaggaaaggaatgaagttgtactagagcttgccaaattagatttcaatctgattcaaatcttgcaccaaagagagctgagggacatcacaacgtggtggaatgagattgacctcgcagcaaagctaccatttattagggataggttggtggagtgctactattggatcatgggtgtctattttgaaccaatattctcaagggctagagttttttcgaccaaaatgacaattttggtctcagttgtcgacgacatatatgatgtctacgctacagaggatgagctccaacttttcactgacgcaatctataggtgggatgccgaggacattgagcagcttccacagtacttgaaagattcttttcttgtactctataacaccgtgaaggacttagaagaggagctgaaaccagaaggaaactcatatcgtggagactatgtaaaagatgcgatgaaggttttggcaagagattactttgtggagcacaaatggtataacagaaaaattgtaccgtcagtagaggactacctacgaatttcttgcattagtgttgccgttcatatggctacagttcattgttgtgctgggatggatgaaattgcaaccaaagaggcattcgaatggttgaagaccgaacctaaacttgttatagatgcatcactgattgggcgtctcctcgatgacatgcagtccacctcgtttgagcaacagagaggtcatgtgtcatcggcggtacagtgttacatgatccaatatggcgtatcacacgaagaagcgtgtgagaagttgacagaaatggctgcaattgcatggaaagatgtaaaccaagcatgccttaggcccactgttttcccaatgcctattcttctgccttcaatcaaccttgcacgtgtggcagaagtcatctacctgcgcggagatggatatacacatgctggtggtgagaccaaaaaacatatcacggccatgcttgttgaaccaatccaagtctga

SEQ ID NO: 111 - protein, 559 aa, Drimys lanceolata
MDVLIPSPVASTLPLPEDGNLDVVRRSAGFHPTVWGDHFLAYSPDPTKIDAWTKRVEELKQEVKRILSNVKGSLEELNLLDAIQHLGIGYHFEKEIDDALQLIFDSHIDAFPTDDLYVAALRFSLLRRQGHCVSSDVFKKFKDEQGNFKAELSTDAKGLLSLYDAAYLSVRGEDILDEAIPFTREHLRTCISHVDSHLAAKIEHSLELPLHHRIPRLENRHYISVYEGEKERNEVVLELAKLDFNLIQILHQRELRDITTWWNEIDLAAKLPFIRDRLVECYYWIMGVYFEPIFSRARVFSTKMTILVSVVDDIYDVYATEDELQLFTDAIYRWDAEDIEQLPQYLKDSFLVLYNTVKDLEEELKPEGNSYRGDYVKDAMKVLARDYFVEHKWYNRKIVPSVEDYLRISCISVAVHMATVHCCAGMDEIATKEAFEWLKTEPKLVIDASLIGRLLDDMQSTSFEQQRGHVSSAVQCYMIQYGVSHEEACEKLTEMAAIAWKDVNQACLRPTVFPMPILLPSINLARVAEVIYLRGDGYTHAGGETKKHITAMLVEPIQV

SEQ ID NO: 112 - DNA, 1680 bp, synthetic construct
  misc_feature 1..1680: note = Codon optimized DNA sequence of SCH51-13163-6
  source 1..1680: mol_type = other DNA
atggatgttctgattccgagtccggttgcaagcaccctgccgctgccggaagatggtaatctggatgttgttcgtcgtagcgcaggttttcatccgaccgtttggggtgatcattttctggcatatagtccggatccgaccaaaattgatgcatggaccaaacgtgttgaggaactgaaacaagaagtgaaacgtattctgagcaatgtgaaaggtagcctggaagaactgaatctgctggatgcaattcagcatctgggtattggttatcacttcgagaaagaaattgatgatgcactgcagctgatctttgatagccatattgatgcctttccgaccgatgatctgtatgttgcagcactgcgttttagcctgctgcgtcgtcagggtcattgtgttagcagtgatgttttcaaaaaattcaaagacgagcagggcaactttaaagcagaactgagcaccgatgcaaaaggtctgctgagcctgtatgatgccgcatatctgagcgttcgtggtgaagatattctggatgaagcaattccgtttacccgtgaacatctgcgtacctgtattagccatgtggatagccatctggcagcaaaaattgaacatagtctggaactgcctctgcatcatcgtattccgcgtctggaaaatcgtcactatattagcgtttatgaaggcgaaaaagaacgcaatgaagttgtgctggaactggcaaaactggattttaacctgattcagattctgcatcagcgtgaactgcgtgatattaccacctggtggaatgaaattgacctggcagccaaactgccgtttattcgtgatcgtctggttgaatgctattattggattatgggcgtgtattttgaaccgatttttagccgtgcacgtgtgtttagcaccaaaatgaccattctggttagcgtggtggatgatatctatgatgtttatgcaaccgaagatgagctgcaactgtttaccgatgccatttatcgttgggatgcagaagatattgaacagctgcctcagtatctgaaagatagctttctggttctgtacaacaccgtgaaagatctggaagaagaactgaaaccggaaggtaatagctatcgtggtgattatgttaaagacgccatgaaagttctggcacgcgattattttgttgagcacaaatggtataaccgcaaaattgttccgagcgtggaagattatctgcgtattagctgcattagcgttgcagttcacatggcaaccgttcattgttgtgcaggtatggatgaaattgcaaccaaagaagcatttgagtggctgaaaaccgaaccgaaactggttattgatgcaagcctgattggtcgtctgctggacgatatgcagtcaaccagctttgaacagcagcgtggtcatgttagcagcgcagttcagtgttatatgattcagtatggtgttagccatgaagaagcatgcgaaaaactgaccgaaatggcagcaattgcatggaaagatgttaatcaggcatgtctgcgtccgaccgtgtttcctatgccgattctgctgccgagcattaatctggcacgtgttgccgaagttatctatctgcgtggtgatggttatacccatgccggtggtgaaaccaaaaaacatattaccgcaatgctggtagaaccgattcaggtttaa

SEQ ID NO: 113 - DNA, 1671 bp, Valeriana amurensis
  source 1..1671: mol_type = genomic DNA
atgtctactgcattaaacagtgagcatgaaactgttcgtccattagcaagttttaaaccgagtacatggggcgatcttttcatctcttattctgaagatagccagcttaaggaagtatatggtaaagagcacgaatgtctgaaacaacaagtgaaaacaatgttgttggatctgacaaattatagaatttcggagaaaatcgctttcataaatacgttggagagattaggggtatctcatgagtttgagaatgagattgaagggctgcttcatcaaatgtttgatgctcattctaaattccaagatggcattcaacactttgatttgttcacattggggatttactttaggattctcaggcaacatggctatagaatctcttgtgatgttttcaacaagttgaaagatagcaacaatgaattcaagaaggaacttaaagaggacgctattggtttgctaagtttgtacgaagcgacacaagtaagagcacacgctgaagaaattttagacgaagccctcattttcacaaaggctcaacttgaatccatagccgcaacctcgagcttaagcccatttgtcgagaagcaaattactcatgctttggtccaagctctccacaaaggaatcccaagagtcgaatcgcgccatttcatctctgtttatgaagaagatcctgacaaaaatgatttgttgttgaggttctcaaagattgattacaatcttgtacaaatgcttcacaagcaagaattgtgccatatctcaaagtggtggagagattcggagctcgaaacaaaactaacttatgtgaggaatagagtggcggaatgctttttatggactctttgtgtgtaccacgaaccaaagtactctccggctcggcttctgttaggcaaactcataaatatcatatcttgcactgatgacacatatgatgcgtatggtacattagaggaagttcagatctttacagatgtcatacaaaggttggataggagttctatggagcagctgccggattacatgaaaatcctctacaaagctgtccttgatctttttgacgaagtagaagttcagctatcgaaccatgaaactaataatacttatcgtatggcttatgcgaaggaagagttaaaagctatcgccaagtgctacgaaaaggagcacatatggttcagaaaatgtcacgtgcccccattcgaagaatatctagagaatgcggtagtgtcaatcggtaatcgtttggccgtacctttttcttttctgggaatggatcaagtagcaggtgttgaagcgttcgagtgggccaaaactgatcccaaaatggtaaaatcgtgcggtaaagtcttacgacttgttgacgatgtaatgagccacgaggaggaagatgtaagaggacacgtggcaacgggagtcgaatgctacatgaaagaacacggagtgagtagggaagaggccatcgtggagttctacaagagggtcgagtacgcgtggaaggatgtgaacgaggaatttataacgccgaaccatctgcatatcgacctcctcaaccgcgttcttaaccttacaagaattgcagacgttgtttacaagtttgaagacggctacacgcatcccgagaagactctgaaacatcatatcatggcgttgttcgtcgaccccgtccccatatag

SEQ ID NO: 114 - protein, 556 aa, Valeriana amurensis
MSTALNSEHETVRPLASFKPSTWGDLFISYSEDSQLKEVYGKEHECLKQQVKTMLLDLTNYRISEKIAFINTLERLGVSHEFENEIEGLLHQMFDAHSKFQDGIQHFDLFTLGIYFRILRQHGYRISCDVFNKLKDSNNEFKKELKEDAIGLLSLYEATQVRAHAEEILDEALIFTKAQLESIAATSSLSPFVEKQITHALVQALHKGIPRVESRHFISVYEEDPDKNDLLLRFSKIDYNLVQMLHKQELCHISKWWRDSELETKLTYVRNRVAECFLWTLCVYHEPKYSPARLLLGKLINIISCTDDTYDAYGTLEEVQIFTDVIQRLDRSSMEQLPDYMKILYKAVLDLFDEVEVQLSNHETNNTYRMAYAKEELKAIAKCYEKEHIWFRKCHVPPFEEYLENAVVSIGNRLAVPFSFLGMDQVAGVEAFEWAKTDPKMVKSCGKVLRLVDDVMSHEEEDVRGHVATGVECYMKEHGVSREEAIVEFYKRVEYAWKDVNEEFITPNHLHIDLLNRVLNLTRIADVVYKFEDGYTHPEKTLKHHIMALFVDPVPI

SEQ ID NO: 115 - DNA, 1671 bp, synthetic construct
  misc_feature 1..1671: note = Codon optimised sequence
  source 1..1671: mol_type = other DNA
atgagcaccgcgttgaactccgagcatgaaaccgtccgtccgctggctagctttaaaccgagcacgtggggtgacctgttcatcagctacagcgaggacagccagctgaaagaagtgtatggtaaagagcatgaatgtcttaagcaacaagttaagaccatgctgctggacctgacgaattaccgtatcagcgagaagattgccttcatcaatacgctggagcgcctgggtgtttctcacgagttcgagaatgaaatcgaaggcctcctgcatcagatgttcgacgcgcactccaagtttcaagatggcattcagcactttgacctgtttaccctgggcatttacttccgtattttgcgccagcacggttatcgtatctcgtgcgatgtgtttaacaagctgaaggactctaataacgaattcaagaaagaactgaaagaagatgcaattggtctgctgtctctgtatgaagcgacccaagtgcgtgcccatgcagaagagattttggacgaagcgctgatcttcaccaaggctcagctggagagcatcgcggcgacgagcagcctgagcccgtttgtcgagaaacagattacccacgccttggtgcaagcgttgcataaaggcatcccacgcgtggagagccgccacttcattagcgtgtacgaagaggacccggacaagaacgatttgctgctgcgtttttccaagattgactacaatttagttcaaatgctgcacaaacaagagttgtgtcatattagcaaatggtggcgtgactccgagctggagactaaactgacctacgtccgtaatcgcgtggcagagtgttttctgtggaccctgtgtgtttaccacgagccgaagtatagcccggcacgtctgctgctgggtaaactgatcaacatcatttcttgcacggacgacacctatgatgcatacggtacgctggaagaagtccaaatctttaccgacgtgatccagcgtttggaccgtagctcgatggagcagctgccggattacatgaagattctgtataaagctgttctggatctgttcgatgaagttgaggttcagctgagcaaccatgagactaacaatacctaccgcatggcgtacgcaaaagaagaactgaaggctattgcgaaatgctacgagaaagagcacatctggtttcgcaagtgtcatgttccaccgttcgaagagtatctggagaacgccgtggtgagcatcggtaatcgtctggcggtcccgttcagcttcttgggtatggaccaggttgcgggcgtcgaggcctttgagtgggcaaagaccgatcctaaaatggttaaaagctgcggtaaggttctgcgcctggtcgatgatgtcatgagccatgaagaagaagatgtgcgtggtcacgtggcgacgggcgttgagtgctacatgaaagagcacggtgtcagccgtgaagaggcgatcgttgaattctataagcgtgtcgagtatgcatggaaagacgtcaacgaagagttcattactccgaatcacttgcacattgatctgctgaaccgtgttctgaacttaacccgcattgccgatgtcgtatacaagtttgaagatggctatacccacccggaaaagacgctgaaacaccatatcatggcgctgttcgtggacccggtgccgatctaa

SEQ ID NO: 116 - DNA, 1332 bp, Dryopteris fragrans
  misc_feature 1..1332: note = DfACT13 native nucleotide sequence
  source 1..1332: mol_type = genomic DNA
atggcctgtggaggcggcgtgaggaatattgaggtgaaggcgcaggagcccgttttggtgcagcctttatctacagcacaaagctctgcctataatcttcttacaaaactggaccagacgctcgcacaattggtggtgcagattgtctttgtcttcgatgtgaaaaaccccgtaacccgccgaccccacgatggcgccgatcctgccaagcttctgaaggaggctctgaggaaggtccttgtgcccttctacccgctcgctgggcgtctttgcctctcgcccgatgacggcagtctgttcattgactgcaatgctcagggggtttcctttgttgaggccaatgccgacgcggatatctccgaactcggcgacttctcgcagcccgatttcgcgactctgggctcccttgtcttccccttacctcctatagcttcggatgatggtcctcttctatctgcgcaggtgaccagattcaagtgtggaggatttgtgctgggcttcgtattccatcattgtttatttgatggattcgcactttcggaattcctaaatgcgtgggcggagactgcatgcggcgtgcctctttctacacctcctgtcctcgacagaacttttccgagggcgcgttctcccttgcaaatcaagtatccccacaccgagttcctggaagtcgaagatgtctctttgactcaaaatatctctaacgatgccatcaaccggtctttctgctttacttcggcaagtctagagatactcaagaagaaagccttggaggatggggtgctatcaaaatgcactacttttgaagctttatctgggctgatatggagggcccgaactagagccctgtggagcgattacccggagcacaaactaaaggtgctcattgtcgtcgacccgagagcacgttttgagcctcgtgtggtgccaaaagggtatgtgggcaatgcggtgctttttacatgtgctttcgcaagcgcaagggagctggaagaaaatcccttgtcacatgcagtgaagcatgtgcaacatgccatcgggcgtatgacggaggagtacatgttgtcgcaaatcgactacatggagcatcagaaggtatggtgcccaccgctaggagctagtacatctttcatgaccaaatggtctaggttggccttcaatattctagactttgggtggggcaggccgaagtatgtggggccggccacgtcgctgtcgatggagacgactacttttgtatcttatggaaagggcatgagtgtggtgttggctcttcctccagaagcaatgcgcaaatttgaaaaaatcgtacatccctacctcaatccatga

SEQ ID NO: 117 - DNA, 1332 bp, synthetic construct
  misc_feature 1..1332: note = DfACT13_codon optimized for its expression in S. cerevisiae
  source 1..1332: mol_type = other DNA
  CDS 1..1332
atggcatgtggcggtggggtcaggaatatcgaagtgaaggctcaagagccagtcttggtgcaaccgttaagcactgctcaatcgtcagcctacaatttgttaacaaagttagaccagactttggcgcaattggttgtccaaatagtgtttgttttcgatgtgaagaaccctgtgacaagaaggcctcatgatggagccgatccagctaagttattgaaagaggccttaaggaaagtactagtaccattctatccccttgccggaagactatgtctttccccagacgatggtagtctattcatagactgcaacgctcaaggtgtttcatttgtcgaagcaaacgctgatgccgacatcagtgagttaggtgatttctcccaaccggattttgcgaccttaggttctttagtcttccctcttccaccaattgcgtctgacgatggaccattgttaagtgctcaagtcacaagattcaaatgtggtggcttcgttctaggtttcgtcttccatcactgtctatttgatggttttgccttatctgaatttcttaatgcatgggctgagacagcgtgtggagtcccgttatcaaccccacctgttttagatagaacatttcctcgtgctagatctcccctgcaaataaagtacccacatacagagttcctagaggtagaagatgtttcattaactcagaacatctctaacgatgctatcaacagatccttctgttttacatcagcctctttggaaattttgaagaagaaggcgctagaagacggggtgttaagcaaatgtacgaccttcgaagctctatctggcttaatatggagagcacgtaccagagccttatggagcgactaccccgagcacaagttaaaggtcctgattgtcgtggaccctcgtgccagatttgaaccgagagtggtgccaaaaggctacgtagggaatgcagtcttgtttacttgtgcatttgcttcagccagagaactagaagaaaatccattatctcatgctgttaaacacgtacagcacgcgatcggccgtatgactgaggaatatatgctatcacaaattgattacatggagcaccagaaggtttggtgtcccccactaggtgcgtcgacttcttttatgactaagtggtcaaggcttgcctttaacatcttagattttggttggggtagacctaagtatgttggtccggctacttcattgtctatggaaacaactacattcgtctcctatggaaagggtatgagcgtggtgttagccctacccccagaggcaatgagaaagttcgaaaagattgtacacccttatttgaacccttaa

SEQ ID NO: 118 - protein, 443 aa, synthetic construct
  REGION 1..443: note = Synthetic Construct
MACGGGVRNIEVKAQEPVLVQPLSTAQSSAYNLLTKLDQTLAQLVVQIVFVFDVKNPVTRRPHDGADPAKLLKEALRKVLVPFYPLAGRLCLSPDDGSLFIDCNAQGVSFVEANADADISELGDFSQPDFATLGSLVFPLPPIASDDGPLLSAQVTRFKCGGFVLGFVFHHCLFDGFALSEFLNAWAETACGVPLSTPPVLDRTFPRARSPLQIKYPHTEFLEVEDVSLTQNISNDAINRSFCFTSASLEILKKKALEDGVLSKCTTFEALSGLIWRARTRALWSDYPEHKLKVLIVVDPRARFEPRVVPKGYVGNAVLFTCAFASARELEENPLSHAVKHVQHAIGRMTEEYMLSQIDYMEHQKVWCPPLGASTSFMTKWSRLAFNILDFGWGRPKYVGPATSLSMETTTFVSYGKGMSVVLALPPEAMRKFEKIVHPYLNP

SEQ ID NO: 119 - DNA, 1440 bp, Aspergillus sclerotiicarbonarius
  misc_feature 1..1440: note = PYI04555.1 native nucleotide sequence
  source 1..1440: mol_type = genomic DNA
atgggtgccagcgtctctttccagccctttgtgcccactcccctggaccatgctatgcccccaatttacgtgtctcaatttctatgctttcctaccacaactccgcaatccgctattcaaagtctccaagtgggaatcgagagattattcgaacgcctgccattcctggcgggagagattctcatcaatgaacacaccggagccatcaaagtccaggctcccagtgctttgatccgggaaattccctacatggccctccgagcccatcctgatctttaccttccagctaagcaatgtgcaaccacaccaatcgagagacagttgaagaccaacagccttgatgaatcctaccatccactcccggcggcacttccactctcccaaccccagcccgtcatccgattccaagcaaacaccctcgcagacggcattctctttgcagtcagctatcatcattgcatattcgatggcactggatgcggacagattctggaaatgttggctcagtgctgttcggcctccgacgataagatctccctgccgactgattgccacaccgatgtgctcctccgcgaatacatctccaatctaagccctactaccaacatcccccacgattacacgcaagcgtatagcactacggtgcaaccggaccccgatgcctcagacccagacacgtcccccgccataccctcctcactctacacagaagcattcacctttccctcccaacaaatcaccactctccgtgatgcatgcaaccacctcttgcccaaattacccagcaccagcaacgcacatccccacaaaccaacaccgaatcccctatcatcaaatgacgtcctcaccgcactaatagccctatgcatcacacgcgccaccaacaccaccaccccacccctccaacccaacaatcacagtctctcaatggccgtcaacctccgaacccgcatccagccccaagtaccagatcactttctgggaaacttcgccacgctactcccaatacactttaccagtccagtccacacccaacagtctgatctcctcctcaccacagaaccccccgaccccgccctcatccacctaaccaccctcgcctcccaaatccggtccagcctatccacagtcaacaccgattacatctgcggtctcatgacggatctccgaacccggcgcaatgcaggagaaaacagcagtcttctaattgaaggcattaagatttccagttggagacatctatccgtctacaagccggactttggccctgggttggggaaaatagcggggtttgagttccaggcggggcttatggataatttggtggtgattttgccttggagaaatggggattgggatgtgcgtgttacgttgttggagagggatatgcgcgggtttagggaggatcggttggttaggtgggcgttggggtctgggtag

SEQ ID NO: 120 - DNA, 1470 bp, synthetic construct
  misc_feature 1..1470: note = PYI04555.1_codon optimized for its expression in S. cerevisiae_C-terminally extended
  source 1..1470: mol_type = other DNA
  CDS 1..1470
atgggtgcgtcagttagttttcaacctttcgttcctacccccttggatcacgccatgccaccgatatacgtgtcacagttcttgtgtttccctactacgacgccgcagtcagcaatacaatccctacaggtcggtattgagcgtttgtttgaaagactaccttttctagccggggaaattctgataaatgagcacacaggtgcaataaaagttcaagctccctctgctctgattagagaaattccgtacatggccttaagggctcatccggatttgtatctgcctgctaagcaatgtgcaactactccaatagagaggcagttgaaaactaacagccttgatgagtcttatcatccattacctgcggcactaccattgtctcaaccacaaccagtcatcagattccaagctaatacacttgctgacggaatacttttcgctgtcagttaccaccattgtatatttgatggaacgggctgcggacagatccttgaaatgttagcccagtgttgttcggcctcagacgataagatcagcttgcctacggactgtcatacagatgtattgttgagagaatatatttcgaatctatctccaaccacgaatatcccgcatgactacactcaagcttattctactacagttcaaccagatcctgacgcatccgatcctgatactagcccagccattccgagttcgctgtacactgaagcatttacgtttccttcacaacagattaccacactaagagatgcgtgcaaccatttattgcctaaattaccatctacttcaaacgcacatccccacaaaccaacaccaaacccgttatcctcaaatgacgttttgacggcactgatcgcattgtgcatcaccagagctactaatacgacgacacccccattacaaccaaacaaccattctcttagcatggccgtcaatctaaggactcgtattcaaccccaagtcccggaccactttcttggtaattttgccaccttgctacctattcacttcacaagtccagtccatacgcagcaatcagatttattattgactactgagccgccagacccagcattgatccaccttacaacccttgcatcgcaaattaggtctagtttatcgaccgttaacaccgattacatatgtggcttgatgacagatctgaggactaggagaaacgccggtgagaattcctctttgttaatagaaggtattaagatttcttcatggagacatcttagcgtttacaagccagatttcggacctggattagggaagattgcaggatttgagtttcaagctggtttaatggacaaccttgttgtgatattgccttggagaaacggtgattgggacgtgcgtgtaactcttctggagagagatatgaggggttttagggaagacagattagtcagatgggcattaggtagcggaacaggccccttttcctttgtcgatatcatgtaa

SEQ ID NO: 121 - protein, 489 aa, synthetic construct
  REGION 1..489: note = Synthetic Construct
MGASVSFQPFVPTPLDHAMPPIYVSQFLCFPTTTPQSAIQSLQVGIERLFERLPFLAGEILINEHTGAIKVQAPSALIREIPYMALRAHPDLYLPAKQCATTPIERQLKTNSLDESYHPLPAALPLSQPQPVIRFQANTLADGILFAVSYHHCIFDGTGCGQILEMLAQCCSASDDKISLPTDCHTDVLLREYISNLSPTTNIPHDYTQAYSTTVQPDPDASDPDTSPAIPSSLYTEAFTFPSQQITTLRDACNHLLPKLPSTSNAHPHKPTPNPLSSNDVLTALIALCITRATNTTTPPLQPNNHSLSMAVNLRTRIQPQVPDHFLGNFATLLPIHFTSPVHTQQSDLLLTTEPPDPALIHLTTLASQIRSSLSTVNTDYICGLMTDLRTRRNAGENSSLLIEGIKISSWRHLSVYKPDFGPGLGKIAGFEFQAGLMDNLVVILPWRNGDWDVRVTLLERDMRGFREDRLVRWALGSGTGPFSFVDIM

SEQ ID NO: 122 - DNA, 1425 bp, Bazzania trilobata
  misc_feature 1..1425: note = ERR364415-1_contig_8546 native nucleotide sequence
  source 1..1425: mol_type = genomic DNA
atggcccgggcaccaccacctccgcctcctggtctcagaatgagagacacagtgctcagcatcgtgaagccgatacggaagacacagcatttggagacgatcgacgcaacgttcgtcgatttgatgagaatggacagctttatacccgtgattttcgcctacaggcctgcggacaagtccgaggccgcgtactcgcgtctcgtgaatcgcatcaaggagtcgctccagaaggttttggtcccctttttcgggttcgcgggccgctgggttccaagcagtggcgggagcaggcggctcttgtgcaacgatgagggcgttcccttcattgaagcgtttgtggacgaagagttggactcggtggtgaaggcttccgccgcattccagccggttacggagctgaatggcttgggcgtcctcggaatggacatgacttcatacgatcaaaggatgccaccggaaggtgggcaaccttgcgtcgttgctcaagtcacacggttcaaatgtgggggagtggttctgggggtggctttcaatcacactcacactgacggccagggattctacaccttcatgcgagcatggtccgacttctctcgaaccaacggaacggcaatcaaggtggaccacaaccgggccctgccagaactggcttccctctcacagttcttcatcaaacagcacgaccgaataggaggcaaaacttctaccgatcgagtcaacgatcattgttctaaagttccggaacggctggctttgaaagctttcgaggttcgtgcgtctaagatcaaagccgcaaagctagcagccgaagatggaggggttgggtatgtcagcacggtagattgcattgtggctcacttatggaaaactcttgccagattgccgcccgtcgtgttggatgggagggagattacggtcttctcgcctgtggaggggaggaacagattcttggacccgccaagacccaatatgtgtggaaattgttttgcagcaatggtgacccccaaaatcccaacccaggagttgctggagatgcctctcgctgcaattgcaggcaagcaacgggagaaattatccacaacccgaagggaggaatggtttggacagcaaagctttagggagctggcctccgcgatgaacaccagcaaatctgctctacttattgtgacctcgtggttcaactttcccatgtatgagatcgactttggagctggcaaaccattttttgcatccactacgaacatgatttctcctatcaacggcgtgtgttgtggagtcattgcacccccaactcctgggagctgctcctccattgccactctgtacattttgtgccttcccgcggtactagaggctcttgaaaatgttccagatttcctatccttcttcgttcctcacccaaatcacaaagataactcgcaatag

SEQ ID NO: 123 - DNA, 1455 bp, synthetic construct
  misc_feature 1..1455: note = ERR364415-1_contig_8546_codon optimized for its expression in S. cerevisiae_C-terminally extended
  source 1..1455: mol_type = other DNA
  CDS 1..1455
atggccagggctccacccccaccaccaccgggcttgcgtatgagagatacggtcctatcaatcgtcaaacctataagaaagactcaacacctagagacaattgatgcaacattcgtagatttaatgagaatggactcatttatccctgtgatcttcgcttatagacctgcagataagagcgaggctgcgtactctagattagttaataggataaaagaaagcttacagaaagtactagtaccattctttggatttgctggaaggtgggtgccttcttccggtggctcaaggcgtctgctatgcaatgacgaaggtgtcccttttattgaggcgtttgtagacgaagaactggactctgtcgttaaggcttcagctgccttccaacctgtaactgaacttaatggtctaggtgtgttgggtatggatatgactagttatgatcaaagaatgccccctgaaggcggtcaaccgtgcgtcgtagctcaggtaacgagatttaaatgcggcggtgtggtattgggtgtagcattcaaccatacccatactgacgggcagggcttttatacttttatgcgtgcatggtcggatttcagtagaacgaatggtaccgcaattaaagtcgatcacaacagggcactacccgaattagcatctttgagtcagttctttattaagcagcatgatagaattggtgggaaaacttccaccgatagagtcaatgaccactgtagcaaagtccctgaaagactagcactaaaggcttttgaagttagggcgtccaaaatcaaggcagcaaaactagccgcagaagatggtggtgtaggctacgtctcgacggtggattgtattgttgctcatctatggaagacactagctcgtttgccaccagttgtgttagacggtcgtgagatcactgtgtttagcccagtagaaggcagaaataggtttcttgatccgccccgtccgaacatgtgtggtaattgtttcgctgcaatggtaacccctaaaatcccaacacaagaattgttagagatgccattagccgccattgccggcaagcagagagagaaactatctaccacgagacgtgaagagtggttcggacagcaatcattcagggagttggcttcagctatgaatacttctaaatcagctttgttaatcgtgacatcttggtttaacttcccgatgtatgaaatcgattttggtgccggtaagccgttcttcgctagtacgactaatatgatttctcctataaatggagtttgttgtggtgtcatagccccgcccacccccggttcctgttcatccatagcgacattatacattttatgtttaccagccgtgttagaagctcttgaaaatgtcccagatttcctttcgttcttcgtaccgcatccaaaccataaagacaacagccaaacaggccccttttcctttgtcgatatcatgtaa

SEQ ID NO: 124 - protein, 484 aa, synthetic construct
  REGION 1..484: note = Synthetic Construct
MARAPPPPPPGLRMRDTVLSIVKPIRKTQHLETIDATFVDLMRMDSFIPVIFAYRPADKSEAAYSRLVNRIKESLQKVLVPFFGFAGRWVPSSGGSRRLLCNDEGVPFIEAFVDEELDSVVKASAAFQPVTELNGLGVLGMDMTSYDQRMPPEGGQPCVVAQVTRFKCGGVVLGVAFNHTHTDGQGFYTFMRAWSDFSRTNGTAIKVDHNRALPELASLSQFFIKQHDRIGGKTSTDRVNDHCSKVPERLALKAFEVRASKIKAAKLAAEDGGVGYVSTVDCIVAHLWKTLARLPPVVLDGREITVFSPVEGRNRFLDPPRPNMCGNCFAAMVTPKIPTQELLEMPLAAIAGKQREKLSTTRREEWFGQQSFRELASAMNTSKSALLIVTSWFNFPMYEIDFGAGKPFFASTTNMISPINGVCCGVIAPPTPGSCSSIATLYILCLPAVLEALENVPDFLSFFVPHPNHKDNSQTGPFSFVDIM

SEQ ID NO: 125 - DNA, 1530 bp, Aspergillus fischeri
  misc_feature 1..1530: note = XP_001258079 native nucleotide sequence
  source 1..1530: mol_type = genomic DNA
atgaccgtgaccatcagtttcgagccatatgtgggctcctctgtcgatgctctaagcatccctctctatcttcgatgtcaactcgtcttcaaactttctaagccacttgctgcggtgcctctgcttgagtctggagttaatcgtcttgtacaagcgttacccttcctctcgggcgagttcacggccgtgccagcatccgacggtgggaaagaaattcttctcgttcgccctgtgctcaacttcgagctcagccgtatactcaagatcaagtaccatgaaacatccctacgacatgtatgcaaacagatgaacaggccaagcagccagggtggtgaccttccgcatgagccatacatgccctacccacgacttccagatccttcacgccctcaacccatcgtcgggttccaagtcaacgttcacacggatggcatcattctctccgttgctacgcatcactgttcctttgacgcaacagggatgggatcaatcgtccaaaacctcgcggcttgttgccgttctcctccgagcgacgagcctgacttgaccacgtcgccagcccaggaagcagaagcaagaaaagtcctctcgcaagtccgcgagacgccttttgatccaaagatgttcccggagtacagacccttggacagtatgctgtcctattacaaaggcgtccagtcagcgctccagggtcgtcaaaccactatcgtcaatcgatgtttcacaatcgccgccgacaagatcaacgcgctcaaaaggcgctgcaatcaactgataccggaaatggtgaagaagtatgggctttcaactgaggatgccattgggagcgcctgggtctcgagtaatgatgttgttgctgccctcttgtggacatgtatcaatcgagcgcgatatcccgaaatccgcgagcgcagcgttcaccagctcccaccagacctcctacatgcgacatctagcctgggtgtgccagtgaacgttcgctctcgactgtcgccgcccttacccaaatcgactttaggtaacgccgtgtgtcttctccgggagaaggtcccgctgcaatttttcgctttgcctagtcatgccaacatggaggccacttctagcgtttgcgcagaccattccggagacgacgaatgggccttgtccttctgtcgagtcgcctacggactcagagcgaagctgaacgcaattgatgacgactatatccgcgactatatctcctacgtgcaaaagtctccgtgccatctgtcagtgacactggatacagagaacctgtacctcagtaactggcgcgagatcggtgtgtatgatgctgattttggaggcatgctgggcaagccgctacggatgagagctccggatggatacaccgatggcctgatttttgtgatggcgcagcggagcgaagataagtctgcaccgtgggagtttaatatctcgctggaggcatcgacaatgaagcgtattgtgcatgatcccctctggtgcaagtatgttgagctggatgcgttctggcatggagaagaatga

SEQ ID NO: 126 - DNA, 1530 bp, synthetic construct
  misc_feature 1..1530: note = XP_001258079_codon optimized for its expression in S. cerevisiae
  source 1..1530: mol_type = other DNA
  CDS 1..1530
atgaccgtcacgatttcattcgaaccatatgtgggatctagcgtggacgcgctgtccatacccttgtatctaaggtgccaactggtgttcaaattatccaagcctcttgcagccgtgcccctgctagaatctggggtaaaccgtctagtacaagcattaccattcttgtctggtgaatttactgctgtccctgcaagcgatggtgggaaggaaatcttacttgttagaccagtcttaaatttcgaactaagtcgtatactgaagatcaaataccacgaaacatccttaagacacgtatgcaagcagatgaacagaccatcctcgcaaggtggtgacttgccacacgaaccgtatatgccataccccaggttaccagatccatctaggcctcaaccaatcgtgggttttcaagtcaatgtccacactgacggtataatcctgagtgtagcaactcaccattgcagttttgacgccacggggatgggaagtattgtacagaacttggccgcatgctgtagatctccgccatcggacgagccagatttaactacaagccctgctcaagaagcagaagctaggaaggtcctgagccaagttagagaaacaccattcgacccaaagatgtttcccgaatataggcccttagactctatgttatcttattacaaaggtgtccagtctgctttgcagggtcgtcaaactactatcgttaacagatgtttcactatcgctgctgataagataaacgccttaaagaggagatgtaaccaacttattccggaaatggtaaagaaatacggattgagtacagaagatgctatcggttctgcatgggtctcttctaatgacgtggttgccgcacttttgtggacctgtattaatcgtgctagatacccagagattagagaaagaagtgtacatcagcttccaccagacttgttgcatgctacatcttcattgggtgtacctgttaatgttagatctagactttcaccacccttacccaaatccactcttgggaatgcggtctgcctgctaagggagaaggtacctttgcaattcttcgctcttccgagtcacgcaaacatggaagcaaccagttcagtttgcgctgaccattcaggggatgatgaatgggcgttgagcttttgtagggtagcttatggtctgcgtgcaaaattgaacgcgatcgatgatgattacataagggattacattagttatgtacagaagtcgccctgtcatctttcagttacactagatactgagaacttatacctgtctaattggagagaaataggcgtctacgatgcagacttcggtggtatgctagggaaaccattgcgtatgagagctccggatggctacactgacggtttgattttcgttatggcccaaagatctgaagacaagtcagctccgtgggaattcaacatatcccttgaggcttctacaatgaaaaggattgtacatgatccgctgtggtgtaaatatgttgaattggatgccttttggcatggggaagagtaa

SEQ ID NO: 127 - protein, 509 aa, synthetic construct
  REGION 1..509: note = Synthetic Construct
MTVTISFEPYVGSSVDALSIPLYLRCQLVFKLSKPLAAVPLLESGVNRLVQALPFLSGEFTAVPASDGGKEILLVRPVLNFELSRILKIKYHETSLRHVCKQMNRPSSQGGDLPHEPYMPYPRLPDPSRPQPIVGFQVNVHTDGIILSVATHHCSFDATGMGSIVQNLAACCRSPPSDEPDLTTSPAQEAEARKVLSQVRETPFDPKMFPEYRPLDSMLSYYKGVQSALQGRQTTIVNRCFTIAADKINALKRRCNQLIPEMVKKYGLSTEDAIGSAWVSSNDVVAALLWTCINRARYPEIRERSVHQLPPDLLHATSSLGVPVNVRSRLSPPLPKSTLGNAVCLLREKVPLQFFALPSHANMEATSSVCADHSGDDEWALSFCRVAYGLRAKLNAIDDDYIRDYISYVQKSPCHLSVTLDTENLYLSNWREIGVYDADFGGMLGKPLRMRAPDGYTDGLIFVMAQRSEDKSAPWEFNISLEASTMKRIVHDPLWCKYVELDAFWHGEE

SEQ ID NO: 128 - DNA, 1323 bp, Aspergillus terreus
  misc_feature 1..1323: note = XP_001217250 native nucleotide sequence
  source 1..1323: mol_type = genomic DNA
atggcaaccttcgaccatatcgaggatgtcatcggccagctacctatgctgaagagctacacccatatcttgctgtgctttcccctcgctgagagccaactcaatgaagccatcgagagccttgaatctgctgtacgtcaggttataaaaaccttttcgttcttggctggcaaagtagtcaacgaaggcaagggacccaacagctcgggtactttcagggtcgctccctgtgagacgtgggagtccccagatcatcaatttgtgcgagtcgttgatcgctctttcatgctggcctcctacgatgagatccgcggagcacaggcacctgcttccatgctcccaggaagtcaactcgggtatcgggtggcttttccagcgcattatcacgagacagaggacgatcccgcgccggtcctggacattcagtgcaatttgatacggggcggactgctacttgacatcgctgcccaacacaacattatcgatgcgagcggtatctttcaaattgccagcttgatcgctctttccatgcgaggcgagtcaattcctgaggatgtcatcaaggaagggaaccgtgatcgacgcaatatcattccactactagaggcagatgagcctcttcttgatcacagcgagctcaaagccagcagcgcggtgcaaaatccgccgcccgtcaacttcctccaggggtataaatggcaaatcttcaagctctctgccgaggtgttaactcgaattaccgctgaaggacgacgacagccacaggagtttgtcccctccgtcacatttgtctcggcaaacgattgcttgacagcctttctgtggcaacgggtgatagccatgcgcctgaagcggctccatacgcccgaggccgtatccaaactgagccgtgctgttgatcttcggcgggcgatgggcattaccccagcatacatgggccatatgattcgtgtcgcaaatactagtctcacttttcaagaaattgtggcatgctctttatccaggcttgcatctctgctccgcaagagcatcatcgatgtcagccagccgtatgcgattcggagctacgtgacctttattgcaaatgagacggataaatcaaagattgcgtatgcgggtgctttcaatccttgcaccgacatgtcgtgctcgtccattgcacacatcactgctcctgaattcggtcgtttgggagcgcctgactttataaggaggcctacctacgggcctctgccgtgctgcacctatgtcgctcccgataagaacgatggagctttggatcaaaaccaggcatggtcagacgttgtgaagcgcatcggttga

SEQ ID NO: 129 - DNA, 1323 bp, synthetic construct
  misc_feature 1..1323: note = XP_001217250_codon optimized for its expression in S. cerevisiae
  source 1..1323: mol_type = other DNA
  CDS 1..1323
atggccaccttcgaccacattgaggacgtgataggacaattgcccatgctaaagagctacacacatattttactttgctttccgttggcagaatctcaactgaacgaggcgattgaatctttggagtcagcagtcagacaagtaattaagacgttttcattccttgcaggtaaggttgttaatgaaggcaaaggtccgaattcctctggtacgttccgtgttgctccgtgtgaaacttgggaatctcctgatcatcaatttgttcgtgttgtagataggagctttatgttggcctcgtatgatgagatccgtggtgctcaagcccccgcttccatgttacctgggtcacaactggggtatagggtagcattcccagcccattatcatgaaactgaagacgatccagctccagttctagatatccagtgtaatttgataagaggtggtctgcttctagatatagcggcccaacacaacataattgatgcttccgggatatttcaaatcgctagtctgatcgccttaagcatgagaggagaaagtatccctgaagatgttatcaaagaaggaaacagagatagaaggaatatcattccgttattggaagccgatgagcctttattagaccatagtgaattgaaggcatccagcgccgttcagaacccgccaccagttaatttcttgcaaggttataaatggcagattttcaaactgtccgctgaggtattgacccgtattactgccgaaggacgtagacaaccacaagaatttgtgccctcagtgacctttgtgtccgcaaacgactgcttgactgctttcttgtggcagcgtgtgatagctatgaggctaaagagattgcatacccccgaggccgtttccaagctatctagagccgtggatttaagaagggccatgggtataacccctgcatacatgggccatatgataagagtcgcaaacacatccttaacctttcaagaaattgtcgcatgtagtttatcacgtcttgcgagcttacttagaaaatctatcattgatgtgtcccaaccatatgcgatcagatcatacgtcaccttcatagcaaacgaaacagacaaatctaagatagcctatgccggggcatttaacccgtgtactgatatgtcatgttcatccatagctcatattacggcaccggagtttggtaggttaggggcgcctgacttcattaggagacccacttatggcccactaccttgctgtacttacgtagctcctgacaagaatgacggtgcattggatcagaatcaagcatggtcagatgtcgtaaagaggataggttaa

SEQ ID NO: 130 - protein, 440 aa, synthetic construct
  REGION 1..440: note = Synthetic Construct
MATFDHIEDVIGQLPMLKSYTHILLCFPLAESQLNEAIESLESAVRQVIKTFSFLAGKVVNEGKGPNSSGTFRVAPCETWESPDHQFVRVVDRSFMLASYDEIRGAQAPASMLPGSQLGYRVAFPAHYHETEDDPAPVLDIQCNLIRGGLLLDIAAQHNIIDASGIFQIASLIALSMRGESIPEDVIKEGNRDRRNIIPLLEADEPLLDHSELKASSAVQNPPPVNFLQGYKWQIFKLSAEVLTRITAEGRRQPQEFVPSVTFVSANDCLTAFLWQRVIAMRLKRLHTPEAVSKLSRAVDLRRAMGITPAYMGHMIRVANTSLTFQEIVACSLSRLASLLRKSIIDVSQPYAIRSYVTFIANETDKSKIAYAGAFNPCTDMSCSSIAHITAPEFGRLGAPDFIRRPTYGPLPCCTYVAPDKNDGALDQNQAWSDVVKRIG

SEQ ID NO: 131 - DNA, 1485 bp, Penicillium simplicissimum
  misc_feature 1..1485: note = Penicillium simplicissimum
  source 1..1485: mol_type = genomic DNA
atgtccaagcccttattcgaagcgtatcctctcacagggcttgatcatacgattcctccatgttatgttcgcttccttctaactttccccgtgccggatgtggcattggctgtcaatcagctgcaaaagggagctgaaaatttaatcgagaaacttcctttcctggccggatatttggcttcatgcgagacccccggcgtacgcccagggcagctcgagattcgacctcccgctggggaaaggagacctgtctgcctcgtcgcacatcactcgaactcctatctcgcagattccagtgcgacgtcgacgacggaacagctgggcaccgccaacgagaactatctccctgtcccgttcttcccggagctagacaagccggtgcccatcttccgggttaaggtgaatgccatgacggacggcatcattctgggatttgcgttccaccatagcgtgatcgatgccaccgggatgggcaccattgtccgggactttgccagatgctgccgtggccctgatggcggtcccctggaaatcagtctggagtctcagcaggactctagagagaagctgagacactccggaggacctcccgatccgcggtttgaccacaatggggagtaccctctcgtggcgtctctgcccgccgacctcgaagccatgaagcaggtcttgatccagacggcccgtctcatgtcaacgcagtatttccgcatccctgccagcctagttaacacgctaaaggaatcctgcaatcggatgcttcgggaatcaccagcgctcagggacgaaggggagaatccatggatttcgagcaacgatctggtggtgtcgctgttgtggctgtgtctgaatcgcgttcggtatcctgaagataataccaacgtcattcctccttccgattcctcggtctgcatggccgtgaatatccgagggcgtttgcagtcgcccattgatccaggatacgttggcaacgccatcgtccttctccgggagagcgttggcatgaatgcttttctgcataaaccgggcgacgatgatcccctgggcgcccaatgttacgaaacagcgaaacggctaggccgagaagcgtgggaagcagccctggtgcgcatcgccctggccatccgccgcaagctcaacaccataaacgcgagttacgtgcgcagtgttatatcctatctggaggacgtgcccgacctgtccactgtggcgtttggccagacggactaccacatcagcagctggcgggatattggcgtctacgaggctgattttggtggccacatgggccatcccagcgaaatgcgagtaccagatgggatggtcgatggcatgttttacatcttacctcgaaggcagggaacacacccttgctgggagatccatgttactatccaccaggacacaatgaagcgactcattgcagaccctgtgtgggcacgatatacagtgagaaagccttcatcactctgccgggatgaatga

SEQ ID NO: 132 - DNA, 1485 bp, synthetic construct
  misc_feature 1..1485: note = BAU61551_codon optimized for its expression in S. cerevisiae
  source 1..1485: mol_type = other DNA
  CDS 1..1485
atgtcaaagcctctgtttgaagcataccccctaactggtctagatcatactatacccccttgttacgtcaggttcttattaactttcccagttccagatgtagcccttgctgttaaccaattacagaaaggcgccgaaaatttaatagagaaattgccttttcttgcagggtacctagctagctgtgaaacacctggcgtaagaccggggcagttagaaatcaggccaccagcaggtgaaagaagaccagtgtgtttggtcgcacatcacagtaactcgtaccttgcggattcttctgcaacaagtacgacggagcaattgggaacagctaacgaaaattaccttcccgtaccattctttccagagttggacaaacctgttccaatattccgtgtcaaagttaacgctatgacagatgggattatcttggggttcgctttccaccatagtgtaatagatgctacgggaatgggtacaatagttagggacttcgccaggtgctgcagaggtcctgatggtgggcccttagagataagtctagaaagccaacaagattcgagagaaaagctgaggcactcaggcggtccgccagatcccagattcgatcataacggagaatacccattggtggcctcactgccagcggacttagaagctatgaaacaagttttaatccaaacagcgaggctgatgagtacacaatactttagaatacctgctagccttgtgaacactttaaaagagtcatgtaatagaatgcttcgtgaatcccctgcactgagggatgaaggtgaaaacccgtggattagttctaacgatttagtagtgagtctactgtggctttgtttgaacagggtgaggtaccccgaagacaatacaaatgtgattccaccctctgacagttctgtttgcatggctgtaaatataagagggagattacagtcgccgatcgatccaggttatgttggtaatgctattgtattattaagagaatctgttggaatgaatgcctttctacataaacctggtgatgacgacccgcttggtgcccaatgttacgagacagctaaaagacttggaagagaagcatgggaagcagcattagtcaggattgctttggcaattaggcgtaagttgaacactattaatgcttcctatgtcagatcagttattagctacttagaggatgtgcccgatctatcaaccgttgccttcgggcaaactgattatcatatctccagttggagagacattggagtttacgaggctgactttggtggtcatatgggccatccatctgagatgagagtccctgacgggatggtcgatggtatgttttacatactacctagaagacaaggtactcacccatgttgggaaattcatgtgactatacaccaggataccatgaaaagactgatcgctgatccggtttgggcaagatataccgttagaaagcctagttctttgtgcagggacgagtaa

SEQ ID NO: 133 - protein, 494 aa, synthetic construct
  REGION 1..494: note = Synthetic Construct
MSKPLFEAYPLTGLDHTIPPCYVRFLLTFPVPDVALAVNQLQKGAENLIEKLPFLAGYLASCETPGVRPGQLEIRPPAGERRPVCLVAHHSNSYLADSSATSTTEQLGTANENYLPVPFFPELDKPVPIFRVKVNAMTDGIILGFAFHHSVIDATGMGTIVRDFARCCRGPDGGPLEISLESQQDSREKLRHSGGPPDPRFDHNGEYPLVASLPADLEAMKQVLIQTARLMSTQYFRIPASLVNTLKESCNRMLRESPALRDEGENPWISSNDLVVSLLWLCLNRVRYPEDNTNVIPPSDSSVCMAVNIRGRLQSPIDPGYVGNAIVLLRESVGMNAFLHKPGDDDPLGAQCYETAKRLGREAWEAALVRIALAIRRKLNTINASYVRSVISYLEDVPDLSTVAFGQTDYHISSWRDIGVYEADFGGHMGHPSEMRVPDGMVDGMFYILPRRQGTHPCWEIHVTIHQDTMKRLIADPVWARYTVRKPSSLCRDE

SEQ ID NO: 134 - DNA, 1425 bp, Papaver somniferum
  misc_feature 1..1425: note = PsSalAT native nucleotide sequence
  source 1..1425: mol_type = genomic DNA
atggcaacaatgtatagtgctgctgttgaagtgatctctaaggaaaccattaaacccacaactccaaccccatctcaacttaaaaacttcaatctgtcacttctcgatcaatgttttcctttatattattatgttccaatcattcttttctacccagccaccgccgctaatagtaccggtagcagtaaccatcatgatgatcttgacttgcttaagagttctctttccaaaacactagttcacttttatccaatggctggtaggatgatagacaatattctggtcgactgtcatgaccaagggattaacttttacaaagttaaaattagaggtaaaatgtgtgagttcatgtcgcaaccggatgtgccactaagccagcttcttccctctgaagttgtttccgcgagtgtccctaaggaagcactggtgatcgttcaagtgaacatgtttgactgtggtggaacagccatttgttcgagtgtatcacataagattgccgatgcagctacaatgagtacgttcattcgtagttgggcaagcaccactaaaacatctcgtagtgggggttcaactgctgccgttacagatcagaaattgattccttctttcgactcggcatctctattcccacctagtgaacgattgacatctccatcagggatgtcagagataccattttccagtaccccagaggatacagaagatgataaaactgtcagcaagagatttgtgttcgattttgcaaagataacatctgtacgtgaaaagttgcaagtattgatgcatgataactacaaaagccgcaggcaaacaagggttgaggtggttacttctctaatatggaagtccgtgatgaaatccactccagccggttttttaccagtggtacatcatgccgtgaaccttagaaagaaaatggacccaccattacaagatgtttcattcggaaatctatctgtaactgtttcggcgttcttaccagcaacaacaacgacaacaacaaatgcggtcaacaagacaatcaatagtacgagtagtgaatcacaagtggtacttcatgagttacatgattttatagctcagatgaggagtgaaatagataaggtcaagggtgataaaggtagcttggagaaagtcattcaaaattttgcttctggtcatgatgcttcaataaagaaaatcaatgatgttgaagtgataaacttttggataagtagctggtgcaggatgggattatacgagattgattttggttggggaaagccaatttgggtaacagttgatccaaatatcaagccgaacaagaattgttttttcatgaatgatacgaaatgtggtgaaggaatagaagtttgggcgagctttcttgaggatgatatggctaagttcgagcttcacctaagtgaaatccttgaattgatttga

SEQ ID NO: 135 - DNA, 1425 bp, synthetic construct
  misc_feature 1..1425: note = PsSalAT_codon optimized for its expression in S. cerevisiae
  source 1..1425: mol_type = other DNA
  CDS 1..1425
atggcaacaatgtactcagctgcagttgaggttatatctaaggaaacgataaaaccaaccactccaaccccaagccaattgaagaatttcaatttatctttattagaccagtgctttcccttgtactactatgtccccatcatcttgttctaccctgcgactgctgcaaactccactggttcctcgaaccaccatgatgatctagatcttctgaagagctcccttagcaagacacttgttcacttctaccctatggccggtaggatgatcgataacatattggttgactgccacgatcagggtatcaatttctataaagttaaaatcaggggcaagatgtgtgaattcatgtctcagcctgatgtgccactgtcgcagctgctacctagtgaagtggtatccgcatctgtcccaaaagaggccttggtcatagtccaagttaatatgttcgattgcggtgggacggccatctgctcgtcggtcagtcataagatcgcagacgccgcaaccatgtcaacatttattagatcttgggcgagtaccaccaaaacttcaaggtctggcgggtcaaccgccgctgttactgaccagaagttgattcctagctttgattcggcaagcttattcccaccttccgaaaggttgacttcaccaagcgggatgtctgagataccattttcttcaacccctgaagataccgaggacgacaaaacagttagcaaaagattcgtgtttgactttgcaaagataacatctgttagagaaaagcttcaggtattaatgcacgacaactacaaaagcaggcgtcagaccagggttgaggtcgtaacgagcctgatctggaagagtgtcatgaagtcaacaccagctgggttccttcccgtcgtgcatcatgcggtaaatttgaggaagaagatggacccaccattgcaggatgtctccttcggcaacctgagtgttactgtctcagcattcttgcccgccacaactaccaccaccacaaatgccgttaataagacaataaattcaacttcatcggagagtcaggtggtgctacatgaattgcacgattttatcgcacaaatgagaagtgagatagacaaagttaaaggcgataagggtagcctagaaaaggtgattcagaactttgcctctggtcatgacgcttcaataaagaaaataaatgacgtagaggtcataaatttctggatatcttcatggtgcagaatgggcttgtatgagatcgacttcggctggggcaagccgatttgggttacagttgaccctaatatcaagccaaataagaattgcttctttatgaacgacacaaaatgcggagaaggaatagaggtatgggcaagcttcctggaagacgacatggccaagtttgaattgcacttatcggagatattggaattgatctaa

SEQ ID NO: 136 - protein, 474 aa, synthetic construct
  REGION 1..474: note = Synthetic Construct
MATMYSAAVEVISKETIKPTTPTPSQLKNFNLSLLDQCFPLYYYVPIILFYPATAANSTGSSNHHDDLDLLKSSLSKTLVHFYPMAGRMIDNILVDCHDQGINFYKVKIRGKMCEFMSQPDVPLSQLLPSEVVSASVPKEALVIVQVNMFDCGGTAICSSVSHKIADAATMSTFIRSWASTTKTSRSGGSTAAVTDQKLIPSFDSASLFPPSERLTSPSGMSEIPFSSTPEDTEDDKTVSKRFVFDFAKITSVREKLQVLMHDNYKSRRQTRVEVVTSLIWKSVMKSTPAGFLPVVHHAVNLRKKMDPPLQDVSFGNLSVTVSAFLPATTTTTTNAVNKTINSTSSESQVVLHELHDFIAQMRSEIDKVKGDKGSLEKVIQNFASGHDASIKKINDVEVINFWISSWCRMGLYEIDFGWGKPIWVTVDPNIKPNKNCFFMNDTKCGEGIEVWASFLEDDMAKFELHLSEILELI

SEQ ID NO: 137 - DNA, 1443 bp, synthetic construct
  misc_feature 1..1443: note = AstC_codon optimized for its expression in S. cerevisiae
  source 1..1443: mol_type = other DNA
  CDS 1..1443
atgactaagatcaacccatacaagggtatcttggttgaattgaaggacatcgttttcacttcttcttctgaccaaatcaagttgccaatcaacactttcaagtctatcttgtgttgtggtgctactgctcaataccaatgtggtaagatcaacagagctcaatactactctagattggctagagacttcgctttgtctttggctgacgttactgctttgttcgacactgttcaagctactatcagaccagaagaatctttcttggctttcttggctgaattgaagtctagattcggtgaacaattgaagttgtacgctgttgctaacatgtctagagaagactacgctatgttgaagtctttgccaatcgactggtctttgttcgacggtgttttcttgtctgctgacttgggtatgagaaagccagaattgagattcttcagacacgttttggaatctatctctatgaagccagaagacactatcttggttgacaacgacactgacaacatcttgtgtgctttgtctatgggtttgaagggtatcttgttcggttctacttctgttccacaagctttgactaacttgttggaatacgaccacatctctagagctgaacaattcttgagatctcacgctaagtctttgcactctgttactcacactggtgttactatcagagaaaacttcgctcaattgttgatcttggaagctactggtgacatcgacttggttgaattggaataccacccaactacttggaactacttcatcggtactccagttttgactcaaactgaattcccacacgacttggacactacttctttggctactactgttttggacagaccaaaggacatcgctaacgaaatcatggacgaaatgttgaagtacagatctgacgacgacttgatgttgactttcttcactgacttcaagaacagagttgacccagttgtttgttgtaacgttttgtctttgttctacaagtacggtagaggtcacgaattgcaccacactttggcttgggttagacaagttttgatcagaagagcttacatcaacggtactgctttctacccaatgccagaagctttcttgtacttcttcttcagattcttgcaacacatcactcacttgccacaattgtacgacggtttgaaggttttgttgaaggaaagattgcaagaaagagttggtgttccagttgacccaatctctttgtctatgagattgatcgcttgtaacggtgttggtatccacgacagaatgggtttgaacgctttgttgtctatgcaaaacccagacggttcttgggacttgggtactatgtaccactacgcttctaagagattgccaatcggtaaccaaggtgtttctactgctatggctatcaaggctatcaagcaatgtcaagctaaccaatgtgctggtatctaa

SEQ ID NO: 138 - protein, 480 aa, synthetic construct
  REGION 1..480: note = Synthetic Construct
MTKINPYKGILVELKDIVFTSSSDQIKLPINTFKSILCCGATAQYQCGKINRAQYYSRLARDFALSLADVTALFDTVQATIRPEESFLAFLAELKSRFGEQLKLYAVANMSREDYAMLKSLPIDWSLFDGVFLSADLGMRKPELRFFRHVLESISMKPEDTILVDNDTDNILCALSMGLKGILFGSTSVPQALTNLLEYDHISRAEQFLRSHAKSLHSVTHTGVTIRENFAQLLILEATGDIDLVELEYHPTTWNYFIGTPVLTQTEFPHDLDTTSLATTVLDRPKDIANEIMDEMLKYRSDDDLMLTFFTDFKNRVDPVVCCNVLSLFYKYGRGHELHHTLAWVRQVLIRRAYINGTAFYPMPEAFLYFFFRFLQHITHLPQLYDGLKVLLKERLQERVGVPVDPISLSMRLIACNGVGIHDRMGLNALLSMQNPDGSWDLGTMYHYASKRLPIGNQGVSTAMAIKAIKQCQANQCAGI

SEQ ID NO: 139 - DNA, 606 bp, synthetic construct
  misc_feature 1..606: note = AstI_codon optimized for its expression in S. cerevisiae
  source 1..606: mol_type = other DNA
  CDS 1..606
atgactagacaatctcactaccaagctatcatcttggacttgggtaacgttgttttcgaatgggacacttctcaaaacccaccaactgctgctccaaaccaaatctctttgttgagaacttctatgaagtctccagtttaccactcttacgaaagaggtcaattgtctactgaagaatgtcacagattgttgggtgaatctttgcacgttgacccaggtcaaatcaaggaagctttcgacttggctagacaatctttgagatctaacccagctttgttggacttcatcagacaattgaagcaaactagaggtgttgctgtttacgctatgtctaacatcccacaagctgaaatcgaatacttgaaggaatctagagctggtgacatggaagttttcgacgaagttttcgcttctggttacgttggttctagaaagccagaaactgaattctacagaagagttatgggtgaaatcggtttgaaggctgaaagagttgttttcgttgacgacaaggaagaaaacgttgacgttgctagaggtttgggtttgtacggtgtttgtttcggtggtgttgaagaattgagaggtcacttgttgggtatctaa

SEQ ID NO: 140 - protein, 201 aa, synthetic construct
  REGION 1..201: note = Synthetic Construct
MTRQSHYQAIILDLGNVVFEWDTSQNPPTAAPNQISLLRTSMKSPVYHSYERGQLSTEECHRLLGESLHVDPGQIKEAFDLARQSLRSNPALLDFIRQLKQTRGVAVYAMSNIPQAEIEYLKESRAGDMEVFDEVFASGYVGSRKPETEFYRRVMGEIGLKAERVVFVDDKEENVDVARGLGLYGVCFGGVEELRGHLLGI

SEQ ID NO: 141 - DNA, 591 bp, synthetic construct
  misc_feature 1..591: note = AstK_codon optimized for its expression in S. cerevisiae
  source 1..591: mol_type = other DNA
  CDS 1..591
atgtgtactactttcaaggctgctatcttcgacatgggtggtgttttgttcacttggaacccaatcgttgacactcaagtttctttgaaggacttgggtactatcatcaactctgaaacttgggaacaattcgaaagaggtaagatcgaaccagacgactgttaccaccaattgggttctcaaatcggtttgccaggttctgaaatcgctgctactttcagacaaactactggttgtttgagaccagacgctagaatgacttctttgttgagagaattgaagggtcaaggtgttgctgtttacatgatgactaacatcccagctccagacttccaccaattgagagaaatgcactacgaatgggacttgttcgacggtatcttcgcttctgctttggaaggtatgagaaagccagacttggaattctacgaacacgttttgaagcaaatcgacacttctgctgctgaaactatcttcgttgacgacaagttggaaaacgttatcgctgctcaagctgttggtatggttggtttgcacttgactgactctttggctacttgtatggaattgagacaattggttggttgttaa

SEQ ID NO: 142 - protein, 196 aa, synthetic construct
  REGION 1..196: note = Synthetic Construct
MCTTFKAAIFDMGGVLFTWNPIVDTQVSLKDLGTIINSETWEQFERGKIEPDDCYHQLGSQIGLPGSEIAATFRQTTGCLRPDARMTSLLRELKGQGVAVYMMTNIPAPDFHQLREMHYEWDLFDGIFASALEGMRKPDLEFYEHVLKQIDTSAAETIFVDDKLENVIAAQAVGMVGLHLTDSLATCMELRQLVGC

SEQ ID NO: 143 - protein, 479 aa, Aspergillus sclerotiicarbonarius
MGASVSFQPFVPTPLDHAMPPIYVSQFLCFPTTTPQSAIQSLQVGIERLFERLPFLAGEILINEHTGAIKVQAPSALIREIPYMALRAHPDLYLPAKQCATTPIERQLKTNSLDESYHPLPAALPLSQPQPVIRFQANTLADGILFAVSYHHCIFDGTGCGQILEMLAQCCSASDDKISLPTDCHTDVLLREYISNLSPTTNIPHDYTQAYSTTVQPDPDASDPDTSPAIPSSLYTEAFTFPSQQITTLRDACNHLLPKLPSTSNAHPHKPTPNPLSSNDVLTALIALCITRATNTTTPPLQPNNHSLSMAVNLRTRIQPQVPDHFLGNFATLLPIHFTSPVHTQQSDLLLTTEPPDPALIHLTTLASQIRSSLSTVNTDYICGLMTDLRTRRNAGENSSLLIEGIKISSWRHLSVYKPDFGPGLGKIAGFEFQAGLMDNLVVILPWRNGDWDVRVTLLERDMRGFREDRLVRWALGSG

SEQ ID NO: 144 - protein, 474 aa, Bazzania trilobata
MARAPPPPPPGLRMRDTVLSIVKPIRKTQHLETIDATFVDLMRMDSFIPVIFAYRPADKSEAAYSRLVNRIKESLQKVLVPFFGFAGRWVPSSGGSRRLLCNDEGVPFIEAFVDEELDSVVKASAAFQPVTELNGLGVLGMDMTSYDQRMPPEGGQPCVVAQVTRFKCGGVVLGVAFNHTHTDGQGFYTFMRAWSDFSRTNGTAIKVDHNRALPELASLSQFFIKQHDRIGGKTSTDRVNDHCSKVPERLALKAFEVRASKIKAAKLAAEDGGVGYVSTVDCIVAHLWKTLARLPPVVLDGREITVFSPVEGRNRFLDPPRPNMCGNCFAAMVTPKIPTQELLEMPLAAIAGKQREKLSTTRREEWFGQQSFRELASAMNTSKSALLIVTSWFNFPMYEIDFGAGKPFFASTTNMISPINGVCCGVIAPPTPGSCSSIATLYILCLPAVLEALENVPDFLSFFVPHPNHKDNSQ